(19) United States
(12) Patent Application Publication
(10) Pub. No.: US 2022/0396809 A1
(43) Pub. Date: Dec. 15, 2022

(54) ENGINEERED NEWCASTLE DISEASE VIRUS VECTOR AND USES THEREOF

(71) Applicant: University of Guelph, Guelph (CA)

(72) Inventors: Sarah Wootton, Guelph (CA); Leonardo Susta, Guelph (CA); Byram Bridle, Guelph (CA); Pierre Major, Ancaster (CA); Lisa Santry, Bedford, MA (US); Yanlong Pei, Guelph (CA)

(21) Appl. No.: 17/831,894

(22) Filed: Jun. 3, 2022

Related U.S. Application Data
(60) Provisional application No. 63/196,489, filed on Jun. 3, 2021.

(51) Int. Cl. C12N 15/86; C07K 14/005; A61K 39/17; A61K 39/215; A61P 35/00; A61P 31/14

(52) U.S. Cl. CPC C12N 15/86; C07K 14/005; A61K 39/17; A61K 39/215; A61P 35/00; A61P 31/14; C12N 2760/18122; C12N 2760/18134; C12N 2760/18132; C12N 2760/18143; C12N 2760/18162; C12N 2760/18171; C12N 2770/18071; C12N 2770/18022; C12N 2760/18322; C07K 2319/00; A61K 2039/5254

(57) ABSTRACT

An engineered Newcastle Disease Virus (NDV) vector is provided. In particular, the present disclosure provides methods of treating or preventing a disease such as cancer, or an infectious disease, or methods for eliciting an immune response, with the engineered NDV vector. The engineered NDV vector provided herein is useful as an immunogenic composition, an oncolytic agent, or a vaccine.

### RELATED APPLICATION

This disclosure claims benefit and priority of U.S. Provisional Patent Application Ser. No. 63/196,489 filed Jun. 3, 2021, incorporated herein by reference in its entirety.

### INCORPORATION OF SEQUENCE LISTING

A computer readable form of the Sequence Listing “P62990US01_Sequence_Listing_ST25” (426,627 bytes), submitted via EFS-WEB and created on Jun. 3, 2022, is herein incorporated by reference.

### FIELD

The present disclosure provides engineered Newcastle Disease Virus (NDV) vectors comprising a nucleic acid having a nucleic acid sequence described herein. The NDV vectors may comprise at least one heterologous nucleic acid segment encoding a therapeutic agent operably linked to a viral promoter capable of expressing the segment in a host cell. Also provided are methods of treating a disease with said engineered NDV vectors and a vaccine comprising an engineered NDV vector described herein, and methods of treating a disease with said vaccine.

### BACKGROUND

Newcastle Disease Virus (NDV), also known as avian orthoavulavirus-1 (AOaV-1), is an enveloped avian paramyxovirus virus with a non-segmented, negative-sense RNA genome. NDV has been studied as a candidate engineered live vaccine platform for human and veterinary infectious diseases. NDV may be useful as a candidate vaccine vector for a few reasons. As an avian virus, NDV is antigenically distinct from common human vaccines and pathogens, averting the problem of pre-existing immunity that would limit its efficacy in people. As an oncolytic agent, NDV has shown an excellent safety profile, whereby direct intravenous, aerosol, or intratumoral administration of large virus doses is well tolerated in people (Wheelock, E. F. and J. H. Dingle, 1964; Csatary, L. K., et al., 1993; Pecora, A. L, 2002). As a vaccine vector in pre-clinical models, NDV-vectored vaccines have been shown to be safe and protective in non-human primate models of pathogenic avian influenza, Ebola, and SARS-CoV-1 (severe acute respiratory syndrome coronavirus-1) (Bukreyev, A., et al., 2005; DiNapoli, J. M., et al., 2010; DiNapoli, J. M., et al., 2007). Additionally, the NDV viral genome is highly versatile, allowing for stable insertion and high-level expression of foreign genes such as viral antigens. Lastly, NDV is an acute cytoplasmic virus and its genomic RNA is tightly encapsidated by nucleocapsid protein; all features that markedly mitigate concerns about insertional mutagenesis or recombination.

The novel SARS-CoV-2 (severe acute respiratory syndrome coronavirus-2) emerged in late 2019 as the causative agent of a severe respiratory disease named coronavirus disease 2019 (COVID-19). The virus has been classified in the Coronaviridae family, β-coronavirus genus, and Sarbecovirus subgenus (i.e., β-coronavirus subgroup B). Phylogenetic analysis has shown that this virus shares ≈50% genetic similarity with MERS (Middle East Respiratory Syndrome)-CoV, ≈with SARS-CoV-1, and >90% similarity with bat β-coronaviruses. SARS-CoV-2 is transmitted through contact and respiratory route. In people with severe disease, morbidity and mortality are mediated by severe respiratory distress syndrome and vascular disease. The former is caused by diffuse alveolar damage associated with virus replication in type I and II alveolar pneumocytes. Molecular effectors of tissue damage include unchecked production of pro-inflammatory cytokines (i.e., cytokine storm), decreased angiotensin-converting enzyme-2 (ACE2) activity, and activation of a thrombo-inflammatory cascade leading to a hypercoagulable state.

Multiple research groups have been working towards production of several vaccine platforms against SARS-CoV-2, including engineered viral vectors, nucleic acids (DNA, mRNA and self-replicating RNA), protein subunits, virus-like particles, and live-attenuated or inactivated SARS-CoV-2 virions. The vast majority of these vaccines target the SARS-CoV-2 Spike (S) protein, the main neutralizing antigen against the virus. In December 2020, two mRNA based COVID-19 vaccines (Pfizer-BioNTech and Moderna) received emergency use authorization by the U.S. Food and Drug Administration; however, it is unclear whether these vaccines will have reduced efficacy against Variants of Concern (VoC), such as the South African B.1.351 variant, highlighting the need for vaccines that induce sterilizing immunity (Peiris, M. and G. M. Leung, 2020).

Due to the relative advantages and disadvantages of different vaccine types, there is an ongoing need to develop and test novel vaccine platforms and strategies. New vaccines may be critical for potential future pandemics and emerging and re-emerging infections, which will require swift development of vaccine candidates. Live viral vectors may be useful due to their generally high immunogenicity, ability to induce both humoral and cellular immune responses, and the lack of a need for adjuvants.

### SUMMARY

The present inventors produced an engineered (fully synthetic) Newcastle Disease Virus (NDV) vector, which is immune stimulatory and useful as a therapeutic agent for oncolytic viral therapy, or as a vaccine platform for immunoprophylaxis. In particular, the inventors created an intra-nasally delivered, non-virulent NDV vaccine expressing the SARS-CoV-2 spike protein for protecting subjects from COVID-19 or related coronaviruses. The use of a non-virulent NDV strain (i.e., lentogenic pathotype) makes the vaccine safe in both mammals and avian species, including poultry, which are the natural target of NDV. Intra-nasal delivery stimulates both a mucosal and systemic immune response in the host, and a needle-free administration is logistically simpler and can ameliorate concerns associated with vaccine hesitancy. The engineered NDV vector of this disclosure can infect host cells to express an immunogenic agent, for example, the SARS-CoV-2 spike protein (NDV-FLS), which leads to the production of spike protein-specific serum IgG and mucosal IgA antibodies as well as spike protein-specific T cells responses in subjects administered the vaccine intranasally.

Accordingly, the present disclosure provides an engineered Newcastle Disease Virus (NDV) vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises or further comprises at least one heterologous nucleic acid segment encoding a therapeutic agent operably linked to a promoter capable of expressing the segment in a host cell, wherein the nucleic acid comprises a nucleic acid sequence encoding an L protein having a stabilizing segment, and wherein the nucleic acid comprises XbaI and MluI restriction endonuclease sites between nucleic acid sequences encoding phosphoprotein and matrix protein.

The present disclosure also provides a method of treating or preventing a disease in a subject, comprising administering an engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises or further comprises at least one heterologous nucleic acid segment encoding a therapeutic agent operably linked to a promoter capable of expressing the segment in a host cell, wherein the nucleic acid comprises a nucleic acid sequence encoding an L protein having a stabilizing segment, and wherein the nucleic acid comprises XbaI and MluI restriction endonuclease sites between nucleic acid sequence encoding phosphoprotein and matrix protein.

Also provided is use of an engineered NDV vector for treating or preventing a disease in a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the the nucleic acid comprises or further comprises at least one heterologous nucleic acid segment encoding a therapeutic agent operably linked to a promoter capable of expressing the segment in a host cell, wherein the nucleic acid comprises a nucleic acid sequence encoding an L protein having a stabilizing segment, and wherein the nucleic acid comprises XbaI and MluI restriction endonuclease sites between nucleic acid sequence encoding phosphoprotein and matrix protein.

Further provided is use of an engineered NDV vector in the manufacture of a medicament for treating or preventing a disease in a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein.

Even further provided is an engineered NDV vector for use in treating or preventing a disease in a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein.

In an embodiment, the engineered NDV vector comprises a nucleic acid having a nucleic acid sequence that is at least 75%, 80%, 85%, 90%, 95%, 96%, 97%, 98%, 99%, 99.5%, 99.9% A or 100% identical to the nucleic acid sequence of SEQ ID NO: 9, 10, 23, or 27. In an embodiment, the engineered NDV vector comprises a nucleic acid having a nucleic acid sequence that is at least 95% identical to the nucleic acid sequence any one of SEQ ID NO: 1-5, 9, 10, 18, 19, 23, 27, or 42. In an embodiment, the engineered NDV vector comprises a nucleic acid having a nucleic acid sequence that is at least 99% identical to the nucleic acid sequence any one of SEQ ID NO: 1-5, 9, 10, 18, 19, 23, 27, or 42. In an embodiment, the engineered NDV vector comprises a nucleic acid sequence consisting of the nucleic acid sequence any one of SEQ ID NO: 1-5, 9, 10, 18, 19, 23, 27, or 42.

In an embodiment, the engineered NDV vector comprises a nucleic acid having a nucleic acid sequence a chimeric F protein, and a chimeric HN protein, wherein the chimeric F protein comprises avian paramyxovirus 5 (APMV5) F protein segment thereof at the N-terminus and an NDV F protein segment at the C-terminus, and wherein the chimeric HN protein comprises an NDV HN protein segment at the N-terminus and an AMPV5 HN protein segment at the C-terminus. In an embodiment, the stabilizing segment comprises an amino acid sequence as set forth in SEQ ID NO: 20. In an embodiment, the stabilizing segment is encoded by a nucleic acid comprising a nucleic acid sequence as set forth in SEQ ID NO: 35. In an embodiment, the chimeric F protein comprises at the C-terminus 53 amino acid of NDV F protein from amino acid positions 501 to 553 of SEQ ID NO: 28. In an embodiment, the chimeric HN protein comprises at the N-terminus 53 amino acids of NDV HN protein from amino acid positions 1 to 53 of SEQ ID NO: 34. In an embodiment, the engineered NDV vector of any one of claims 8 to 11, wherein the L protein comprises an amino acid sequence having at least 85%, 90%, 95%, 96%, 97%, 98%, 99%, 99.5%, 99.9%, or 100% identity to the amino acid sequence as set forth in SEQ ID NO: 11. In an embodiment, the chimeric F protein comprises an amino acid sequence having at least 85%, 90%, 95%, 96%, 97%, 98%, 99%, 99.5%, 99.9%, or 100% identity to the amino acid sequence of SEQ ID NO: 12. In an embodiment, the chimeric HN protein comprises an amino acid sequence having at least 85%, 90%, 95%, 96%, 97%, 98%, 99%, 99.5%, 99.9%, or 100% identity to the amino acid sequence of SEQ ID NO: 13.

In an embodiment, the NDV vector is lentogenic, and wherein the nucleic acid comprises a nucleic acid sequence of SEQ ID NO: 25.

Also provided is an engineered Newcastle Disease Virus (NDV) vector comprising a nucleic acid having a nucleic acid sequence encoding an L protein having a stabilizing segment, a chimeric F protein, and a chimeric HN protein, wherein the chimeric F protein comprises avian paramyxovirus 5 (APMV5) F protein segment thereof at the N-terminus and an NDV F protein segment at the C-terminus, and wherein the chimeric HN protein comprises an NDV HN protein segment at the N-terminus and an AMPV5 HN protein segment at the C-terminus. In an embodiment, the nucleic acid comprises XbaI and MluI restriction endonuclease sites between nucleic acid sequence encoding phosphoprotein and matrix protein. In an embodiment, the stabilizing segment comprises an amino acid sequence as set forth in SEQ ID NO: 20. In an embodiment, the stabilizing segment is encoded by a nucleic acid comprising a nucleic acid sequence as set forth in SEQ ID NO: 35. In an embodiment, the chimeric F protein comprises at the C-terminus 53 amino acid of NDV F protein from amino acid positions 501 to 553 of SEQ ID NO: 28. In an embodiment, the chimeric HN protein comprises at the N-terminus 53 amino acids of NDV HN protein from amino acid positions 1 to 53 of SEQ ID NO: 34. In an embodiment, the L protein comprises an amino acid sequence having at least 85%, 90%, 95%, 96%, 97%, 98%, 99%, 99.5%, 99.9%, or 100% identity to the amino acid sequence as set forth in SEQ ID NO: 11. In an embodiment, the chimeric F protein comprises an amino acid sequence having at least 85% identity to the amino acid sequence of SEQ ID NO: 12. In an embodiment, the chimeric HN protein comprises an amino acid sequence having at least 85% identity to the amino acid sequence of SEQ ID NO: 13. In an embodiment, the NDV vector is lentogenic, and wherein the nucleic acid comprises a nucleic acid sequence of SEQ ID NO: 25. In an embodiment, the nucleic acid further comprises at least one heterologous nucleic acid segment encoding a therapeutic agent operably linked to a promoter capable of expressing the segment in a host cell.

In another embodiment, the host cell is selected from the group consisting of a human, primate, murine, feline, canine, ovine, bovine, porcine, caprine, equine, lupine, vulpine, mustelid host cell and. In a further embodiment, the promoter is capable of expressing the at least one heterologous nucleic acid segment encoding the therapeutic agent in muscle, airways, or lung cells.

In an embodiment, the disease is an infectious disease. In an embodiment, the infectious disease is selected from the group consisting of viral diseases such as viral hemorrhagic fevers, Ebola, Marburg virus disease, gastroenteritis, dengue fever, West Nile fever, yellow fever, influenza, respiratory syncytial virus disease, Lassa fever, rabies, smallpox, cowpox, horsepox, monkeypox, Hantavirus pulmonary syndrome, Hendra virus disease, Nipah virus disease, human immunodeficiency virus infection and acquired immunodeficiency disease syndrome, Hepatitis, Zika fever, Severe Acute Respiratory Syndrome (SARS), Middle East Respiratory Syndrome (MERS), Coronavirus disease 2019 (COVID-19), infectious bronchitis, infectious laryngotracheitis, Rift Valley fever, porcine epidemic diarrhea, porcine transmissible gastroenteritis, swine acute diarrhea syndrome, feline infectious peritonitis, African swine fever, classical swine fever, and bacterial diseases including drug resistant bacterial diseases such as tuberculosis and methicillin-resistant Staphylococcus aureus infection, and drug resistant parasitic diseases such as malaria. In an embodiment, the infectious disease is COVID-19.

In an embodiment, the therapeutic agent comprises a SARS-CoV-2 spike protein. In an embodiment, the SARS-CoV-2 spike protein comprises an amino acid sequence having at least 85%, 90%, 95%, 96%, 97%, 98%, 99%, 99.5%, 99.9%, or 100% identity to the amino acid sequence of SEQ ID NO: 6, 7, 29, 30, 31, or 41.

In an embodiment, the subject is an animal. In an embodiment, the animal is human or a veterinary animal. In an embodiment, the subject is human. In an embodiment, the subject is a veterinary animal. In an embodiment, the veterinary animal is a primate, a murine, a feline, a canine, an ovine, a bovine, a porcine, a caprine, an equine, a lupine, a vulpine, or a mustelid. In an embodiment, the subject is a mustelid.

In another embodiment, the engineered NDV vector is administered or co-administered intravenously, intranasally, intratracheally, intramuscularly, or via aerosol. In an embodiment, the viral vector is delivered to lung cells or tissues. In an embodiment, the viral vector is delivered intranasally or intramuscularly. In an embodiment, the viral vector is delivered to an animal. In an embodiment, the viral vector is delivered to a human or a veterinary animal. In an embodiment, the veterinary animal is a primate, a murine, a feline, a canine, an ovine, a bovine, a porcine, a caprine, an equine, a lupine, a vulpine, or a mustelid. In an embodiment, the viral vector is delivered to a human. In an embodiment, the viral vector is delivered to a mustelid.

The present disclosure also provides an isolated nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises XbaI and MluI restriction endonuclease sites between nucleic acid sequence encoding phosphoprotein and matrix protein. In an embodiment, the nucleic acid sequence is at least 75%, 80%, 85%, 90%, 95%, 96%, 97%, 98%, 99%, 99.5%, 99.9% or 100% identical to the nucleic acid sequence of SEQ ID NO: 9, 10, 23, or 27, wherein the nucleic acid comprises XbaI and MluI restriction endonuclease sites between nucleic acid sequence encoding phosphoprotein and matrix protein. In an embodiment, the nucleic acid comprises or further comprises at least one heterologous nucleic acid segment encoding a therapeutic agent operably linked to a promoter capable of expressing the segment in a host cell.

In an embodiment, the nucleic acid further comprises at least one heterologous nucleic acid segment encoding a therapeutic agent operably linked to a promoter capable of expressing the segment in a host cell.

Further provided is a pharmaceutical composition comprising an engineered NDV vector described herein, and a pharmaceutically acceptable carrier. In an embodiment, the pharmaceutical composition is lyophilized.

Further provided is a method of producing a protein in vivo in a subject, comprising delivering or introducing into the subject an engineered NDV vector comprising a nucleic acid having a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a protein operably linked to a promoter capable of expressing the segment in a host cell, and wherein the nucleic acid comprises XbaI and MluI restriction endonuclease sites between nucleic acid sequence encoding phosphoprotein and matrix protein.

Further provided is an immunogenic composition, an oncolytic agent, or a vaccine comprising an engineered NDV vector described herein for treating a disease described herein.

Further provided is a method of eliciting an immune response, comprising administering to a subject an engineered NDV vector described herein, for treating a disease described herein.

Further provided is a method of treating cancer, comprising administering to a subject an engineered NDV vector described herein, wherein the NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1, 5, 9, 10, 23, or 27.

Further provided is a method for selecting an engineered NDV vector genome comprising a stabilizing segment in L gene, the method comprises:
a) growing bacterial cells comprising an engineered NDV vector genome in a growth medium broth;
b) growing the bacterial cells on an agar-growth medium, wherein the agar-growth medium comprises a selection agent;
c) identifying small bacterial cells colonies having about 0.5 mm to about 1 mm in diameter after at least 24 hours of growth;
d) repeating step a) to step c) two to nine times to enrich for small bacterial cell colonies; and
e) isolating the engineered NDV vector genome from the small bacterial cells colonies,
wherein the small bacterial cells colonies comprise stable engineered NDV vector genome having the stabilizing segment in L gene.

Other features and advantages of the present disclosure will become apparent from the following detailed description. It should be understood, however, that the detailed description and the specific Examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments are described below in relation to the drawings in which:

FIG. 1A shows a schematic representation of an engineered NDV vector with XbaI and MluI restriction endonuclease sites introduced between the P and M genes. GFP, full-length spike protein (FLS) or the C-terminal truncated spike (Δ19S) genes were inserted into this site.

FIG. 1B shows virus replication and cytopathic effect in cells. DF-1 cells were infected with NDV-FLS, NDV-Δ19S, or NDV-GFP virus at a multiplicity of infection (MOI) of 10. The first row shows immunofluorescence staining for NDV ribonucleoprotein. The second row shows bright field. Both NDV-FLS and NDV-Δ19S replicated in cells, showing accumulation of NDV nucleoprotein, and caused cytopathic effect (syncytia), similar to the NDV-GFP control.

FIG. 1C shows results of agarose gel electrophoresis of PCR amplified products from DF-1 cells infected with engineered NDV expressing SARS-CoV-2 spike protein to confirm spike protein expression. DF-1 cells were infected with either NDV-FLS, NDV-Δ19S, or NDV-GFP. RNA was extracted from cells 12 hours later and reverse transcribed to cDNA with M-MuLV-RT. Primers were used to target both the FLS and the 119S (lanes 1-6); or only the full-length spike (lanes 7-12). Lanes 1 and 7: NDV-FLS; lanes 2 and 8: NDV-Δ19S; Lanes 3 and 9: NDV-GFP; Lanes 4 and 10: plasmid clone of NDV-full-length spike protein (positive control); Lanes 5 and 11: uninfected DF1 cells (negative control); Lanes 6 and 12: no-template control. M=GeneRuler 50 bp DNA Ladder (Thermo Fisher Scientific).

FIG. 1D shows Western blots of whole cell lysates from DF-1 cells infected with an MOI of 5, with either (1) NDV-FLS; (2) NDV-Δ19S; (3) NDV-GFP; or (4) uninfected negative control to confirm spike protein expression. Immunoblotting was done with rabbit-anti-spike protein (NB100-56578; Novus Biologicals), mouse-anti-NDV (NBP2-11633; Novus Biologicals), and mouse-anti-actin (MA5-15739; ThermoFisher). A strong band at around 180 kDa corresponding to the spike protein is detected in the lysate of cells infected with NDV-FLS and -SΔ19, but not in cells infected with NDV-GFP or uninfected cells (control). Infection was confirmed by the presence of bands corresponding to the ribonucleoprotein for NDV in infected cells.

FIG. 1E shows Western blots of purified viruses. 1.0×10⁷ focus forming units (FFU) of (1) NDV-FLS; (2) NDV-Δ19S; or (3) NDV-GFP vectors were used for Western blotting using a primary rabbit anti-spike protein antibody (top), or a primary mouse anti-NDV ribonucleoprotein antibody (bottom), with the same antibodies described for FIG. 1D. The blot shows incorporation of the spike protein into the purified virions, while NDV-Δ19S and NDV-GFP control shows no transgene expression.

FIG. 1F shows crystal violet staining of DF-1 cells infected with NDV-GFP, NDV-FLS, or NDV-Δ19S vector. DF-1 cells in 6-well plates were infected with each of NDV-GFP, NDV-FLS, or NDV-Δ19S virus at an MOI of 0.1. Cells were grown in DMEM with 2% FBS supplemented with 5% allantoic fluid. 24 hours post-infection (hpi), media was removed, cells were washed in PBS, fixed with methanol/acetone for 20 minutes at −20° C., and stained with crystal violet.

FIG. 1G shows fusogenicity score of NDV-GFP, NDV-FLS, and NDV-Δ19S. Fusogenicity score was calculated by dividing the number of nuclei by the number of cells in four fields of view per each of the three biological replicates. Counting was assisted using ImageJ (U.S. National Institutes of Health, Bethesda, Md., USA). The score for each virus was normalized to the non-infected negative control, and averages were compared using an ANOVA and a Kruskal-Wallis multiple comparisons test. NDV-FLS showed less fusogenicity compared to the other viruses (***p<0.001).

FIG. 2 shows an immunoblot from cell lysates infected with NDV-FLS, NDV-Δ19S, and NDV-GFP, as well as the purified viruses. The blot shows efficient incorporation of spike protein into the NDV virion (first lane of top and middle blots, after molecular weight marker [MW]). Additionally, overexposure of a Western blot for spike protein reveals the presence of C-terminal truncated spike protein in the NDV-Δ19S virion (middle blot, rectangular box), albeit at much lower intensity than the full-length spike protein in the NDV-FLS virion. This shows that specific cytoplasmic transport signals are needed to enable efficient incorporation of the transgene on the NDV virion's surface.

FIG. 3 shows that neutralizing antibodies directed against SARS-CoV-2 spike protein do not block NDV-FLS or NDV-Δ19S infection of HEK293T-hACE2 cells. 1000 focus-forming units (FFU) of NDV-FLS, NDV-Δ19S or NDV-GFP were incubated with an antibody against the SARS-CoV-2 spike protein receptor binding domain (MA5-35958) at multiple dilutions (10 ug/mL, 5 ug/mL, 2.5 ug/mL down to 0.31 ug/mL [1/25]) for 1 h at room temperature with rocking plus 30 min at 37° C. HEK293T-hACE2 cells (2% FBS, DMEM, 5% allantoic fluid) were infected with the virus-Ab mixture and immunofluorescence assay was performed three days post infection. Images for the first three antibody dilutions are shown. These results show that neutralizing antibodies against SARS-CoV-2 spike protein do not affect NDV-FLS or NDV-Δ19S infection. When cells were incubated with hyperimmune serum from chickens vaccinated against NDV, the NDV-FLS was fully neutralized, suggesting that additional S protein on the surface does not functionally allow the virus to enter the cells.

FIG. 4 shows lyophilized NDV-FLS virus retains infectivity. Triplicate samples of NDV-FLS were either left untreated or adjusted to a final concentration of 5% sucrose, 5% sucrose/5% Iodixanol or mixed 1:1 with a stabilizing agent comprised of 10% lactose, 2% peptone, 10 mM Tris-HCl, pH 7.6 and lyophilized at 44×10⁻³ MBAR and −52° C. for 16 hr. Lyophilized samples were stored at 4° C. for 48 hours before being resuspended in 1 mL 5% sucrose/PBS and titered by TCID50 on DF-1 cells. Statistical analysis was completed by using a two-way analysis of variance with Tukey's multiple comparisons test with significance set at p<0.05.

FIG. 5 shows quantification of spike protein-specific CD8+ T cell responses. Groups of male Balb/c mice were administered with 5×10⁵, 1×10⁶ or 1×10⁶ PFU of NDV-FLS in either sucrose of iodixanol intranasally. After 32 days, mice were boosted with the same dose of vaccine via the same route (intranasal). Five days after boost, the mice were euthanized and spike protein-specific CD8 T cell responses were quantified in the blood, spleen, bronchoalveolar fluid (BALF), and lung.

FIG. 6 shows quantification of spike protein-specific CD4+ T cell responses. Groups of male Balb/c mice were administered with 5×10⁵, 1×10⁶ or 1×10⁶ PFU of NDV-FLS in either sucrose of iodixanol intranasally. After 32 days, mice were boosted with the same dose of vaccine via the same route (intranasal). After 32 days, mice were boosted with the same dose of vaccine via the same route of administration. Five days after boost, the mice were euthanized and spike protein-specific CD8 T cell responses were quantified in the blood, spleen, bronchoalveolar fluid (BALF), and lung.

FIG. 7 shows the kinetics of spike protein-specific CD8+ and CD4+ T cells in the blood of vaccinated mice. Male C57BL/6 or Balb/c mice were vaccinated using either intranasal or intramuscular delivery of 5×10⁶ FFU NDV-FLS, with a boost delivered through the same route and same dose 32 days post prime. At day 10 post-vaccine administration, a subset (n=4) of mice were terminally bled and the spike protein specific CD8+ and CD4+ T cell responses quantified. Mice were non-terminally bled prior to being boosted on day 28, and then bled again on days 5 and 10 post-boost. Spike protein specific CD8+ and CD4+ T cell responses were quantified in the collected blood.

FIG. 8 shows killing of murine acute myeloid leukemia (AML) C1498 cells in vitro by mesogenic NDV-GFP-GM (i.e., the mesogenic version of the NDV backbone expressing the GFP protein). C1498 cells were infected at different MOIs, spanning 0.0001 to 100, and after 72 days, the metabolic activity of infected cells was evaluated by Resazurin assay as an indication of the cytolytic potential of the tested viruses. Tested viruses include the mesogenic NDV-GFP-GM (Guelph mesogenic), the lentogenic NDV-GFP-GL, and a hyper-fusogenic mesogenic NDV-GFP-NY. Results show that NDV-GFP-GM caused a significantly higher drop in metabolic activity compared to the other two tested viruses (*p<0.05, **p<0.01, ****p<0.0001).

FIG. 9A shows the percentage of NK cells expressing the early activation marker CD69, in the blood of ID8 ovarian tumor bearing mice 36 hours after intravenous injection of 1×10⁸ PFU NDV-F3aa-GFP (mesogenic). NDV: Newcastle disease virus; PBS: phosphate-buffered saline mock control group; * p<0.05; **p<0.01; ***p<0.001; ****p<0.0001; ns=not significant.

FIG. 9B shows a graph depicting the percentage of NK cells in the blood of ID8 ovarian tumor bearing mice that are IFNy+, 36 hours post intravenous injection of 1×10⁸ PFU NDV-F3aa-GFP (mesogenic). NDV: Newcastle disease virus; PBS: phosphate-buffered saline mock control group; *p<0.05; **p<0.01; ***p<0.001; ****p<0.0001; ns=not significant.

FIG. 10 shows an immunoblot of prefusion stabilized SARS-CoV-2 spike (PFS) in the allantoic fluid of embryonated eggs inoculated with NDV-PFS. A 6% SDS-PAGE gel and rabbit anti-SARS-CoV-2 S1 (dilution: 1:1000; PA5-81795; ThermoFisher) was used for detection of SARS-CoV-2 spike (black arrow). A 10% SDS-PAGE gel and mouse anti-NDV ribonucleoprotein (dilution: 1:5000; NBP2-11633; Novus Biologicals) was used for detection of NDV. 20 μL of allantoic fluid was loaded in for samples. NDV-GFP was loaded as a control. MW used was the PageRuler™ Plus Prestained Protein Ladder (Thermo Scientific).

FIG. 11 shows graphs of results on protection from weight loss in NDV-COVID-19 vaccinated hamsters challenged with SARS-CoV-2. Groups of eight Syrian Golden hamsters were anaesthetized with inhalation isoflurane and administered 1E7 PFU/animal of recombinant NDV-GFP, NDV-FLS, or NDV-PFS via the intranasal (IN) route. For the prime/boost groups, 28 days following the initial vaccine administration, hamsters were administered a second dose of the homologous vaccine (1E7 PFU/animal by IN route). At 28 days post-prime or 28 days post-prime/boost, hamsters were moved into a CL-3 facility, anaesthetized with inhaled isoflurane and infected SARS-CoV-2. Challenge dose: Alpha variant @ 8.5E4 PFU/animal by IN, Ancestral (Wuhan) @ 1E5 PFU/animal by IN. After recovery from anesthetic hamsters were monitored daily throughout the course of infection. Body weights of hamsters were recorded daily. Error bars represent mean+/−SEM.

FIG. 12 shows graphs depicting SARS-CoV-2 viral RNA copies in the lung and nasal turbinates of vaccinated and challenged Syrian hamsters. At 5 days post challenge with Alpha variant @ 8.5E4 PFU/animal by IN or Ancestral (Wuhan) @ 1E5 PFU/animal by IN, vaccinated hamsters were euthanized and viral RNA copies in the lung and nasal turbinates quantified by qRT-PCR. A standard curve produced with synthesized target DNA was run with every plate and used for the interpolation of viral genome copy numbers. Viral RNA levels are reported as genome copy number. Error bars represent mean+/−SEM. Differences in the magnitude of virus copy number were assessed by Kruskall-Wallis test with Dunn's test for multiple comparisons.

FIG. 13 shows graphs depicting infectious SARS-CoV-2 in the lung and nasal turbinates of vaccinated and challenged Syrian hamsters. At 5 days post challenge with Alpha variant @ 8.5E4 PFU/animal by IN or Ancestral (Wuhan) @ 1E5 PFU/animal by IN, vaccinated hamsters were euthanized and infectious titers of SARS-CoV-2 in the lung and nasal turbinates determined. Homogenized tissue samples were serially diluted 10-fold in media and dilutions were then added to 96-well plates of 95% confluent Vero cells containing 504 of the media in replicates of three and incubated for five days at 37° C. with 5% CO₂. Plates were scored for the presence of cytopathic effect on day five after infection. Titers were calculated using the Reed-Muench method, converted to PFU after multiplying by 0.69 and reported as PFU/g of tissue.

### DETAILED DESCRIPTION

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

As used herein, the singular forms “a”, “an” and “the” include plural references unless the content clearly dictates otherwise.

### Compositions

The term “Newcastle Disease Virus” (NDV), as used herein, includes without limitation, avian orthoavulavirus-1 (AOaV-1) and variants thereof. The genome of NDV is single-stranded, negative-sense, non-segmented RNA comprising six genes in the order 3′-NP-P-M-F-HN-L-S′ encoding six structural proteins: nucleocapsid protein (NP), phosphoprotein (P), matrix protein (M), fusion protein (F), haemagglutinin-neuraminidase (HN), and a large polymerase protein (L). The NDV vector genome is packaged within an envelope (membrane), which is made of lipid bilayer, HN protein, and F protein. The M protein forms a grid-like array on the inner surface of the viral envelope. Inside the envelope the NP protein is tightly bound to the vector genome, forming a nucleocapsid complex. The L protein and P protein are loosely bound to nucleocapsid complex. NDV strains can be pathotypically categorized into three groups: velogenic (i.e. highly virulent), mesogenic (i.e. intermediate virulence), and lentogenic (i.e. non-virulent). Velogenic strains produce severe nervous and respiratory signs, spread rapidly, and have high mortality rate in birds. Mesogenic strains cause coughing, affect egg quality and production, and have low mortality rate in birds. Lentogenic strains produce mild signs with negligible mortality in birds. Although NDV can infect humans, most cases are non-symptomatic, and only very rarely it causes a mild fever and/or conjunctivitis. A nucleic acid sequence that defines a strain as lentogenic is GGGAGACAGGGGCGCC (SEQ ID NO: 25), which is translated to GRQGRL (SEQ ID NO: 26) found in the F protein encoded by a nucleic acid sequence in Genbank accession number AF077761.1. A strain is mesogenic when there is a 3 amino acid change in the F gene, i.e. from GRQGRL to RRQRRF at amino acid positions 112, 115, and 117 in reference SEQ ID NO: 28. In some embodiments of this disclosure, the NDV vector is lentogenic. In some embodiments, the NDV vector comprises a nucleic acid comprising a nucleic acid sequence of SEQ ID NO: 25 or encodes the amino acid sequence of SEQ ID NO: 26. In some embodiments, the NDV vector is mesogenic. In some embodiments, the NDV vector comprises a nucleic acid comprising a nucleic acid sequence of SEQ ID NO: 23 or 27, or encodes the amino acid sequence RRQRRF (SEQ ID NO: 36).

As used herein, “transduction” of a cell by a viral vector means entry of the viral vector into the cell and transfer of genetic material into the cell by which nucleic acid incorporated in the viral vector is transferred into the cell.

The term “nucleic acid”, “nucleic acid molecule” or its derivatives, as used herein, is intended to include unmodified DNA or RNA or modified DNA or RNA. For example, the nucleic acid molecules of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

As used herein, the term “polypeptide” encompasses both peptides and proteins, and fragments thereof of peptides and proteins, unless indicated otherwise. In one embodiment, the therapeutic agent is a polypeptide.

As used herein, the term “vector”, “viral vector”, “viral particle”, or “delivery vector”, and their derivatives, refer to a particle that functions as a nucleic acid delivery vehicle, and which comprises the viral nucleic acid (i.e., the viral vector genome) packaged within the particle. Viral vectors according to the present disclosure package a NDV vector genome. A “heterologous nucleic acid” or “heterologous nucleotide sequence” is a sequence that is not naturally occurring in the virus, i.e. a transgene. In general, the heterologous nucleic acid or nucleotide sequence comprises an open reading frame that encodes a polypeptide and/or a non-translated RNA.

The term “engineered Newcastle Disease Virus vector” or “engineered NDV vector” comprises an engineered (also interchangeably referred as “recombinant”) NDV vector genome packaged within an envelope, i.e. a DNA copy of the NDV antigenome comprised in an expression plasmid. The engineered NDV vector genome is capable of generating mRNA much like a native negative-sense NDV genome is capable of generating mRNA. The engineered NDV vector genome has a promoter, for example, an RNA promoter such as T7 immediately upstream of the 5′ end of the antigenome, or any suitable promoter known in the art, which drives expression of the virus RNA genome. The expression of a heterologous nucleic acid (transgene) such as one that encodes an immunogenic agent is driven by a typical NDV genome promoter. The T7 promoter, followed by 3 non-template guanines, is placed immediately upstream of the first nucleotide of the NDV vector genome. The engineered NDV vector genome described herein contains unique restriction sites for endonucleases such as XbaI and MluI for use in molecular biology techniques, for example, to facilitate efficient insertion of a heterologous nucleic acid. The skilled person would readily recognize endonuclease restriction sites such as XbaI and MluI. Engineered NDV vector genome can also contain an L289A mutation in the fusion (F) protein for enhanced fusion, a self-cleaving hepatitis delta virus (HDV) ribozyme sequence to ensure adherence to the “rule of six” by self-cleaving immediately at the end of the viral antigenomic transcript, and a T7 terminator sequence. An engineered NDV vector genome can also encode a F protein that has been mutated to contain a multi-basic cleavage site. The F protein and/or the HN protein of an engineered NDV vector genome can be substituted with the corresponding avian paramyxovirus (APMV) F protein and/or HN protein, or part thereof. Modification of F, HN or both, can be done using additional unique restriction endonuclease sites that flank these genes such as PacI, AgeI and AscI, which for example have been purposefully added in exemplified embodiments of this disclosure. When the substitution occurs in part, the resulting protein would be a chimeric protein, for example, a chimeric F protein and/or a chimeric HN protein containing sequence from NDV and APMV. The APMV can be APMV5.

The term “promoter,” as used herein, refers to a nucleotide sequence that directs the transcription of a gene or coding sequence to which it is operably linked.

The term “operably linked”, as used herein, refers to an arrangement of two or more components, wherein the components so described are in a relationship permitting them to function in a coordinated manner. For example, a transcriptional regulatory sequence or a promoter is operably linked to a coding sequence if the transcriptional regulatory sequence or promoter facilitates aspects of the transcription of the coding sequence. The skilled person can readily recognize aspects of the transcription process, which include, but not limited to, initiation, elongation, attenuation and termination. In general, an operably linked transcriptional regulatory sequence is joined in cis with the coding sequence, but it is not necessarily directly adjacent to it.

A “segment” of a nucleotide sequence is a sequence of contiguous nucleotides. A segment can be at least about 5, 10, 15, 20, 25, 30, 35, 40, 45, 50, 60, 75, 85, 100, 110, 120, 130, 145, 150, 160, 175, 200, 250, 300, 350, 400, 450, 500 or more contiguous nucleotides.

A “fragment” of an amino acid sequence is a sequence of contiguous amino acids. A segment can be at least about 5, 10, 15, 20, 25, 30, 35, 40, 45, 50, 60, 75, 85, 100, 110, 120, 130, 145, 150, 160, 175, 200, 250, 300, 350, 400, 450, 500 or more contiguous amino acids.

The presence of the NDV vector genome can be tracked by a marker. In another embodiment, the NDV vector genome further comprises a nucleotide sequence encoding a marker. In another embodiment, the marker comprises GFP.

A “therapeutic agent” can be an agent that can alleviate or reduce symptoms that result from an absence or defect in a protein in a cell, tissue or subject. In addition, a “therapeutic agent” can be an agent that otherwise confers a benefit to a subject, e.g., anti-disease effects or improvement in survivability upon exposure to a causative agent of an infectious. A “therapeutic agent” can be a polypeptide, a therapeutic protein, an antigen, an antibody, or an antigen binding fragment. The antibody can be a monoclonal, polyclonal, chimeric, humanized antibody, or a fragment thereof, or a combination thereof. The antigen binding fragment is a Fab, Fab′, F(ab′)2, scFv, dsFv, ds-scFv, dimer, minibody, diabody, or multimer thereof or bispecific antibody fragment, or a combination thereof. A “therapeutic agent” can be an immunogenic agent.

The term “immunogenic agent” as used herein refers to a molecule that can elicit an immune response in a subject. The immunogenic agent can be an antigenic molecule such as a polypeptide that can induce, for example, humoral and/or cellular response, by activating B cells for the production of antibodies, CD4+ T cells for helper cell functions, and CD8+ T cells for their cytotoxic functions. An immunogenic agent can be encoded by a heterologous nucleic acid comprised in the engineered NDV vector or vaccine of the present disclosure. An immunogenic agent can be a protein or fragment thereof from an infectious agent for a disease, for example, such as influenza, SARS, MERS, or COVID-19.

SARS-CoV-2 is the causative agent of COVID-19. An immunogenic agent can be, for example, the spike protein (also referred as “spike”) or fragment thereof of SARS-CoV-2. SARS-CoV-2 includes Variants of Concern (VoC) such as the South African B.1.351 variant (Peiris, M. and G. M. Leung, 2020). Other variants include variant B.1.1.7 having spike protein mutations delta69-70, delta144Y, N501Y, A570D, D614G, P681H, T716I, S982A, and D1118H; variant B.1.351 having spike protein mutations L18F, D80A, D215G, delta241-243, R246I, K417N, E484K, N501Y, D614G, and A701V; and variant B.1.351 2P having spike protein mutation L18F, D80A, D215G, delta241-243, R246I, K417N, E484K, N501Y, D614G, A701V, and KV986-987PP. The spike protein can be modified to enhance its stabilization. For example, proline mutations, such as two of F817P, A892P, A899P, A942P, K986P, and V987P, and in particular K986P and V987P (Hsieh, C.-L., et al., Science 2020), can be introduced to create a pre-fusion stabilized spike protein immunogen, however, when there is only 2 proline mutations, it is relatively unstable and difficult to produce in mammalian cells. The present inventors found that when all six prolines are introduced (i.e. when the engineered NDV expresses HexaPro (6 prolines)), version of prefusion stabilized spike, that retains the prefusion conformation of the spike protein, is retained and it shows higher expression than only two prolines. The six proline spike protein can also withstand heating and freezing better than the two prolines spike protein. In addition, the furin-cleavage site (RRAR) in the spike protein can be mutated to GSAS to render it furin-cleavage deficient, thereby increases its half-life. The immunogenic agent can be for priming and/or boosting an immune response against an antigen. Engineered NDV vectors of the present disclosure that express the spike protein include the constructs having the sequence in SEQ ID NO: 2-4, 18 or 19, with those comprising the proline mutations and/or deficient furin-cleavage site shown in SEQ ID NO: 18 and 19. In an embodiment, the engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence any one of SEQ ID NO: 2-4, 18, or 19. In an embodiment, the engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of SEQ ID NO: 18 or 19. In an embodiment, the immunogenic agent is a SARS-CoV-2 spike protein or fragment thereof. In an embodiment, the SARS-CoV-2 spike protein is encoded by the nucleic acid sequence having at least 75%, 80%, 85%, 90%, 95%, 96%, 97%, 98%, 99%, 99.5%, 99.9%, or 100% identity to the nucleic acid sequence of SEQ ID NO: 8 or 17. In an embodiment, the SARS-CoV-2 spike protein comprises an amino acid sequence having at least 85%, 90%, 95%, 96%, 97%, 98%, 99%, 99.5%, 99.9%, or 100% identity to the amino acid sequence of SEQ ID NO: 6, 7, 29, 30, 31, or 41. In an embodiment, the SARS-CoV-2 spike protein comprises an amino acid sequence having at least 85%, 90%, 95%, 96%, 97%, 98%, 99%, 99.5%, 99.9%, or 100% identity to a sequence of GenBank reference QHD43416.1 or QIZ15537.1, or variant B1.1.7 having spike protein mutations delta69-70, delta144Y, N501Y, A570D, D614G, P681H, T716I, S982A, D1118H, and L18F; variant B.1.351 having spike protein mutations D80A, D215G, delta241-243, R246I, K417N, E484K, N501Y, D614G, and A701V; or variant B.1.351 2P having spike protein mutations L18F, D80A, D215G, delta241-243, R246I, K417N, E484K, N501Y, D614G, A701V, and KV986-987PP. In an embodiment, the SARS-CoV-2 spike protein comprises an amino acid sequence having at least 85%, 90%, 95%, 96%, 97%, 98%, 99%, 99.5%, 99.9%, or 100% identity to a sequence of GenBank reference QHD43416.1. In an embodiment, the SARS-CoV-2 spike protein comprises an amino acid sequence having at least 85%, 90%, 95%, 96%, 97%, 98%, 99%, 99.5%, 99.9%, or 100% identity to a sequence of GenBank reference QIZ15537.1. In an embodiment, the SARS-CoV-2 spike protein comprises an amino acid sequence that is at least 85%, 90%, 95%, 96%, 97%, 98%, or 99% identical to the amino acid sequence of SEQ ID NO: 6, 7, 29, 30, 31, or 41, comprising any two mutations selected from the group consisting of F817P, A892P, A899P, A942P, K986P, and V987P at the positions corresponding to positions of SEQ ID NO: 6. In an embodiment, the mutations are K986P and V987P. In an embodiment, the SARS-CoV-2 spike protein comprises an amino acid sequence that is at least 85%, 90%, 95%, 96%, 97%, 98%, or 99% identical to the amino acid sequence of SEQ ID NO: 6, 7, 29, 30, 31, or 41, comprising mutations F817P, A892P, A899P, A942P, K986P, and V987P at the positions corresponding to positions of SEQ ID NO: 6. In an embodiment, the SARS-CoV-2 spike protein comprises an amino acid sequence that is at least 85%, 90%, 95%, 96%, 97%, 98%, or 99% identical to the amino acid sequence of SEQ ID NO: 6, 7, 29, 30, 31, or 41, comprising mutations 682-RRAR-685 to 682-GSAS-685, and any two mutations selected from the group consisting of F817P, A892P, A899P, A942P, K986P, and V987P at the positions corresponding to positions of SEQ ID NO: 6. In an embodiment, the mutations are K986P and V987P. In an embodiment, the SARS-CoV-2 spike protein comprises an amino acid sequence that is at least 85%, 90%, 95%, 96%, 97%, 98%, or 99% identical to the amino acid sequence of SEQ ID NO: 6, 7, 29, 30, 31, or 41, comprising mutations 682-RRAR-685 to 682-GSAS-685, F817P, A892P, A899P, A942P, K986P, and V987P at the positions corresponding to positions of SEQ ID NO: 6. The term “pharmaceutically acceptable” in referring to diluent, buffer, carrier, or excipient, as used herein, includes any and all solvents, dispersion media, coatings, antibacterial and antifungal agents, isotonic and absorption delaying agents, that are physiologically compatible. Pharmaceutically acceptable diluent, buffer, carrier, or excipient includes sterile aqueous solutions or dispersions and sterile powders for the extemporaneous preparation of sterile injectable solutions or dispersion. The skilled person can readily recognize the use of such media and agents for pharmaceutically active substances. In one embodiment, the engineered NDV vector is comprised in a pharmaceutical composition that includes a pharmaceutically acceptable diluent, buffer, carrier, or excipient.

The present inventors have provided an engineered Newcastle Disease Virus (NDV) vector comprising a nucleic acid comprises at least one heterologous nucleic acid segment encoding a therapeutic agent operably linked to a promoter capable of expressing the segment in a host cell. The present inventors have further provided a vaccine comprising an engineered NDV vector having a nucleic acid that comprises at least one heterologous nucleic acid segment encoding an immunogenic agent operably linked to a promoter capable of expressing the segment in a host cell, and methods of treating or preventing a disease, for example, an infectious disease, with said vaccine or engineered NDV vector.

Accordingly, herein provided is an engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of SEQ ID NO: 9, 10, 23, or 27.

Also provided is an isolated nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises XbaI and MluI restriction endonuclease sites between nucleic acid sequence encoding phosphoprotein and matrix protein. In an embodiment, the isolated nucleic acid comprises a nucleic acid sequence that is at least 75%, 80%, 85%, 90%, 95%, 96%, 97%, 98%, 99%, 99.5%, 99.9% or 100% identical to the nucleic acid sequence of SEQ ID NO: 9, 10, 23, or 27.

In an embodiment, the at least one heterologous nucleic acid segment encodes a therapeutic agent operably linked to a promoter capable of expressing the segment in a host cell.

In another aspect, also provided is an engineered chimeric NDV vector comprising a nucleic acid having a nucleic acid sequence encoding a L protein having a stabilizing segment, a chimeric F protein, and a chimeric HN protein, wherein the chimeric F protein comprises avian paramyxovirus 5 (APMV5) F protein segment thereof at the N-terminus and an NDV F protein segment at the C-terminus, and wherein the chimeric HN protein comprises an NDV HN protein segment at the N-terminus and an AMPV5 HN protein segment at the C-terminus. In an embodiment, the nucleic acid comprises XbaI and MluI restriction endonuclease sites between nucleic acid sequence encoding phosphoprotein and matrix protein. The stabilizing segment in L protein provides stability to molecular clones in a host cell such as a bacterial cell. In an embodiment, the L protein comprises a stabilizing segment. In an embodiment, the L protein comprises an amino acid sequence having at least 85%, 90%, 95%, 96%, 97%, 98%, 99%, 99.5%, 99.9%, or 100% identity to the amino acid sequence as set forth in SEQ ID NO: 11. In an embodiment, the stabilizing segment in the L protein comprises the sequence 1287-VSPYIHISNDSQRLFTEEGVKEGNVVYQQI-1316 (SEQ ID NO: 20). In an embodiment, the host cell is a bacterial cell.

The chimeric F protein is a chimeric with N-terminus APMV5 F protein and C-terminus NDV F protein, for example, NDV F protein from amino acid positions 501 to 553 (SEQ ID NO: 28; encoded by SEQ ID NO: 32, i.e. F gene in accession AF077761.1), which once incorporated into the chimeric protein become amino acid positions 494 to 546 in the chimeric protein, such as shown in SEQ ID NO: 12. In an embodiment, the chimeric F protein comprises at the C-terminus 53 amino acids of NDV F protein from amino acid positions 501 to 553 of SEQ ID NO: 28. In an embodiment, the chimeric F protein comprises an amino acid sequence having at least 85%, 90%, 95%, 96%, 97%, 98%, 99%, 99.5%, 99.9%, or 100% identity to the amino acid sequence of SEQ ID NO: 12. In an embodiment, the chimeric HN protein comprises at the N-terminus 53 amino acids of NDV HN protein from amino acid positions 1 to 53 of SEQ ID NO: 34. In an embodiment, the chimeric HN protein comprises an amino acid sequence having at least 85%, 90%, 95%, 96%, 97%, 98%, 99%, 99.5%, 99.9%, or 100% identity to the amino acid sequence of SEQ ID NO: 13. In an embodiment, the nucleic acid further comprises at least one heterologous nucleic acid segment encoding a therapeutic agent operably linked to a promoter capable of expressing the segment in a host cell.

In an embodiment, the therapeutic agent comprises a SARS-CoV-2 spike protein or a fragment thereof. In an embodiment, the SARS-CoV-2 spike protein is encoded by the nucleic acid sequence having at least 75%, 80%, 85%, 90%, 95%, 96%, 97%, 98%, 99%, 99.5%, 99.9%, or 100% identity to the nucleic acid sequence of SEQ ID NO: 8 or 17. In an embodiment, the SARS-CoV-2 spike protein comprises an amino acid sequence having at least 85%, 90%, 95%, 96%, 97%, 98%, 99%, 99.5%, 99.9%, or 100% identity to the amino acid sequence of SEQ ID NO: 6, 7, 29, 30, 31, or 41. In an embodiment, the SARS-CoV-2 spike protein comprises an amino acid sequence having at least 85%, 90%, 95%, 96%, 97%, 98%, 99%, 99.5%, 99.9%, or 100% identity to the amino acid sequence of GenBank reference QHD43416.1 or QIZ15537.1, or variant B1.1.7 having spike protein mutation of one or more of delta69-70, delta144Y, N501Y, A570D, D614G, P681H, T716I, S982A, D1118H, and L18F; variant B.1.351 having spike protein mutation of one or more of D80A, D215G, delta241-243, R246I, K417N, E484K, N501Y, D614G, and A701V; or variant B.1.351 2P having spike protein mutation of one or more of L18F, D80A, D215G, delta241-243, R246I, K417N, E484K, N501Y, D614G, A701V, and KV986-987PP. In an embodiment, the SARS-CoV-2 spike protein comprises an amino acid sequence having a sequence of GenBank reference QHD43416.1. In an embodiment, the SARS-CoV-2 spike protein comprises an amino acid sequence having at least 85%, 90%, 95%, 96%, 97%, 98%, 99%, 99.5%, 99.9%, or 100% identity to the amino acid sequence of GenBank reference QIZ15537.1. In an embodiment, the SARS-CoV-2 spike protein comprises an amino acid sequence that is at least 85%, 90%, 95%, 96%, 97%, 98%, or 99% identical to the amino acid sequence of SEQ ID NO: 6, 7, 29, 30, 31, or 41, comprising any two mutations selected from the group consisting of F817P, A892P, A899P, A942P, K986P, and V987P at the positions corresponding to positions of SEQ ID NO: 6. In an embodiment, the SARS-CoV-2 spike protein comprises an amino acid sequence that is at least 85%, 90%, 95%, 96%, 97%, 98%, or 99% identical to the amino acid sequence of SEQ ID NO: 6, 7, 29, 30, 31, or 41, comprising mutations F817P, A892P, A899P, A942P, K986P, and V987P at the positions corresponding to positions of SEQ ID NO: 6. In an embodiment, the SARS-CoV-2 spike protein comprises an amino acid sequence having at least 85%, 90%, 95%, 96%, 96%, 97%, 98%, or 99% identity to the amino acid sequence of SEQ ID NO: 6, 7, 29, 30, 31, or 41, comprising mutations 682-RRAR-685 to 682-GSAS-685, and any two mutations selected from the group consisting of F817P, A892P, A899P, A942P, K986P, and V987P at the positions corresponding to positions of SEQ ID NO: 6. In an embodiment, the SARS-CoV-2 spike protein comprises an amino acid sequence that is at least 85%, 90%, 95%, 96%, 97%, 98%, or 99% identical to the amino acid sequence of SEQ ID NO: 6, 7, 29, 30, 31, or 41, comprising mutations 682-RRAR-685 to 682-GSAS-685, F817P, A892P, A899P, A942P, K986P, and V987P at the positions corresponding to positions of SEQ ID NO: 6.

The engineered NDV vector of the present disclosure can activate an immune response which is useful for its use as an immunogenic composition, an oncolytic agent, or a vaccine. Accordingly, also provided is an immunogenic composition, an oncolytic agent, or a vaccine, wherein the immunogenic composition, oncolytic agent, or vaccine comprises an engineered NDV vector comprising a nucleic acid having a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In some embodiments, the oncolytic agent comprises an engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein.

Also provided in the present disclosure is a pharmaceutical composition comprising an engineered NDV vector having a nucleic acid comprising a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, and a pharmaceutically acceptable carrier.

The engineered NDV vector, vaccine, immunogenic composition, or pharmaceutical composition described herein can be lyophilized without significant negative effects. In some embodiments, the engineered NDV vector, vaccine, immunogenic composition, or pharmaceutical composition is lyophilized. In some embodiments, the lyophilized engineered NDV vector, vaccine, immunogenic composition, or pharmaceutical composition is comprised in a solution comprising 1) 5% sucrose, 2) 5% sucrose and 5% lodixanol, 3) 2.5% sucrose, 5% lactose, 1 peptone, 5 mM Tris-HCl, pH 7.6, or 4) 2.5% sucrose, 2.5% lodixanol, 5% lactose, 1% peptone, 5 mM Tris-HCl, pH 7.6, prior to lyophilization.

Nucleic acid and amino acid sequences described herein are set out in Table 1.

TABLE 1
Sequences

| | |
|---|---|
| SEQ ID NO: | TAATACGACTCACTATAGGGACCAAACAGAGAATCCGTGAGTTACGATAAAAGGCGAAGG |
| 1; nucleic | AGCAATTGAAGTCGCACGGGTAGAAGGTGTGAATCTCGAGTGCGAGCCCGAAGCACAAAC |
| sequence of | TCGAGAAAGCCTTCTGCCAACATGTCTTCCGTATTTGATGAGTACGAACAGCTCCTCGCG |
| NDV-GFP | GCTCAGACTCGCCCCAATGGAGCTCATGGAGGGGGAGAAAAAGGGAGTACCTTAAAAGTA |
| Molecular | GACGTCCCGGTATTCACTCTTAACAGTGATGACCCAGAAGATAGATGGAGCTTTGTGGTA |
| Clone | TTCTGCCTCCGGATTGCTGTTAGCGAAGATGCCAACAAACCACTCAGGCAAGGTGCTCTC |
| AF077761.1_ | ATATCTCTTTTATGCTCCCACTCACAGGTAATGAGGAACCATGTTGCCATTGCAGGGAAA |
| LaSota_Kan | CAGAATGAAGCCACATTGGCCGTGCTTGAGATTGATGGCTTTGCCAACGGCACGCCCCAG |
| R (with | TTCAACAATAGGAGTGGAGTGTCTGAAGAGAGAGCACAGAGATTTGCGATGATAGCAGGA |
| stabilizing | TCTCTCCCTCGGGCATGCAGCAACGGAACCCCGTTCGTCACAGCCGGGGCAGAAGATGAT |
| sequence in | GCACCAGAAGACATCACCGATACCCTGGAGAGGATCCTCTCTATCCAGGCTCAAGTATGG |
| L) | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaTTAGAAAAAATA |
| | CGGGTAGAACCGCCACCATGGTGAGCAAGGGCGAGGAGCTGTTCACCGGGGTGGTGCCCA |
| | TCCTGGTCGAGCTGGACGGCGACGTAAACGGCCACAAGTTCAGCGTGTCCGGCGAGGGCG |
| | AGGGCGATGCCACCTACGGCAAGCTGACCCTGAAGTTCATCTGCACCACCGGCAAGCTGC |
| | CCGTGCCCTGGCCCACCCTCGTGACCACCCTGACCTACGGCGTGCAGTGCTTCAGCCGCT |
| | ACCCCGACCACATGAAGCAGCACGACTTCTTCAAGTCCGCCATGCCCGAAGGCTACGTCC |
| | AGGAGCGCACCATCTTCTTCAAGGACGACGGCAACTACAAGACCCGCGCCGAGGTGAAGT |
| | TCGAGGGCGACACCCTGGTGAACCGCATCGAGCTGAAGGGCATCGACTTCAAGGAGGACG |
| | GCAACATCCTGGGGCACAAGCTGGAGTACAACTACAACAGCCACAACGTCTATATCATGG |
| | CCGACAAGCAGAAGAACGGCATCAAGGTGAACTTCAAGATCCGCCACAACATCGAGGACG |
| | GCAGCGTGCAGCTCGCCGACCACTACCAGCAGAACACCCCCATCGGCGACGGCCCCGTGC |
| | TGCTGCCCGACAACCACTACCTGAGCACCCAGTCCGCCCTGAGCAAAGACCCCAACGAGA |
| | AGCGCGATCACATGGTCCTGCTGGAGTTCGTGACCGCCGCCGGGATCACTCTCGGCATGG |
| | ACGAGCTGTACAAGTaATaaacgcgtACCCAAGGTCCAACTCTCCAAGCGGCAATCCTCT |
| | CTCGCTTCCTCAGCCCCACTGAATGGTCGCGTAACCGTAATTAATCTAGCTACATTTAAG |
| | ATTAAGAAAAAATACGGGTAGAATTGGAGTGCCCCAATTGTGCCAAGATGGACTCATCTA |
| | GGACAATTGGGCTGTACTTTGATTCTGCCCATTCTTCTAGCAACCTGTTAGCATTTCCGA |
| | TCGTCCTACAAGGCACAGGAGATGGGAAGAAGCAAATCGCCCCGCAATATAGGATCCAGC |
| | GCCTTGACTTGTGGACTGATAGTAAGGAGGACTCAGTATTCATCACCACCTATGGATTCA |
| | TCTTTCAAGTTGGGAATGAAGAAGCCACTGTCGGCATGATCGATGATAAACCCAAGCGCG |
| | AGTTACTTTCCGCTGCGATGCTCTGCCTAGGAAGCGTCCCAAATACCGGAGACCTTATTG |
| | AGCTGGCAAGGGCCTGTCTCACTATGATAGTCACATGCAAGAAGAGTGCAACTAATACTG |
| | AGAGAATGGTTTTCTCAGTAGTGCAGGCACCCCAAGTGCTGCAAAGCTGTAGGGTTGTGG |
| | CAAACAAATACTCATCAGTGAATGCAGTCAAGCACGTGAAAGCGCCAGAGAAGATTCCCG |
| | GGAGTGGAACCCTAGAATACAAGGTGAACTTTGTCTCCTTGACTGTGGTACCGAAGAAGG |
| | ATGTCTACAAGATCCCAGCTGCAGTATTGAAGGTTTCTGGCTCGAGTCTGTACAATCTTG |
| | CGCTCAATGTCACTATTAATGTGGAGGTAGACCCGAGGAGTCCTTTGGTTAAATCTTTGT |
| | CTAAGTCTGACAGCGGATACTATGCTAACCTCTTCTTGCATATTGGACTTATGACCACCG |
| | TAGATAGGAAGGGGAAGAAAGTGACATTTGACAAGCTGGAAAAGAAAATAAGGAGCCTTG |
| | ATCTATCTGTCGGGCTCAGTGATGTGCTCGGGCCTTCCGTGTTGGTAAAAGCAAGAGGTG |
| | CACGGACTAAGCTTTTGGCACCTTTCTTCTCTAGCAGTGGGACAGCCTGCTATCCCATAG |
| | CAAATGCTTCTCCTCAGGTGGCCAAGATACTCTGGAGTCAAACCGCGTGCCTGCGGAGCG |
| | TTAAAATCATTATCCAAGCAGGTACCCAACGCGCTGTCGCAGTGACCGCCGACCACGAGG |
| | TTACCTCTACTAAGCTGGAGAAGGGGCACACCCTTGCCAAATACAATCCTTTTAAGAAAT |
| | AAGCTGCGTCTCTGAGATTGCGCTCCGCCCACTCACCCAGATCATCATGACACAAAAAAC |
| | TAATCTGTCTTGATTATTTACAGTTAGTTTACCTGTCTATCAAGTTAGAAAAAACACGGG |
| | TAGAAGATTCTGGATCCCGGTTGGCGCCCTCCAGGTGCAAGttaattaaATGGGCTCCAG |
| | ACCTTCTACCAAGAACCCAGCACCTATGATGCTGACTATCCGGGTTGCGCTGGTACTGAG |
| | TTGCATCTGTCCGGCAAACTCCATTGATGGCAGGCCTCTTGCAGCTGCAGGAATTGTGGT |
| | TAGAGGAGACAAAGCCGTCAACATATACACCTCATCCCAGACAGGATCAATCATAGTTAA |
| | GCTCCTCCCGAATCTGCCCAAGGATAAGGAGGCATGTGCGAAAGCCCCCTTGGATGCATA |
| | CAACAGGACATTGACCACTTTGCTCACCCCCCTTGGTGACTCTATCCGTAGGATACAAGA |
| | GTCTGTGACTACATCTGGAGGGGGGAGACAGGGGCGCCTTATAGGCGCCATTATTGGCGG |
| | TGTGGCTCTTGGGGTTGCAACTGCCGCACAAATAACAGCGGCCGCAGCTCTGATACAAGC |
| | CAAACAAAATGCTGCCAACATCCTCCGACTTAAAGAGAGCATTGCCGCAACCAATGAGGC |
| | TGTGCATGAGGTCACTGACGGATTATCGCAACTAGCAGTGGCAGTTGGGAAGATGCAGCA |
| | GTTTGTTAATGACCAATTTAATAAAACAGCTCAGGAATTAGACTGCATCAAAATTGCACA |
| | GCAAGTTGGTGTAGAGCTCAACCTGTACCTAACCGAATTGACTACAGTATTCGGACCACA |
| | AATCACTTCACCTGCTTTAAACAAGCTGACTATTCAGGCACTTTACAATCTAGCTGGTGG |
| | AAATATGGATTACTTATTGACTAAGTTAGGTGTAGGGAACAATCAACTCAGCTCATTAAT |
| | CGGTAGCGGCTTAATCACtGGCAACCCTATTCTATACGACTCACAGACTCAACTCTTGGG |
| | TATACAGGTAACTgcaCCTTCAGTCGGGAACCTAAATAATATGCGTGCCACCTACTTGGA |
| | AACCTTATCCGTAAGCACAACCAGGGGATTTGCCTCGGCACTTGTCCCCAAAGTGGTGAC |
| | ACAGGTCGGTTCTGTGATAGAAGAACTTGACACCTCATACTGTATAGAAACTGACTTAGA |
| | TTTATATTGTACAAGAATAGTAACGTTCCCTATGTCCCCTGGTATTTATTCCTGCTTGAG |
| | CGGCAATACGTCGGCCTGTATGTACTCAAAGACCGAAGGCGCACTTACTACACCATACAT |
| | GACTATCAAAGGTTCAGTCATCGCCAACTGCAAGATGACAACATGTAGATGTGTAAACCC |
| | CCCGGGTATCATATCGCAAAACTATGGAGAAGCCGTGTCTCTAATAGATAAACAATCATG |
| | CAATGTTTTATCCTTAGGCGGGATAACTTTAAGGCTCAGTGGGGAATTCGATGTAACTTA |
| | TCAGAAGAATATCTCAATACAAGATTCTCAAGTAATAATAACAGGCAATCTTGATATCTC |
| | AACTGAGCTTGGGAATGTCAACAACTCGATCAGTAATGCTTTGAATAAGTTAGAGGAAAG |
| | CAACAGAAAACTAGACAAAGTCAATGTCAAACTGACTAGCACATCTGCTCTCATTACgTA |
| | TATCGTTTTGACTATCATATCTCTTGTTTTTGGTATACTTAGCCTGATTCTAGCATGCTA |
| | CCTAATGTACAAGCAAAAGGCGCAACAAAAGACCTTATTATGGCTTGGGAATAATACaCT |
| | cGATCAGATGAGAGCCACTACAAAAATGTGAACACAGATGAGGAACGAAGGTTTCCCTAA |
| | TAGTAATTTGTGTGAAAGTTCTGGTAGTCTGTCAGTTCAGAGAGTTAAGAAAAAACTACC |
| | GGTTGTAGATGACCAAAGGACGATATACGGGTAGAACGGTAAGAGAGGCCGCCCCTCAAT |
| | TGCGAGCCAGGCTTCACAACCTCCGTTCTACCGCTTCACCGACAACAGTCCTCAATCATG |
| | GACCGCGCCGTTAGCCAAGTTGCGTTAGAGAATGATGAAAGAGAGGCAAAAAATACATGG |
| | CGCTTGATATTCCGGATTGCAATCTTATTCTTAACAGTAGTGACCTTGGCTATATCTGTA |
| | GCCTCCCTTTTATATAGCATGGGGGCTAGCACACCTAGCGATCTTGTAGGCATACCGACT |
| | AGGATTTCCAGGGCAGAAGAAAAGATTACATCTACACTTGGTTCCAATCAAGATGTAGTA |
| | GATAGGATATATAAGCAAGTGGCCCTTGAGTCTCCGTTGGCATTGTTAAATACTGAGACC |
| | ACAATTATGAACGCAATAACATCTCTCTCTTATCAGATTAATGGAGCTGCAAACAACAGT |
| | GGGTGGGGGGCACCTATCCATGACCCAGATTATATAGGGGGGATAGGCAAAGAACTCATT |
| | GTAGATGATGCTAGTGATGTCACATCATTCTATCCCTCTGCATTTCAAGAACATCTGAAT |
| | TTTATCCCGGCGCCTACTACAGGATCAGGTTGCACTCGAATACCCTCATTTGACATGAGT |
| | GCTACCCATTACTGCTACACCCATAATGTAATATTGTCTGGATGCAGAGATCACTCACAT |
| | TCATATCAGTATTTAGCACTTGGTGTGCTCCGGACATCTGCAACAGGGAGGGTATTCTTT |
| | TCTACTCTGCGTTCCATCAACCTGGACGACACCCAAAATCGGAAGTCTTGCAGTGTGAGT |
| | GCAACTCCCCTGGGTTGTGATATGCTGTGCTCGAAAGTCACGGAGACAGAGGAAGAAGAT |
| | TATAACTCAGCTGTCCCTACGCGGATGGTACATGGGAGGTTAGGGTTCGACGGCCAGTAC |
| | CACGAAAAGGACCTAGATGTCACAACATTATTCGGGGACTGGGTGGCCAACTACCCAGGA |
| | GTAGGGGGTGGATCTTTTATTGACAGCCGCGTATGGTTCTCAGTCTACGGAGGGTTAAAA |
| | CCCAATTCACCCAGTGACACTGTACAGGAAGGGAAATATGTGATATACAAGCGATACAAT |
| | GACACATGCCCAGATGAGCAAGACTACCAGATTCGAATGGCCAAGTCTTCGTATAAGCCT |
| | GGACGGTTTGGTGGGAAACGCATACAGCAGGCTATCTTATCTATCAAGGTGTCAACATCC |
| | TTAGGCGAAGACCCGGTACTGACTGTACCGCCCAACACAGTCACACTCATGGGGGCCGAA |
| | GGCAGAATTCTCACAGTAGGGACATCTCATTTCTTGTATCAACGAGGGTCATCATACTTC |
| | TCTCCCGCGTTATTATATCCTATGACAGTCAGCAACAAAACAGCCACTCTTCATAGTCCT |
| | TATACATTCAATGCCTTCACTCGGCCAGGTAGTATCCCTTGCCAGGCTTCAGCAAGATGC |
| | CCCAACTCGTGTGTTACTGGAGTCTATACAGATCCATATCCCCTAATCTTCTATAGAAAC |
| | CACACCTTGCGAGGGGTATTCGGGACAATGCTTGATGGTGTACAAGCAAGACTTAACCCT |
| | GCGTCTGCAGTATTCGATAGCACATCCCGCAGTCGCATTACTCGAGTGAGTTCAAGCAGT |
| | ACCAAAGCAGCATACACAACATCAACTTGTTTTAAAGTGGTCAAGACTAATAAGACCTAT |
| | TGTCTCAGCATTGCTGAAATATCTAATACTCTCTTCGGAGAATTCAGAATCGTCCCGTTA |
| | CTAGTTGAGATCCTCAAAGATGACGGGGTTAGAGAAGCCAGGTCTGGCTAGggcgcgccT |
| | TGAGTCAATTATAAAGGAGTTGGAAAGATGGCATTGTATCACCTATCTTCTGCGACATCA |
| | AGAATCAAACCGAATGCCGGCGCGTGCTCGAATTCCATGTTGCCAGTTGACCACAATCAG |
| | CCAGTGCTCATGCGATCAGATTAAGCCTTGTCATTAATCTCTTGATTAAGAAAAAATGTA |
| | AGTGGCAATGAGATACAAGGCAAAACAGCTCATGGTAAATAATACGGGTAGGACATGGCG |
| | AGCTCCGGTCCTGAAAGGGCAGAGCATCAGATTATCCTACCAGAGCCACACCTGTCTTCA |
| | CCATTGGTCAAGCACAAACTACTCTATTACTGGAAATTAACTGGGCTACCGCTTCCTGAT |
| | GAATGTGACTTCGACCACCTCATTCTCAGCCGACAATGGAAAAAAATACTTGAATCGGCC |
| | TCTCCTGATACTGAGAGAATGATAAAACTCGGAAGGGCAGTACACCAAACTCTTAACCAC |
| | AATTCCAGAATAACCGGAGTGCTCCACCCCAGGTGTTTAGAACAACTGGCTAATATTGAG |
| | GTCCCAGATTCAACCAACAAATTTCGGAAGATTGAGAAGAAGATCCAAATTCACAACACG |
| | AGATATGGAGAACTGTTCACAAGGCTGTGTACGCATATAGAGAAGAAACTGCTGGGGTCA |
| | TCTTGGTCTAACAATGTCCCCCGGTCAGAGGAGTTCAGCAGCATTCGTACGGATCCGGCA |
| | TTCTGGTTTCACTCAAAATGGTCCACAGCCAAGTTTGCATGGCTCCATATAAAACAGATC |
| | CAGAGGCATCTGATGGTGGCAGCTAAGACAAGGTCTGCGGCCAACAAATTGGTGATGCTA |
| | ACCCATAAGGTAGGCCAAGTCTTTGTCACTCCTGAACTTGTCGTTGTGACGCATACGAAT |
| | GAGAACAAGTTCACATGTCTTACCCAGGAACTTGTATTGATGTATGCAGATATGATGGAG |
| | GGCAGAGATATGGTCAACATAATATCAACCACGGCGGTGCATCTCAGAAGCTTATCAGAG |
| | AAAATTGATGACATTTTGCGGTTAATAGACGCTCTGGCAAAAGACTTGGGTAATCAAGTC |
| | TACGATGTTGTATCACTAATGGAGGGATTTGCATACGGAGCTGTCCAGCTACTCGAGCCG |
| | TCAGGTACATTTGCAGGAGATTTCTTCGCATTCAACCTGCAGGAGCTTAAAGACATTCTA |
| | ATTGGCCTCCTCCCCAATGATATAGCAGAATCCGTGACTCATGCAATCGCTACTGTATTC |
| | TCTGGTTTAGAACAGAATCAAGCAGCTGAGATGTTGTGTCTGTTGCGTCTGTGGGGTCAC |
| | CCACTGCTTGAGTCCCGTATTGCAGCAAAGGCAGTCAGGAGCCAAATGTGCGCACCGAAA |
| | ATGGTAGACTTTGATATGATCCTTCAGGTACTGTCTTTCTTCAAGGGAACAATCATCAAC |
| | GGGTACAGAAAGAAGAATGCAGGTGTGTGGCCGCGAGTCAAAGTGGATACAATATATGGG |
| | AAGGTCATTGGGCAACTACATGCAGATTCAGCAGAGATTTCACACGATATCATGTTGAGA |
| | GAGTATAAGAGTTTATCTGCACTTGAATTTGAGCCATGTATAGAATATGACCCTGTCACC |
| | AACCTGAGCATGTTCCTAAAAGACAAGGCAATCGCACACCCCAACGATAATTGGCTTGCC |
| | TCGTTTAGGCGGAACCTTCTCTCCGAAGACCAGAAGAAACATGTAAAAGAAGCAACTTCG |
| | ACTAATCGCCTCTTGATAGAGTTTTTAGAGTCAAATGATTTTGATCCATATAAAGAGATG |
| | GAATATCTGACGACCCTTGAGTACCTTAGAGATGACAATGTGGCAGTATCATACTCGCTC |
| | AAGGAGAAGGAAGTGAAAGTTAATGGACGGATCTTCGCTAAGCTGACAAAGAAGTTAAGG |
| | AACTGTCAGGTGATGGCGGAAGGGATCCTAGCCGATCAGATTGCACCTTTCTTTCAGGGA |
| | AATGGAGTCATTCAGGATAGCATATCCTTGACCAAGAGTATGCTAGCGATGAGTCAACTG |
| | TCTTTTAACAGCAATAAGAAACGTATCACTGACTGTAAAGAAAGAGTATCTTCAAACCGC |
| | AATCATGATCCGAAAAGCAAGAACCGTCGGAGAGTTGCAACCTTCATAACAACTGACCTG |
| | CAAAAGTACTGTCTTAATTGGAGATATCAGACAATCAAATTGTTCGCTCATGCCATCAAT |
| | CAGTTGATGGGCCTACCTCACTTCTTCGAATGGATTCACCTAAGACTGATGGACACTACG |
| | ATGTTCGTAGGAGACCCTTTCAATCCTCCAAGTGACCCTACTGACTGTGACCTCTCAAGA |
| | GTCCCTAATGATGACATATATATTGTCAGTGCCAGAGGGGGTATCGAAGGATTATGCCAG |
| | AAGCTATGGACAATGATCTCAATTGCTGCAATCCAACTTGCTGCAGCTAGATCGCATTGT |
| | CGTGTTGCCTGTATGGTACAGGGTGATAATCAAGTAATAGCAGTAACGAGAGAGGTAAGA |
| | TCAGACGACTCTCCGGAGATGGTGTTGACACAGTTGCATCAAGCCAGTGATAATTTCTTC |
| | AAGGAATTAATTCATGTCAATCATTTGATTGGCCATAATTTGAAGGATCGTGAAACCATC |
| | AGGTCAGACACATTCTTCATATACAGCAAACGAATCTTCAAAGATGGAGCAATCCTCAGT |
| | CAAGTCCTCAAAAATTCATCTAAATTAGTGCTAGTGTCAGGTGATCTCAGTGAAAACACC |
| | GTAATGTCCTGTGCCAACATTGCCTCTACTGTAGCACGGCTATGCGAGAACGGGCTTCCC |
| | AAAGACTTCTGTTACTATTTAAACTATATAATGAGTTGTGTGCAGACATACTTTGACTCT |
| | GAGTTCTCCATCACCAACAATTCGCACCCCGATCTTAATCAGTCGTGGATTGAGGACATC |
| | TCTTTTGTGCACTCATATGTTCTGACTCCTGCCCAATTAGGGGGACTGAGTAACCTTCAA |
| | TACTCAAGGCTCTACACTAGAAATATCGGTGACCCGGGGACTACTGCTTTTGCAGAGATC |
| | AAGCGACTAGAAGCAGTGGGATTACTGAGTCCTAACATTATGACTAATATCTTAACTAGG |
| | CCGCCTGGGAATGGAGATTGGGCCAGTCTGTGCAACGACCCATACTCTTTCAATTTTGAG |
| | ACTGTTGCAAGCCCAAATATTGTTCTTAAGAAACATACGCAAAGAGTCCTATTTGAAACT |
| | TGTTCAAATCCCTTATTGTCTGGAGTGCACACAGAGGATAATGAGGCAGAAGAGAAGGCA |
| | TTGGCTGAATTCTTGCTTAATCAAGAGGTGATTCATCCCCGCGTTGCGCATGCCATCATG |
| | GAGGCAAGCTCTGTAGGTAGGAGAAAGCAAATTCAAGGGCTTGTTGACACAACAAACACC |
| | GTAATTAAGATTGCGCTTACTAGGAGGCCATTAGGCATCAAGAGGCTGATGCGGATAGTC |
| | AATTATTCTAGCATGCATGCAATGCTGTTTAGAGACGATGTTTTTTCCTCCAGTAGATCC |
| | AACCACCCCTTAGTCTCTTCTAATATGTGTTCTCTGACACTGGCAGACTATGCACGGAAT |
| | AGAAGCTGGTCACCTTTGACGGGAGGCAGGAAAATACTGGGTGTATCTAATCCTGATACG |
| | ATAGAACTCGTAGAGGGTGAGATTCTTAGTGTAAGCGGAGGGTGTACAAGATGTGACAGC |
| | GGAGATGAACAATTTACTTGGTTCCATCTTCCAAGCAATATAGAATTGACCGATGACACC |
| | AGCAAGAATCCTCCGATGAGGGTACCATATCTCGGGTCAAAGACACAGGAGAGGAGAGCT |
| | GCCTCACTTGCAAAAATAGCTCATATGTCGCCACATGTAAAGGCTGCCCTAAGGGCATCA |
| | TCCGTGTTGATCTGGGCTTATGGGGATAATGAAGTAAATTGGACTGCTGCTCTTACGATT |
| | GCAAAATCTCGGTGTAATGTAAACTTAGAGTATCTTCGGTTACTGTCCCCTTTACCCACG |
| | GCTGGGAATCTTCAACATAGACTAGATGATGGTATAACTCAGATGACATTCACCCCTGCA |
| | TCTCTCTACAGGgtgtcaccttacattcacatatccaatgattctcaaaggctgttcact |
| | gaagaaggagtcaaagaggggaatgtggtttaccaacagatcATGCTCTTGGGTTTATCT |
| | CTAATCGAATCGATCTTTCCAATGACAACAACCAGGACATATGATGAGATCACACTGCAC |
| | CTACATAGTAAATTTAGTTGCTGTATCAGAGAAGCACCTGTTGCGGTTCCTTTCGAGCTA |
| | CTTGGGGTGGTACCGGAACTGAGGACAGTGACCTCAAATAAGTTTATGTATGATCCTAGC |
| | CCTGTATCGGAGGGAGACTTTGCGAGACTTGACTTAGCTATCTTCAAGAGTTATGAGCTT |
| | AATCTGGAGTCATATCCCACGATAGAGCTAATGAACATTCTTTCAATATCCAGCGGGAAG |
| | TTGATTGGCCAGTCTGTGGTTTCTTATGATGAAGATACCTCCATAAAGAATGACGCCATA |
| | ATAGTGTATGACAATACCCGAAATTGGATCAGTGAAGCTCAGAATTCAGATGTGGTCCGC |
| | CTATTTGAATATGCAGCACTTGAAGTGCTCCTCGACTGTTCTTACCAACTCTATTACCTG |
| | AGAGTAAGAGGCCTAGACAATATTGTCTTATATATGGGTGATTTATACAAGAATATGCCA |
| | GGAATTCTACTTTCCAACATTGCAGCTACAATATCTCATCCCGTCATTCATTCAAGGTTA |
| | CATGCAGTGGGCCTGGTCAACCATGACGGATCACACCAACTTGCAGATACGGATTTTATC |
| | GAAATGTCTGCAAAACTATTAGTATCTTGCACCCGACGTGTGATCTCCGGCTTATATTCA |
| | GGAAATAAGTATGATCTGCTGTTCCCATCTGTCTTAGATGATAACCTGAATGAGAAGATG |
| | CTTCAGCTGATATCCCGGTTATGCTGTCTGTACACGGTACTCTTTGCTACAACAAGAGAA |
| | ATCCCGAAAATAAGAGGCTTAACTGCAGAAGAGAAATGTTCAATACTCACTGAGTATTTA |
| | CTGTCGGATGCTGTGAAACCATTACTTAGCCCCGATCAAGTGAGCTCTATCATGTCTCCT |
| | AACATAATTACATTCCCAGCTAATCTGTACTACATGTCTCGGAAGAGCCTCAATTTGATC |
| | AGGGAAAGGGAGGACAGGGATACTATCCTGGCGTTGTTGTTCCCCCAAGAGCCATTATTA |
| | GAGTTCCCTTCTGTGCAAGATATTGGTGCTCGAGTGAAAGATCCATTCACCCGACAACCT |
| | GCGGCATTTTTGCAAGAGTTAGATTTGAGTGCTCCAGCAAGGTATGACGCATTCACACTT |
| | AGTCAGATTCATCCTGAACTCACATCTCCAAATCCGGAGGAAGACTACTTAGTACGATAC |
| | TTGTTCAGAGGGATAGGGACTGCATCTTCCTCTTGGTATAAGGCATCTCATCTCCTTTCT |
| | GTACCCGAGGTAAGATGTGCAAGACACGGGAACTCCTTATACTTAGCTGAAGGGAGCGGA |
| | GCCATCATGAGTCTTCTCGAACTGCATGTACCACATGAAACTATCTATTACAATACGCTC |
| | TTTTCAAATGAGATGAACCCCCCGCAACGACATTTCGGGCCGACCCCAACTCAGTTTTTG |
| | AATTCGGTTGTTTATAGGAATCTACAGGCGGAGGTAACATGCAAAGATGGATTTGTCCAA |
| | GAGTTCCGTCCATTATGGAGAGAAAATACAGAGGAAAGTGACCTGACCTCAGATAAAGCA |
| | GTGGGGTATATTACATCTGCAGTGCCCTACAGATCTGTATCATTGCTGCATTGTGACATT |
| | GAAATTCCTCCAGGGTCCAATCAAAGCTTACTAGATCAACTAGCTATCAATTTATCTCTG |
| | ATTGCCATGCATTCTGTAAGGGAGGGCGGGGTAGTAATCATCAAAGTGTTGTATGCAATG |
| | GGATACTACTTTCATCTACTCATGAACTTGTTTGCTCCGTGTTCCACAAAAGGATATATT |
| | CTCTCTAATGGTTATGCATGTCGAGGAGATATGGAGTGTTACCTGGTATTTGTCATGGGT |
| | TACCTGGGCGGGCCTACATTTGTACATGAGGTGGTGAGGATGGCAAAAACTCTGGTGCAG |
| | CGGCACGGTACGCTCTTGTCTAAATCAGATGAGATCACACTGACCAGGTTATTCACCTCA |
| | CAGCGGCAGCGTGTGACAGACATCCTATCCAGTCCTTTACCAAGATTAATAAAGTACTTG |
| | AGGAAGAATATTGACACTGCGCTGATTGAAGCCGGGGGACAGCCCGTCCGTCCATTCTGT |
| | GCGGAGAGTCTGGTGAGCACGCTAGCGAACATAACTCAGATAACCCAGATTATCGCTAGT |
| | CACATTGACACAGTTATCCGGTCTGTGATATATATGGAAGCTGAGGGTGATCTCGCTGAC |
| | ACAGTATTTCTATTTACCCCTTACAATCTCTCTACTGACGGGAAAAAGAGGACATCACTT |
| | ATACAGTGCACGAGACAGATCCTAGAGGTTACAATACTAGGTCTTAGAGTCGAAAATCTC |
| | AATAAAATAGGCGATATAATCAGCCTAGTGCTTAAAGGCATGATCTCCATGGAGGACCTT |
| | ATCCCACTAAGGACATACTTGAAGCATAGTACCTGCCCTAAATATTTGAAGGCTGTCCTA |
| | GGTATTACCAAACTCAAAGAAATGTTTACAGACACTTCTGTATTGTACTTGACTCGTGCT |
| | CAACAAAAATTCTACATGAAAACTATAGGCAATGCAGTCAAAGGATATTACAGTAACTGT |
| | GACTCTTAACGAAAATCACATATTAATAGGCTCCTTTTTTGGCCAATTGTATTCTTGTTG |
| | ATTTAATCATATTATGTTAGAAAAAAGTTGAACCCTGACTCCTTAGGACTCGAATTCGAA |
| | CTCAAATAAATGTCTTAAAAAAAGGTTGCGCACAATTATTCTTGAGTGTAGTCTCGTCAT |
| | TCACCAAATCTTTGTTTGGTGGCCGGCATGGTCCCAGCCTCCTCGCTGGCGCCGGCTGGG |
| | CAACATTCCGAGGGGACCGTCCCCTCGGTAATGGCGAATGGGACGTCGACTGCTAACAAA |
| | GCCCGAAAGGAAGCTGAGTTGGCTGCTGCCACCGCTGAGCAATAACTAGCATAACCCCTT |
| | GGGGCCTCTAAACGGGTCTTGAGGGGTTTTTTGCTGAAAGGAGGAACTATAT |
| SEQ ID NO: | TAATACGACTCACTATAGGGACCAAACAGAGAATCCGTGAGTTACGATAAAAGGCGAAGG |
| 2; nucleotide | AGCAATTGAAGTCGCACGGGTAGAAGGTGTGAATCTCGAGTGCGAGCCCGAAGCACAAAC |
| sequence of | TCGAGAAAGCCTTCTGCCAACATGTCTTCCGTATTTGATGAGTACGAACAGCTCCTCGCG |
| NDV-FLS | GCTCAGACTCGCCCCAATGGAGCTCATGGAGGGGGAGAAAAAGGGAGTACCTTAAAAGTA |
| Molecular | GACGTCCCGGTATTCACTCTTAACAGTGATGACCCAGAAGATAGATGGAGCTTTGTGGTA |
| Clone | TTCTGCCTCCGGATTGCTGTTAGCGAAGATGCCAACAAACCACTCAGGCAAGGTGCTCTC |
| AF077761.1_ | ATATCTCTTTTATGCTCCCACTCACAGGTAATGAGGAACCATGTTGCCATTGCAGGGAAA |
| LaSota_Kan | CAGAATGAAGCCACATTGGCCGTGCTTGAGATTGATGGCTTTGCCAACGGCACGCCCCAG |
| R (with | TTCAACAATAGGAGTGGAGTGTCTGAAGAGAGAGCACAGAGATTTGCGATGATAGCAGGA |
| stabilizing | TCTCTCCCTCGGGCATGCAGCAACGGAACCCCGTTCGTCACAGCCGGGGCAGAAGATGAT |
| sequence in | GCACCAGAAGACATCACCGATACCCTGGAGAGGATCCTCTCTATCCAGGCTCAAGTATGG |
| L) | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaTTAGAAAAAATA |
| | CGGGTAGAACCGCCACCATGTTCGTGTTCCTGGTCCTGCTGCCACTGGTAAGCTCCCAAT |
| | GTGTAAACTTAACCACAAGAACCCAGCTCCCACCTGCCTACACCAACAGCTTCACCAGAG |
| | GCGTTTATTACCCCGACAAGGTATTCCGGTCTTCTGTTCTGCACTCTACCCAGGACCTGT |
| | TTCTGCCCTTTTTCAGCAACGTGACATGGTTCCACGCCATCCACGTGTCTGGCACAAACG |
| | GCACCAAGCGGTTTGATAATCCTGTGCTCCCTTTCAATGACGGCGTGTACTTCGCCTCTA |
| | CTGAGAAGAGCAACATCATCCGGGGCTGGATCTTTGGCACAACACTGGACTCTAAAACCC |
| | AGAGCCTGCTGATCGTGAACAACGCCACCAACGTGGTGATTAAGGTGTGCGAGTTCCAGT |
| | TCTGCAATGACCCTTTCCTCGGCGTGTACTACCACAAGAACAACAAAAGTTGGATGGAAA |
| | GCGAATTCAGGGTGTACTCAAGCGCCAACAACTGTACCTTCGAGTACGTGAGCCAGCCTT |
| | TCCTGATGGACCTAGAAGGTAAGCAGGGCAATTTCAAGAACCTCAGAGAGTTCGTGTTCA |
| | AGAATATTGACGGCTACTTCAAAATCTACAGCAAGCACACCCCAATCAACCTGGTGCGGG |
| | ACCTGCCCCAGGGCTTTAGCGCGCTGGAGCCTCTGGTGGACCTGCCTATCGGCATCAACA |
| | TCACCCGGTTCCAGACACTGCTGGCTCTGCATAGAAGCTACCTGACACCTGGCGACAGTT |
| | CTTCTGGCTGGACAGCCGGCGCCGCCGCCTACTACGTGGGCTACCTGCAGCCTAGAACAT |
| | TCCTGCTGAAATACAACGAGAACGGCACGATCACAGACGCCGTGGACTGCGCCCTGGATC |
| | CCCTGTCTGAGACAAAGTGCACCCTGAAGTCTTTCACCGTGGAGAAGGGCATCTACCAGA |
| | CCTCCAACTTCAGAGTGCAGCCTACCGAATCCATCGTGCGCTTTCCCAACATCACCAACC |
| | TGTGCCCCTTCGGCGAGGTCTTTAATGCCACGAGATTCGCCAGCGTGTATGCCTGGAACA |
| | GAAAGAGAATCAGCAACTGCGTGGCCGACTACAGCGTGCTGTACAACAGCGCCTCTTTCA |
| | GCACATTTAAGTGCTACGGAGTGTCTCCTACCAAACTCAACGATCTGTGCTTCACGAACG |
| | TGTATGCCGACAGCTTCGTGATCCGAGGAGATGAGGTGCGGCAGATCGCTCCAGGACAGA |
| | CAGGCAAGATCGCCGACTACAACTACAAGCTGCCCGACGACTTTACCGGCTGCGTGATCG |
| | CTTGGAACAGCAATAACCTGGACTCAAAGGTTGGAGGAAACTACAACTACCTGTACAGAC |
| | TGTTCAGAAAGTCCAACCTGAAGCCCTTCGAGAGAGACATCTCTACAGAAATCTACCAGG |
| | CCGGCAGCACCCCATGTAACGGCGTGGAAGGCTTCAACTGCTACTTCCCTCTGCAGTCTT |
| | ATGGCTTCCAGCCCACAAACGGAGTGGGCTATCAGCCTTACCGCGTGGTTGTCCTGAGCT |
| | TTGAGCTGCTGCATGCCCCTGCTACGGTGTGTGGACCTAAGAAGTCCACCAACCTGGTGA |
| | AGAACAAGTGTGTGAACTTCAACTTCAACGGCCTGACCGGCACCGGGGTGCTGACAGAGT |
| | CTAACAAGAAATTCCTGCCATTCCAGCAATTCGGCCGGGACATCGCCGACACCACCGACG |
| | CCGTGCGGGATCCTCAGACCCTCGAAATCCTGGACATCACCCCCTGTAGCTTCGGCGGCG |
| | TGAGCGTGATCACCCCTGGCACAAACACCAGCAATCAAGTGGCTGTCCTGTACCAGGATG |
| | TCAATTGCACAGAAGTGCCTGTGGCCATCCACGCCGATCAGCTGACCCCCACCTGGCGGG |
| | TGTACTCGACAGGAAGCAACGTGTTTCAAACAAGAGCCGGCTGCCTGATCGGGGCCGAGC |
| | ACGTGAACAATTCCTACGAGTGCGACATCCCCATCGGCGCCGGCATCTGTGCCTCTTACC |
| | AGACACAGACCAATTCCCCTCGTAGAGCCAGATCCGTGGCCAGCCAGAGCATCATCGCCT |
| | ACACCATGAGCCTGGGCGCCGAAAACAGCGTTGCATATTCCAACAACAGCATCGCCATCC |
| | CTACCAACTTCACCATCAGCGTGACCACAGAAATCCTGCCTGTGTCCATGACCAAGACAA |
| | GCGTTGATTGCACCATGTACATCTGCGGCGATAGCACAGAGTGCAGCAATCTGCTGCTGC |
| | AGTACGGTAGCTTCTGCACCCAGCTGAATAGAGCCCTGACCGGCATCGCTGTGGAACAGG |
| | ACAAAAACACCCAGGAGGTCTTCGCCCAGGTGAAGCAAATCTACAAGACCCCTCCAATCA |
| | AGGACTTCGGAGGCTTTAACTTTAGCCAGATCCTGCCTGATCCCTCCAAGCCTAGCAAAC |
| | GGAGTTTCATCGAGGACCTGCTCTTCAACAAGGTGACCCTGGCTGACGCCGGCTTCATTA |
| | AGCAGTACGGCGATTGCCTCGGCGACATCGCTGCAAGAGACCTGATCTGCGCCCAGAAGT |
| | TCAACGGCCTGACCGTGCTGCCTCCTCTCCTGACAGACGAGATGATCGCCCAGTACACCT |
| | CTGCCCTTCTGGCTGGCACCATCACCAGCGGATGGACCTTTGGAGCCGGAGCCGCCCTGC |
| | AGATCCCTTTCGCTATGCAGATGGCCTACAGATTCAACGGGATCGGAGTGACCCAAAACG |
| | TGCTGTATGAAAACCAGAAACTGATCGCCAATCAGTTTAACAGCGCCATCGGCAAAATCC |
| | AGGATAGCCTGTCCAGCACCGCCAGCGCCCTCGGCAAGCTGCAAGATGTGGTGAATCAAA |
| | ATGCCCAAGCCCTGAACACACTGGTGAAGCAGCTGAGCAGCAACTTCGGCGCCATCAGCA |
| | GCGTGCTGAACGACATCCTGAGCAGACTGGACAAGGTGGAAGCCGAGGTGCAGATCGACA |
| | GACTGATCACAGGCAGACTGCAGTCCCTGCAGACCTACGTGACCCAGCAGTTGATTAGAG |
| | CCGCTGAGATTAGAGCCAGTGCCAACCTGGCTGCCACAAAGATGTCAGAATGCGTGCTGG |
| | GCCAGAGCAAGAGAGTGGACTTCTGCGGCAAAGGCTACCACCTGATGAGCTTTCCTCAGT |
| | CTGCACCCCACGGCGTGGTGTTTCTCCACGTGACATACGTGCCCGCGCAAGAAAAGAACT |
| | TTACAACCGCCCCAGCGATCTGCCACGACGGCAAGGCCCACTTCCCTCGGGAGGGTGTGT |
| | TCGTGAGCAATGGAACACACTGGTTCGTCACCCAGCGGAACTTCTACGAGCCTCAGATCA |
| | TTACCACCGACAACACCTTCGTGAGCGGCAACTGTGACGTCGTTATCGGCATCGTGAACA |
| | ATACCGTGTACGACCCCCTGCAGCCTGAGCTGGATAGCTTCAAAGAGGAACTGGACAAGT |
| | ACTTCAAGAACCACACAAGCCCCGACGTGGACCTAGGCGACATCTCTGGAATCAACGCCA |
| | GCGTGGTGAACATCCAAAAGGAAATCGACAGACTGAACGAGGTGGCCAAGAATCTGAATG |
| | AAAGCCTGATCGATCTGCAGGAGCTGGGCAAGTACGAGCAGTACATCAAATGGCCTTGGT |
| | ACATCTGGCTGGGCTTCATCGCTGGTCTGATCGCTATCGTGATGGTGACCATTATGCTGT |
| | GCTGCATGACCTCCTGCTGCTCTTGCCTGAAGGGCTGTTGTTCTTGCGGCTCTTGCTGCA |
| | AGTTCGACGAGGATGACTCTGAACCTGTTCTGAAGGGCGTGAAGCTGCACTACACCTGAT |
| | aaacgcgtACCCAAGGTCCAACTCTCCAAGCGGCAATCCTCTCTCGCTTCCTCAGCCCCA |
| | CTGAATGGTCGCGTAACCGTAATTAATCTAGCTACATTTAAGATTAAGAAAAAATACGGG |
| | TAGAATTGGAGTGCCCCAATTGTGCCAAGATGGACTCATCTAGGACAATTGGGCTGTACT |
| | TTGATTCTGCCCATTCTTCTAGCAACCTGTTAGCATTTCCGATCGTCCTACAAGGCACAG |
| | GAGATGGGAAGAAGCAAATCGCCCCGCAATATAGGATCCAGCGCCTTGACTTGTGGACTG |
| | ATAGTAAGGAGGACTCAGTATTCATCACCACCTATGGATTCATCTTTCAAGTTGGGAATG |
| | AAGAAGCCACTGTCGGCATGATCGATGATAAACCCAAGCGCGAGTTACTTTCCGCTGCGA |
| | TGCTCTGCCTAGGAAGCGTCCCAAATACCGGAGACCTTATTGAGCTGGCAAGGGCCTGTC |
| | TCACTATGATAGTCACATGCAAGAAGAGTGCAACTAATACTGAGAGAATGGTTTTCTCAG |
| | TAGTGCAGGCACCCCAAGTGCTGCAAAGCTGTAGGGTTGTGGCAAACAAATACTCATCAG |
| | TGAATGCAGTCAAGCACGTGAAAGCGCCAGAGAAGATTCCCGGGAGTGGAACCCTAGAAT |
| | ACAAGGTGAACTTTGTCTCCTTGACTGTGGTACCGAAGAAGGATGTCTACAAGATCCCAG |
| | CTGCAGTATTGAAGGTTTCTGGCTCGAGTCTGTACAATCTTGCGCTCAATGTCACTATTA |
| | ATGTGGAGGTAGACCCGAGGAGTCCTTTGGTTAAATCTTTGTCTAAGTCTGACAGCGGAT |
| | ACTATGCTAACCTCTTCTTGCATATTGGACTTATGACCACCGTAGATAGGAAGGGGAAGA |
| | AAGTGACATTTGACAAGCTGGAAAAGAAAATAAGGAGCCTTGATCTATCTGTCGGGCTCA |
| | GTGATGTGCTCGGGCCTTCCGTGTTGGTAAAAGCAAGAGGTGCACGGACTAAGCTTTTGG |
| | CACCTTTCTTCTCTAGCAGTGGGACAGCCTGCTATCCCATAGCAAATGCTTCTCCTCAGG |
| | TGGCCAAGATACTCTGGAGTCAAACCGCGTGCCTGCGGAGCGTTAAAATCATTATCCAAG |
| | CAGGTACCCAACGCGCTGTCGCAGTGACCGCCGACCACGAGGTTACCTCTACTAAGCTGG |
| | AGAAGGGGCACACCCTTGCCAAATACAATCCTTTTAAGAAATAAGCTGCGTCTCTGAGAT |
| | TGCGCTCCGCCCACTCACCCAGATCATCATGACACAAAAAACTAATCTGTCTTGATTATT |
| | TACAGTTAGTTTACCTGTCTATCAAGTTAGAAAAAACACGGGTAGAAGATTCTGGATCCC |
| | GGTTGGCGCCCTCCAGGTGCAAGttaattaaATGGGCTCCAGACCTTCTACCAAGAACCC |
| | AGCACCTATGATGCTGACTATCCGGGTTGCGCTGGTACTGAGTTGCATCTGTCCGGCAAA |
| | CTCCATTGATGGCAGGCCTCTTGCAGCTGCAGGAATTGTGGTTACAGGAGACAAAGCCGT |
| | CAACATATACACCTCATCCCAGACAGGATCAATCATAGTTAAGCTCCTCCCGAATCTGCC |
| | CAAGGATAAGGAGGCATGTGCGAAAGCCCCCTTGGATGCATACAACAGGACATTGACCAC |
| | TTTGCTCACCCCCCTTGGTGACTCTATCCGTAGGATACAAGAGTCTGTGACTACATCTGG |
| | AGGGGGGAGACAGGGGCGCCTTATAGGCGCCATTATTGGCGGTGTGGCTCTTGGGGTTGC |
| | AACTGCCGCACAAATAACAGCGGCCGCAGCTCTGATACAAGCCAAACAAAATGCTGCCAA |
| | CATCCTCCGACTTAAAGAGAGCATTGCCGCAACCAATGAGGCTGTGCATGAGGTCACTGA |
| | CGGATTATCGCAACTAGCAGTGGCAGTTGGGAAGATGCAGCAGTTTGTTAATGACCAATT |
| | TAATAAAACAGCTCAGGAATTAGACTGCATCAAAATTGCACAGCAAGTTGGTGTAGAGCT |
| | CAACCTGTACCTAACCGAATTGACTACAGTATTCGGACCACAAATCACTTCACCTGCTTT |
| | AAACAAGCTGACTATTCAGGCACTTTACAATCTAGCTGGTGGAAATATGGATTACTTATT |
| | GACTAAGTTAGGTGTAGGGAACAATCAACTCAGCTCATTAATCGGTAGCGGCTTAATCAC |
| | tGGcAACCCTATTCTATACGACTCACAGACTCAACTCTTGGGTATACAGGTAACTgcaCC |
| | TTCAGTCGGGAACCTAAATAATATGCGTGCCACCTACTTGGAAACCTTATCCGTAAGCAC |
| | AACCAGGGGATTTGCCTCGGCACTTGTCCCCAAAGTGGTGACACAGGTCGGTTCTGTGAT |
| | AGAAGAACTTGACACCTCATACTGTATAGAAACTGACTTAGATTTATATTGTACAAGAAT |
| | AGTAACGTTCCCTATGTCCCCTGGTATTTATTCCTGCTTGAGCGGCAATACGTCGGCCTG |
| | TATGTACTCAAAGACCGAAGGCGCACTTACTACACCATACATGACTATCAAAGGTTCAGT |
| | CATCGCCAACTGCAAGATGACAACATGTAGATGTGTAAACCCCCCGGGTATCATATCGCA |
| | AAACTATGGAGAAGCCGTGTCTCTAATAGATAAACAATCATGCAATGTTTTATCCTTAGG |
| | CGGGATAACTTTAAGGCTCAGTGGGGAATTCGATGTAACTTATCAGAAGAATATCTCAAT |
| | ACAAGATTCTCAAGTAATAATAACAGGCAATCTTGATATCTCAACTGAGCTTGGGAATGT |
| | CAACAACTCGATCAGTAATGCTTTGAATAAGTTAGAGGAAAGCAACAGAAAACTAGACAA |
| | AGTCAATGTCAAACTGACTAGCACATCTGCTCTCATTACgTATATCGTTTTGACTATCAT |
| | ATCTCTTGTTTTTGGTATACTTAGCCTGATTCTAGCATGCTACCTAATGTACAAGCAAAA |
| | GGCGCAACAAAAGACCTTATTATGGCTTGGGAATAATACaCTcGATCAGATGAGAGCCAC |
| | TACAAAAATGTGAACACAGATGAGGAACGAAGGTTTCCCTAATAGTAATTTGTGTGAAAG |
| | TTCTGGTAGTCTGTCAGTTCAGAGAGTTAAGAAAAAACTACCGGTTGTAGATGACCAAAG |
| | GACGATATACGGGTAGAACGGTAAGAGAGGCCGCCCCTCAATTGCGAGCCAGGCTTCACA |
| | ACCTCCGTTCTACCGCTTCACCGACAACAGTCCTCAATCATGGACCGCGCCGTTAGCCAA |
| | GTTGCGTTAGAGAATGATGAAAGAGAGGCAAAAAATACATGGCGCTTGATATTCCGGATT |
| | GCAATCTTATTCTTAACAGTAGTGACCTTGGCTATATCTGTAGCCTCCCTTTTATATAGC |
| | ATGGGGGCTAGCACACCTAGCGATCTTGTAGGCATACCGACTAGGATTTCCAGGGCAGAA |
| | GAAAAGATTACATCTACACTTGGTTCCAATCAAGATGTAGTAGATAGGATATATAAGCAA |
| | GTGGCCCTTGAGTCTCCGTTGGCATTGTTAAATACTGAGACCACAATTATGAACGCAATA |
| | ACATCTCTCTCTTATCAGATTAATGGAGCTGCAAACAACAGTGGGTGGGGGGCACCTATC |
| | CATGACCCAGATTATATAGGGGGGATAGGCAAAGAACTCATTGTAGATGATGCTAGTGAT |
| | GTCACATCATTCTATCCCTCTGCATTTCAAGAACATCTGAATTTTATCCCGGCGCCTACT |
| | ACAGGATCAGGTTGCACTCGAATACCCTCATTTGACATGAGTGCTACCCATTACTGCTAC |
| | ACCCATAATGTAATATTGTCTGGATGCAGAGATCACTCACATTCATATCAGTATTTAGCA |
| | CTTGGTGTGCTCCGGACATCTGCAACAGGGAGGGTATTCTTTTCTACTCTGCGTTCCATC |
| | AACCTGGACGACACCCAAAATCGGAAGTCTTGCAGTGTGAGTGCAACTCCCCTGGGTTGT |
| | GATATGCTGTGCTCGAAAGTCACGGAGACAGAGGAAGAAGATTATAACTCAGCTGTCCCT |
| | ACGCGGATGGTACATGGGAGGTTAGGGTTCGACGGCCAGTACCACGAAAAGGACCTAGAT |
| | GTCACAACATTATTCGGGGACTGGGTGGCCAACTACCCAGGAGTAGGGGGTGGATCTTTT |
| | ATTGACAGCCGCGTATGGTTCTCAGTCTACGGAGGGTTAAAACCCAATTCACCCAGTGAC |
| | ACTGTACAGGAAGGGAAATATGTGATATACAAGCGATACAATGACACATGCCCAGATGAG |
| | CAAGACTACCAGATTCGAATGGCCAAGTCTTCGTATAAGCCTGGACGGTTTGGTGGGAAA |
| | CGCATACAGCAGGCTATCTTATCTATCAAGGTGTCAACATCCTTAGGCGAAGACCCGGTA |
| | CTGACTGTACCGCCCAACACAGTCACACTCATGGGGGCCGAAGGCAGAATTCTCACAGTA |
| | GGGACATCTCATTTCTTGTATCAACGAGGGTCATCATACTTCTCTCCCGCGTTATTATAT |
| | CCTATGACAGTCAGCAACAAAACAGCCACTCTTCATAGTCCTTATACATTCAATGCCTTC |
| | ACTCGGCCAGGTAGTATCCCTTGCCAGGCTTCAGCAAGATGCCCCAACTCGTGTGTTACT |
| | GGAGTCTATACAGATCCATATCCCCTAATCTTCTATAGAAACCACACCTTGCGAGGGGTA |
| | TTCGGGACAATGCTTGATGGTGTACAAGCAAGACTTAACCCTGCGTCTGCAGTATTCGAT |
| | AGCACATCCCGCAGTCGCATTACTCGAGTGAGTTCAAGCAGTACCAAAGCAGCATACACA |
| | ACATCAACTTGTTTTAAAGTGGTCAAGACTAATAAGACCTATTGTCTCAGCATTGCTGAA |
| | ATATCTAATACTCTCTTCGGAGAATTCAGAATCGTCCCGTTACTAGTTGAGATCCTCAAA |
| | GATGACGGGGTTAGAGAAGCCAGGTCTGGCTAGggcgcgccTTGAGTCAATTATAAAGGA |
| | GTTGGAAAGATGGCATTGTATCACCTATCTTCTGCGACATCAAGAATCAAACCGAATGCC |
| | GGCGCGTGCTCGAATTCCATGTTGCCAGTTGACCACAATCAGCCAGTGCTCATGCGATCA |
| | GATTAAGCCTTGTCATTAATCTCTTGATTAAGAAAAAATGTAAGTGGCAATGAGATACAA |
| | GGCAAAACAGCTCATGGTAAATAATACGGGTAGGACATGGCGAGCTCCGGTCCTGAAAGG |
| | GCAGAGCATCAGATTATCCTACCAGAGCCACACCTGTCTTCACCATTGGTCAAGCACAAA |
| | CTACTCTATTACTGGAAATTAACTGGGCTACCGCTTCCTGATGAATGTGACTTCGACCAC |
| | CTCATTCTCAGCCGACAATGGAAAAAAATACTTGAATCGGCCTCTCCTGATACTGAGAGA |
| | ATGATAAAACTCGGAAGGGCAGTACACCAAACTCTTAACCACAATTCCAGAATAACCGGA |
| | GTGCTCCACCCCAGGTGTTTAGAACAACTGGCTAATATTGAGGTCCCAGATTCAACCAAC |
| | AAATTTCGGAAGATTGAGAAGAAGATCCAAATTCACAACACGAGATATGGAGAACTGTTC |
| | ACAAGGCTGTGTACGCATATAGAGAAGAAACTGCTGGGGTCATCTTGGTCTAACAATGTC |
| | CCCCGGTCAGAGGAGTTCAGCAGCATTCGTACGGATCCGGCATTCTGGTTTCACTCAAAA |
| | TGGTCCACAGCCAAGTTTGCATGGCTCCATATAAAACAGATCCAGAGGCATCTGATGGTG |
| | GCAGCTAAGACAAGGTCTGCGGCCAACAAATTGGTGATGCTAACCCATAAGGTAGGCCAA |
| | GTCTTTGTCACTCCTGAACTTGTCGTTGTGACGCATACGAATGAGAACAAGTTCACATGT |
| | CTTACCCAGGAACTTGTATTGATGTATGCAGATATGATGGAGGGCAGAGATATGGTCAAC |
| | ATAATATCAACCACGGCGGTGCATCTCAGAAGCTTATCAGAGAAAATTGATGACATTTTG |
| | CGGTTAATAGACGCTCTGGCAAAAGACTTGGGTAATCAAGTCTACGATGTTGTATCACTA |
| | ATGGAGGGATTTGCATACGGAGCTGTCCAGCTACTCGAGCCGTCAGGTACATTTGCAGGA |
| | GATTTCTTCGCATTCAACCTGCAGGAGCTTAAAGACATTCTAATTGGCCTCCTCCCCAAT |
| | GATATAGCAGAATCCGTGACTCATGCAATCGCTACTGTATTCTCTGGTTTAGAACAGAAT |
| | CAAGCAGCTGAGATGTTGTGTCTGTTGCGTCTGTGGGGTCACCCACTGCTTGAGTCCCGT |
| | ATTGCAGCAAAGGCAGTCAGGAGCCAAATGTGCGCACCGAAAATGGTAGACTTTGATATG |
| | ATCCTTCAGGTACTGTCTTTCTTCAAGGGAACAATCATCAACGGGTACAGAAAGAAGAAT |
| | GCAGGTGTGTGGCCGCGAGTCAAAGTGGATACAATATATGGGAAGGTCATTGGGCAACTA |
| | CATGCAGATTCAGCAGAGATTTCACACGATATCATGTTGAGAGAGTATAAGAGTTTATCT |
| | GCACTTGAATTTGAGCCATGTATAGAATATGACCCTGTCACCAACCTGAGCATGTTCCTA |
| | AAAGACAAGGCAATCGCACACCCCAACGATAATTGGCTTGCCTCGTTTAGGCGGAACCTT |
| | CTCTCCGAAGACCAGAAGAAACATGTAAAAGAAGCAACTTCGACTAATCGCCTCTTGATA |
| | GAGTTTTTAGAGTCAAATGATTTTGATCCATATAAAGAGATGGAATATCTGACGACCCTT |
| | GAGTACCTTAGAGATGACAATGTGGCAGTATCATACTCGCTCAAGGAGAAGGAAGTGAAA |
| | GTTAATGGACGGATCTTCGCTAAGCTGACAAAGAAGTTAAGGAACTGTCAGGTGATGGCG |
| | GAAGGGATCCTAGCCGATCAGATTGCACCTTTCTTTCAGGGAAATGGAGTCATTCAGGAT |
| | AGCATATCCTTGACCAAGAGTATGCTAGCGATGAGTCAACTGTCTTTTAACAGCAATAAG |
| | AAACGTATCACTGACTGTAAAGAAAGAGTATCTTCAAACCGCAATCATGATCCGAAAAGC |
| | AAGAACCGTCGGAGAGTTGCAACCTTCATAACAACTGACCTGCAAAAGTACTGTCTTAAT |
| | TGGAGATATCAGACAATCAAATTGTTCGCTCATGCCATCAATCAGTTGATGGGCCTACCT |
| | CACTTCTTCGAATGGATTCACCTAAGACTGATGGACACTACGATGTTCGTAGGAGACCCT |
| | TTCAATCCTCCAAGTGACCCTACTGACTGTGACCTCTCAAGAGTCCCTAATGATGACATA |
| | TATATTGTCAGTGCCAGAGGGGGTATCGAAGGATTATGCCAGAAGCTATGGACAATGATC |
| | TCAATTGCTGCAATCCAACTTGCTGCAGCTAGATCGCATTGTCGTGTTGCCTGTATGGTA |
| | CAGGGTGATAATCAAGTAATAGCAGTAACGAGAGAGGTAAGATCAGACGACTCTCCGGAG |
| | ATGGTGTTGACACAGTTGCATCAAGCCAGTGATAATTTCTTCAAGGAATTAATTCATGTC |
| | AATCATTTGATTGGCCATAATTTGAAGGATCGTGAAACCATCAGGTCAGACACATTCTTC |
| | ATATACAGCAAACGAATCTTCAAAGATGGAGCAATCCTCAGTCAAGTCCTCAAAAATTCA |
| | TCTAAATTAGTGCTAGTGTCAGGTGATCTCAGTGAAAACACCGTAATGTCCTGTGCCAAC |
| | ATTGCCTCTACTGTAGCACGGCTATGCGAGAACGGGCTTCCCAAAGACTTCTGTTACTAT |
| | TTAAACTATATAATGAGTTGTGTGCAGACATACTTTGACTCTGAGTTCTCCATCACCAAC |
| | AATTCGCACCCCGATCTTAATCAGTCGTGGATTGAGGACATCTCTTTTGTGCACTCATAT |
| | GTTCTGACTCCTGCCCAATTAGGGGGACTGAGTAACCTTCAATACTCAAGGCTCTACACT |
| | AGAAATATCGGTGACCCGGGGACTACTGCTTTTGCAGAGATCAAGCGACTAGAAGCAGTG |
| | GGATTACTGAGTCCTAACATTATGACTAATATCTTAACTAGGCCGCCTGGGAATGGAGAT |
| | TGGGCCAGTCTGTGCAACGACCCATACTCTTTCAATTTTGAGACTGTTGCAAGCCCAAAT |
| | ATTGTTCTTAAGAAACATACGCAAAGAGTCCTATTTGAAACTTGTTCAAATCCCTTATTG |
| | TCTGGAGTGCACACAGAGGATAATGAGGCAGAAGAGAAGGCATTGGCTGAATTCTTGCTT |
| | AATCAAGAGGTGATTCATCCCCGCGTTGCGCATGCCATCATGGAGGCAAGCTCTGTAGGT |
| | AGGAGAAAGCAAATTCAAGGGCTTGTTGACACAACAAACACCGTAATTAAGATTGCGCTT |
| | ACTAGGAGGCCATTAGGCATCAAGAGGCTGATGCGGATAGTCAATTATTCTAGCATGCAT |
| | GCAATGCTGTTTAGAGACGATGTTTTTTCCTCCAGTAGATCCAACCACCCCTTAGTCTCT |
| | TCTAATATGTGTTCTCTGACACTGGCAGACTATGCACGGAATAGAAGCTGGTCACCTTTG |
| | ACGGGAGGCAGGAAAATACTGGGTGTATCTAATCCTGATACGATAGAACTCGTAGAGGGT |
| | GAGATTCTTAGTGTAAGCGGAGGGTGTACAAGATGTGACAGCGGAGATGAACAATTTACT |
| | TGGTTCCATCTTCCAAGCAATATAGAATTGACCGATGACACCAGCAAGAATCCTCCGATG |
| | AGGGTACCATATCTCGGGTCAAAGACACAGGAGAGGAGAGCTGCCTCACTTGCAAAAATA |
| | GCTCATATGTCGCCACATGTAAAGGCTGCCCTAAGGGCATCATCCGTGTTGATCTGGGCT |
| | TATGGGGATAATGAAGTAAATTGGACTGCTGCTCTTACGATTGCAAAATCTCGGTGTAAT |
| | GTAAACTTAGAGTATCTTCGGTTACTGTCCCCTTTACCCACGGCTGGGAATCTTCAACAT |
| | AGACTAGATGATGGTATAACTCAGATGACATTCACCCCTGCATCTCTCTACAGGgtgtca |
| | ccttacattcacatatccaatgattctcaaaggctgttcactgaagaaggagtcaaagag |
| | gggaatgtggtttaccaacagatcATGCTCTTGGGTTTATCTCTAATCGAATCGATCTTT |
| | CCAATGACAACAACCAGGACATATGATGAGATCACACTGCACCTAGATAGTAAATTTAGT |
| | TGCTGTATCAGAGAAGCACCTGTTGCGGTTCCTTTCGAGCTACTTGGGGTGGTACCGGAA |
| | CTGAGGACAGTGACCTCAAATAAGTTTATGTATGATCCTAGCCCTGTATCGGAGGGAGAC |
| | TTTGCGAGACTTGACTTAGCTATCTTCAAGAGTTATGAGCTTAATCTGGAGTCATATCCC |
| | ACGATAGAGCTAATGAACATTCTTTCAATATCCAGCGGGAAGTTGATTGGCCAGTCTGTG |
| | GTTTCTTATGATGAAGATACCTCCATAAAGAATGACGCCATAATAGTGTATGACAATACC |
| | CGAAATTGGATCAGTGAAGCTCAGAATTCAGATGTGGTCCGCCTATTTGAATATGCAGCA |
| | CTTGAAGTGCTCCTCGACTGTTCTTACCAACTCTATTACCTGAGAGTAAGAGGCCTAGAC |
| | AATATTGTCTTATATATGGGTGATTTATACAAGAATATGCCAGGAATTCTACTTTCCAAC |
| | ATTGCAGCTACAATATCTCATCCCGTCATTCATTCAAGGTTACATGCAGTGGGCCTGGTC |
| | AACCATGACGGATCACACCAACTTGCAGATACGGATTTTATCGAAATGTCTGCAAAACTA |
| | TTAGTATCTTGCACCCGACGTGTGATCTCCGGCTTATATTCAGGAAATAAGTATGATCTG |
| | CTGTTCCCATCTGTCTTAGATGATAACCTGAATGAGAAGATGCTTCAGCTGATATCCCGG |
| | TTATGCTGTCTGTACACGGTACTCTTTGCTACAACAAGAGAAATCCCGAAAATAAGAGGC |
| | TTAACTGCAGAAGAGAAATGTTCAATACTCACTGAGTATTTACTGTCGGATGCTGTGAAA |
| | CCATTACTTAGCCCCGATCAAGTGAGCTCTATCATGTCTCCTAACATAATTACATTCCCA |
| | GCTAATCTGTACTACATGTCTCGGAAGAGCCTCAATTTGATCAGGGAAAGGGAGGACAGG |
| | GATACTATCCTGGCGTTGTTGTTCCCCCAAGAGCCATTATTAGAGTTCCCTTCTGTGCAA |
| | GATATTGGTGCTCGAGTGAAAGATCCATTCACCCGACAACCTGCGGCATTTTTGCAAGAG |
| | TTAGATTTGAGTGCTCCAGCAAGGTATGACGCATTCACACTTAGTCAGATTCATCCTGAA |
| | CTCACATCTCCAAATCCGGAGGAAGACTACTTAGTACGATACTTGTTCAGAGGGATAGGG |
| | ACTGCATCTTCCTCTTGGTATAAGGCATCTCATCTCCTTTCTGTACCCGAGGTAAGATGT |
| | GCAAGACACGGGAACTCCTTATACTTAGCTGAAGGGAGCGGAGCCATCATGAGTCTTCTC |
| | GAACTGCATGTACCACATGAAACTATCTATTACAATACGCTCTTTTCAAATGAGATGAAC |
| | CCCCCGCAACGACATTTCGGGCCGACCCCAACTCAGTTTTTGAATTCGGTTGTTTATAGG |
| | AATCTACAGGCGGAGGTAACATGCAAAGATGGATTTGTCCAAGAGTTCCGTCCATTATGG |
| | AGAGAAAATACAGAGGAAAGTGACCTGACCTCAGATAAAGCAGTGGGGTATATTACATCT |
| | GCAGTGCCCTACAGATCTGTATCATTGCTGCATTGTGACATTGAAATTCCTCCAGGGTCC |
| | AATCAAAGCTTACTAGATCAACTAGCTATCAATTTATCTCTGATTGCCATGCATTCTGTA |
| | AGGGAGGGCGGGGTAGTAATCATCAAAGTGTTGTATGCAATGGGATACTACTTTCATCTA |
| | CTCATGAACTTGTTTGCTCCGTGTTCCACAAAAGGATATATTCTCTCTAATGGTTATGCA |
| | TGTCGAGGAGATATGGAGTGTTACCTGGTATTTGTCATGGGTTACCTGGGCGGGCCTACA |
| | TTTGTACATGAGGTGGTGAGGATGGCAAAAACTCTGGTGCAGCGGCACGGTACGCTCTTG |
| | TCTAAATCAGATGAGATCACACTGACCAGGTTATTCACCTCACAGCGGCAGCGTGTGACA |
| | GACATCCTATCCAGTCCTTTACCAAGATTAATAAAGTACTTGAGGAAGAATATTGACACT |
| | GCGCTGATTGAAGCCGGGGGACAGCCCGTCCGTCCATTCTGTGCGGAGAGTCTGGTGAGC |
| | ACGCTAGCGAACATAACTCAGATAACCCAGATTATCGCTAGTCACATTGACACAGTTATC |
| | CGGTCTGTGATATATATGGAAGCTGAGGGTGATCTCGCTGACACAGTATTTCTATTTACC |
| | CCTTACAATCTCTCTACTGACGGGAAAAAGAGGACATCACTTATACAGTGCACGAGACAG |
| | ATCCTAGAGGTTACAATACTAGGTCTTAGAGTCGAAAATCTCAATAAAATAGGCGATATA |
| | ATCAGCCTAGTGCTTAAAGGCATGATCTCCATGGAGGACCTTATCCCACTAAGGACATAC |
| | TTGAAGCATAGTACCTGCCCTAAATATTTGAAGGCTGTCCTAGGTATTACCAAACTCAAA |
| | GAAATGTTTACAGACACTTCTGTATTGTACTTGACTCGTGCTCAACAAAAATTCTACATG |
| | AAAACTATAGGCAATGCAGTCAAAGGATATTACAGTAACTGTGACTCTTAACGAAAATCA |
| | CATATTAATAGGCTCCTTTTTTGGCCAATTGTATTCTTGTTGATTTAATCATATTATGTT |
| | AGAAAAAAGTTGAACCCTGACTCCTTAGGACTCGAATTCGAACTCAAATAAATGTCTTAA |
| | AAAAAGGTTGCGCACAATTATTCTTGAGTGTAGTCTCGTCATTCACCAAATCTTTGTTTG |
| | GTGGCCGGCATGGTCCCAGCCTCCTCGCTGGCGCCGGCTGGGCAACATTCCGAGGGGACC |
| | GTCCCCTCGGTAATGGCGAATGGGACGTCGACTGCTAACAAAGCCCGAAAGGAAGCTGAG |
| | TTGGCTGCTGCCACCGCTGAGCAATAACTAGCATAACCCCTTGGGGCCTCTAAACGGGTC |
| | TTGAGGGGTTTTTTGCTGAAAGGAGGAACTATA |
| SEQ ID NO: | TAATACGACTCACTATAGGGACCAAACAGAGAATCCGTGAGTTACGATAAAAGGCGAAGG |
| 3; nucleotide | AGCAATTGAAGTCGCACGGGTAGAAGGTGTGAATCTCGAGTGCGAGCCCGAAGCACAAAC |
| sequence of | TCGAGAAAGCCTTCTGCCAACATGTCTTCCGTATTTGATGAGTACGAACAGCTCCTCGCG |
| NDV-Δ19-S | GCTCAGACTCGCCCCAATGGAGCTCATGGAGGGGGAGAAAAAGGGAGTACCTTAAAAGTA |
| Molecular | GACGTCCCGGTATTCACTCTTAACAGTGATGACCCAGAAGATAGATGGAGCTTTGTGGTA |
| Clone | TTCTGCCTCCGGATTGCTGTTAGCGAAGATGCCAACAAACCACTCAGGCAAGGTGCTCTC |
| AF077761.1_ | ATATCTCTTTTATGCTCCCACTCACAGGTAATGAGGAACCATGTTGCCATTGCAGGGAAA |
| LaSota_Kan | CAGAATGAAGCCACATTGGCCGTGCTTGAGATTGATGGCTTTGCCAACGGCACGCCCCAG |
| R (with | TTCAACAATAGGAGTGGAGTGTCTGAAGAGAGAGCACAGAGATTTGCGATGATAGCAGGA |
| stabilizing | TCTCTCCCTCGGGCATGCAGCAACGGAACCCCGTTCGTCACAGCCGGGGCAGAAGATGAT |
| sequence in | GCACCAGAAGACATCACCGATACCCTGGAGAGGATCCTCTCTATCCAGGCTCAAGTATGG |
| L) | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaTTAGAAAAAATA |
| | CGGGTAGAACCGCCACCATGTTCGTGTTCCTGGTCCTGCTGCCACTGGTAAGCTCCCAAT |
| | GTGTAAACTTAACCACAAGAACCCAGCTCCCACCTGCCTACACCAACAGCTTCACCAGAG |
| | GCGTTTATTACCCCGACAAGGTATTCCGGTCTTCTGTTCTGCACTCTACCCAGGACCTGT |
| | TTCTGCCCTTTTTCAGCAACGTGACATGGTTCCACGCCATCCACGTGTCTGGCACAAACG |
| | GCACCAAGCGGTTTGATAATCCTGTGCTCCCTTTCAATGACGGCGTGTACTTCGCCTCTA |
| | CTGAGAAGAGCAACATCATCCGGGGCTGGATCTTTGGCACAACACTGGACTCTAAAACCC |
| | AGAGCCTGCTGATCGTGAACAACGCCACCAACGTGGTGATTAAGGTGTGCGAGTTCCAGT |
| | TCTGCAATGACCCTTTCCTCGGCGTGTACTACCACAAGAACAACAAAAGTTGGATGGAAA |
| | GCGAATTCAGGGTGTACTCAAGCGCCAACAACTGTACCTTCGAGTACGTGAGCCAGCCTT |
| | TCCTGATGGACCTAGAAGGTAAGCAGGGCAATTTCAAGAACCTCAGAGAGTTCGTGTTCA |
| | AGAATATTGACGGCTACTTCAAAATCTACAGCAAGCACACCCCAATCAACCTGGTGCGGG |
| | ACCTGCCCCAGGGCTTTAGCGCGCTGGAGCCTCTGGTGGACCTGCCTATCGGCATCAACA |
| | TCACCCGGTTCCAGACACTGCTGGCTCTGCATAGAAGCTACCTGACACCTGGCGACAGTT |
| | CTTCTGGCTGGACAGCCGGCGCCGCCGCCTACTACGTGGGCTACCTGCAGCCTAGAACAT |
| | TCCTGCTGAAATACAACGAGAACGGCACGATCACAGACGCCGTGGACTGCGCCCTGGATC |
| | CCCTGTCTGAGACAAAGTGCACCCTGAAGTCTTTCACCGTGGAGAAGGGCATCTACCAGA |
| | CCTCCAACTTCAGAGTGCAGCCTACCGAATCCATCGTGCGCTTTCCCAACATCACCAACC |
| | TGTGCCCCTTCGGCGAGGTCTTTAATGCCACGAGATTCGCCAGCGTGTATGCCTGGAACA |
| | GAAAGAGAATCAGCAACTGCGTGGCCGACTACAGCGTGCTGTACAACAGCGCCTCTTTCA |
| | GCACATTTAAGTGCTACGGAGTGTCTCCTACCAAACTCAACGATCTGTGCTTCACGAACG |
| | TGTATGCCGACAGCTTCGTGATCCGAGGAGATGAGGTGCGGCAGATCGCTCCAGGACAGA |
| | CAGGCAAGATCGCCGACTACAACTACAAGCTGCCCGACGACTTTACCGGCTGCGTGATCG |
| | CTTGGAACAGCAATAACCTGGACTCAAAGGTTGGAGGAAACTACAACTACCTGTACAGAC |
| | TGTTCAGAAAGTCCAACCTGAAGCCCTTCGAGAGAGACATCTCTACAGAAATCTACCAGG |
| | CCGGCAGCACCCCATGTAACGGCGTGGAAGGCTTCAACTGCTACTTCCCTCTGCAGTCTT |
| | ATGGCTTCCAGCCCACAAACGGAGTGGGCTATCAGCCTTACCGCGTGGTTGTCCTGAGCT |
| | TTGAGCTGCTGCATGCCCCTGCTACGGTGTGTGGACCTAAGAAGTCCACCAACCTGGTGA |
| | AGAACAAGTGTGTGAACTTCAACTTCAACGGCCTGACCGGCACCGGGGTGCTGACAGAGT |
| | CTAACAAGAAATTCCTGCCATTCCAGCAATTCGGCCGGGACATCGCCGACACCACCGACG |
| | CCGTGCGGGATCCTCAGACCCTCGAAATCCTGGACATCACCCCCTGTAGCTTCGGCGGCG |
| | TGAGCGTGATCACCCCTGGCACAAACACCAGCAATCAAGTGGCTGTCCTGTACCAGGATG |
| | TCAATTGCACAGAAGTGCCTGTGGCCATCCACGCCGATCAGCTGACCCCCACCTGGCGGG |
| | TGTACTCGACAGGAAGCAACGTGTTTCAAACAAGAGCCGGCTGCCTGATCGGGGCCGAGC |
| | ACGTGAACAATTCCTACGAGTGCGACATCCCCATCGGCGCCGGCATCTGTGCCTCTTACC |
| | AGACACAGACCAATTCCCCTCGTAGAGCCAGATCCGTGGCCAGCCAGAGCATCATCGCCT |
| | ACACCATGAGCCTGGGCGCCGAAAACAGCGTTGCATATTCCAACAACAGCATCGCCATCC |
| | CTACCAACTTCACCATCAGCGTGACCACAGAAATCCTGCCTGTGTCCATGACCAAGACAA |
| | GCGTTGATTGCACCATGTACATCTGCGGCGATAGCACAGAGTGCAGCAATCTGCTGCTGC |
| | AGTACGGTAGCTTCTGCACCCAGCTGAATAGAGCCCTGACCGGCATCGCTGTGGAACAGG |
| | ACAAAAACACCCAGGAGGTCTTCGCCCAGGTGAAGCAAATCTACAAGACCCCTCCAATCA |
| | AGGACTTCGGAGGCTTTAACTTTAGCCAGATCCTGCCTGATCCCTCCAAGCCTAGCAAAC |
| | GGAGTTTCATCGAGGACCTGCTCTTCAACAAGGTGACCCTGGCTGACGCCGGCTTCATTA |
| | AGCAGTACGGCGATTGCCTCGGCGACATCGCTGCAAGAGACCTGATCTGCGCCCAGAAGT |
| | TCAACGGCCTGACCGTGCTGCCTCCTCTCCTGACAGACGAGATGATCGCCCAGTACACCT |
| | CTGCCCTTCTGGCTGGCACCATCACCAGCGGATGGACCTTTGGAGCCGGAGCCGCCCTGC |
| | AGATCCCTTTCGCTATGCAGATGGCCTACAGATTCAACGGGATCGGAGTGACCCAAAACG |
| | TGCTGTATGAAAACCAGAAACTGATCGCCAATCAGTTTAACAGCGCCATCGGCAAAATCC |
| | AGGATAGCCTGTCCAGCACCGCCAGCGCCCTCGGCAAGCTGCAAGATGTGGTGAATCAAA |
| | ATGCCCAAGCCCTGAACACACTGGTGAAGCAGCTGAGCAGCAACTTCGGCGCCATCAGCA |
| | GCGTGCTGAACGACATCCTGAGCAGACTGGACAAGGTGGAAGCCGAGGTGCAGATCGACA |
| | GACTGATCACAGGCAGACTGCAGTCCCTGCAGACCTACGTGACCCAGCAGTTGATTAGAG |
| | CCGCTGAGATTAGAGCCAGTGCCAACCTGGCTGCCACAAAGATGTCAGAATGCGTGCTGG |
| | GCCAGAGCAAGAGAGTGGACTTCTGCGGCAAAGGCTACCACCTGATGAGCTTTCCTCAGT |
| | CTGCACCCCACGGCGTGGTGTTTCTCCACGTGACATACGTGCCCGCGCAAGAAAAGAACT |
| | TTACAACCGCCCCAGCGATCTGCCACGACGGCAAGGCCCACTTCCCTCGGGAGGGTGTGT |
| | TCGTGAGCAATGGAACACACTGGTTCGTCACCCAGCGGAACTTCTACGAGCCTCAGATCA |
| | TTACCACCGACAACACCTTCGTGAGCGGCAACTGTGACGTCGTTATCGGCATCGTGAACA |
| | ATACCGTGTACGACCCCCTGCAGCCTGAGCTGGATAGCTTCAAAGAGGAACTGGACAAGT |
| | ACTTCAAGAACCACACAAGCCCCGACGTGGACCTAGGCGACATCTCTGGAATCAACGCCA |
| | GCGTGGTGAACATCCAAAAGGAAATCGACAGACTGAACGAGGTGGCCAAGAATCTGAATG |
| | AAAGCCTGATCGATCTGCAGGAGCTGGGCAAGTACGAGCAGTACATCAAATGGCCTTGGT |
| | ACATCTGGCTGGGCTTCATCGCTGGTCTGATCGCTATCGTGATGGTGACCATTATGCTGT |
| | GCTGCATGACCTCCTGCTGCTCTTGCCTGAAGGGCTGTTGTTCTTGCGGCTCTTGCTGCT |
| | GATGATaaacgcgtACCCAAGGTCCAACTCTCCAAGCGGCAATCCTCTCTCGCTTCCTCA |
| | GCCCCACTGAATGGTCGCGTAACCGTAATTAATCTAGCTACATTTAAGATTAAGAAAAAA |
| | TACGGGTAGAATTGGAGTGCCCCAATTGTGCCAAGATGGACTCATCTAGGACAATTGGGC |
| | TGTACTTTGATTCTGCCCATTCTTCTAGCAACCTGTTAGCATTTCCGATCGTCCTACAAG |
| | GCACAGGAGATGGGAAGAAGCAAATCGCCCCGCAATATAGGATCCAGCGCCTTGACTTGT |
| | GGACTGATAGTAAGGAGGACTCAGTATTCATCACCACCTATGGATTCATCTTTCAAGTTG |
| | GGAATGAAGAAGCCACTGTCGGCATGATCGATGATAAACCCAAGCGCGAGTTACTTTCCG |
| | CTGCGATGCTCTGCCTAGGAAGCGTCCCAAATACCGGAGACCTTATTGAGCTGGCAAGGG |
| | CCTGTCTCACTATGATAGTCACATGCAAGAAGAGTGCAACTAATACTGAGAGAATGGTTT |
| | TCTCAGTAGTGCAGGCACCCCAAGTGCTGCAAAGCTGTAGGGTTGTGGCAAACAAATACT |
| | CATCAGTGAATGCAGTCAAGCACGTGAAAGCGCCAGAGAAGATTCCCGGGAGTGGAACCC |
| | TAGAATACAAGGTGAACTTTGTCTCCTTGACTGTGGTACCGAAGAAGGATGTCTACAAGA |
| | TCCCAGCTGCAGTATTGAAGGTTTCTGGCTCGAGTCTGTACAATCTTGCGCTCAATGTCA |
| | CTATTAATGTGGAGGTAGACCCGAGGAGTCCTTTGGTTAAATCTTTGTCTAAGTCTGACA |
| | GCGGATACTATGCTAACCTCTTCTTGCATATTGGACTTATGACCACCGTAGATAGGAAGG |
| | GGAAGAAAGTGACATTTGACAAGCTGGAAAAGAAAATAAGGAGCCTTGATCTATCTGTCG |
| | GGCTCAGTGATGTGCTCGGGCCTTCCGTGTTGGTAAAAGCAAGAGGTGCACGGACTAAGC |
| | TTTTGGCACCTTTCTTCTCTAGCAGTGGGACAGCCTGCTATCCCATAGCAAATGCTTCTC |
| | CTCAGGTGGCCAAGATACTCTGGAGTCAAACCGCGTGCCTGCGGAGCGTTAAAATCATTA |
| | TCCAAGCAGGTACCCAACGCGCTGTCGCAGTGACCGCCGACCACGAGGTTACCTCTACTA |
| | AGCTGGAGAAGGGGCACACCCTTGCCAAATACAATCCTTTTAAGAAATAAGCTGCGTCTC |
| | TGAGATTGCGCTCCGCCCACTCACCCAGATCATCATGACACAAAAAACTAATCTGTCTTG |
| | ATTATTTACAGTTAGTTTACCTGTCTATCAAGTTAGAAAAAACACGGGTAGAAGATTCTG |
| | GATCCCGGTTGGCGCCCTCCAGGTGCAAGttaattaaATGGGCTCCAGACCTTCTACCAA |
| | GAACCCAGCACCTATGATGCTGACTATCCGGGTTGCGCTGGTACTGAGTTGCATCTGTCC |
| | GGCAAACTCCATTGATGGCAGGCCTCTTGCAGCTGCAGGAATTGTGGTTACAGGAGACAA |
| | AGCCGTCAACATATACACCTCATCCCAGACAGGATCAATCATAGTTAAGCTCCTCCCGAA |
| | TCTGCCCAAGGATAAGGAGGCATGTGCGAAAGCCCCCTTGGATGCATACAACAGGACATT |
| | GACCACTTTGCTCACCCCCCTTGGTGACTCTATCCGTAGGATACAAGAGTCTGTGACTAC |
| | ATCTGGAGGGGGGAGACAGGGGCGCCTTATAGGCGCCATTATTGGCGGTGTGGCTCTTGG |
| | GGTTGCAACTGCCGCACAAATAACAGCGGCCGCAGCTCTGATACAAGCCAAACAAAATGC |
| | TGCCAACATCCTCCGACTTAAAGAGAGCATTGCCGCAACCAATGAGGCTGTGCATGAGGT |
| | CACTGACGGATTATCGCAACTAGCAGTGGCAGTTGGGAAGATGCAGCAGTTTGTTAATGA |
| | CCAATTTAATAAAACAGCTCAGGAATTAGACTGCATCAAAATTGCACAGCAAGTTGGTGT |
| | AGAGCTCAACCTGTACCTAACCGAATTGACTACAGTATTCGGACCACAAATCACTTCACC |
| | TGCTTTAAACAAGCTGACTATTCAGGCACTTTACAATCTAGCTGGTGGAAATATGGATTA |
| | CTTATTGACTAAGTTAGGTGTAGGGAACAATCAACTCAGCTCATTAATCGGTAGCGGCTT |
| | AATCACtGGcAACCCTATTCTATACGACTCACAGACTCAACTCTTGGGTATACAGGTAAC |
| | TgcaCCTTCAGTCGGGAACCTAAATAATATGCGTGCCACCTACTTGGAAACCTTATCCGT |
| | AAGCACAACCAGGGGATTTGCCTCGGCACTTGTCCCCAAAGTGGTGACACAGGTCGGTTC |
| | TGTGATAGAAGAACTTGACACCTCATACTGTATAGAAACTGACTTAGATTTATATTGTAC |
| | AAGAATAGTAACGTTCCCTATGTCCCCTGGTATTTATTCCTGCTTGAGCGGCAATACGTC |
| | GGCCTGTATGTACTCAAAGACCGAAGGCGCACTTACTACACCATACATGACTATCAAAGG |
| | TTCAGTCATCGCCAACTGCAAGATGACAACATGTAGATGTGTAAACCCCCCGGGTATCAT |
| | ATCGCAAAACTATGGAGAAGCCGTGTCTCTAATAGATAAACAATCATGCAATGTTTTATC |
| | CTTAGGCGGGATAACTTTAAGGCTCAGTGGGGAATTCGATGTAACTTATCAGAAGAATAT |
| | GTCAATACAAGATTCTCAAGTAATAATAACAGGCAATCTTGATATCTCAACTGAGGTTGG |
| | GAATGTCAACAACTCGATCAGTAATGCTTTGAATAAGTTAGAGGAAAGCAACAGAAAACT |
| | AGACAAAGTCAATGTCAAACTGACTAGCACATCTGCTCTCATTACgTATATCGTTTTGAC |
| | TATCATATCTCTTGTTTTTGGTATACTTAGCCTGATTCTAGCATGCTACCTAATGTACAA |
| | GCAAAAGGCGCAACAAAAGACCTTATTATGGCTTGGGAATAATACaCTcGATCAGATGAG |
| | AGCCACTACAAAAATGTGAACACAGATGAGGAACGAAGGTTTCCCTAATAGTAATTTGTG |
| | TGAAAGTTCTGGTAGTCTGTCAGTTCAGAGAGTTAAGAAAAAACTACCGGTTGTAGATGA |
| | CCAAAGGACGATATACGGGTAGAACGGTAAGAGAGGCCGCCCCTCAATTGCGAGCCAGGC |
| | TTCACAACCTCCGTTCTACCGCTTCACCGACAACAGTCCTCAATCATGGACCGCGCCGTT |
| | AGCCAAGTTGCGTTAGAGAATGATGAAAGAGAGGCAAAAAATACATGGCGCTTGATATTC |
| | CGGATTGCAATCTTATTCTTAACAGTAGTGACCTTGGCTATATCTGTAGCCTCCCTTTTA |
| | TATAGCATGGGGGCTAGCACACCTAGCGATCTTGTAGGCATACCGACTAGGATTTCCAGG |
| | GCAGAAGAAAAGATTACATCTACACTTGGTTCCAATCAAGATGTAGTAGATAGGATATAT |
| | AAGCAAGTGGCCCTTGAGTCTCCGTTGGCATTGTTAAATACTGAGACCACAATTATGAAC |
| | GCAATAACATCTCTCTCTTATCAGATTAATGGAGCTGCAAACAACAGTGGGTGGGGGGCA |
| | CCTATCCATGACCCAGATTATATAGGGGGGATAGGCAAAGAACTCATTGTAGATGATGCT |
| | AGTGATGTCACATCATTCTATCCCTCTGCATTTCAAGAACATCTGAATTTTATCCCGGCG |
| | CCTACTACAGGATCAGGTTGCACTCGAATACCCTCATTTGACATGAGTGCTACCCATTAC |
| | TGCTACACCCATAATGTAATATTGTCTGGATGCAGAGATCACTCACATTCATATCAGTAT |
| | TTAGCACTTGGTGTGCTCCGGACATCTGCAACAGGGAGGGTATTCTTTTCTACTCTGCGT |
| | TCCATCAACCTGGACGACACCCAAAATCGGAAGTCTTGCAGTGTGAGTGCAACTCCCCTG |
| | GGTTGTGATATGCTGTGCTCGAAAGTCACGGAGACAGAGGAAGAAGATTATAACTCAGCT |
| | GTCCCTACGCGGATGGTACATGGGAGGTTAGGGTTCGACGGCCAGTACCACGAAAAGGAC |
| | CTAGATGTCACAACATTATTCGGGGACTGGGTGGCCAACTACCCAGGAGTAGGGGGTGGA |
| | TCTTTTATTGACAGCCGCGTATGGTTCTCAGTCTACGGAGGGTTAAAACCCAATTCACCC |
| | AGTGACACTGTACAGGAAGGGAAATATGTGATATACAAGCGATACAATGACACATGCCCA |
| | GATGAGCAAGACTACCAGATTCGAATGGCCAAGTCTTCGTATAAGCCTGGACGGTTTGGT |
| | GGGAAACGCATACAGCAGGCTATCTTATCTATCAAGGTGTCAACATCCTTAGGCGAAGAC |
| | CCGGTACTGACTGTACCGCCCAACACAGTCACACTCATGGGGGCCGAAGGCAGAATTCTC |
| | ACAGTAGGGACATCTCATTTCTTGTATCAACGAGGGTCATCATACTTCTCTCCCGCGTTA |
| | TTATATCCTATGACAGTCAGCAACAAAACAGCCACTCTTCATAGTCCTTATACATTCAAT |
| | GCCTTCACTCGGCCAGGTAGTATCCCTTGCCAGGCTTCAGCAAGATGCCCCAACTCGTGT |
| | GTTACTGGAGTCTATACAGATCCATATCCCCTAATCTTCTATAGAAACCACACCTTGCGA |
| | GGGGTATTCGGGACAATGCTTGATGGTGTACAAGCAAGACTTAACCCTGCGTCTGCAGTA |
| | TTCGATAGCACATCCCGCAGTCGCATTACTCGAGTGAGTTCAAGCAGTACCAAAGCAGCA |
| | TACACAACATCAACTTGTTTTAAAGTGGTCAAGACTAATAAGACCTATTGTCTCAGCATT |
| | GCTGAAATATCTAATACTCTCTTCGGAGAATTCAGAATCGTCCCGTTACTAGTTGAGATC |
| | CTCAAAGATGACGGGGTTAGAGAAGCCAGGTCTGGCTAGggcgcgccTTGAGTCAATTAT |
| | AAAGGAGTTGGAAAGATGGCATTGTATCACCTATCTTCTGCGACATCAAGAATCAAACCG |
| | AATGCCGGCGCGTGCTCGAATTCCATGTTGCCAGTTGACCACAATCAGCCAGTGCTCATG |
| | CGATCAGATTAAGCCTTGTCATTAATCTCTTGATTAAGAAAAAATGTAAGTGGCAATGAG |
| | ATACAAGGCAAAACAGCTCATGGTAAATAATACGGGTAGGACATGGCGAGCTCCGGTCCT |
| | GAAAGGGCAGAGCATCAGATTATCCTACCAGAGCCACACCTGTCTTCACCATTGGTCAAG |
| | CACAAACTACTCTATTACTGGAAATTAACTGGGCTACCGCTTCCTGATGAATGTGACTTC |
| | GACCACCTCATTCTCAGCCGACAATGGAAAAAAATACTTGAATCGGCCTCTCCTGATACT |
| | GAGAGAATGATAAAACTCGGAAGGGCAGTACACCAAACTCTTAACCACAATTCCAGAATA |
| | ACCGGAGTGCTCCACCCCAGGTGTTTAGAACAACTGGCTAATATTGAGGTCCCAGATTCA |
| | ACCAACAAATTTCGGAAGATTGAGAAGAAGATCCAAATTCACAACACGAGATATGGAGAA |
| | CTGTTCACAAGGCTGTGTACGCATATAGAGAAGAAACTGCTGGGGTCATCTTGGTCTAAC |
| | AATGTCCCCCGGTCAGAGGAGTTCAGCAGCATTCGTACGGATCCGGCATTCTGGTTTCAC |
| | TCAAAATGGTCCACAGCCAAGTTTGCATGGCTCCATATAAAACAGATCCAGAGGCATCTG |
| | ATGGTGGCAGCTAAGACAAGGTCTGCGGCCAACAAATTGGTGATGCTAACCCATAAGGTA |
| | GGCCAAGTCTTTGTCACTCCTGAACTTGTCGTTGTGACGCATACGAATGAGAACAAGTTC |
| | ACATGTCTTACCCAGGAACTTGTATTGATGTATGCAGATATGATGGAGGGCAGAGATATG |
| | GTCAACATAATATCAACCACGGCGGTGCATCTCAGAAGCTTATCAGAGAAAATTGATGAC |
| | ATTTTGCGGTTAATAGACGCTCTGGCAAAAGACTTGGGTAATCAAGTCTACGATGTTGTA |
| | TCACTAATGGAGGGATTTGCATACGGAGCTGTCCAGCTACTCGAGCCGTCAGGTACATTT |
| | GCAGGAGATTTCTTCGCATTCAACCTGCAGGAGCTTAAAGACATTCTAATTGGCCTCCTC |
| | CCCAATGATATAGCAGAATCCGTGACTCATGCAATCGCTACTGTATTCTCTGGTTTAGAA |
| | CAGAATCAAGCAGCTGAGATGTTGTGTCTGTTGCGTCTGTGGGGTCACCCACTGCTTGAG |
| | TCCCGTATTGCAGCAAAGGCAGTCAGGAGCCAAATGTGCGCACCGAAAATGGTAGACTTT |
| | GATATGATCCTTCAGGTACTGTCTTTCTTCAAGGGAACAATCATCAACGGGTACAGAAAG |
| | AAGAATGCAGGTGTGTGGCCGCGAGTCAAAGTGGATACAATATATGGGAAGGTCATTGGG |
| | CAACTACATGCAGATTCAGCAGAGATTTCACACGATATCATGTTGAGAGAGTATAAGAGT |
| | TTATCTGCACTTGAATTTGAGCCATGTATAGAATATGACCCTGTCACCAACCTGAGCATG |
| | TTCCTAAAAGACAAGGCAATCGCACACCCCAACGATAATTGGCTTGCCTCGTTTAGGCGG |
| | AACCTTCTCTCCGAAGACCAGAAGAAACATGTAAAAGAAGCAACTTCGACTAATCGCCTC |
| | TTGATAGAGTTTTTAGAGTCAAATGATTTTGATCCATATAAAGAGATGGAATATCTGACG |
| | ACCCTTGAGTACCTTAGAGATGACAATGTGGCAGTATCATACTCGCTCAAGGAGAAGGAA |
| | GTGAAAGTTAATGGACGGATCTTCGCTAAGCTGACAAAGAAGTTAAGGAACTGTCAGGTG |
| | ATGGCGGAAGGGATCCTAGCCGATCAGATTGCACCTTTCTTTCAGGGAAATGGAGTCATT |
| | CAGGATAGCATATCCTTGACCAAGAGTATGCTAGCGATGAGTCAACTGTCTTTTAACAGC |
| | AATAAGAAACGTATCACTGACTGTAAAGAAAGAGTATCTTCAAACCGCAATCATGATCCG |
| | AAAAGCAAGAACCGTCGGAGAGTTGCAACCTTCATAACAACTGACCTGCAAAAGTACTGT |
| | CTTAATTGGAGATATCAGACAATCAAATTGTTCGCTCATGCCATCAATCAGTTGATGGGC |
| | CTACCTCACTTCTTCGAATGGATTCACCTAAGACTGATGGACACTACGATGTTCGTAGGA |
| | GACCCTTTCAATCCTCCAAGTGACCCTACTGACTGTGACCTCTCAAGAGTCCCTAATGAT |
| | GACATATATATTGTCAGTGCCAGAGGGGGTATCGAAGGATTATGCCAGAAGCTATGGACA |
| | ATGATCTCAATTGCTGCAATCCAACTTGCTGCAGCTAGATCGCATTGTCGTGTTGCCTGT |
| | ATGGTACAGGGTGATAATCAAGTAATAGCAGTAACGAGAGAGGTAAGATCAGACGACTCT |
| | CCGGAGATGGTGTTGACACAGTTGCATCAAGCCAGTGATAATTTCTTCAAGGAATTAATT |
| | CATGTCAATCATTTGATTGGCCATAATTTGAAGGATCGTGAAACCATCAGGTCAGACACA |
| | TTCTTCATATACAGCAAACGAATCTTCAAAGATGGAGCAATCCTCAGTCAAGTCCTCAAA |
| | AATTCATCTAAATTAGTGCTAGTGTCAGGTGATCTCAGTGAAAACACCGTAATGTCCTGT |
| | GCCAACATTGCCTCTACTGTAGCACGGCTATGCGAGAACGGGCTTCCCAAAGACTTCTGT |
| | TACTATTTAAACTATATAATGAGTTGTGTGCAGACATACTTTGACTCTGAGTTCTCCATC |
| | ACCAACAATTCGCACCCCGATCTTAATCAGTCGTGGATTGAGGACATCTCTTTTGTGCAC |
| | TCATATGTTCTGACTCCTGCCCAATTAGGGGGACTGAGTAACCTTCAATACTCAAGGCTC |
| | TACACTAGAAATATCGGTGACCCGGGGACTACTGCTTTTGCAGAGATCAAGCGACTAGAA |
| | GCAGTGGGATTACTGAGTCCTAACATTATGACTAATATCTTAACTAGGCCGCCTGGGAAT |
| | GGAGATTGGGCCAGTCTGTGCAACGACCCATACTCTTTCAATTTTGAGACTGTTGCAAGC |
| | CCAAATATTGTTCTTAAGAAACATACGCAAAGAGTCCTATTTGAAACTTGTTCAAATCCC |
| | TTATTGTCTGGAGTGCACACAGAGGATAATGAGGCAGAAGAGAAGGCATTGGCTGAATTC |
| | TTGCTTAATCAAGAGGTGATTCATCCCCGCGTTGCGCATGCCATCATGGAGGCAAGCTCT |
| | GTAGGTAGGAGAAAGCAAATTCAAGGGCTTGTTGACACAACAAACACCGTAATTAAGATT |
| | GCGCTTACTAGGAGGCCATTAGGCATCAAGAGGCTGATGCGGATAGTCAATTATTCTAGC |
| | ATGCATGCAATGCTGTTTAGAGACGATGTTTTTTCCTCCAGTAGATCCAACCACCCCTTA |
| | GTCTCTTCTAATATGTGTTCTCTGACACTGGCAGACTATGCACGGAATAGAAGCTGGTCA |
| | CCTTTGACGGGAGGCAGGAAAATACTGGGTGTATCTAATCCTGATACGATAGAACTCGTA |
| | GAGGGTGAGATTCTTAGTGTAAGCGGAGGGTGTACAAGATGTGACAGCGGAGATGAACAA |
| | TTTACTTGGTTCCATCTTCCAAGCAATATAGAATTGACCGATGACACCAGCAAGAATCCT |
| | CCGATGAGGGTACCATATCTCGGGTCAAAGACACAGGAGAGGAGAGCTGCCTCACTTGCA |
| | AAAATAGCTCATATGTCGCCACATGTAAAGGCTGCCCTAAGGGCATCATCCGTGTTGATC |
| | TGGGCTTATGGGGATAATGAAGTAAATTGGACTGCTGCTCTTACGATTGCAAAATCTCGG |
| | TGTAATGTAAACTTAGAGTATCTTCGGTTACTGTCCCCTTTACCCACGGCTGGGAATCTT |
| | CAACATAGACTAGATGATGGTATAACTCAGATGACATTCACCCCTGCATCTCTCTACAGG |
| | gtgtcaccttacattcacatatccaatgattctcaaaggctgttcactgaagaaggagtc |
| | aaagaggggaatgtggtttaccaacagatcATGCTCTTGGGTTTATCTCTAATCGAATCG |
| | ATCTTTCCAATGACAACAACCAGGACATATGATGAGATCACACTGCACCTACATAGTAAA |
| | TTTAGTTGCTGTATCAGAGAAGCACCTGTTGCGGTTCCTTTCGAGCTACTTGGGGTGGTA |
| | CCGGAACTGAGGACAGTGACCTCAAATAAGTTTATGTATGATCCTAGCCCTGTATCGGAG |
| | GGAGACTTTGCGAGACTTGACTTAGCTATCTTCAAGAGTTATGAGCTTAATCTGGAGTCA |
| | TATCCCACGATAGAGCTAATGAACATTCTTTCAATATCCAGCGGGAAGTTGATTGGCCAG |
| | TCTGTGGTTTCTTATGATGAAGATACCTCCATAAAGAATGACGCCATAATAGTGTATGAC |
| | AATACCCGAAATTGGATCAGTGAAGCTCAGAATTCAGATGTGGTCCGCCTATTTGAATAT |
| | GCAGCACTTGAAGTGCTCCTCGACTGTTCTTACCAACTCTATTACCTGAGAGTAAGAGGC |
| | CTAGACAATATTGTCTTATATATGGGTGATTTATACAAGAATATGCCAGGAATTCTACTT |
| | TCCAACATTGCAGCTACAATATCTCATCCCGTCATTCATTCAAGGTTACATGCAGTGGGC |
| | CTGGTCAACCATGACGGATCACACCAACTTGCAGATACGGATTTTATCGAAATGTCTGCA |
| | AAACTATTAGTATCTTGCACCCGACGTGTGATCTCCGGCTTATATTCAGGAAATAAGTAT |
| | GATCTGCTGTTCCCATCTGTCTTAGATGATAACCTGAATGAGAAGATGCTTCAGCTGATA |
| | TCCCGGTTATGCTGTCTGTACACGGTACTCTTTGCTACAACAAGAGAAATCCCGAAAATA |
| | AGAGGCTTAACTGCAGAAGAGAAATGTTCAATACTCACTGAGTATTTACTGTCGGATGCT |
| | GTGAAACCATTACTTAGCCCCGATCAAGTGAGCTCTATCATGTCTCCTAACATAATTACA |
| | TTCCCAGCTAATCTGTACTACATGTCTCGGAAGAGCCTCAATTTGATCAGGGAAAGGGAG |
| | GACAGGGATACTATCCTGGCGTTGTTGTTCCCCCAAGAGCCATTATTAGAGTTCCCTTCT |
| | GTGCAAGATATTGGTGCTCGAGTGAAAGATCCATTCACCCGACAACCTGCGGCATTTTTG |
| | CAAGAGTTAGATTTGAGTGCTCCAGCAAGGTATGACGCATTCACACTTAGTCAGATTCAT |
| | CCTGAACTCACATCTCCAAATCCGGAGGAAGACTACTTAGTACGATACTTGTTCAGAGGG |
| | ATAGGGACTGCATCTTCCTCTTGGTATAAGGCATCTCATCTCCTTTCTGTACCCGAGGTA |
| | AGATGTGCAAGACACGGGAACTCCTTATACTTAGCTGAAGGGAGCGGAGCCATCATGAGT |
| | CTTCTCGAACTGCATGTACCACATGAAACTATCTATTACAATACGCTCTTTTCAAATGAG |
| | ATGAACCCCCCGCAACGACATTTCGGGCCGACCCCAACTCAGTTTTTGAATTCGGTTGTT |
| | TATAGGAATCTACAGGCGGAGGTAACATGCAAAGATGGATTTGTCCAAGAGTTCCGTCCA |
| | TTATGGAGAGAAAATACAGAGGAAAGTGACCTGACCTCAGATAAAGCAGTGGGGTATATT |
| | ACATCTGCAGTGCCCTACAGATCTGTATCATTGCTGCATTGTGACATTGAAATTCCTCCA |
| | GGGTCCAATCAAAGCTTACTAGATCAACTAGCTATCAATTTATCTCTGATTGCCATGCAT |
| | TCTGTAAGGGAGGGCGGGGTAGTAATCATCAAAGTGTTGTATGCAATGGGATACTACTTT |
| | CATCTACTCATGAACTTGTTTGCTCCGTGTTCCACAAAAGGATATATTCTCTCTAATGGT |
| | TATGCATGTCGAGGAGATATGGAGTGTTACCTGGTATTTGTCATGGGTTACCTGGGCGGG |
| | CCTACATTTGTACATGAGGTGGTGAGGATGGCAAAAACTCTGGTGCAGCGGCACGGTACG |
| | CTCTTGTCTAAATCAGATGAGATCACACTGACCAGGTTATTCACCTCACAGCGGCAGCGT |
| | GTGACAGACATCCTATCCAGTCCTTTACCAAGATTAATAAAGTACTTGAGGAAGAATATT |
| | GACACTGCGCTGATTGAAGCCGGGGGACAGCCCGTCCGTCCATTCTGTGCGGAGAGTCTG |
| | GTGAGCACGCTAGCGAACATAACTCAGATAACCCAGATTATCGCTAGTCACATTGACACA |
| | GTTATCCGGTCTGTGATATATATGGAAGCTGAGGGTGATCTCGCTGACACAGTATTTCTA |
| | TTTACCCCTTACAATCTCTCTACTGACGGGAAAAAGAGGACATCACTTATACAGTGCACG |
| | AGACAGATCCTAGAGGTTACAATACTAGGTCTTAGAGTCGAAAATCTCAATAAAATAGGC |
| | GATATAATCAGCCTAGTGCTTAAAGGCATGATCTCCATGGAGGACCTTATCCCACTAAGG |
| | ACATACTTGAAGCATAGTACCTGCCCTAAATATTTGAAGGCTGTCCTAGGTATTACCAAA |
| | CTCAAAGAAATGTTTACAGACACTTCTGTATTGTACTTGACTCGTGCTCAACAAAAATTC |
| | TACATGAAAACTATAGGCAATGCAGTCAAAGGATATTACAGTAACTGTGACTCTTAACGA |
| | AAATCACATATTAATAGGCTCCTTTTTTGGCCAATTGTATTCTTGTTGATTTAATCATAT |
| | TATGTTAGAAAAAAGTTGAACCCTGACTCCTTAGGACTCGAATTCGAACTCAAATAAATG |
| | TCTTAAAAAAAGGTTGCGCACAATTATTCTTGAGTGTAGTCTCGTCATTCACCAAATCTT |
| | TGTTTGGTGGCCGGCATGGTCCCAGCCTCCTCGCTGGCGCCGGCTGGGCAACATTCCGAG |
| | GGGACCGTCCCCTCGGTAATGGCGAATGGGACGTCGACTGCTAACAAAGCCCGAAAGGAA |
| | GCTGAGTTGGCTGCTGCCACCGCTGAGCAATAACTAGCATAACCCCTTGGGGCCTCTAAA |
| | CGGGTCTTGAGGGGTTTTTTGCTGAAAGGAGGAACTATAT |
| SEQ ID NO: | TAATACGACTCACTATAGGGACCAAACAGAGAATCCGTGAGTTACGATAAAAGGCGAAGG |
| 4; NDV- | AGCAATTGAAGTCGCACGGGTAGAAGGTGTGAATCTCGAGTGCGAGCCCGAAGCACAAAC |
| COVID19- | TCGAGAAAGCCTTCTGCCAACATGTCTTCCGTATTTGATGAGTACGAACAGCTCCTCGCG |
| Prefusion | GCTCAGACTCGCCCCAATGGAGCTCATGGAGGGGGAGAAAAAGGGAGTACCTTAAAAGTA |
| Chimeric S | GACGTCCCGGTATTCACTCTTAACAGTGATGACCCAGAAGATAGATGGAGCTTTGTGGTA |
| Molecular | TTCTGCCTCCGGATTGCTGTTAGCGAAGATGCCAACAAACCACTCAGGCAAGGTGCTCTC |
| Clone | ATATCTCTTTTATGCTCCCACTCACAGGTAATGAGGAACCATGTTGCCATTGCAGGGAAA |
| AF077761.1_ | CAGAATGAAGCCACATTGGCCGTGCTTGAGATTGATGGCTTTGCCAACGGCACGCCCCAG |
| LaSota_Kan | TTCAACAATAGGAGTGGAGTGTCTGAAGAGAGAGCACAGAGATTTGCGATGATAGCAGGA |
| R (with | TCTCTCCCTCGGGCATGCAGCAACGGAACCCCGTTCGTCACAGCCGGGGCAGAAGATGAT |
| stabilizing | GCACCAGAAGACATCACCGATACCCTGGAGAGGATCCTCTCTATCCAGGCTCAAGTATGG |
| sequence in | GTCACAGTAGCAAAAGCCATGACTGCGTATGAGACTGCAGATGAGTCGGAAACAAGGCGA |
| L) | ATCAATAAGTATATGCAGCAAGGCAGGGTCCAAAAGAAATACATCCTCTACCCCGTATGC |
| Sequence of | AGGAGCACAATCCAACTCACGATCAGACAGTCTCTTGCAGTCCGCATCTTTTTGGTTAGC |
| the Pre- | GAGCTCAAGAGAGGCCGCAACACGGCAGGTGGTACCTCTACTTATTATAACCTGGTAGGG |
| fusion | GACGTAGACTCATACATCAGGAATACCGGGCTTACTGCATTCTTCTTGACACTCAAGTAC |
| stabilized | GGAATCAACACCAAGACATCAGCCCTTGCACTTAGTAGCCTCTCAGGCGACATCCAGAAG |
| (HexaPro) | ATGAAGCAGCTCATGCGTTTGTATCGGATGAAAGGAGATAATGCGCCGTACATGACATTA |
| with | CTTGGTGATAGTGACCAGATGAGCTTTGCGCCTGCCGAGTATGCACAACTTTACTCCTTT |
| cytoplasmic | GCCATGGGTATGGCATCAGTCCTAGATAAAGGTACTGGGAAATACCAATTTGCCAGGGAC |
| and | TTTATGAGCACATCATTCTGGAGACTTGGAGTAGAGTACGCTCAGGCTCAGGGAAGTAGC |
| transmembrane | ATTAACGAGGATATGGCTGCCGAGCTAAAGCTAACCCCAGCAGCAATGAAGGGCCTGGCA |
| domains | GCTGCTGCCCAACGGGTCTCCGACGATACCAGCAGCATATACATGCCTACTCAACAAGTC |
| from NDV | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaTTAGAAAAAATA |
| | CGGGTAGAACCGCCACCATGTTCGTGTTCCTGGTCCTGCTGCCACTGGTAAGCTCCCAAT |
| | GTGTAAACTTAACCACAAGAACCCAGCTCCCACCTGCCTACACCAACAGCTTCACCAGAG |
| | GCGTTTATTACCCCGACAAGGTATTCCGGTCTTCTGTTCTGCACTCTACCCAGGACCTGT |
| | TTCTGCCCTTTTTCAGCAACGTGACATGGTTCCACGCCATCCACGTGTCTGGCACAAACG |
| | GCACCAAGCGGTTTGATAATCCTGTGCTCCCTTTCAATGACGGCGTGTACTTCGCCTCTA |
| | CTGAGAAGAGCAACATCATCCGGGGCTGGATCTTTGGCACAACACTGGACTCTAAAACCC |
| | AGAGCCTGCTGATCGTGAACAACGCCACCAACGTGGTGATTAAGGTGTGCGAGTTCCAGT |
| | TCTGCAATGACCCTTTCCTCGGCGTGTACTACCACAAGAACAACAAAAGTTGGATGGAAA |
| | GCGAATTCAGGGTGTACTCAAGCGCCAACAACTGTACCTTCGAGTACGTGAGCCAGCCTT |
| | TCCTGATGGACCTAGAAGGTAAGCAGGGCAATTTCAAGAACCTCAGAGAGTTCGTGTTCA |
| | AGAATATTGACGGCTACTTCAAAATCTACAGCAAGCACACCCCAATCAACCTGGTGCGGG |
| | ACCTGCCCCAGGGCTTTAGCGCGCTGGAGCCTCTGGTGGACCTGCCTATCGGCATCAACA |
| | TCACCCGGTTCCAGACACTGCTGGCTCTGCATAGAAGCTACCTGACACCTGGCGACAGTT |
| | CTTCTGGCTGGACAGCCGGCGCCGCCGCCTACTACGTGGGCTACCTGCAGCCTAGAACAT |
| | TCCTGCTGAAATACAACGAGAACGGCACGATCACAGACGCCGTGGACTGCGCCCTGGATC |
| | CCCTGTCTGAGACAAAGTGCACCCTGAAGTCTTTCACCGTGGAGAAGGGCATCTACCAGA |
| | CCTCCAACTTCAGAGTGCAGCCTACCGAATCCATCGTGCGCTTTCCCAACATCACCAACC |
| | TGTGCCCCTTCGGCGAGGTCTTTAATGCCACGAGATTCGCCAGCGTGTATGCCTGGAACA |
| | GAAAGAGAATCAGCAACTGCGTGGCCGACTACAGCGTGCTGTACAACAGCGCCTCTTTCA |
| | GCACATTTAAGTGCTACGGAGTGTCTCCTACCAAACTCAACGATCTGTGCTTCACGAACG |
| | TGTATGCCGACAGCTTCGTGATCCGAGGAGATGAGGTGCGGCAGATCGCTCCAGGACAGA |
| | CAGGCAAGATCGCCGACTACAACTACAAGCTGCCCGACGACTTTACCGGCTGCGTGATCG |
| | CTTGGAACAGCAATAACCTGGACTCAAAGGTTGGAGGAAACTACAACTACCTGTACAGAC |
| | TGTTCAGAAAGTCCAACCTGAAGCCCTTCGAGAGAGACATCTCTACAGAAATCTACCAGG |
| | CCGGCAGCACCCCATGTAACGGCGTGGAAGGCTTCAACTGCTACTTCCCTCTGCAGTCTT |
| | ATGGCTTCCAGCCCACAAACGGAGTGGGCTATCAGCCTTACCGCGTGGTTGTCCTGAGCT |
| | TTGAGCTGCTGCATGCCCCTGCTACGGTGTGTGGACCTAAGAAGTCCACCAACCTGGTGA |
| | AGAACAAGTGTGTGAACTTCAACTTCAACGGCCTGACCGGCACCGGGGTGCTGACAGAGT |
| | CTAACAAGAAATTCCTGCCATTCCAGCAATTCGGCCGGGACATCGCCGACACCACCGACG |
| | CCGTGCGGGATCCTCAGACCCTCGAAATCCTGGACATCACCCCCTGTAGCTTCGGCGGCG |
| | TGAGCGTGATCACCCCTGGCACAAACACCAGCAATCAAGTGGCTGTCCTGTACCAGGATG |
| | TCAATTGCACAGAAGTGCCTGTGGCCATCCACGCCGATCAGCTGACCCCCACCTGGCGGG |
| | TGTACTCGACAGGAAGCAACGTGTTTCAAACAAGAGCCGGCTGCCTGATCGGGGCCGAGC |
| | ACGTGAACAATTCCTACGAGTGCGACATCCCCATCGGCGCCGGCATCTGTGCCTCTTACC |
| | AGACACAGACCAATTCCCCTggtagtgcaagtTCCGTGGCCAGCCAGAGCATCATCGCCT |
| | ACACCATGAGCCTGGGCGCCGAAAACAGCGTTGCATATTCCAACAACAGCATCGCCATCC |
| | CTACCAACTTCACCATCAGCGTGACCACAGAAATCCTGCCTGTGTCCATGACCAAGACAA |
| | GCGTTGATTGCACCATGTACATCTGCGGCGATAGCACAGAGTGCAGCAATCTGCTGCTGC |
| | AGTACGGTAGCTTCTGCACCCAGCTGAATAGAGCCCTGACCGGCATCGCTGTGGAACAGG |
| | ACAAAAACACCCAGGAGGTCTTCGCCCAGGTGAAGCAAATCTACAAGACCCCTCCAATCA |
| | AGGACTTCGGAGGCTTTAACTTTAGCCAGATCCTGCCTGATCCCTCCAAGCCTAGCAAAC |
| | GGAGTcctATCGAGGACCTGCTCTTCAACAAGGTGACCCTGGCTGACGCCGGCTTCATTA |
| | AGCAGTACGGCGATTGCCTCGGCGACATCGCTGCAAGAGACCTGATCTGCGCCCAGAAGT |
| | TCAACGGCCTGACCGTGCTGCCTCCTCTCCTGACAGACGAGATGATCGCCCAGTACACCT |
| | CTGCCCTTCTGGCTGGCACCATCACCAGCGGATGGACCTTTGGAGCCGGAcctGCCCTGC |
| | AGATCCCTTTCcctATGCAGATGGCCTACAGATTCAACGGGATCGGAGTGACCCAAAACG |
| | TGCTGTATGAAAACCAGAAACTGATCGCCAATCAGTTTAACAGCGCCATCGGCAAAATCC |
| | AGGATAGCCTGTCCAGCACCccaAGCGCCCTCGGCAAGCTGCAAGATGTGGTGAATCAAA |
| | ATGCCCAAGCCCTGAACACACTGGTGAAGCAGCTGAGCAGCAACTTCGGCGCCATCAGCA |
| | GCGTGCTGAACGACATCCTGAGCAGACTGGACccacctGAAGCCGAGGTGCAGATCGACA |
| | GACTGATCACAGGCAGACTGCAGTCCCTGCAGACCTACGTGACCCAGCAGTTGATTAGAG |
| | CCGCTGAGATTAGAGCCAGTGCCAACCTGGCTGCCACAAAGATGTCAGAATGCGTGCTGG |
| | GCCAGAGCAAGAGAGTGGACTTCTGCGGCAAAGGCTACCACCTGATGAGCTTTCCTCAGT |
| | CTGCACCCCACGGCGTGGTGTTTCTCCACGTGACATACGTGCCCGCGCAAGAAAAGAACT |
| | TTACAACCGCCCCAGCGATCTGCCACGACGGCAAGGCCCACTTCCCTCGGGAGGGTGTGT |
| | TCGTGAGCAATGGAACACACTGGTTCGTCACCCAGCGGAACTTCTACGAGCCTCAGATCA |
| | TTACCACCGACAACACCTTCGTGAGCGGCAACTGTGACGTCGTTATCGGCATCGTGAACA |
| | ATACCGTGTACGACCCCCTGCAGCCTGAGCTGGATAGCTTCAAAGAGGAACTGGACAAGT |
| | ACTTCAAGAACCACACAAGCCCCGACGTGGACCTAGGCGACATCTCTGGAATCAACGCCA |
| | GCGTGGTGAACATCCAAAAGGAAATCGACAGACTGAACGAGGTGGCCAAGAATCTGAATG |
| | AAAGCCTGATCGATCTGCAGGAGCTGGGCAAGTACGAGCAGggtggcggtggctcgCTGA |
| | TTACCTATATCGTCCTGACTATTATCTCCCTGGTGTTTGGCATTCTGTCCCTGATTCTGG |
| | CCTGTTACCTGATGTACAAGCAGAAGGCCCAGCAGAAGACCCTGCTGTGGCTGGGCAATA |
| | ATACACTGGATCAGATGCGGGCTACAACTAAGATGTGAacgcgtACCCAAGGTCCAACTC |
| | TCCAAGCGGCAATCCTCTCTCGCTTCCTCAGCCCCACTGAATGGTCGCGTAACCGTAATT |
| | AATCTAGCTACATTTAAGATTAAGAAAAAATACGGGTAGAATTGGAGTGCCCCAATTGTG |
| | CCAAGATGGACTCATCTAGGACAATTGGGCTGTACTTTGATTCTGCCCATTCTTCTAGCA |
| | ACCTGTTAGCATTTCCGATCGTCCTACAAGGCACAGGAGATGGGAAGAAGCAAATCGCCC |
| | CGCAATATAGGATCCAGCGCCTTGACTTGTGGACTGATAGTAAGGAGGACTCAGTATTCA |
| | TCACCACCTATGGATTCATCTTTCAAGTTGGGAATGAAGAAGCCACTGTCGGCATGATCG |
| | ATGATAAACCCAAGCGCGAGTTACTTTCCGCTGCGATGCTCTGCCTAGGAAGCGTCCCAA |
| | ATACCGGAGACCTTATTGAGCTGGCAAGGGCCTGTCTCACTATGATAGTCACATGCAAGA |
| | AGAGTGCAACTAATACTGAGAGAATGGTTTTCTCAGTAGTGCAGGCACCCCAAGTGCTGC |
| | AAAGCTGTAGGGTTGTGGCAAACAAATACTCATCAGTGAATGCAGTCAAGCACGTGAAAG |
| | CGCCAGAGAAGATTCCCGGGAGTGGAACCCTAGAATACAAGGTGAACTTTGTCTCCTTGA |
| | CTGTGGTACCGAAGAAGGATGTCTACAAGATCCCAGCTGCAGTATTGAAGGTTTCTGGCT |
| | CGAGTCTGTACAATCTTGCGCTCAATGTCACTATTAATGTGGAGGTAGACCCGAGGAGTC |
| | CTTTGGTTAAATCTTTGTCTAAGTCTGACAGCGGATACTATGCTAACCTCTTCTTGCATA |
| | TTGGACTTATGACCACCGTAGATAGGAAGGGGAAGAAAGTGACATTTGACAAGCTGGAAA |
| | AGAAAATAAGGAGCCTTGATCTATCTGTCGGGCTCAGTGATGTGCTCGGGCCTTCCGTGT |
| | TGGTAAAAGCAAGAGGTGCACGGACTAAGCTTTTGGCACCTTTCTTCTCTAGCAGTGGGA |
| | CAGCCTGCTATCCCATAGCAAATGCTTCTCCTCAGGTGGCCAAGATACTCTGGAGTCAAA |
| | CCGCGTGCCTGCGGAGCGTTAAAATCATTATCCAAGCAGGTACCCAACGCGCTGTCGCAG |
| | TGACCGCCGACCACGAGGTTACCTCTACTAAGCTGGAGAAGGGGCACACCCTTGCCAAAT |
| | ACAATCCTTTTAAGAAATAAGCTGCGTCTCTGAGATTGCGCTCCGCCCACTCACCCAGAT |
| | CATCATGACACAAAAAACTAATCTGTCTTGATTATTTACAGTTAGTTTACCTGTCTATCA |
| | AGTTAGAAAAAACACGGGTAGAAGATTCTGGATCCCGGTTGGCGCCCTCCAGGTGCAAGt |
| | taattaaATGGGCTCCAGACCTTCTACCAAGAACCCAGCACCTATGATGCTGACTATCCG |
| | GGTTGCGCTGGTACTGAGTTGCATCTGTCCGGCAAACTCCATTGATGGCAGGCCTCTTGC |
| | AGCTGCAGGAATTGTGGTTACAGGAGACAAAGCCGTCAACATATACACCTCATCCCAGAC |
| | AGGATCAATCATAGTTAAGCTCCTCCCGAATCTGCCCAAGGATAAGGAGGCATGTGCGAA |
| | AGCCCCCTTGGATGCATACAACAGGACATTGACCACTTTGCTCACCCCCCTTGGTGACTC |
| | TATCCGTAGGATACAAGAGTCTGTGACTACATCTGGAGGGGGGAGACAGGGGCGCCTTAT |
| | AGGCGCCATTATTGGCGGTGTGGCTCTTGGGGTTGCAACTGCCGCACAAATAACAGCGGC |
| | CGCAGCTCTGATACAAGCCAAACAAAATGCTGCCAACATCCTCCGACTTAAAGAGAGCAT |
| | TGCCGCAACCAATGAGGCTGTGCATGAGGTCACTGACGGATTATCGCAACTAGCAGTGGC |
| | AGTTGGGAAGATGCAGCAGTTTGTTAATGACCAATTTAATAAAACAGCTCAGGAATTAGA |
| | CTGCATCAAAATTGCACAGCAAGTTGGTGTAGAGCTCAACCTGTACCTAACCGAATTGAC |
| | TACAGTATTCGGACCACAAATCACTTCACCTGCTTTAAACAAGCTGACTATTCAGGCACT |
| | TTACAATCTAGCTGGTGGAAATATGGATTACTTATTGACTAAGTTAGGTGTAGGGAACAA |
| | TCAACTCAGCTCATTAATCGGTAGCGGCTTAATCACtGGcAACCCTATTCTATACGACTC |
| | ACAGACTCAACTCTTGGGTATACAGGTAACTgcaCCTTCAGTCGGGAACCTAAATAATAT |
| | GCGTGCCACCTACTTGGAAACCTTATCCGTAAGCACAACCAGGGGATTTGCCTCGGCACT |
| | TGTCCCCAAAGTGGTGACACAGGTCGGTTCTGTGATAGAAGAACTTGACACCTCATACTG |
| | TATAGAAACTGACTTAGATTTATATTGTACAAGAATAGTAACGTTCCCTATGTCCCCTGG |
| | TATTTATTCCTGCTTGAGCGGCAATACGTCGGCCTGTATGTACTCAAAGACCGAAGGCGC |
| | ACTTACTACACCATACATGACTATCAAAGGTTCAGTCATCGCCAACTGCAAGATGACAAC |
| | ATGTAGATGTGTAAACCCCCCGGGTATCATATCGCAAAACTATGGAGAAGCCGTGTCTCT |
| | AATAGATAAACAATCATGCAATGTTTTATCCTTAGGCGGGATAACTTTAAGGCTCAGTGG |
| | GGAATTCGATGTAACTTATCAGAAGAATATCTCAATACAAGATTCTCAAGTAATAATAAC |
| | AGGCAATCTTGATATCTCAACTGAGCTTGGGAATGTCAACAACTCGATCAGTAATGCTTT |
| | GAATAAGTTAGAGGAAAGCAACAGAAAACTAGACAAAGTCAATGTCAAACTGACTAGCAC |
| | ATCTGCTCTCATTACgTATATCGTTTTGACTATCATATCTCTTGTTTTTGGTATACTTAG |
| | CCTGATTCTAGCATGCTACCTAATGTACAAGCAAAAGGCGCAACAAAAGACCTTATTATG |
| | GCTTGGGAATAATACaCTcGATCAGATGAGAGCCACTACAAAAATGTGAACACAGATGAG |
| | GAACGAAGGTTTCCCTAATAGTAATTTGTGTGAAAGTTCTGGTAGTCTGTCAGTTCAGAG |
| | AGTTAAGAAAAAACTACCGGTTGTAGATGACCAAAGGACGATATACGGGTAGAACGGTAA |
| | GAGAGGCCGCCCCTCAATTGCGAGCCAGGCTTCACAACCTCCGTTCTACCGCTTCACCGA |
| | CAACAGTCCTCAATCATGGACCGCGCCGTTAGCCAAGTTGCGTTAGAGAATGATGAAAGA |
| | GAGGCAAAAAATACATGGCGCTTGATATTCCGGATTGCAATCTTATTCTTAACAGTAGTG |
| | ACCTTGGCTATATCTGTAGCCTCCCTTTTATATAGCATGGGGGCTAGCACACCTAGCGAT |
| | CTTGTAGGCATACCGACTAGGATTTCCAGGGCAGAAGAAAAGATTACATCTACACTTGGT |
| | TCCAATCAAGATGTAGTAGATAGGATATATAAGCAAGTGGCCCTTGAGTCTCCGTTGGCA |
| | TTGTTAAATACTGAGACCACAATTATGAACGCAATAACATCTCTCTCTTATCAGATTAAT |
| | GGAGCTGCAAACAACAGTGGGTGGGGGGCACCTATCCATGACCCAGATTATATAGGGGGG |
| | ATAGGCAAAGAACTCATTGTAGATGATGCTAGTGATGTCACATCATTCTATCCCTCTGCA |
| | TTTCAAGAACATCTGAATTTTATCCCGGCGCCTACTACAGGATCAGGTTGCACTCGAATA |
| | CCCTCATTTGACATGAGTGCTACCCATTACTGCTACACCCATAATGTAATATTGTCTGGA |
| | TGCAGAGATCACTCACATTCATATCAGTATTTAGCACTTGGTGTGCTCCGGACATCTGCA |
| | ACAGGGAGGGTATTCTTTTCTACTCTGCGTTCCATCAACCTGGACGACACCCAAAATCGG |
| | AAGTCTTGCAGTGTGAGTGCAACTCCCCTGGGTTGTGATATGCTGTGCTCGAAAGTCACG |
| | GAGACAGAGGAAGAAGATTATAACTCAGCTGTCCCTACGCGGATGGTACATGGGAGGTTA |
| | GGGTTCGACGGCCAGTACCACGAAAAGGACCTAGATGTCACAACATTATTCGGGGACTGG |
| | GTGGCCAACTACCCAGGAGTAGGGGGTGGATCTTTTATTGACAGCCGCGTATGGTTCTCA |
| | GTCTACGGAGGGTTAAAACCCAATTCACCCAGTGACACTGTACAGGAAGGGAAATATGTG |
| | ATATACAAGCGATACAATGACACATGCCCAGATGAGCAAGACTACCAGATTCGAATGGCC |
| | AAGTCTTCGTATAAGCCTGGACGGTTTGGTGGGAAACGCATACAGCAGGCTATCTTATCT |
| | ATCAAGGTGTCAACATCCTTAGGCGAAGACCCGGTACTGACTGTACCGCCCAACACAGTC |
| | ACACTCATGGGGGCCGAAGGCAGAATTCTCACAGTAGGGACATCTCATTTCTTGTATCAA |
| | CGAGGGTCATCATACTTCTCTCCCGCGTTATTATATCCTATGACAGTCAGCAACAAAACA |
| | GCCACTCTTCATAGTCCTTATACATTCAATGCCTTCACTCGGCCAGGTAGTATCCCTTGC |
| | CAGGCTTCAGCAAGATGCCCCAACTCGTGTGTTACTGGAGTCTATACAGATCCATATCCC |
| | CTAATCTTCTATAGAAACCACACCTTGCGAGGGGTATTCGGGACAATGCTTGATGGTGTA |
| | CAAGCAAGACTTAACCCTGCGTCTGCAGTATTCGATAGCACATCCCGCAGTCGCATTACT |
| | CGAGTGAGTTCAAGCAGTACCAAAGCAGCATACACAACATCAACTTGTTTTAAAGTGGTC |
| | AAGACTAATAAGACCTATTGTCTCAGCATTGOTGAAATATCTAATACTCTCTTCGGAGAA |
| | TTCAGAATCGTCCCGTTACTAGTTGAGATCCTCAAAGATGACGGGGTTAGAGAAGCCAGG |
| | TCTGGCTAGggcgcgccTTGAGTCAATTATAAAGGAGTTGGAAAGATGGCATTGTATCAC |
| | CTATCTTCTGCGACATCAAGAATCAAACCGAATGCCGGCGCGTGCTCGAATTCCATGTTG |
| | CCAGTTGACCACAATCAGCCAGTGCTCATGCGATCAGATTAAGCCTTGTCATTAATCTCT |
| | TGATTAAGAAAAAATGTAAGTGGCAATGAGATACAAGGCAAAACAGCTCATGGTAAATAA |
| | TACGGGTAGGACATGGCGAGCTCCGGTCCTGAAAGGGCAGAGCATCAGATTATCCTACCA |
| | GAGCCACACCTGTCTTCACCATTGGTCAAGCACAAACTACTCTATTACTGGAAATTAACT |
| | GGGCTACCGCTTCCTGATGAATGTGACTTCGACCACCTCATTCTCAGCCGACAATGGAAA |
| | AAAATACTTGAATCGGCCTCTCCTGATACTGAGAGAATGATAAAACTCGGAAGGGCAGTA |
| | CACCAAACTCTTAACCACAATTCCAGAATAACCGGAGTGCTCCACCCCAGGTGTTTAGAA |
| | CAACTGGCTAATATTGAGGTCCCAGATTCAACCAACAAATTTCGGAAGATTGAGAAGAAG |
| | ATCCAAATTCACAACACGAGATATGGAGAACTGTTCACAAGGCTGTGTACGCATATAGAG |
| | AAGAAACTGCTGGGGTCATCTTGGTCTAACAATGTCCCCCGGTCAGAGGAGTTCAGCAGC |
| | ATTCGTACGGATCCGGCATTCTGGTTTCACTCAAAATGGTCCACAGCCAAGTTTGCATGG |
| | CTCCATATAAAACAGATCCAGAGGCATCTGATGGTGGCAGCTAAGACAAGGTCTGCGGCC |
| | AACAAATTGGTGATGCTAACCCATAAGGTAGGCCAAGTCTTTGTCACTCCTGAACTTGTC |
| | GTTGTGACGCATACGAATGAGAACAAGTTCACATGTCTTACCCAGGAACTTGTATTGATG |
| | TATGCAGATATGATGGAGGGCAGAGATATGGTCAACATAATATCAACCACGGCGGTGCAT |
| | CTCAGAAGCTTATCAGAGAAAATTGATGACATTTTGCGGTTAATAGACGCTCTGGCAAAA |
| | GACTTGGGTAATCAAGTCTACGATGTTGTATCACTAATGGAGGGATTTGCATACGGAGCT |
| | GTCCAGCTACTCGAGCCGTCAGGTACATTTGCAGGAGATTTCTTCGCATTCAACCTGCAG |
| | GAGCTTAAAGACATTCTAATTGGCCTCCTCCCCAATGATATAGCAGAATCCGTGACTCAT |
| | GCAATCGCTACTGTATTCTCTGGTTTAGAACAGAATCAAGCAGCTGAGATGTTGTGTCTG |
| | TTGCGTCTGTGGGGTCACCCACTGCTTGAGTCCCGTATTGCAGCAAAGGCAGTCAGGAGC |
| | CAAATGTGCGCACCGAAAATGGTAGACTTTGATATGATCCTTCAGGTACTGTCTTTCTTC |
| | AAGGGAACAATCATCAACGGGTACAGAAAGAAGAATGCAGGTGTGTGGCCGCGAGTCAAA |
| | GTGGATACAATATATGGGAAGGTCATTGGGCAACTACATGCAGATTCAGCAGAGATTTCA |
| | CACGATATCATGTTGAGAGAGTATAAGAGTTTATCTGCACTTGAATTTGAGCCATGTATA |
| | GAATATGACCCTGTCACCAACCTGAGCATGTTCCTAAAAGACAAGGCAATCGCACACCCC |
| | AACGATAATTGGCTTGCCTCGTTTAGGCGGAACCTTCTCTCCGAAGACCAGAAGAAACAT |
| | GTAAAAGAAGCAACTTCGACTAATCGCCTCTTGATAGAGTTTTTAGAGTCAAATGATTTT |
| | GATCCATATAAAGAGATGGAATATCTGACGACCCTTGAGTACCTTAGAGATGACAATGTG |
| | GCAGTATCATACTCGCTCAAGGAGAAGGAAGTGAAAGTTAATGGACGGATCTTCGCTAAG |
| | CTGACAAAGAAGTTAAGGAACTGTCAGGTGATGGCGGAAGGGATCCTAGCCGATCAGATT |
| | GCACCTTTCTTTCAGGGAAATGGAGTCATTCAGGATAGCATATCCTTGACCAAGAGTATG |
| | CTAGCGATGAGTCAACTGTCTTTTAACAGCAATAAGAAACGTATCACTGACTGTAAAGAA |
| | AGAGTATCTTCAAACCGCAATCATGATCCGAAAAGCAAGAACCGTCGGAGAGTTGCAACC |
| | TTCATAACAACTGACCTGCAAAAGTACTGTCTTAATTGGAGATATCAGACAATCAAATTG |
| | TTCGCTCATGCCATCAATCAGTTGATGGGCCTACCTCACTTCTTCGAATGGATTCACCTA |
| | AGACTGATGGACACTACGATGTTCGTAGGAGACCCTTTCAATCCTCCAAGTGACCCTACT |
| | GACTGTGACCTCTCAAGAGTCCCTAATGATGACATATATATTGTCAGTGCCAGAGGGGGT |
| | ATCGAAGGATTATGCCAGAAGCTATGGACAATGATCTCAATTGCTGCAATCCAACTTGCT |
| | GCAGCTAGATCGCATTGTCGTGTTGCCTGTATGGTACAGGGTGATAATCAAGTAATAGCA |
| | GTAACGAGAGAGGTAAGATCAGACGACTCTCCGGAGATGGTGTTGACACAGTTGCATCAA |
| | GCCAGTGATAATTTCTTCAAGGAATTAATTCATGTCAATCATTTGATTGGCCATAATTTG |
| | AAGGATCGTGAAACCATCAGGTCAGACACATTCTTCATATACAGCAAACGAATCTTCAAA |
| | GATGGAGCAATCCTCAGTCAAGTCCTCAAAAATTCATCTAAATTAGTGCTAGTGTCAGGT |
| | GATCTCAGTGAAAACACCGTAATGTCCTGTGCCAACATTGCCTCTACTGTAGCACGGCTA |
| | TGCGAGAACGGGCTTCCCAAAGACTTCTGTTACTATTTAAACTATATAATGAGTTGTGTG |
| | CAGACATACTTTGACTCTGAGTTCTCCATCACCAACAATTCGCACCCCGATCTTAATCAG |
| | TCGTGGATTGAGGACATCTCTTTTGTGCACTCATATGTTCTGACTCCTGCCCAATTAGGG |
| | GGACTGAGTAACCTTCAATACTCAAGGCTCTACACTAGAAATATCGGTGACCCGGGGACT |
| | ACTGCTTTTGCAGAGATCAAGCGACTAGAAGCAGTGGGATTACTGAGTCCTAACATTATG |
| | ACTAATATCTTAACTAGGCCGCCTGGGAATGGAGATTGGGCCAGTCTGTGCAACGACCCA |
| | TACTCTTTCAATTTTGAGACTGTTGCAAGCCCAAATATTGTTCTTAAGAAACATACGCAA |
| | AGAGTCCTATTTGAAACTTGTTCAAATCCCTTATTGTCTGGAGTGCACACAGAGGATAAT |
| | GAGGCAGAAGAGAAGGCATTGGCTGAATTCTTGCTTAATCAAGAGGTGATTCATCCCCGC |
| | GTTGCGCATGCCATCATGGAGGCAAGCTCTGTAGGTAGGAGAAAGCAAATTCAAGGGCTT |
| | GTTGACACAACAAACACCGTAATTAAGATTGCGCTTACTAGGAGGCCATTAGGCATCAAG |
| | AGGCTGATGCGGATAGTCAATTATTCTAGCATGCATGCAATGCTGTTTAGAGACGATGTT |
| | TTTTCCTCCAGTAGATCCAACCACCCCTTAGTCTCTTCTAATATGTGTTCTCTGACACTG |
| | GCAGACTATGCACGGAATAGAAGCTGGTCACCTTTGACGGGAGGCAGGAAAATACTGGGT |
| | GTATCTAATCCTGATACGATAGAACTCGTAGAGGGTGAGATTCTTAGTGTAAGCGGAGGG |
| | TGTACAAGATGTGACAGCGGAGATGAACAATTTACTTGGTTCCATCTTCCAAGCAATATA |
| | GAATTGACCGATGACACCAGCAAGAATCCTCCGATGAGGGTACCATATCTCGGGTCAAAG |
| | ACACAGGAGAGGAGAGCTGCCTCACTTGCAAAAATAGCTCATATGTCGCCACATGTAAAG |
| | GCTGCCCTAAGGGCATCATCCGTGTTGATCTGGGCTTATGGGGATAATGAAGTAAATTGG |
| | ACTGCTGCTCTTACGATTGCAAAATCTCGGTGTAATGTAAACTTAGAGTATCTTCGGTTA |
| | CTGTCCCCTTTACCCACGGCTGGGAATCTTCAACATAGACTAGATGATGGTATAACTCAG |
| | ATGACATTCACCCCTGCATCTCTCTACAGGgtgtcaccttacattcacatatccaatgat |
| | tctcaaaggctgttcactgaagaaggagtcaaagaggggaatgtggtttaccaacagatc |
| | ATGCTCTTGGGTTTATCTCTAATCGAATCGATCTTTCCAATGACAACAACCAGGACATAT |
| | GATGAGATCACACTGCACCTACATAGTAAATTTAGTTGCTGTATCAGAGAAGCACCTGTT |
| | GCGGTTCCTTTCGAGCTACTTGGGGTGGTACCGGAACTGAGGACAGTGACCTCAAATAAG |
| | TTTATGTATGATCCTAGCCCTGTATCGGAGGGAGACTTTGCGAGACTTGACTTAGCTATC |
| | TTCAAGAGTTATGAGCTTAATCTGGAGTCATATCCCACGATAGAGCTAATGAACATTCTT |
| | TCAATATCCAGCGGGAAGTTGATTGGCCAGTCTGTGGTTTCTTATGATGAAGATACCTCC |
| | ATAAAGAATGACGCCATAATAGTGTATGACAATACCCGAAATTGGATCAGTGAAGCTCAG |
| | AATTCAGATGTGGTCCGCCTATTTGAATATGCAGCACTTGAAGTGCTCCTCGACTGTTCT |
| | TACCAACTCTATTACCTGAGAGTAAGAGGCCTAGACAATATTGTCTTATATATGGGTGAT |
| | TTATACAAGAATATGCCAGGAATTCTACTTTCCAACATTGCAGCTACAATATCTCATCCC |
| | GTCATTCATTCAAGGTTACATGCAGTGGGCCTGGTCAACCATGACGGATCACACCAACTT |
| | GCAGATACGGATTTTATCGAAATGTCTGCAAAACTATTAGTATCTTGCACCCGACGTGTG |
| | ATCTCCGGCTTATATTCAGGAAATAAGTATGATCTGCTGTTCCCATCTGTCTTAGATGAT |
| | AACCTGAATGAGAAGATGCTTCAGCTGATATCCCGGTTATGCTGTCTGTACACGGTACTC |
| | TTTGCTACAACAAGAGAAATCCCGAAAATAAGAGGCTTAACTGCAGAAGAGAAATGTTCA |
| | ATACTCACTGAGTATTTACTGTCGGATGCTGTGAAACCATTACTTAGCCCCGATCAAGTG |
| | AGCTCTATCATGTCTCCTAACATAATTACATTCCCAGCTAATCTGTACTACATGTCTCGG |
| | AAGAGCCTCAATTTGATCAGGGAAAGGGAGGACAGGGATACTATCCTGGCGTTGTTGTTC |
| | CCCCAAGAGCCATTATTAGAGTTCCCTTCTGTGCAAGATATTGGTGCTCGAGTGAAAGAT |
| | CCATTCACCCGACAACCTGCGGCATTTTTGCAAGAGTTAGATTTGAGTGCTCCAGCAAGG |
| | TATGACGCATTCACACTTAGTCAGATTCATCCTGAACTCACATCTCCAAATCCGGAGGAA |
| | GACTACTTAGTACGATACTTGTTCAGAGGGATAGGGACTGCATCTTCCTCTTGGTATAAG |
| | GCATCTCATCTCCTTTCTGTACCCGAGGTAAGATGTGCAAGACACGGGAACTCCTTATAC |
| | TTAGCTGAAGGGAGCGGAGCCATCATGAGTCTTCTCGAACTGCATGTACCACATGAAACT |
| | ATCTATTACAATACGCTCTTTTCAAATGAGATGAACCCCCCGCAACGACATTTCGGGCCG |
| | ACCCCAACTCAGTTTTTGAATTCGGTTGTTTATAGGAATCTACAGGCGGAGGTAACATGC |
| | AAAGATGGATTTGTCCAAGAGTTCCGTCCATTATGGAGAGAAAATACAGAGGAAAGTGAC |
| | CTGACCTCAGATAAAGCAGTGGGGTATATTACATCTGCAGTGCCCTACAGATCTGTATCA |
| | TTGCTGCATTGTGACATTGAAATTCCTCCAGGGTCCAATCAAAGCTTACTAGATCAACTA |
| | GCTATCAATTTATCTCTGATTGCCATGCATTCTGTAAGGGAGGGCGGGGTAGTAATCATC |
| | AAAGTGTTGTATGCAATGGGATACTACTTTCATCTACTCATGAACTTGTTTGCTCCGTGT |
| | TCCACAAAAGGATATATTCTCTCTAATGGTTATGCATGTCGAGGAGATATGGAGTGTTAC |
| | CTGGTATTTGTCATGGGTTACCTGGGCGGGCCTACATTTGTACATGAGGTGGTGAGGATG |
| | GCAAAAACTCTGGTGCAGCGGCACGGTACGCTCTTGTCTAAATCAGATGAGATCACACTG |
| | ACCAGGTTATTCACCTCACAGCGGCAGCGTGTGACAGACATCCTATCCAGTCCTTTACCA |
| | AGATTAATAAAGTACTTGAGGAAGAATATTGACACTGCGCTGATTGAAGCCGGGGGACAG |
| | CCCGTCCGTCCATTCTGTGCGGAGAGTCTGGTGAGCACGCTAGCGAACATAACTCAGATA |
| | ACCCAGATTATCGCTAGTCACATTGACACAGTTATCCGGTCTGTGATATATATGGAAGCT |
| | GAGGGTGATCTCGCTGACACAGTATTTCTATTTACCCCTTACAATCTCTCTACTGACGGG |
| | AAAAAGAGGACATCACTTATACAGTGCACGAGACAGATCCTAGAGGTTACAATACTAGGT |
| | CTTAGAGTCGAAAATCTCAATAAAATAGGCGATATAATCAGCCTAGTGCTTAAAGGCATG |
| | ATCTCCATGGAGGACCTTATCCCACTAAGGACATACTTGAAGCATAGTACCTGCCCTAAA |
| | TATTTGAAGGCTGTCCTAGGTATTACCAAACTCAAAGAAATGTTTACAGACACTTCTGTA |
| | TTGTACTTGACTCGTGCTCAACAAAAATTCTACATGAAAACTATAGGCAATGCAGTCAAA |
| | GGATATTACAGTAACTGTGACTCTTAACGAAAATCACATATTAATAGGCTCCTTTTTTGG |
| | CCAATTGTATTCTTGTTGATTTAATCATATTATGTTAGAAAAAAGTTGAACCCTGACTCC |
| | TTAGGACTCGAATTCGAACTCAAATAAATGTCTTAAAAAAAGGTTGCGCACAATTATTCT |
| | TGAGTGTAGTCTCGTCATTCACCAAATCTTTGTTTGGTGGCCGGCATGGTCCCAGCCTCC |
| | TCGCTGGCGCCGGCTGGGCAACATTCCGAGGGGACCGTCCCCTCGGTAATGGCGAATGGG |
| | ACGTCGACTGCTAACAAAGCCCGAAAGGAAGCTGAGTTGGCTGCTGCCACCGCTGAGCAA |
| | TAACTAGCATAACCCCTTGGGGCCTCTAAACGGGTCTTGAGGGGTTTTTTGCTGAAAGGA |
| | GGAACTATA |
| SEQ ID NO: | TAATACGACTCACTATAGGGACCAAACAGAGAATCCGTGAGTTACGATAAAAGGCGAAGG |
| 5; NDV- | AGCAATTGAAGTCGCACGGGTAGAAGGTGTGAATCTCGAGTGCGAGCCCGAAGCACAAAC |
| APMV5 F-HN | TCGAGAAAGCCTTCTGCCAACATGTCTTCCGTATTTGATGAGTACGAACAGCTCCTCGCG |
| Chimeric - | GCTCAGACTCGCCCCAATGGAGCTCATGGAGGGGGAGAAAAAGGGAGTACCTTAAAAGTA |
| expressing | GACGTCCCGGTATTCACTCTTAACAGTGATGACCCAGAAGATAGATGGAGCTTTGTGGTA |
| GFP | TTCTGCCTCCGGATTGCTGTTAGCGAAGATGCCAACAAACCACTCAGGCAAGGTGCTCTC |
| | ATATCTCTTTTATGCTCCCACTCACAGGTAATGAGGAACCATGTTGCCATTGCAGGGAAA |
| | CAGAATGAAGCCACATTGGCCGTGCTTGAGATTGATGGCTTTGCCAACGGCACGCCCCAG |
| | TTCAACAATAGGAGTGGAGTGTCTGAAGAGAGAGCACAGAGATTTGCGATGATAGCAGGA |
| | TCTCTCCCTCGGGCATGCAGCAACGGAACCCCGTTCGTCACAGCCGGGGCAGAAGATGAT |
| | GCACCAGAAGACATCACCGATACCCTGGAGAGGATCCTCTCTATCCAGGCTCAAGTATGG |
| | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaTTAGAAAAAATA |
| | CGGGTAGAACCGCCACCATGGTGAGCAAGGGCGAGGAGCTGTTCACCGGGGTGGTGCCCA |
| | TCCTGGTCGAGCTGGACGGCGACGTAAACGGCCACAAGTTCAGCGTGTCCGGCGAGGGCG |
| | AGGGCGATGCCACCTACGGCAAGCTGACCCTGAAGTTCATCTGCACCACCGGCAAGCTGC |
| | CCGTGCCCTGGCCCACCCTCGTGACCACCCTGACCTACGGCGTGCAGTGCTTCAGCCGCT |
| | ACCCCGACCACATGAAGCAGCACGACTTCTTCAAGTCCGCCATGCCCGAAGGCTACGTCC |
| | AGGAGCGCACCATCTTCTTCAAGGACGACGGCAACTACAAGACCCGCGCCGAGGTGAAGT |
| | TCGAGGGCGACACCCTGGTGAACCGCATCGAGCTGAAGGGCATCGACTTCAAGGAGGACG |
| | GCAACATCCTGGGGCACAAGCTGGAGTACAACTACAACAGCCACAACGTCTATATCATGG |
| | CCGACAAGCAGAAGAACGGCATCAAGGTGAACTTCAAGATCCGCCACAACATCGAGGACG |
| | GCAGCGTGCAGCTCGCCGACCACTACCAGCAGAACACCCCCATCGGCGACGGCCCCGTGC |
| | TGCTGCCCGACAACCACTACCTGAGCACCCAGTCCGCCCTGAGCAAAGACCCCAACGAGA |
| | AGCGCGATCACATGGTCCTGCTGGAGTTCGTGACCGCCGCCGGGATCACTCTCGGCATGG |
| | ACGAGCTGTACAAGTaATaaacgcgtACCCAAGGTCCAACTCTCCAAGCGGCAATCCTCT |
| | CTCGCTTCCTCAGCCCCACTGAATGGTCGCGTAACCGTAATTAATCTAGCTACATTTAAG |
| | ATTAAGAAAAAATACGGGTAGAATTGGAGTGCCCCAATTGTGCCAAGATGGACTCATCTA |
| | GGACAATTGGGCTGTACTTTGATTCTGCCCATTCTTCTAGCAACCTGTTAGCATTTCCGA |
| | TCGTCCTACAAGGCACAGGAGATGGGAAGAAGCAAATCGCCCCGCAATATAGGATCCAGC |
| | GCCTTGACTTGTGGACTGATAGTAAGGAGGACTCAGTATTCATCACCACCTATGGATTCA |
| | TCTTTCAAGTTGGGAATGAAGAAGCCACTGTCGGCATGATCGATGATAAACCCAAGCGCG |
| | AGTTACTTTCCGCTGCGATGCTCTGCCTAGGAAGCGTCCCAAATACCGGAGACCTTATTG |
| | AGCTGGCAAGGGCCTGTCTCACTATGATAGTCACATGCAAGAAGAGTGCAACTAATACTG |
| | AGAGAATGGTTTTCTCAGTAGTGCAGGCACCCCAAGTGCTGCAAAGCTGTAGGGTTGTGG |
| | CAAACAAATACTCATCAGTGAATGCAGTCAAGCACGTGAAAGCGCCAGAGAAGATTCCCG |
| | GGAGTGGAACCCTAGAATACAAGGTGAACTTTGTCTCCTTGACTGTGGTACCGAAGAAGG |
| | ATGTCTACAAGATCCCAGCTGCAGTATTGAAGGTTTCTGGCTCGAGTCTGTACAATCTTG |
| | CGCTCAATGTCACTATTAATGTGGAGGTAGACCCGAGGAGTCCTTTGGTTAAATCTTTGT |
| | CTAAGTCTGACAGCGGATACTATGCTAACCTCTTCTTGCATATTGGACTTATGACCACCG |
| | TAGATAGGAAGGGGAAGAAAGTGACATTTGACAAGCTGGAAAAGAAAATAAGGAGCCTTG |
| | ATCTATCTGTCGGGCTCAGTGATGTGCTCGGGCCTTCCGTGTTGGTAAAAGCAAGAGGTG |
| | CACGGACTAAGCTTTTGGCACCTTTCTTCTCTAGCAGTGGGACAGCCTGCTATCCCATAG |
| | CAAATGCTTCTCCTCAGGTGGCCAAGATACTCTGGAGTCAAACCGCGTGCCTGCGGAGCG |
| | TTAAAATCATTATCCAAGCAGGTACCCAACGCGCTGTCGCAGTGACCGCCGACCACGAGG |
| | TTACCTCTACTAAGCTGGAGAAGGGGCACACCCTTGCCAAATACAATCCTTTTAAGAAAT |
| | AAGCTGCGTCTCTGAGATTGCGCTCCGCCCACTCACCCAGATCATCATGACACAAAAAAC |
| | TAATCTGTCTTGATTATTTACAGTTAGTTTACCTGTCTATCAAGTTAGAAAAAACACGGG |
| | TAGAAGATTCTGGATCCCGGTTGGCGCCCTCCAGGTGCAAGttaattaaATGTTACAACT |
| | TCCCTTGACCATTCTTCTTAGCATTCTTAGTGCTCACCAGTCGCTTTGTCTAGACAACAG |
| | TAAGCTCATTCATGCAGGAATCATGAGTACTACTGAGAGAGAAGTTAATGTTTATGCACA |
| | ATCTATTACTGGGTCAATAGTGGTGAGATTGATTCCAAATATCCCAAGTAACCATAAATC |
| | TTGTGCAACTAGCCAAATCAAATTATACAATGACACGTTAACAAGATTGTTGACCCCAAT |
| | TAAAGCTAATCTAGAAGGACTTATTAGTGCTGTTTCTCAGGACCAATCGCAGAATTCTGG |
| | GAAGAGAAAGAAGCGTTTTGTAGGCGCAGTAATTGGAGCAGCTGCCCTTGGTTTGGCAAC |
| | TGCTGCACAGGTGACTGCCACTGTAGCATTAAATCAAGCGCAAGAAAACGCTCGGAATAT |
| | CCTAAGGCTTAAAAACTCGATTCAGAAGACAAACGAGGCGGTGATGGAACTTAAAGATGC |
| | TGTGGGCCAAACAGCAGTAGCTATTGACAAAACTCAGGCCTTCATAAATAATCAAATCTT |
| | GCCTGCAATTTCAAATCTCTCATGTGAGGTCCTAGGGAATAAAATTGGGGTCCAATTATC |
| | TTTGTACCTTACTGAATTAACAACAGTATTCGGCAACCAACTGACAAACCCAGCCCTTAC |
| | CACACTGTCATTACAAGCCTTGTACAATCTTTGTGGAGATGACTTCAATTACTTAATCAA |
| | CCTATTAAATGCAAAAAATCGTAACTTAGCCTCACTTTATGAAGCAAACCTAATTCAGGG |
| | GAGAATTACTCAATATGACTCAATGAATCAGTTATTAATTATTCAGGTACAAATACCAAG |
| | CATCTCCACAGTGTCAGGAATGAGGGTCACAGAATTGTTCACACTTAGTGTTGACACACC |
| | TATAGGAGAGGGAAAGGCCCTAGTACCAAAATATGTCCTATCCTCAGGGAGAATAATGGA |
| | AGAGGTTGACCTAAGCAGTTGCGCTATAACATCAACATCAGTTTTCTGTTCCTCTATCAT |
| | CTCTAGACCCCTTCCACTTGAAACAATAAATTGCCTGAATGGGAATGTTACACAGTGTCA |
| | ATTTACCGCCAACACAGGAACCCTTGAATCGAGATACGCTGTTATAGGAGGATTGGTGAT |
| | TGCTAACTGTAAGGCTATAGTATGCAGGTGCCTAAATCCACCAGGTGTCATTGCGCAAAA |
| | TCTTGGCTTACCAATTACAATCATCTCATCCAATACTTGTCAGCGAATTAATTTAGAACA |
| | AATCACTTTGTCTCTTGGGAACAGCATATTATCTACATACAGTGCCAATTTATCCCAAGT |
| | TGAGATGAATTTAGCTCCATCAAATCCTCTGGATATCTCAGTTGAATTGAATCGAGTCAA |
| | CACCAGTCTCTCTAAAGTGGAATCTCTAATAAAAGAAAGCAATAGTATCCTGGACTCAGT |
| | AAACCCTCAAATTTTAAATGTCAAGACACTCATTACgTATATCGTTTTGACTATCATATC |
| | TCTTGTTTTTGGTATACTTAGCCTGATTCTAGCATGCTACCTAATGTACAAGCAAAAGGC |
| | GCAACAAAAGACCTTATTATGGCTTGGGAATAATACaCTcGATCAGATGAGAGCCACTAC |
| | AAAAATGTGAACACAGATGAGGAACGAAGGTTTCCCTAATAGTAATTTGTGTGAAAGTTC |
| | TGGTAGTCTGTCAGTTCAGAGAGTTAAGAAAAAACTACCGGTTGTAGATGACCAAAGGAC |
| | GATATACGGGTAGAACGGTAAGAGAGGCCGCCCCTCAATTGCGAGCCAGGCTTCACAACC |
| | TCCGTTCTACCGCTTCACCGACAACAGTCCTCAATCATGGACCGCGCCGTTAGCCAAGTT |
| | GCGTTAGAGAATGATGAAAGAGAGGCAAAAAATACATGGCGCTTGATATTCCGGATTGCA |
| | ATCTTATTCTTAACAGTAGTGACCTTGGCTATATCTGTAGCCTCCCTTTTATATAGCATG |
| | GGGGCTAGCACACCTTCCACTCTGATCAGCCTAAATAACTCAATTATCACAAGCAGCAAT |
| | GGTCTCAAAAAGGAAATCCTGAACCAGAACATAAAAGAGGACCTCATATATAGAGAAGTT |
| | GCTATAAATATACCTTTAACATTAGATAGGGTTACTGTTGAGGTAGGGACTGCAGTAAAC |
| | CAGATTACTGATGCACTCAGGCAACTCCAGTCAGTTAATGGATCTGCTGCATTCGCCTCA |
| | TCAAACTCTCCTGATTATAGTGGGGGAATAGAACACCTGATTTTCCAAAGGAATACGCTT |
| | ATTAATCGCTCAGTGAGTGTCTCAGATTTAATAGAACACCCCAGTTTCATACCAACTCCT |
| | ACTACACAGCATGGTTGTACCAGAATCCCCACATTCCACCTAGGAACTCGCCACTGGTGC |
| | TATAGTCACAATATAATAGGTCAGGGATGTGCTGATTCTAGAGCTAGTGTGATGTATATT |
| | TCAATGGGAGCACTGGGTGTCAGTTCATTGGGAACCCCGACCTTCACAACATCTGCTGCA |
| | TCAATATTATCTGATAGCCTCAATCGGAAGAGTTGCAGTATAGTAGCAACAACTGAGGGT |
| | TGTGACGTACTCTGCAGTATAGTTACACAAACAGAAGACCAAGATTATGCTGATCACACT |
| | CCTACTCCAATGATACATGGTAGATTATGGTTTAATGGCACATACACAGAGAGATCCTTA |
| | TCCCAGAGTTTATTCCTTGGAACATGGGCTGCGCAATATCCGGCTGTAGGATCTGGTATA |
| | ATGACACCTGGGCGAGTTATATTCCCTTTCTATGGAGGTGTGATCCCTAACTCTCCTCTC |
| | TTCTTGGATCTCGAAAGATTCGCTTTATTCACACATAATGGAGACTTAGAATGCATGAAC |
| | TTAACACAATATCAGAAAGAAGCAATTTACTCTGCATATAAGCCTCCCAAGATTAGAGGA |
| | TCACTGTGGGCACAAGGCTTCATAGTATGTTCAGTAGGAGACATGGGGAATTGCTCTCTT |
| | AAAGTGATCAATACAAGCACAGTTATGATGGGTGCAGAAGGTCGGCTACAATTAGTTGGG |
| | GACTCCGTTATGTACTATCAGAGATCATCATCCTGGTGGCCTGTAGGAATTCTTTATCGG |
| | TTGAGTCTTGTAGACATCATCGCCGGAGATATACAGGTCGTCATAAACAGTGAACCACTC |
| | CCTCTGAGCAAGTTCCCGCGGCCAACCTGGACTCCAGGAGTGTGTCAAAAACCAAATGTA |
| | TGCCCTGCAGTTTGTGTAACTGGGGTCTATCAAGACCTTTGGGCAATTTCCGCAGGGGAG |
| | ACACTATCTGAAATGACATTCTTTGGAGGATATTTAGAGGCATCCACCCAACGAAAAGAT |
| | CCATGGATAGGCGTTGCTAATCAATATAGTTGGTTCATGAGAAGAAGATTATTCAAGACA |
| | AGCACTGAAGCTGCATATTCGTCATCAACGTGTTTTAGGAACACTAGACTGGATCGAAAT |
| | TTCTGCCTATTAGTCTTTGAATTAACTGATAACTTACTTGGAGACTGGAGAATTGTCCCC |
| | CTCTTATTTGAATTAACCATCGTATAAggcgcgccTTGAGTCAATTATAAAGGAGTTGGA |
| | AAGATGGCATTGTATCACCTATCTTCTGCGACATCAAGAATCAAACCGAATGCCGGCGCG |
| | TGCTCGAATTCCATGTTGCCAGTTGACCACAATCAGCCAGTGCTCATGCGATCAGATTAA |
| | GCCTTGTCATTAATCTCTTGATTAAGAAAAAATGTAAGTGGCAATGAGATACAAGGCAAA |
| | ACAGCTCATGGTAAATAATACGGGTAGGACATGGCGAGCTCCGGTCCTGAAAGGGCAGAG |
| | CATCAGATTATCCTACCAGAGCCACACCTGTCTTCACCATTGGTCAAGCACAAACTACTC |
| | TATTACTGGAAATTAACTGGGCTACCGCTTCCTGATGAATGTGACTTCGACCACCTCATT |
| | CTCAGCCGACAATGGAAAAAAATACTTGAATCGGCCTCTCCTGATACTGAGAGAATGATA |
| | AAACTCGGAAGGGCAGTACACCAAACTCTTAACCACAATTCCAGAATAACCGGAGTGCTC |
| | CACCCCAGGTGTTTAGAACAACTGGCTAATATTGAGGTCCCAGATTCAACCAACAAATTT |
| | CGGAAGATTGAGAAGAAGATCCAAATTCACAACACGAGATATGGAGAACTGTTCACAAGG |
| | CTGTGTACGCATATAGAGAAGAAACTGCTGGGGTCATCTTGGTCTAACAATGTCCCCCGG |
| | TCAGAGGAGTTCAGCAGCATTCGTACGGATCCGGCATTCTGGTTTCACTCAAAATGGTCC |
| | ACAGCCAAGTTTGCATGGCTCCATATAAAACAGATCCAGAGGCATCTGATGGTGGCAGCT |
| | AAGACAAGGTCTGCGGCCAACAAATTGGTGATGCTAACCCATAAGGTAGGCCAAGTCTTT |
| | GTCACTCCTGAACTTGTCGTTGTGACGCATACGAATGAGAACAAGTTCACATGTCTTACC |
| | CAGGAACTTGTATTGATGTATGCAGATATGATGGAGGGCAGAGATATGGTCAACATAATA |
| | TCAACCACGGCGGTGCATCTCAGAAGCTTATCAGAGAAAATTGATGACATTTTGCGGTTA |
| | ATAGAGGCTCTGGCAAAAGACTTGGGTAATCAAGTCTACGATGTTGTATCACTAATGGAG |
| | GGATTTGCATACGGAGCTGTCCAGCTACTCGAGCCGTCAGGTACATTTGCAGGAGATTTC |
| | TTCGCATTCAACCTGCAGGAGCTTAAAGACATTCTAATTGGCCTCCTCCCCAATGATATA |
| | GCAGAATCCGTGACTCATGCAATCGCTACTGTATTCTCTGGTTTAGAACAGAATCAAGCA |
| | GCTGAGATGTTGTGTCTGTTGCGTCTGTGGGGTCACCCACTGCTTGAGTCCCGTATTGCA |
| | GCAAAGGCAGTCAGGAGCCAAATGTGCGCACCGAAAATGGTAGACTTTGATATGATCCTT |
| | CAGGTACTGTCTTTCTTCAAGGGAACAATCATCAACGGGTACAGAAAGAAGAATGCAGGT |
| | GTGTGGCCGCGAGTCAAAGTGGATACAATATATGGGAAGGTCATTGGGCAACTACATGCA |
| | GATTCAGCAGAGATTTCACACGATATCATGTTGAGAGAGTATAAGAGTTTATCTGCACTT |
| | GAATTTGAGCCATGTATAGAATATGACCCTGTCACCAACCTGAGCATGTTCCTAAAAGAC |
| | AAGGCAATCGCACACCCCAACGATAATTGGCTTGCCTCGTTTAGGCGGAACCTTCTCTCC |
| | GAAGACCAGAAGAAACATGTAAAAGAAGCAACTTCGACTAATCGCCTCTTGATAGAGTTT |
| | TTAGAGTCAAATGATTTTGATCCATATAAAGAGATGGAATATCTGACGACCCTTGAGTAC |
| | CTTAGAGATGACAATGTGGCAGTATCATACTCGCTCAAGGAGAAGGAAGTGAAAGTTAAT |
| | GGACGGATCTTCGCTAAGCTGACAAAGAAGTTAAGGAACTGTCAGGTGATGGCGGAAGGG |
| | ATCCTAGCCGATCAGATTGCACCTTTCTTTCAGGGAAATGGAGTCATTCAGGATAGCATA |
| | TCCTTGACCAAGAGTATGCTAGCGATGAGTCAACTGTCTTTTAACAGCAATAAGAAACGT |
| | ATCACTGACTGTAAAGAAAGAGTATCTTCAAACCGCAATCATGATCCGAAAAGCAAGAAC |
| | CGTCGGAGAGTTGCAACCTTCATAACAACTGACCTGCAAAAGTACTGTCTTAATTGGAGA |
| | TATCAGACAATCAAATTGTTCGCTCATGCCATCAATCAGTTGATGGGCCTACCTCACTTC |
| | TTCGAATGGATTCACCTAAGACTGATGGACACTACGATGTTCGTAGGAGACCCTTTCAAT |
| | CCTCCAAGTGACCCTACTGACTGTGACCTCTCAAGAGTCCCTAATGATGACATATATATT |
| | GTCAGTGCCAGAGGGGGTATCGAAGGATTATGCCAGAAGCTATGGACAATGATCTCAATT |
| | GCTGCAATCCAACTTGCTGCAGCTAGATCGCATTGTCGTGTTGCCTGTATGGTACAGGGT |
| | GATAATCAAGTAATAGCAGTAACGAGAGAGGTAAGATCAGACGACTCTCCGGAGATGGTG |
| | TTGACACAGTTGCATCAAGCCAGTGATAATTTCTTCAAGGAATTAATTCATGTCAATCAT |
| | TTGATTGGCCATAATTTGAAGGATCGTGAAACCATCAGGTCAGACACATTCTTCATATAC |
| | AGCAAACGAATCTTCAAAGATGGAGCAATCCTCAGTCAAGTCCTCAAAAATTCATCTAAA |
| | TTAGTGCTAGTGTCAGGTGATCTCAGTGAAAACACCGTAATGTCCTGTGCCAACATTGCC |
| | TCTACTGTAGCACGGCTATGCGAGAACGGGCTTCCCAAAGACTTCTGTTACTATTTAAAC |
| | TATATAATGAGTTGTGTGCAGACATACTTTGACTCTGAGTTCTCCATCACCAACAATTCG |
| | CACCCCGATCTTAATCAGTCGTGGATTGAGGACATCTCTTTTGTGCACTCATATGTTCTG |
| | ACTCCTGCCCAATTAGGGGGACTGAGTAACCTTCAATACTCAAGGCTCTACACTAGAAAT |
| | ATCGGTGACCCGGGGACTACTGCTTTTGCAGAGATCAAGCGACTAGAAGCAGTGGGATTA |
| | CTGAGTCCTAACATTATGACTAATATCTTAACTAGGCCGCCTGGGAATGGAGATTGGGCC |
| | AGTCTGTGCAACGACCCATACTCTTTCAATTTTGAGACTGTTGCAAGCCCAAATATTGTT |
| | CTTAAGAAACATACGCAAAGAGTCCTATTTGAAACTTGTTCAAATCCCTTATTGTCTGGA |
| | GTGCACACAGAGGATAATGAGGCAGAAGAGAAGGCATTGGCTGAATTCTTGCTTAATCAA |
| | GAGGTGATTCATCCCCGCGTTGCGCATGCCATCATGGAGGCAAGCTCTGTAGGTAGGAGA |
| | AAGCAAATTCAAGGGCTTGTTGACACAACAAACACCGTAATTAAGATTGCGCTTACTAGG |
| | AGGCCATTAGGCATCAAGAGGCTGATGCGGATAGTCAATTATTCTAGCATGCATGCAATG |
| | CTGTTTAGAGACGATGTTTTTTCCTCCAGTAGATCCAACCACCCCTTAGTCTCTTCTAAT |
| | ATGTGTTCTCTGACACTGGCAGACTATGCACGGAATAGAAGCTGGTCACCTTTGACGGGA |
| | GGCAGGAAAATACTGGGTGTATCTAATCCTGATACGATAGAACTCGTAGAGGGTGAGATT |
| | CTTAGTGTAAGCGGAGGGTGTACAAGATGTGACAGCGGAGATGAACAATTTACTTGGTTC |
| | CATCTTCCAAGCAATATAGAATTGACCGATGACACCAGCAAGAATCCTCCGATGAGGGTA |
| | CCATATCTCGGGTCAAAGACACAGGAGAGGAGAGCTGCCTCACTTGCAAAAATAGCTCAT |
| | ATGTCGCCACATGTAAAGGCTGCCCTAAGGGCATCATCCGTGTTGATCTGGGCTTATGGG |
| | GATAATGAAGTAAATTGGACTGCTGCTCTTACGATTGCAAAATCTCGGTGTAATGTAAAC |
| | TTAGAGTATCTTCGGTTACTGTCCCCTTTACCCACGGCTGGGAATCTTCAACATAGACTA |
| | GATGATGGTATAACTCAGATGACATTCACCCCTGCATCTCTCTACAGGgtgtcaccttac |
| | attcacatatccaatgattctcaaaggctgttcactgaagaaggagtcaaagaggggaat |
| | gtggtttaccaacagatcATGCTCTTGGGTTTATCTCTAATCGAATCGATCTTTCCAATG |
| | ACAACAACCAGGACATATGATGAGATCACACTGCACCTACATAGTAAATTTAGTTGCTGT |
| | ATCAGAGAAGCACCTGTTGCGGTTCCTTTCGAGCTACTTGGGGTGGTACCGGAACTGAGG |
| | ACAGTGACCTCAAATAAGTTTATGTATGATCCTAGCCCTGTATCGGAGGGAGACTTTGCG |
| | AGACTTGACTTAGCTATCTTCAAGAGTTATGAGCTTAATCTGGAGTCATATCCCACGATA |
| | GAGCTAATGAACATTCTTTCAATATCCAGCGGGAAGTTGATTGGCCAGTCTGTGGTTTCT |
| | TATGATGAAGATACCTCCATAAAGAATGACGCCATAATAGTGTATGACAATACCCGAAAT |
| | TGGATCAGTGAAGCTCAGAATTCAGATGTGGTCCGCCTATTTGAATATGCAGCACTTGAA |
| | GTGCTCCTCGACTGTTCTTACCAACTCTATTACCTGAGAGTAAGAGGCCTAGACAATATT |
| | GTCTTATATATGGGTGATTTATACAAGAATATGCCAGGAATTCTACTTTCCAACATTGCA |
| | GCTACAATATCTCATCCCGTCATTCATTCAAGGTTACATGCAGTGGGCCTGGTCAACCAT |
| | GACGGATCACACCAACTTGCAGATACGGATTTTATCGAAATGTCTGCAAAACTATTAGTA |
| | TCTTGCACCCGACGTGTGATCTCCGGCTTATATTCAGGAAATAAGTATGATCTGCTGTTC |
| | CCATCTGTCTTAGATGATAACCTGAATGAGAAGATGCTTCAGCTGATATCCCGGTTATGC |
| | TGTCTGTACACGGTACTCTTTGCTACAACAAGAGAAATCCCGAAAATAAGAGGCTTAACT |
| | GCAGAAGAGAAATGTTCAATACTCACTGAGTATTTACTGTCGGATGCTGTGAAACCATTA |
| | CTTAGCCCCGATCAAGTGAGCTCTATCATGTCTCCTAACATAATTACATTCCCAGCTAAT |
| | CTGTACTACATGTCTCGGAAGAGCCTCAATTTGATCAGGGAAAGGGAGGACAGGGATACT |
| | ATCCTGGCGTTGTTGTTCCCCCAAGAGCCATTATTAGAGTTCCCTTCTGTGCAAGATATT |
| | GGTGCTCGAGTGAAAGATCCATTCACCCGACAACCTGCGGCATTTTTGCAAGAGTTAGAT |
| | TTGAGTGCTCCAGCAAGGTATGACGCATTCACACTTAGTCAGATTCATCCTGAACTCACA |
| | TCTCCAAATCCGGAGGAAGACTACTTAGTACGATACTTGTTCAGAGGGATAGGGACTGCA |
| | TCTTCCTCTTGGTATAAGGCATCTCATCTCCTTTCTGTACCCGAGGTAAGATGTGCAAGA |
| | CACGGGAACTCCTTATACTTAGCTGAAGGGAGCGGAGCCATCATGAGTCTTCTCGAACTG |
| | CATGTACCACATGAAACTATCTATTACAATACGCTCTTTTCAAATGAGATGAACCCCCCG |
| | CAACGACATTTCGGGCCGACCCCAACTCAGTTTTTGAATTCGGTTGTTTATAGGAATCTA |
| | CAGGCGGAGGTAACATGCAAAGATGGATTTGTCCAAGAGTTCCGTCCATTATGGAGAGAA |
| | AATACAGAGGAAAGTGACCTGACCTCAGATAAAGCAGTGGGGTATATTACATCTGCAGTG |
| | CCCTACAGATCTGTATCATTGCTGCATTGTGACATTGAAATTCCTCCAGGGTCCAATCAA |
| | AGCTTACTAGATCAACTAGCTATCAATTTATCTCTGATTGCCATGCATTCTGTAAGGGAG |
| | GGCGGGGTAGTAATCATCAAAGTGTTGTATGCAATGGGATACTACTTTCATCTACTCATG |
| | AACTTGTTTGCTCCGTGTTCCACAAAAGGATATATTCTCTCTAATGGTTATGCATGTCGA |
| | GGAGATATGGAGTGTTACCTGGTATTTGTCATGGGTTACCTGGGCGGGCCTACATTTGTA |
| | CATGAGGTGGTGAGGATGGCAAAAACTCTGGTGCAGCGGCACGGTACGCTCTTGTCTAAA |
| | TCAGATGAGATCACACTGACCAGGTTATTCACCTCACAGCGGCAGCGTGTGACAGACATC |
| | CTATCCAGTCCTTTACCAAGATTAATAAAGTACTTGAGGAAGAATATTGACACTGCGCTG |
| | ATTGAAGCCGGGGGACAGCCCGTCCGTCCATTCTGTGCGGAGAGTCTGGTGAGCACGCTA |
| | GCGAACATAACTCAGATAACCCAGATTATCGCTAGTCACATTGACACAGTTATCCGGTCT |
| | GTGATATATATGGAAGCTGAGGGTGATCTCGCTGACACAGTATTTCTATTTACCCCTTAC |
| | AATCTCTCTACTGACGGGAAAAAGAGGACATCACTTATACAGTGCACGAGACAGATCCTA |
| | GAGGTTACAATACTAGGTCTTAGAGTCGAAAATCTCAATAAAATAGGCGATATAATCAGC |
| | CTAGTGCTTAAAGGCATGATCTCCATGGAGGACCTTATCCCACTAAGGACATACTTGAAG |
| | CATAGTACCTGCCCTAAATATTTGAAGGCTGTCCTAGGTATTACCAAACTCAAAGAAATG |
| | TTTACAGACACTTCTGTATTGTACTTGACTCGTGCTCAACAAAAATTCTACATGAAAACT |
| | ATAGGCAATGCAGTCAAAGGATATTACAGTAACTGTGACTCTTAACGAAAATCACATATT |
| | AATAGGCTCCTTTTTTGGCCAATTGTATTCTTGTTGATTTAATCATATTATGTTAGAAAA |
| | AAGTTGAACCCTGACTCCTTAGGACTCGAATTCGAACTCAAATAAATGTCTTAAAAAAAG |
| | GTTGCGCACAATTATTCTTGAGTGTAGTCTCGTCATTCACCAAATCTTTGTTTGGTGGCC |
| | GGCATGGTCCCAGCCTCCTCGCTGGCGCCGGCTGGGCAACATTCCGAGGGGACCGTCCCC |
| | TCGGTAATGGCGAATGGGACGTCGACTGCTAACAAAGCCCGAAAGGAAGCTGAGTTGGCT |
| | GCTGCCACCGCTGAGCAATAACTAGCATAACCCCTTGGGGCCTCTAAACGGGTCTTGAGG |
| | GGTTTTTTGCTGAAAGGAGGAACTATAT |
| SEQ ID NO: | MFVFLVLLPLVSSQCVNLTTRTQLPPAYTNSFTRGVYYPDKVFRSSVLHSTQDLFLPFFS |
| 6; Spike | NVTWFHAIHVSGTNGTKRFDNPVLPFNDGVYFASTEKSNIIRGWIFGTTLDSKTQSLLIV |
| protein | NNATNVVIKVCEFQFCNDPFLGVYYHKNNKSWMESEFRVYSSANNCTFEYVSQPFLMDLE |
| (surface | GKQGNFKNLREFVFKNIDGYFKIYSKHTPINLVRDLPQGFSALEPLVDLPIGINITRFQT |
| glycoprotein) | LLALHRSYLTPGDSSSGWTAGAAAYYVGYLQPRTFLLKYNENGTITDAVDCALDPLSETK |
| (from | CTLKSFTVEKGIYQTSNFRVQPTESIVRFPNITNLCPFGEVFNATRFASVYAWNRKRISN |
| genome | CVADYSVLYNSASFSTFKCYGVSPTKLNDLCFTNVYADSFVIRGDEVRQIAPGQTGKIAD |
| MN908947) | YNYKLPDDFTGCVIAWNSNNLDSKVGGNYNYLYRLFRKSNLKPFERDISTEIYQAGSTPC |
| (QHD43416. | NGVEGFNCYFPLQSYGFQPTNGVGYQPYRVVVLSFELLHAPATVCGPKKSTNLVKNKCVN |
| 1) | FNFNGLTGTGVLTESNKKFLPFQQFGRDIADTTDAVRDPQTLEILDITPCSFGGVSVITP |
| (1273 aa) | GTNTSNQVAVLYQDVNCTEVPVAIHADQLTPTWRVYSTGSNVFQTRAGCLIGAEHVNNSY |
| | ECDIPIGAGICASYQTQTNSPRRARSVASQSIIAYTMSLGAENSVAYSNNSIAIPTNFTI |
| | SVTTEILPVSMTKTSVDCTMYICGDSTECSNLLLQYGSFCTQLNRALTGIAVEQDKNTQE |
| | VFAQVKQIYKTPPIKDFGGFNFSQILPDPSKPSKRSFIEDLLFNKVTLADAGFIKQYGDC |
| | LGDIAARDLICAQKFNGLTVLPPLLTDEMIAQYTSALLAGTITSGWTFGAGAALQIPFAM |
| | QMAYRFNGIGVTQNVLYENQKLIANQFNSAIGKIQDSLSSTASALGKLQDVVNQNAQALN |
| | TLVKQLSSNFGAISSVLNDILSRLDKVEAEVQIDRLITGRLQSLQTYVTQQLIRAAEIRA |
| | SANLAATKMSECVLGQSKRVDFCGKGYHLMSFPQSAPHGVVFLHVTYVPAQEKNFTTAPA |
| | ICHDGKAHFPREGVFVSNGTHWFVTQRNFYEPQIITTDNTFVSGNCDVVIGIVNNTVYDP |
| | LQPELDSFKEELDKYFKNHTSPDVDLGDISGINASVVNIQKEIDRLNEVAKNLNESLIDL |
| | QELGKYEQYIKWPWYIWLGFIAGLIAIVMVTIMLCCMTSCCSCLKGCCSCGSCCKFDEDD |
| | SEPVLKGVKLHYT |
| SEQ ID NO: | MFVFLVLLPLVSSQCVNLTTRTQLPPAYTNSFTRGVYYPDKVFRSSVLHSTQDLFLPFFS |
| 7: Spike | NVTWFHAIHVSGTNGTKRFDNPVLPFNDGVYFASTEKSNIIRGWIFGTTLDSKTQSLLIV |
| protein | NNATNVVIKVCEFQFCNDPFLGVYYHKNNKSWMESEFRVYSSANNCTFEYVSQPFLMDLE |
| (surface | GKQGNFKNLREFVFKNIDGYFKIYSKHTPINLVRDLPQGFSALEPLVDLPIGINITRFQT |
| glycoprotein), | LLALHRSYLTPGDSSSGWTAGAAAYYVGYLQPRTFLLKYNENGTITDAVDCALDPLSETK |
| South Africa | CTLKSFTVEKGIYQTSNFRVQPTESIVRFPNITNLCPFGEVFNATRFASVYAWNRKRISN |
| (QIZ15537.1; | CVADYSVLYNSASFSTFKCYGVSPTKLNDLCFTNVYADSFVIRGDEVRQIAPGQTGKIAD |
| 1273 aa) | YNYKLPDDFTGCVIAWNSNNLDSKVGGNYNYLYRLFRKSNLKPFERDISTEIYQAGSTPC |
| | NGVEGFNCYFPLQSYGFQPTNGVGYQPYRVVVLSFELLHAPATVCGPKKSTNLVKNKCVN |
| | FNFNGLTGTGVLTESNKKFLPFQQFGRDIADTTDAVRDPQTLEILDITPCSFGGVSVITP |
| | GTNTSNQVAVLYQGVNCTEVPVAIHADQLTPTWRVYSTGSNVFQTRAGCLIGAEHVNNSY |
| | ECDIPIGAGICASYQTQTNSPRRARSVASQSIIAYTMSLGAENSVAYSNNSIAIPTNFTI |
| | SVTTEILPVSMTKTSVDCTMYICGDSTECSNLLLQYGSFCTQLNRALTGIAVEQDKNTQE |
| | VFAQVKQIYKTPPIKDFGGFNFSQILPDPSKPSKRSFIEDLLFNKVTLADAGFIKQYGDC |
| | LGDIAARDLICAQKFNGLTVLPPLLTDEMIAQYTSALLAGTITSGWTFGAGAALQIPFAM |
| | QMAYRFNGIGVTQNVLYENQKLIANQFNSAIGKIQDSLSSTASALGKLQDVVNQNAQALN |
| | TLVKQLSSNFGAISSVLNDILSRLDKVEAEVQIDRLITGRLQSLQTYVTQQLIRAAEIRA |
| | SANLAATKMSECVLGQSKRVDFCGKGYHLMSFPQSAPHGVVFLHVTYVPAQEKNFTTAPA |
| | ICHDGKAHFPREGVFVSNGTHWFVTQRNFYEPQIITTDNTFVSGNCDVVIGIVNNTVYDP |
| | LQPELDSFKEELDKYFKNHTSPDVDLGDISGINASVVNIQKEIDRLNEVAKNLNESLIDL |
| | QELGKYEQYIKWPWYIWLGFIAGLIAIVMVTIMLCCMTSCCSCLKGCCSCGSCCKFDEDD |
| | SEPVLKGVKLHYT |
| SEQ ID NO: | ATGTTCGTGTTCCTGGTCCTGCTGCCACTGGTAAGCTCCCAATGTGTAAACTTAACCACA |
| 8; Codon | AGAACCCAGCTCCCACCTGCCTACACCAACAGCTTCACCAGAGGCGTTTATTACCCCGAC |
| Optimized | AAGGTATTCCGGTCTTCTGTTCTGCACTCTACCCAGGACCTGTTTCTGCCCTTTTTCAGC |
| Spike Fusion | AACGTGACATGGTTCCACGCCATCCACGTGTCTGGCACAAACGGCACCAAGCGGTTTGAT |
| (segment | AATCCTGTGCTCCCTTTCAATGACGGCGTGTACTTCGCCTCTACTGAGAAGAGCAACATC |
| that went into | ATCCGGGGCTGGATCTTTGGCACAACACTGGACTCTAAAACCCAGAGCCTGCTGATCGTG |
| the NDV- | AACAACGCCACCAACGTGGTGATTAAGGTGTGCGAGTTCCAGTTCTGCAATGACCCTTTC |
| FLS) | CTCGGCGTGTACTACCACAAGAACAACAAAAGTTGGATGGAAAGCGAATTCAGGGTGTAC |
| The product | TCAAGCGCCAACAACTGTACCTTCGAGTACGTGAGCCAGCCTTTCCTGATGGACCTAGAA |
| of this codon | GGTAAGCAGGGCAATTTCAAGAACCTCAGAGAGTTCGTGTTCAAGAATATTGACGGCTAC |
| optimized | TTCAAAATCTACAGCAAGCACACCCCAATCAACCTGGTGCGGGACCTGCCCCAGGGCTTT |
| gene is SEQ | AGCGCGCTGGAGCCTCTGGTGGACCTGCCTATCGGCATCAACATCACCCGGTTCCAGACA |
| ID NO: 6 | CTGCTGGCTCTGCATAGAAGCTACCTGACACCTGGCGACAGTTCTTCTGGCTGGACAGCC |
| | GGCGCCGCCGCCTACTACGTGGGCTACCTGCAGCCTAGAACATTCCTGCTGAAATACAAC |
| | GAGAACGGCACGATCACAGACGCCGTGGACTGCGCCCTGGATCCCCTGTCTGAGACAAAG |
| | TGCACCCTGAAGTCTTTCACCGTGGAGAAGGGCATCTACCAGACCTCCAACTTCAGAGTG |
| | CAGCCTACCGAATCCATCGTGCGCTTTCCCAACATCACCAACCTGTGCCCCTTCGGCGAG |
| | GTCTTTAATGCCACGAGATTCGCCAGCGTGTATGCCTGGAACAGAAAGAGAATCAGCAAC |
| | TGCGTGGCCGACTACAGCGTGCTGTACAACAGCGCCTCTTTCAGCACATTTAAGTGCTAC |
| | GGAGTGTCTCCTACCAAACTCAACGATCTGTGCTTCACGAACGTGTATGCCGACAGCTTC |
| | GTGATCCGAGGAGATGAGGTGCGGCAGATCGCTCCAGGACAGACAGGCAAGATCGCCGAC |
| | TACAACTACAAGCTGCCCGACGACTTTACCGGCTGCGTGATCGCTTGGAACAGCAATAAC |
| | CTGGACTCAAAGGTTGGAGGAAACTACAACTACCTGTACAGACTGTTCAGAAAGTCCAAC |
| | CTGAAGCCCTTCGAGAGAGACATCTCTACAGAAATCTACCAGGCCGGCAGCACCCCATGT |
| | AACGGCGTGGAAGGCTTCAACTGCTACTTCCCTCTGCAGTCTTATGGCTTCCAGCCCACA |
| | AACGGAGTGGGCTATCAGCCTTACCGCGTGGTTGTCCTGAGCTTTGAGCTGCTGCATGCC |
| | CCTGCTACGGTGTGTGGACCTAAGAAGTCCACCAACCTGGTGAAGAACAAGTGTGTGAAC |
| | TTCAACTTCAACGGCCTGACCGGCACCGGGGTGCTGACAGAGTCTAACAAGAAATTCCTG |
| | CCATTCCAGCAATTCGGCCGGGACATCGCCGACACCACCGACGCCGTGCGGGATCCTCAG |
| | ACCCTCGAAATCCTGGACATCACCCCCTGTAGCTTCGGCGGCGTGAGCGTGATCACCCCT |
| | GGCACAAACACCAGCAATCAAGTGGCTGTCCTGTACCAGGATGTCAATTGCACAGAAGTG |
| | CCTGTGGCCATCCACGCCGATCAGCTGACCCCCACCTGGCGGGTGTACTCGACAGGAAGC |
| | AACGTGTTTCAAACAAGAGCCGGCTGCCTGATCGGGGCCGAGCACGTGAACAATTCCTAC |
| | GAGTGCGACATCCCCATCGGCGCCGGCATCTGTGCCTCTTACCAGACACAGACCAATTCC |
| | CCTCGTAGAGCCAGATCCGTGGCCAGCCAGAGCATCATCGCCTACACCATGAGCCTGGGC |
| | GCCGAAAACAGCGTTGCATATTCCAACAACAGCATCGCCATCCCTACCAACTTCACCATC |
| | AGCGTGACCACAGAAATCCTGCCTGTGTCCATGACCAAGACAAGCGTTGATTGCACCATG |
| | TACATCTGCGGCGATAGCACAGAGTGCAGCAATCTGCTGCTGCAGTACGGTAGCTTCTGC |
| | ACCCAGCTGAATAGAGCCCTGACCGGCATCGCTGTGGAACAGGACAAAAACACCCAGGAG |
| | GTCTTCGCCCAGGTGAAGCAAATCTACAAGACCCCTCCAATCAAGGACTTCGGAGGCTTT |
| | AACTTTAGCCAGATCCTGCCTGATCCCTCCAAGCCTAGCAAACGGAGTTTCATCGAGGAC |
| | CTGCTCTTCAACAAGGTGACCCTGGCTGACGCCGGCTTCATTAAGCAGTACGGCGATTGC |
| | CTCGGCGACATCGCTGCAAGAGACCTGATCTGCGCCCAGAAGTTCAACGGCCTGACCGTG |
| | CTGCCTCCTCTCCTGACAGACGAGATGATCGCCCAGTACACCTCTGCCCTTCTGGCTGGC |
| | ACCATCACCAGCGGATGGACCTTTGGAGCCGGAGCCGCCCTGCAGATCCCTTTCGCTATG |
| | CAGATGGCCTACAGATTCAACGGGATCGGAGTGACCCAAAACGTGCTGTATGAAAACCAG |
| | AAACTGATCGCCAATCAGTTTAACAGCGCCATCGGCAAAATCCAGGATAGCCTGTCCAGC |
| | ACCGCCAGCGCCCTCGGCAAGCTGCAAGATGTGGTGAATCAAAATGCCCAAGCCCTGAAC |
| | ACACTGGTGAAGCAGCTGAGCAGCAACTTCGGCGCCATCAGCAGCGTGCTGAACGACATC |
| | CTGAGCAGACTGGACAAGGTGGAAGCCGAGGTGCAGATCGACAGACTGATCACAGGCAGA |
| | CTGCAGTCCCTGCAGACCTACGTGACCCAGCAGTTGATTAGAGCCGCTGAGATTAGAGCC |
| | AGTGCCAACCTGGCTGCCACAAAGATGTCAGAATGCGTGCTGGGCCAGAGCAAGAGAGTG |
| | GACTTCTGCGGCAAAGGCTACCACCTGATGAGCTTTCCTCAGTCTGCACCCCACGGCGTG |
| | GTGTTTCTCCACGTGACATACGTGCCCGCGCAAGAAAAGAACTTTACAACCGCCCCAGCG |
| | ATCTGCCACGACGGCAAGGCCCACTTCCCTCGGGAGGGTGTGTTCGTGAGCAATGGAACA |
| | CACTGGTTCGTCACCCAGCGGAACTTCTACGAGCCTCAGATCATTACCACCGACAACACC |
| | TTCGTGAGCGGCAACTGTGACGTCGTTATCGGCATCGTGAACAATACCGTGTACGACCCC |
| | CTGCAGCCTGAGCTGGATAGCTTCAAAGAGGAACTGGACAAGTACTTCAAGAACCACACA |
| | AGCCCCGACGTGGACCTAGGCGACATCTCTGGAATCAACGCCAGCGTGGTGAACATCCAA |
| | AAGGAAATCGACAGACTGAACGAGGTGGCCAAGAATCTGAATGAAAGCCTGATCGATCTG |
| | CAGGAGCTGGGCAAGTACGAGCAGTACATCAAATGGCCTTGGTACATCTGGCTGGGCTTC |
| | ATCGCTGGTCTGATCGCTATCGTGATGGTGACCATTATGCTGTGCTGCATGACCTCCTGC |
| | TGCTCTTGCCTGAAGGGCTGTTGTTCTTGCGGCTCTTGCTGCAAGTTCGACGAGGATGAC |
| | TCTGAACCTGTTCTGAAGGGCGTGAAGCTGCACTACACCTGATaa |
| SEQ ID NO: | TAATACGACTCACTATAGGGACCAAACAGAGAATCCGTGAGTTACGATAAAAGGCGAAGG |
| 9; Chimeric | AGCAATTGAAGTCGCACGGGTAGAAGGTGTGAATCTCGAGTGCGAGCCCGAAGCACAAAC |
| NDV with | TCGAGAAAGCCTTCTGCCAACATGTCTTCCGTATTTGATGAGTACGAACAGCTCCTCGCG |
| APMV-5 F | GCTCAGACTCGCCCCAATGGAGCTCATGGAGGGGGAGAAAAAGGGAGTACCTTAAAAGTA |
| and HN | GACGTCCCGGTATTCACTCTTAACAGTGATGACCCAGAAGATAGATGGAGCTTTGTGGTA |
| without GFP | TTCTGCCTCCGGATTGCTGTTAGCGAAGATGCCAACAAACCACTCAGGCAAGGTGCTCTC |
| | ATATCTCTTTTATGCTCCCACTCACAGGTAATGAGGAACCATGTTGCCATTGCAGGGAAA |
| | CAGAATGAAGCCACATTGGCCGTGCTTGAGATTGATGGCTTTGCCAACGGCACGCCCCAG |
| | TTCAACAATAGGAGTGGAGTGTCTGAAGAGAGAGCACAGAGATTTGCGATGATAGCAGGA |
| | TCTCTCCCTCGGGCATGCAGCAACGGAACCCCGTTCGTCACAGCCGGGGCAGAAGATGAT |
| | GCACCAGAAGACATCACCGATACCCTGGAGAGGATCCTCTCTATCCAGGCTCAAGTATGG |
| | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaaaacgcgtACCC |
| | AAGGTCCAACTCTCCAAGCGGCAATCCTCTCTCGCTTCCTCAGCCCCACTGAATGGTCGC |
| | GTAACCGTAATTAATCTAGCTACATTTAAGATTAAGAAAAAATACGGGTAGAATTGGAGT |
| | GCCCCAATTGTGCCAAGATGGACTCATCTAGGACAATTGGGCTGTACTTTGATTCTGCCC |
| | ATTCTTCTAGCAACCTGTTAGCATTTCCGATCGTCCTACAAGGCACAGGAGATGGGAAGA |
| | AGCAAATCGCCCCGCAATATAGGATCCAGCGCCTTGACTTGTGGACTGATAGTAAGGAGG |
| | ACTCAGTATTCATCACCACCTATGGATTCATCTTTCAAGTTGGGAATGAAGAAGCCACTG |
| | TCGGCATGATCGATGATAAACCCAAGCGCGAGTTACTTTCCGCTGCGATGCTCTGCCTAG |
| | GAAGCGTCCCAAATACCGGAGACCTTATTGAGCTGGCAAGGGCCTGTCTCACTATGATAG |
| | TCACATGCAAGAAGAGTGCAACTAATACTGAGAGAATGGTTTTGTCAGTAGTGCAGGCAC |
| | CCCAAGTGCTGCAAAGCTGTAGGGTTGTGGCAAACAAATACTCATCAGTGAATGCAGTCA |
| | AGCACGTGAAAGCGCCAGAGAAGATTCCCGGGAGTGGAACCCTAGAATACAAGGTGAACT |
| | TTGTCTCCTTGACTGTGGTACCGAAGAAGGATGTCTACAAGATCCCAGCTGCAGTATTGA |
| | AGGTTTCTGGCTCGAGTCTGTACAATCTTGCGCTCAATGTCACTATTAATGTGGAGGTAG |
| | ACCCGAGGAGTCCTTTGGTTAAATCTTTGTCTAAGTCTGACAGCGGATACTATGCTAACC |
| | TCTTCTTGCATATTGGACTTATGACCACCGTAGATAGGAAGGGGAAGAAAGTGACATTTG |
| | ACAAGCTGGAAAAGAAAATAAGGAGCCTTGATCTATCTGTCGGGCTCAGTGATGTGCTCG |
| | GGCCTTCCGTGTTGGTAAAAGCAAGAGGTGCACGGACTAAGCTTTTGGCACCTTTCTTCT |
| | CTAGCAGTGGGACAGCCTGCTATCCCATAGCAAATGCTTCTCCTCAGGTGGCCAAGATAC |
| | TCTGGAGTCAAACCGCGTGCCTGCGGAGCGTTAAAATCATTATCCAAGCAGGTACCCAAC |
| | GCGCTGTCGCAGTGACCGCCGACCACGAGGTTACCTCTACTAAGCTGGAGAAGGGGCACA |
| | CCCTTGCCAAATACAATCCTTTTAAGAAATAAGCTGCGTCTCTGAGATTGCGCTCCGCCC |
| | ACTCACCCAGATCATCATGACACAAAAAACTAATCTGTCTTGATTATTTACAGTTAGTTT |
| | ACCTGTCTATCAAGTTAGAAAAAACACGGGTAGAAGATTCTGGATCCCGGTTGGCGCCCT |
| | CCAGGTGCAAGttaattaaATGTTACAACTTCCCTTGACCATTCTTCTTAGCATTCTTAG |
| | TGCTCACCAGTCGCTTTGTCTAGACAACAGTAAGCTCATTCATGCAGGAATCATGAGTAC |
| | TACTGAGAGAGAAGTTAATGTTTATGCACAATCTATTACTGGGTCAATAGTGGTGAGATT |
| | GATTCCAAATATCCCAAGTAACCATAAATCTTGTGCAACTAGCCAAATCAAATTATACAA |
| | TGACACGTTAACAAGATTGTTGACCCCAATTAAAGCTAATCTAGAAGGACTTATTAGTGC |
| | TGTTTCTCAGGACCAATCGCAGAATTCTGGGAAGAGAAAGAAGCGTTTTGTAGGCGCAGT |
| | AATTGGAGCAGCTGCCCTTGGTTTGGCAACTGCTGCACAGGTGACTGCCACTGTAGCATT |
| | AAATCAAGCGCAAGAAAACGCTCGGAATATCCTAAGGCTTAAAAACTCGATTCAGAAGAC |
| | AAACGAGGCGGTGATGGAACTTAAAGATGCTGTGGGCCAAACAGCAGTAGCTATTGACAA |
| | AACTCAGGCCTTCATAAATAATCAAATCTTGCCTGCAATTTCAAATCTCTCATGTGAGGT |
| | CCTAGGGAATAAAATTGGGGTCCAATTATCTTTGTACCTTACTGAATTAACAACAGTATT |
| | CGGCAACCAACTGACAAACCCAGCCCTTACCACACTGTCATTACAAGCCTTGTACAATCT |
| | TTGTGGAGATGACTTCAATTACTTAATCAACCTATTAAATGCAAAAAATCGTAACTTAGC |
| | CTCACTTTATGAAGCAAACCTAATTCAGGGGAGAATTACTCAATATGACTCAATGAATCA |
| | GTTATTAATTATTCAGGTACAAATACCAAGCATCTCCACAGTGTCAGGAATGAGGGTCAC |
| | AGAATTGTTCACACTTAGTGTTGACACACCTATAGGAGAGGGAAAGGCCCTAGTACCAAA |
| | ATATGTCCTATCCTCAGGGAGAATAATGGAAGAGGTTGACCTAAGCAGTTGCGCTATAAC |
| | ATCAACATCAGTTTTCTGTTCCTCTATCATCTCTAGACCCCTTCCACTTGAAACAATAAA |
| | TTGCCTGAATGGGAATGTTACACAGTGTCAATTTACCGCCAACACAGGAACCCTTGAATC |
| | GAGATACGCTGTTATAGGAGGATTGGTGATTGCTAACTGTAAGGCTATAGTATGCAGGTG |
| | CCTAAATCCACCAGGTGTCATTGCGCAAAATCTTGGCTTACCAATTACAATCATCTCATC |
| | CAATACTTGTCAGCGAATTAATTTAGAACAAATCACTTTGTCTCTTGGGAACAGCATATT |
| | ATCTACATACAGTGCCAATTTATCCCAAGTTGAGATGAATTTAGCTCCATCAAATCCTCT |
| | GGATATCTCAGTTGAATTGAATCGAGTCAACACCAGTCTCTCTAAAGTGGAATCTCTAAT |
| | AAAAGAAAGCAATAGTATCCTGGACTCAGTAAACCCTCAAATTTTAAATGTCAAGACACT |
| | CATTACgTATATCGTTTTGACTATCATATCTCTTGTTTTTGGTATACTTAGCCTGATTCT |
| | AGCATGCTACCTAATGTACAAGCAAAAGGCGCAACAAAAGACCTTATTATGGCTTGGGAA |
| | TAATACaCTcGATCAGATGAGAGCCACTACAAAAATGTGAACACAGATGAGGAACGAAGG |
| | TTTCCCTAATAGTAATTTGTGTGAAAGTTCTGGTAGTCTGTCAGTTCAGAGAGTTAAGAA |
| | AAAACTACCGGTTGTAGATGACCAAAGGACGATATACGGGTAGAACGGTAAGAGAGGCCG |
| | CCCCTCAATTGCGAGCCAGGCTTCACAACCTCCGTTCTACCGCTTCACCGACAACAGTCC |
| | TCAATCATGGACCGCGCCGTTAGCCAAGTTGCGTTAGAGAATGATGAAAGAGAGGCAAAA |
| | AATACATGGCGCTTGATATTCCGGATTGCAATCTTATTCTTAACAGTAGTGACCTTGGCT |
| | ATATCTGTAGCCTCCCTTTTATATAGCATGGGGGCTAGCACACCTTCCACTCTGATCAGC |
| | CTAAATAACTCAATTATCACAAGCAGCAATGGTCTCAAAAAGGAAATCCTGAACCAGAAC |
| | ATAAAAGAGGACCTCATATATAGAGAAGTTGCTATAAATATACCTTTAACATTAGATAGG |
| | GTTACTGTTGAGGTAGGGACTGCAGTAAACCAGATTACTGATGCACTCAGGCAACTCCAG |
| | TCAGTTAATGGATCTGCTGCATTCGCCTCATCAAACTCTCCTGATTATAGTGGGGGAATA |
| | GAACACCTGATTTTCCAAAGGAATACGCTTATTAATCGCTCAGTGAGTGTCTCAGATTTA |
| | ATAGAACACCCCAGTTTCATACCAACTCCTACTACACAGCATGGTTGTACCAGAATCCCC |
| | ACATTCCACCTAGGAACTCGCCACTGGTGCTATAGTCACAATATAATAGGTCAGGGATGT |
| | GCTGATTCTAGAGCTAGTGTGATGTATATTTCAATGGGAGCACTGGGTGTCAGTTCATTG |
| | GGAACCCCGACCTTCACAACATCTGCTGCATCAATATTATCTGATAGCCTCAATCGGAAG |
| | AGTTGCAGTATAGTAGCAACAACTGAGGGTTGTGACGTACTCTGCAGTATAGTTACACAA |
| | ACAGAAGACCAAGATTATGCTGATCACACTCCTACTCCAATGATACATGGTAGATTATGG |
| | TTTAATGGCACATACACAGAGAGATCCTTATCCCAGAGTTTATTCCTTGGAACATGGGCT |
| | GCGCAATATCCGGCTGTAGGATCTGGTATAATGACACCTGGGCGAGTTATATTCCCTTTC |
| | TATGGAGGTGTGATCCCTAACTCTCCTCTCTTCTTGGATCTCGAAAGATTCGCTTTATTC |
| | ACACATAATGGAGACTTAGAATGCATGAACTTAACACAATATCAGAAAGAAGCAATTTAC |
| | TCTGCATATAAGCCTCCCAAGATTAGAGGATCACTGTGGGCACAAGGCTTCATAGTATGT |
| | TCAGTAGGAGACATGGGGAATTGCTCTCTTAAAGTGATCAATACAAGCACAGTTATGATG |
| | GGTGCAGAAGGTCGGCTACAATTAGTTGGGGACTCCGTTATGTACTATCAGAGATCATCA |
| | TCCTGGTGGCCTGTAGGAATTCTTTATCGGTTGAGTCTTGTAGACATCATCGCCGGAGAT |
| | ATACAGGTCGTCATAAACAGTGAACCACTCCCTCTGAGCAAGTTCCCGCGGCCAACCTGG |
| | ACTCCAGGAGTGTGTCAAAAACCAAATGTATGCCCTGCAGTTTGTGTAACTGGGGTCTAT |
| | CAAGACCTTTGGGCAATTTCCGCAGGGGAGACACTATCTGAAATGACATTCTTTGGAGGA |
| | TATTTAGAGGCATCCACCCAACGAAAAGATCCATGGATAGGCGTTGCTAATCAATATAGT |
| | TGGTTCATGAGAAGAAGATTATTCAAGACAAGCACTGAAGCTGCATATTCGTCATCAACG |
| | TGTTTTAGGAACACTAGACTGGATCGAAATTTCTGCCTATTAGTCTTTGAATTAACTGAT |
| | AACTTACTTGGAGACTGGAGAATTGTCCCCCTCTTATTTGAATTAACCATCGTATAAggc |
| | gcgccTTGAGTCAATTATAAAGGAGTTGGAAAGATGGCATTGTATCACCTATCTTCTGCG |
| | ACATCAAGAATCAAACCGAATGCCGGCGCGTGCTCGAATTCCATGTTGCCAGTTGACCAC |
| | AATCAGCCAGTGCTCATGCGATCAGATTAAGCCTTGTCATTAATCTCTTGATTAAGAAAA |
| | AATGTAAGTGGCAATGAGATACAAGGCAAAACAGCTCATGGTAAATAATACGGGTAGGAC |
| | ATGGCGAGCTCCGGTCCTGAAAGGGCAGAGCATCAGATTATCCTACCAGAGCCACACCTG |
| | TCTTCACCATTGGTCAAGCACAAACTACTCTATTACTGGAAATTAACTGGGCTACCGCTT |
| | CCTGATGAATGTGACTTCGACCACCTCATTCTCAGCCGACAATGGAAAAAAATACTTGAA |
| | TCGGCCTCTCCTGATACTGAGAGAATGATAAAACTCGGAAGGGCAGTACACCAAACTCTT |
| | AACCACAATTCCAGAATAACCGGAGTGCTCCACCCCAGGTGTTTAGAACAACTGGCTAAT |
| | ATTGAGGTCCCAGATTCAACCAACAAATTTCGGAAGATTGAGAAGAAGATCCAAATTCAC |
| | AACACGAGATATGGAGAACTGTTCACAAGGCTGTGTACGCATATAGAGAAGAAACTGCTG |
| | GGGTCATCTTGGTCTAACAATGTCCCCCGGTCAGAGGAGTTCAGCAGCATTCGTACGGAT |
| | CCGGCATTCTGGTTTCACTCAAAATGGTCCACAGCCAAGTTTGCATGGCTCCATATAAAA |
| | CAGATCCAGAGGCATCTGATGGTGGCAGCTAAGACAAGGTCTGCGGCCAACAAATTGGTG |
| | ATGCTAACCCATAAGGTAGGCCAAGTCTTTGTCACTCCTGAACTTGTCGTTGTGACGCAT |
| | ACGAATGAGAACAAGTTCACATGTCTTACCCAGGAACTTGTATTGATGTATGCAGATATG |
| | ATGGAGGGCAGAGATATGGTCAACATAATATCAACCACGGCGGTGCATCTCAGAAGCTTA |
| | TCAGAGAAAATTGATGACATTTTGCGGTTAATAGACGCTCTGGCAAAAGACTTGGGTAAT |
| | CAAGTCTACGATGTTGTATCACTAATGGAGGGATTTGCATACGGAGCTGTCCAGCTACTC |
| | GAGCCGTCAGGTACATTTGCAGGAGATTTCTTCGCATTCAACCTGCAGGAGCTTAAAGAC |
| | ATTCTAATTGGCCTCCTCCCCAATGATATAGCAGAATCCGTGACTCATGCAATCGCTACT |
| | GTATTCTCTGGTTTAGAACAGAATCAAGCAGCTGAGATGTTGTGTCTGTTGCGTCTGTGG |
| | GGTCACCCACTGCTTGAGTCCCGTATTGCAGCAAAGGCAGTCAGGAGCCAAATGTGCGCA |
| | CCGAAAATGGTAGACTTTGATATGATCCTTCAGGTACTGTCTTTCTTCAAGGGAACAATC |
| | ATCAACGGGTACAGAAAGAAGAATGCAGGTGTGTGGCCGCGAGTCAAAGTGGATACAATA |
| | TATGGGAAGGTCATTGGGCAACTACATGCAGATTCAGCAGAGATTTCACACGATATCATG |
| | TTGAGAGAGTATAAGAGTTTATCTGCACTTGAATTTGAGCCATGTATAGAATATGACCCT |
| | GTCACCAACCTGAGCATGTTCCTAAAAGACAAGGCAATCGCACACCCCAACGATAATTGG |
| | CTTGCCTCGTTTAGGCGGAACCTTCTCTCCGAAGACCAGAAGAAACATGTAAAAGAAGCA |
| | ACTTCGACTAATCGCCTCTTGATAGAGTTTTTAGAGTCAAATGATTTTGATCCATATAAA |
| | GAGATGGAATATCTGACGACCCTTGAGTACCTTAGAGATGACAATGTGGCAGTATCATAC |
| | TCGCTCAAGGAGAAGGAAGTGAAAGTTAATGGACGGATCTTCGCTAAGCTGACAAAGAAG |
| | TTAAGGAACTGTCAGGTGATGGCGGAAGGGATCCTAGCCGATCAGATTGCACCTTTCTTT |
| | CAGGGAAATGGAGTCATTCAGGATAGCATATCCTTGACCAAGAGTATGCTAGCGATGAGT |
| | CAACTGTCTTTTAACAGCAATAAGAAACGTATCACTGACTGTAAAGAAAGAGTATCTTCA |
| | AACCGCAATCATGATCCGAAAAGCAAGAACCGTCGGAGAGTTGCAACCTTCATAACAACT |
| | GACCTGCAAAAGTACTGTCTTAATTGGAGATATCAGACAATCAAATTGTTCGCTCATGCC |
| | ATCAATCAGTTGATGGGCCTACCTCACTTCTTCGAATGGATTCACCTAAGACTGATGGAC |
| | ACTACGATGTTCGTAGGAGACCCTTTCAATCCTCCAAGTGACCCTACTGACTGTGACCTC |
| | TCAAGAGTCCCTAATGATGACATATATATTGTCAGTGCCAGAGGGGGTATCGAAGGATTA |
| | TGCCAGAAGCTATGGACAATGATCTCAATTGCTGCAATCCAACTTGCTGCAGCTAGATCG |
| | CATTGTCGTGTTGCCTGTATGGTACAGGGTGATAATCAAGTAATAGCAGTAACGAGAGAG |
| | GTAAGATCAGACGACTCTCCGGAGATGGTGTTGACACAGTTGCATCAAGCCAGTGATAAT |
| | TTCTTCAAGGAATTAATTCATGTCAATCATTTGATTGGCCATAATTTGAAGGATCGTGAA |
| | ACCATCAGGTCAGACACATTCTTCATATACAGCAAACGAATCTTCAAAGATGGAGCAATC |
| | CTCAGTCAAGTCCTCAAAAATTCATCTAAATTAGTGCTAGTGTCAGGTGATCTCAGTGAA |
| | AACACCGTAATGTCCTGTGCCAACATTGCCTCTACTGTAGCACGGCTATGCGAGAACGGG |
| | CTTCCCAAAGACTTCTGTTACTATTTAAACTATATAATGAGTTGTGTGCAGACATACTTT |
| | GACTCTGAGTTCTCCATCACCAACAATTCGCACCCCGATCTTAATCAGTCGTGGATTGAG |
| | GACATCTCTTTTGTGCACTCATATGTTCTGACTCCTGCCCAATTAGGGGGACTGAGTAAC |
| | CTTCAATACTCAAGGCTCTACACTAGAAATATCGGTGACCCGGGGACTACTGCTTTTGCA |
| | GAGATCAAGCGACTAGAAGCAGTGGGATTACTGAGTCCTAACATTATGACTAATATCTTA |
| | ACTAGGCCGCCTGGGAATGGAGATTGGGCCAGTCTGTGCAACGACCCATACTCTTTCAAT |
| | TTTGAGACTGTTGCAAGCCCAAATATTGTTCTTAAGAAACATACGCAAAGAGTCCTATTT |
| | GAAACTTGTTCAAATCCCTTATTGTCTGGAGTGCACACAGAGGATAATGAGGCAGAAGAG |
| | AAGGCATTGGCTGAATTCTTGCTTAATCAAGAGGTGATTCATCCCCGCGTTGCGCATGCC |
| | ATCATGGAGGCAAGCTCTGTAGGTAGGAGAAAGCAAATTCAAGGGCTTGTTGACACAACA |
| | AACACCGTAATTAAGATTGCGCTTACTAGGAGGCCATTAGGCATCAAGAGGCTGATGCGG |
| | ATAGTCAATTATTCTAGCATGCATGCAATGCTGTTTAGAGACGATGTTTTTTCCTCCAGT |
| | AGATCCAACCACCCCTTAGTCTCTTCTAATATGTGTTCTCTGACACTGGCAGACTATGCA |
| | CGGAATAGAAGCTGGTCACCTTTGACGGGAGGCAGGAAAATACTGGGTGTATCTAATCCT |
| | GATACGATAGAACTCGTAGAGGGTGAGATTCTTAGTGTAAGCGGAGGGTGTACAAGATGT |
| | GACAGCGGAGATGAACAATTTACTTGGTTCCATCTTCCAAGCAATATAGAATTGACCGAT |
| | GACACCAGCAAGAATCCTCCGATGAGGGTACCATATCTCGGGTCAAAGACACAGGAGAGG |
| | AGAGCTGCCTCACTTGCAAAAATAGCTCATATGTCGCCACATGTAAAGGCTGCCCTAAGG |
| | GCATCATCCGTGTTGATCTGGGCTTATGGGGATAATGAAGTAAATTGGACTGCTGCTCTT |
| | ACGATTGCAAAATCTCGGTGTAATGTAAACTTAGAGTATCTTCGGTTACTGTCCCCTTTA |
| | CCCACGGCTGGGAATCTTCAACATAGACTAGATGATGGTATAACTCAGATGACATTCACC |
| | CCTGCATCTCTCTACAGGgtgtcaccttacattcacatatccaatgattctcaaaggctg |
| | ttcactgaagaaggagtcaaagaggggaatgtggtttaccaacagatcATGCTCTTGGGT |
| | TTATCTCTAATCGAATCGATCTTTCCAATGACAACAACGAGGACATATGATGAGATCACA |
| | CTGCACCTACATAGTAAATTTAGTTGCTGTATCAGAGAAGCACCTGTTGCGGTTCCTTTC |
| | GAGCTACTTGGGGTGGTACCGGAACTGAGGACAGTGACCTCAAATAAGTTTATGTATGAT |
| | CCTAGCCCTGTATCGGAGGGAGACTTTGCGAGACTTGACTTAGCTATCTTCAAGAGTTAT |
| | GAGCTTAATCTGGAGTCATATCCCACGATAGAGCTAATGAACATTCTTTCAATATCCAGC |
| | GGGAAGTTGATTGGCCAGTCTGTGGTTTCTTATGATGAAGATACCTCCATAAAGAATGAC |
| | GCCATAATAGTGTATGACAATACCCGAAATTGGATCAGTGAAGCTCAGAATTCAGATGTG |
| | GTCCGCCTATTTGAATATGCAGCACTTGAAGTGCTCCTCGACTGTTCTTACCAACTCTAT |
| | TACCTGAGAGTAAGAGGCCTAGACAATATTGTCTTATATATGGGTGATTTATACAAGAAT |
| | ATGCCAGGAATTCTACTTTCCAACATTGCAGCTACAATATCTCATCCCGTCATTCATTCA |
| | AGGTTACATGCAGTGGGCCTGGTCAACCATGACGGATCACACCAACTTGCAGATACGGAT |
| | TTTATCGAAATGTCTGCAAAACTATTAGTATCTTGCACCCGACGTGTGATCTCCGGCTTA |
| | TATTCAGGAAATAAGTATGATCTGCTGTTCCCATCTGTCTTAGATGATAACCTGAATGAG |
| | AAGATGCTTCAGCTGATATCCCGGTTATGCTGTCTGTACACGGTACTCTTTGCTACAACA |
| | AGAGAAATCCCGAAAATAAGAGGCTTAACTGCAGAAGAGAAATGTTCAATACTCACTGAG |
| | TATTTACTGTCGGATGCTGTGAAACCATTACTTAGCCCCGATCAAGTGAGCTCTATCATG |
| | TCTCCTAACATAATTACATTCCCAGCTAATCTGTACTACATGTCTCGGAAGAGCCTCAAT |
| | TTGATCAGGGAAAGGGAGGACAGGGATACTATCCTGGCGTTGTTGTTCCCCCAAGAGCCA |
| | TTATTAGAGTTCCCTTCTGTGCAAGATATTGGTGCTCGAGTGAAAGATCCATTCACCCGA |
| | CAACCTGCGGCATTTTTGCAAGAGTTAGATTTGAGTGCTCCAGCAAGGTATGACGCATTC |
| | ACACTTAGTCAGATTCATCCTGAACTCACATCTCCAAATCCGGAGGAAGACTACTTAGTA |
| | CGATACTTGTTCAGAGGGATAGGGACTGCATCTTCCTCTTGGTATAAGGCATCTCATCTC |
| | CTTTCTGTACCCGAGGTAAGATGTGCAAGACACGGGAACTCCTTATACTTAGCTGAAGGG |
| | AGCGGAGCCATCATGAGTCTTCTCGAACTGCATGTACCACATGAAACTATCTATTACAAT |
| | ACGCTCTTTTCAAATGAGATGAACCCCCCGCAACGACATTTCGGGCCGACCCCAACTCAG |
| | TTTTTGAATTCGGTTGTTTATAGGAATCTACAGGCGGAGGTAACATGCAAAGATGGATTT |
| | GTCCAAGAGTTCCGTCCATTATGGAGAGAAAATACAGAGGAAAGTGACCTGACCTCAGAT |
| | AAAGCAGTGGGGTATATTACATCTGCAGTGCCCTACAGATCTGTATCATTGCTGCATTGT |
| | GACATTGAAATTCCTCCAGGGTCCAATCAAAGCTTACTAGATCAACTAGCTATCAATTTA |
| | TCTCTGATTGCCATGCATTCTGTAAGGGAGGGCGGGGTAGTAATCATCAAAGTGTTGTAT |
| | GCAATGGGATACTACTTTCATCTACTCATGAACTTGTTTGCTCCGTGTTCCACAAAAGGA |
| | TATATTCTCTCTAATGGTTATGCATGTCGAGGAGATATGGAGTGTTACCTGGTATTTGTC |
| | ATGGGTTACCTGGGCGGGCCTACATTTGTACATGAGGTGGTGAGGATGGCAAAAACTCTG |
| | GTGCAGCGGCACGGTACGCTCTTGTCTAAATCAGATGAGATCACACTGACCAGGTTATTC |
| | ACCTCACAGCGGCAGCGTGTGACAGACATCCTATCCAGTCCTTTACCAAGATTAATAAAG |
| | TACTTGAGGAAGAATATTGACACTGCGCTGATTGAAGCCGGGGGACAGCCCGTCCGTCCA |
| | TTCTGTGCGGAGAGTCTGGTGAGCACGCTAGCGAACATAACTCAGATAACCCAGATTATC |
| | GCTAGTCACATTGACACAGTTATCCGGTCTGTGATATATATGGAAGCTGAGGGTGATCTC |
| | GCTGACACAGTATTTCTATTTACCCCTTACAATCTCTCTACTGACGGGAAAAAGAGGACA |
| | TCACTTATACAGTGCACGAGACAGATCCTAGAGGTTACAATACTAGGTCTTAGAGTCGAA |
| | AATCTCAATAAAATAGGCGATATAATCAGCCTAGTGCTTAAAGGCATGATCTCCATGGAG |
| | GACCTTATCCCACTAAGGACATACTTGAAGCATAGTACCTGCCCTAAATATTTGAAGGCT |
| | GTCCTAGGTATTACCAAACTCAAAGAAATGTTTACAGACACTTCTGTATTGTACTTGACT |
| | CGTGCTCAACAAAAATTCTACATGAAAACTATAGGCAATGCAGTCAAAGGATATTACAGT |
| | AACTGTGACTCTTAACGAAAATCACATATTAATAGGCTCCTTTTTTGGCCAATTGTATTC |
| | TTGTTGATTTAATCATATTATGTTAGAAAAAAGTTGAACCCTGACTCCTTAGGACTCGAA |
| | TTCGAACTCAAATAAATGTCTTAAAAAAAGGTTGCGCACAATTATTCTTGAGTGTAGTCT |
| | CGTCATTCACCAAATCTTTGTTTGGTGGCCGGCATGGTCCCAGCCTCCTCGCTGGCGCCG |
| | GCTGGGCAACATTCCGAGGGGACCGTCCCCTCGGTAATGGCGAATGGGACGTCGACTGCT |
| | AACAAAGCCCGAAAGGAAGCTGAGTTGGCTGCTGCCACCGCTGAGCAATAACTAGCATAA |
| | CCCCTTGGGGCCTCTAAACGGGTCTTGAGGGGTTTTTTGCTGAAAGGAGGAACTATAT |
| SEQ ID NO: | TAATACGACTCACTATAGGGACCAAACAGAGAATCCGTGAGTTACGATAAAAGGCGAAGG |
| 10; NDV | AGCAATTGAAGTCGCACGGGTAGAAGGTGTGAATCTCGAGTGCGAGCCCGAAGCACAAAC |
| vector | TCGAGAAAGCCTTCTGCCAACATGTCTTCCGTATTTGATGAGTACGAACAGCTCCTCGCG |
| lentogenic | GCTCAGACTCGCCCCAATGGAGCTCATGGAGGGGGAGAAAAAGGGAGTACCTTAAAAGTA |
| without GFP | GACGTCCCGGTATTCACTCTTAACAGTGATGACCCAGAAGATAGATGGAGCTTTGTGGTA |
| | TTCTGCCTCCGGATTGCTGTTAGCGAAGATGCCAACAAACCACTCAGGCAAGGTGCTCTC |
| | ATATCTCTTTTATGCTCCCACTCACAGGTAATGAGGAACCATGTTGCCATTGCAGGGAAA |
| | CAGAATGAAGCCACATTGGCCGTGCTTGAGATTGATGGCTTTGCCAACGGCACGCCCCAG |
| | TTCAACAATAGGAGTGGAGTGTCTGAAGAGAGAGCACAGAGATTTGCGATGATAGCAGGA |
| | TCTCTCCCTCGGGCATGCAGCAACGGAACCCCGTTCGTCACAGCCGGGGCAGAAGATGAT |
| | GCACCAGAAGACATCACCGATACCCTGGAGAGGATCCTCTCTATCCAGGCTCAAGTATGG |
| | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaaaacgcgtACCC |
| | AAGGTCCAACTCTCCAAGCGGCAATCCTCTCTCGCTTCCTCAGCCCCACTGAATGGTCGC |
| | GTAACCGTAATTAATCTAGCTACATTTAAGATTAAGAAAAAATACGGGTAGAATTGGAGT |
| | GCCCCAATTGTGCCAAGATGGACTCATCTAGGACAATTGGGCTGTACTTTGATTCTGCCC |
| | ATTCTTCTAGCAACCTGTTAGCATTTCCGATCGTCCTACAAGGCACAGGAGATGGGAAGA |
| | AGCAAATCGCCCCGCAATATAGGATCCAGCGCCTTGACTTGTGGACTGATAGTAAGGAGG |
| | ACTCAGTATTCATCACCACCTATGGATTCATCTTTCAAGTTGGGAATGAAGAAGCCACTG |
| | TCGGCATGATCGATGATAAACCCAAGCGCGAGTTACTTTCCGCTGCGATGCTCTGCCTAG |
| | GAAGCGTCCCAAATACCGGAGACCTTATTGAGCTGGCAAGGGCCTGTCTCACTATGATAG |
| | TCACATGCAAGAAGAGTGCAACTAATACTGAGAGAATGGTTTTCTCAGTAGTGCAGGCAC |
| | CCCAAGTGCTGCAAAGCTGTAGGGTTGTGGCAAACAAATACTCATCAGTGAATGCAGTCA |
| | AGCACGTGAAAGCGCCAGAGAAGATTCCCGGGAGTGGAACCCTAGAATACAAGGTGAACT |
| | TTGTCTCCTTGACTGTGGTACCGAAGAAGGATGTCTACAAGATCCCAGCTGCAGTATTGA |
| | AGGTTTCTGGCTCGAGTCTGTACAATCTTGCGCTCAATGTCACTATTAATGTGGAGGTAG |
| | ACCCGAGGAGTCCTTTGGTTAAATCTTTGTCTAAGTCTGACAGCGGATACTATGCTAACC |
| | TCTTCTTGCATATTGGACTTATGACCACCGTAGATAGGAAGGGGAAGAAAGTGACATTTG |
| | ACAAGCTGGAAAAGAAAATAAGGAGCCTTGATCTATCTGTCGGGCTCAGTGATGTGCTCG |
| | GGCCTTCCGTGTTGGTAAAAGCAAGAGGTGCACGGACTAAGCTTTTGGCACCTTTCTTCT |
| | CTAGCAGTGGGACAGCCTGCTATCCCATAGCAAATGCTTCTCCTCAGGTGGCCAAGATAC |
| | TCTGGAGTCAAACCGCGTGCCTGCGGAGCGTTAAAATCATTATCCAAGCAGGTACCCAAC |
| | GCGCTGTCGCAGTGACCGCCGACCACGAGGTTACCTCTACTAAGCTGGAGAAGGGGCACA |
| | CCCTTGCCAAATACAATCCTTTTAAGAAATAAGCTGCGTCTCTGAGATTGCGCTCCGCCC |
| | ACTCACCCAGATCATCATGACACAAAAAACTAATCTGTCTTGATTATTTACAGTTAGTTT |
| | ACCTGTCTATCAAGTTAGAAAAAACACGGGTAGAAGATTCTGGATCCCGGTTGGCGCCCT |
| | CCAGGTGCAAGttaattaaATGGGCTCCAGACCTTCTACCAAGAACCCAGCACCTATGAT |
| | GCTGACTATCCGGGTTGCGCTGGTACTGAGTTGCATCTGTCCGGCAAACTCCATTGATGG |
| | CAGGCCTCTTGCAGCTGCAGGAATTGTGGTTACAGGAGACAAAGCCGTCAACATATACAC |
| | CTCATCCCAGACAGGATCAATCATAGTTAAGCTCCTCCCGAATCTGCCCAAGGATAAGGA |
| | GGCATGTGCGAAAGCCCCCTTGGATGCATACAACAGGACATTGACCACTTTGCTCACCCC |
| | CCTTGGTGACTCTATCCGTAGGATACAAGAGTCTGTGACTACATCTGGAGGGGGGAGACA |
| | GGGGCGCCTTATAGGCGCCATTATTGGCGGTGTGGCTCTTGGGGTTGCAACTGCCGCACA |
| | AATAACAGCGGCCGCAGCTCTGATACAAGCCAAACAAAATGCTGCCAACATCCTCCGACT |
| | TAAAGAGAGCATTGCCGCAACCAATGAGGCTGTGCATGAGGTCACTGACGGATTATCGCA |
| | ACTAGCAGTGGCAGTTGGGAAGATGCAGCAGTTTGTTAATGACCAATTTAATAAAACAGC |
| | TCAGGAATTAGACTGCATCAAAATTGCACAGCAAGTTGGTGTAGAGCTCAACCTGTACCT |
| | AACCGAATTGACTACAGTATTCGGACCACAAATCACTTCACCTGCTTTAAACAAGCTGAC |
| | TATTCAGGCACTTTACAATCTAGCTGGTGGAAATATGGATTACTTATTGACTAAGTTAGG |
| | TGTAGGGAACAATCAACTCAGCTCATTAATCGGTAGCGGCTTAATCACtGGcAACCCTAT |
| | TCTATACGACTCACAGACTCAACTCTTGGGTATACAGGTAACTgcaCCTTCAGTCGGGAA |
| | CCTAAATAATATGCGTGCCACCTACTTGGAAACCTTATCCGTAAGCACAACCAGGGGATT |
| | TGCCTCGGCACTTGTCCCCAAAGTGGTGACACAGGTCGGTTCTGTGATAGAAGAACTTGA |
| | CACCTCATACTGTATAGAAACTGACTTAGATTTATATTGTACAAGAATAGTAACGTTCCC |
| | TATGTCCCCTGGTATTTATTCCTGCTTGAGCGGCAATACGTCGGCCTGTATGTACTCAAA |
| | GACCGAAGGCGCACTTACTACACCATACATGACTATCAAAGGTTCAGTCATCGCCAACTG |
| | CAAGATGACAACATGTAGATGTGTAAACCCCCCGGGTATCATATCGCAAAACTATGGAGA |
| | AGCCGTGTCTCTAATAGATAAACAATCATGCAATGTTTTATCCTTAGGCGGGATAACTTT |
| | AAGGCTCAGTGGGGAATTCGATGTAACTTATCAGAAGAATATCTCAATACAAGATTCTCA |
| | AGTAATAATAACAGGCAATCTTGATATCTCAACTGAGCTTGGGAATGTCAACAACTCGAT |
| | CAGTAATGCTTTGAATAAGTTAGAGGAAAGCAACAGAAAACTAGACAAAGTCAATGTCAA |
| | ACTGACTAGCACATCTGCTCTCATTACgTATATCGTTTTGACTATCATATCTCTTGTTTT |
| | TGGTATACTTAGCCTGATTCTAGCATGCTACCTAATGTACAAGCAAAAGGCGCAACAAAA |
| | GACCTTATTATGGCTTGGGAATAATACaCTcGATCAGATGAGAGCCACTACAAAAATGTG |
| | AACACAGATGAGGAACGAAGGTTTCCCTAATAGTAATTTGTGTGAAAGTTCTGGTAGTCT |
| | GTCAGTTCAGAGAGTTAAGAAAAAACTACCGGTTGTAGATGACCAAAGGACGATATACGG |
| | GTAGAACGGTAAGAGAGGCCGCCCCTCAATTGCGAGCCAGGCTTCACAACCTCCGTTCTA |
| | CCGCTTCACCGACAACAGTCCTCAATCATGGACCGCGCCGTTAGCCAAGTTGCGTTAGAG |
| | AATGATGAAAGAGAGGCAAAAAATACATGGCGCTTGATATTCCGGATTGCAATCTTATTC |
| | TTAACAGTAGTGACCTTGGCTATATCTGTAGCCTCCCTTTTATATAGCATGGGGGCTAGC |
| | ACACCTAGCGATCTTGTAGGCATACCGACTAGGATTTCCAGGGCAGAAGAAAAGATTACA |
| | TCTACACTTGGTTCCAATCAAGATGTAGTAGATAGGATATATAAGCAAGTGGCCCTTGAG |
| | TCTCCGTTGGCATTGTTAAATACTGAGACCACAATTATGAACGCAATAACATCTCTCTCT |
| | TATCAGATTAATGGAGCTGCAAACAACAGTGGGTGGGGGGCACCTATCCATGACCCAGAT |
| | TATATAGGGGGGATAGGCAAAGAACTCATTGTAGATGATGCTAGTGATGTCACATCATTC |
| | TATCCCTCTGCATTTCAAGAACATCTGAATTTTATCCCGGCGCCTACTACAGGATCAGGT |
| | TGCACTCGAATACCCTCATTTGACATGAGTGCTACCCATTACTGCTACACCCATAATGTA |
| | ATATTGTCTGGATGCAGAGATCACTCACATTCATATCAGTATTTAGCACTTGGTGTGCTC |
| | CGGACATCTGCAACAGGGAGGGTATTCTTTTCTACTCTGCGTTCCATCAACCTGGACGAC |
| | ACCCAAAATCGGAAGTCTTGCAGTGTGAGTGCAACTCCCCTGGGTTGTGATATGCTGTGC |
| | TCGAAAGTCACGGAGACAGAGGAAGAAGATTATAACTCAGCTGTCCCTACGCGGATGGTA |
| | CATGGGAGGTTAGGGTTCGACGGCCAGTACCACGAAAAGGACCTAGATGTCACAACATTA |
| | TTCGGGGACTGGGTGGCCAACTACCCAGGAGTAGGGGGTGGATCTTTTATTGACAGCCGC |
| | GTATGGTTCTCAGTCTACGGAGGGTTAAAACCCAATTCACCCAGTGACACTGTACAGGAA |
| | GGGAAATATGTGATATACAAGCGATACAATGACACATGCCCAGATGAGCAAGACTACCAG |
| | ATTCGAATGGCCAAGTCTTCGTATAAGCCTGGACGGTTTGGTGGGAAACGCATACAGCAG |
| | GCTATCTTATCTATCAAGGTGTCAACATCCTTAGGCGAAGACCCGGTACTGACTGTACCG |
| | CCCAACACAGTCACACTCATGGGGGCCGAAGGCAGAATTCTCACAGTAGGGACATCTCAT |
| | TTCTTGTATCAACGAGGGTCATCATACTTCTCTCCCGCGTTATTATATCCTATGACAGTC |
| | AGCAACAAAACAGCCACTCTTCATAGTCCTTATACATTCAATGCCTTCACTCGGCCAGGT |
| | AGTATCCCTTGCCAGGCTTCAGCAAGATGCCCCAACTCGTGTGTTACTGGAGTCTATACA |
| | GATCCATATCCCCTAATCTTCTATAGAAACCACACCTTGCGAGGGGTATTCGGGACAATG |
| | CTTGATGGTGTACAAGCAAGACTTAACCCTGCGTCTGCAGTATTCGATAGCACATCCCGC |
| | AGTCGCATTACTCGAGTGAGTTCAAGCAGTACCAAAGCAGCATACACAACATCAACTTGT |
| | TTTAAAGTGGTCAAGACTAATAAGACCTATTGTCTCAGCATTGCTGAAATATCTAATACT |
| | CTCTTCGGAGAATTCAGAATCGTCCCGTTACTAGTTGAGATCCTCAAAGATGACGGGGTT |
| | AGAGAAGCCAGGTCTGGCTAGggcgcgccTTGAGTCAATTATAAAGGAGTTGGAAAGATG |
| | GCATTGTATCACCTATCTTCTGCGACATCAAGAATCAAACCGAATGCCGGCGCGTGCTCG |
| | AATTCCATGTTGCCAGTTGACCACAATCAGCCAGTGCTCATGCGATCAGATTAAGCCTTG |
| | TCATTAATCTCTTGATTAAGAAAAAATGTAAGTGGCAATGAGATACAAGGCAAAACAGCT |
| | CATGGTAAATAATACGGGTAGGACATGGCGAGCTCCGGTCCTGAAAGGGCAGAGCATCAG |
| | ATTATCCTACCAGAGCCACACCTGTCTTCACCATTGGTCAAGCACAAACTACTCTATTAC |
| | TGGAAATTAACTGGGCTACCGCTTCCTGATGAATGTGACTTCGACCACCTCATTCTCAGC |
| | CGACAATGGAAAAAAATACTTGAATCGGCCTCTCCTGATACTGAGAGAATGATAAAACTC |
| | GGAAGGGCAGTACACCAAACTCTTAACCACAATTCCAGAATAACCGGAGTGCTCCACCCC |
| | AGGTGTTTAGAACAACTGGCTAATATTGAGGTCCCAGATTCAACCAACAAATTTCGGAAG |
| | ATTGAGAAGAAGATCCAAATTCACAACACGAGATATGGAGAACTGTTCACAAGGCTGTGT |
| | ACGCATATAGAGAAGAAACTGCTGGGGTCATCTTGGTCTAACAATGTCCCCCGGTCAGAG |
| | GAGTTCAGCAGCATTCGTACGGATCCGGCATTCTGGTTTCACTCAAAATGGTCCACAGCC |
| | AAGTTTGCATGGCTCCATATAAAACAGATCCAGAGGCATCTGATGGTGGCAGCTAAGACA |
| | AGGTCTGCGGCCAACAAATTGGTGATGCTAACCCATAAGGTAGGCCAAGTCTTTGTCACT |
| | CCTGAACTTGTCGTTGTGACGCATACGAATGAGAACAAGTTCACATGTCTTACCCAGGAA |
| | CTTGTATTGATGTATGCAGATATGATGGAGGGCAGAGATATGGTCAACATAATATCAACC |
| | ACGGCGGTGCATCTCAGAAGCTTATCAGAGAAAATTGATGACATTTTGCGGTTAATAGAC |
| | GCTCTGGCAAAAGACTTGGGTAATCAAGTCTACGATGTTGTATCACTAATGGAGGGATTT |
| | GCATACGGAGCTGTCCAGCTACTCGAGCCGTCAGGTACATTTGCAGGAGATTTCTTCGCA |
| | TTCAACCTGCAGGAGCTTAAAGACATTCTAATTGGCCTCCTCCCCAATGATATAGCAGAA |
| | TCCGTGACTCATGCAATCGCTACTGTATTCTCTGGTTTAGAACAGAATCAAGCAGCTGAG |
| | ATGTTGTGTCTGTTGCGTCTGTGGGGTCACCCACTGCTTGAGTCCCGTATTGCAGCAAAG |
| | GCAGTCAGGAGCCAAATGTGCGCACCGAAAATGGTAGACTTTGATATGATCCTTCAGGTA |
| | CTGTCTTTCTTCAAGGGAACAATCATCAACGGGTACAGAAAGAAGAATGCAGGTGTGTGG |
| | CCGCGAGTCAAAGTGGATACAATATATGGGAAGGTCATTGGGCAACTACATGCAGATTCA |
| | GCAGAGATTTCACACGATATCATGTTGAGAGAGTATAAGAGTTTATCTGCACTTGAATTT |
| | GAGCCATGTATAGAATATGACCCTGTCACCAACCTGAGCATGTTCCTAAAAGACAAGGCA |
| | ATCGCACACCCCAACGATAATTGGCTTGCCTCGTTTAGGCGGAACCTTCTCTCCGAAGAC |
| | CAGAAGAAACATGTAAAAGAAGCAACTTCGACTAATCGCCTCTTGATAGAGTTTTTAGAG |
| | TCAAATGATTTTGATCCATATAAAGAGATGGAATATCTGACGACCCTTGAGTACCTTAGA |
| | GATGACAATGTGGCAGTATCATACTCGCTCAAGGAGAAGGAAGTGAAAGTTAATGGACGG |
| | ATCTTCGCTAAGCTGACAAAGAAGTTAAGGAACTGTCAGGTGATGGCGGAAGGGATCCTA |
| | GCCGATCAGATTGCACCTTTCTTTCAGGGAAATGGAGTCATTCAGGATAGCATATCCTTG |
| | ACCAAGAGTATGCTAGCGATGAGTCAACTGTCTTTTAACAGCAATAAGAAACGTATCACT |
| | GACTGTAAAGAAAGAGTATCTTCAAACCGCAATCATGATCCGAAAAGCAAGAACCGTCGG |
| | AGAGTTGCAACCTTCATAACAACTGACCTGCAAAAGTACTGTCTTAATTGGAGATATCAG |
| | ACAATCAAATTGTTCGCTCATGCCATCAATCAGTTGATGGGCCTACCTCACTTCTTCGAA |
| | TGGATTCACCTAAGACTGATGGACACTACGATGTTCGTAGGAGACCCTTTCAATCCTCCA |
| | AGTGACCCTACTGACTGTGACCTCTCAAGAGTCCCTAATGATGACATATATATTGTCAGT |
| | GCCAGAGGGGGTATCGAAGGATTATGCCAGAAGCTATGGACAATGATCTCAATTGCTGCA |
| | ATCCAACTTGCTGCAGCTAGATCGCATTGTCGTGTTGCCTGTATGGTACAGGGTGATAAT |
| | CAAGTAATAGCAGTAACGAGAGAGGTAAGATCAGACGACTCTCCGGAGATGGTGTTGACA |
| | CAGTTGCATCAAGCCAGTGATAATTTCTTCAAGGAATTAATTCATGTCAATCATTTGATT |
| | GGCCATAATTTGAAGGATCGTGAAACCATCAGGTCAGACACATTCTTCATATACAGCAAA |
| | CGAATCTTCAAAGATGGAGCAATCCTCAGTCAAGTCCTCAAAAATTCATCTAAATTAGTG |
| | CTAGTGTCAGGTGATCTCAGTGAAAACACCGTAATGTCCTGTGCCAACATTGCCTCTACT |
| | GTAGCACGGCTATGCGAGAACGGGCTTCCCAAAGACTTCTGTTACTATTTAAACTATATA |
| | ATGAGTTGTGTGCAGACATACTTTGACTCTGAGTTCTCCATCACCAACAATTCGCACCCC |
| | GATCTTAATCAGTCGTGGATTGAGGACATCTCTTTTGTGCACTCATATGTTCTGACTCCT |
| | GCCCAATTAGGGGGACTGAGTAACCTTCAATACTCAAGGCTCTACACTAGAAATATCGGT |
| | GACCCGGGGACTACTGCTTTTGCAGAGATCAAGCGACTAGAAGCAGTGGGATTACTGAGT |
| | CCTAACATTATGACTAATATCTTAACTAGGCCGCCTGGGAATGGAGATTGGGCCAGTCTG |
| | TGCAACGACCCATACTCTTTCAATTTTGAGACTGTTGCAAGCCCAAATATTGTTCTTAAG |
| | AAACATACGCAAAGAGTCCTATTTGAAACTTGTTCAAATCCCTTATTGTCTGGAGTGCAC |
| | ACAGAGGATAATGAGGCAGAAGAGAAGGCATTGGCTGAATTCTTGCTTAATCAAGAGGTG |
| | ATTCATCCCCGCGTTGCGCATGCCATCATGGAGGCAAGCTCTGTAGGTAGGAGAAAGCAA |
| | ATTCAAGGGCTTGTTGACACAACAAACACCGTAATTAAGATTGCGCTTACTAGGAGGCCA |
| | TTAGGCATCAAGAGGCTGATGCGGATAGTCAATTATTCTAGCATGCATGCAATGCTGTTT |
| | AGAGACGATGTTTTTTCCTCCAGTAGATCCAACCACCCCTTAGTCTCTTCTAATATGTGT |
| | TCTCTGACACTGGCAGACTATGCACGGAATAGAAGCTGGTCACCTTTGACGGGAGGCAGG |
| | AAAATACTGGGTGTATCTAATCCTGATACGATAGAACTCGTAGAGGGTGAGATTCTTAGT |
| | GTAAGCGGAGGGTGTACAAGATGTGACAGCGGAGATGAACAATTTACTTGGTTCCATCTT |
| | CCAAGCAATATAGAATTGACCGATGACACCAGCAAGAATCCTCCGATGAGGGTACCATAT |
| | CTCGGGTCAAAGACACAGGAGAGGAGAGCTGCCTCACTTGCAAAAATAGCTCATATGTCG |
| | CCACATGTAAAGGCTGCCCTAAGGGCATCATCCGTGTTGATCTGGGCTTATGGGGATAAT |
| | GAAGTAAATTGGACTGCTGCTCTTACGATTGCAAAATCTCGGTGTAATGTAAACTTAGAG |
| | TATCTTCGGTTACTGTCCCCTTTACCCACGGCTGGGAATCTTCAACATAGACTAGATGAT |
| | GGTATAACTCAGATGACATTCACCCCTGCATCTCTCTACAGGgtgtcaccttacattcac |
| | atatccaatgattctcaaaggctgttcactgaagaaggagtcaaagaggggaatgtggtt |
| | taccaacagatcATGCTCTTGGGTTTATCTCTAATCGAATCGATCTTTCCAATGACAACA |
| | ACCAGGACATATGATGAGATCACACTGCACCTACATAGTAAATTTAGTTGCTGTATCAGA |
| | GAAGCACCTGTTGCGGTTCCTTTCGAGCTACTTGGGGTGGTACCGGAACTGAGGACAGTG |
| | ACCTCAAATAAGTTTATGTATGATCCTAGCCCTGTATCGGAGGGAGACTTTGCGAGACTT |
| | GACTTAGCTATCTTCAAGAGTTATGAGCTTAATCTGGAGTCATATCCCACGATAGAGCTA |
| | ATGAACATTCTTTCAATATCCAGCGGGAAGTTGATTGGCCAGTCTGTGGTTTCTTATGAT |
| | GAAGATACCTCCATAAAGAATGACGCCATAATAGTGTATGACAATACCCGAAATTGGATC |
| | AGTGAAGCTCAGAATTCAGATGTGGTCCGCCTATTTGAATATGCAGCACTTGAAGTGCTC |
| | CTCGACTGTTCTTACCAACTCTATTACCTGAGAGTAAGAGGCCTAGACAATATTGTCTTA |
| | TATATGGGTGATTTATACAAGAATATGCCAGGAATTCTACTTTCCAACATTGCAGCTACA |
| | ATATCTCATCCCGTCATTCATTCAAGGTTACATGCAGTGGGCCTGGTCAACCATGACGGA |
| | TCACACCAACTTGCAGATACGGATTTTATCGAAATGTCTGCAAAACTATTAGTATCTTGC |
| | ACCCGACGTGTGATCTCCGGCTTATATTCAGGAAATAAGTATGATCTGCTGTTCCCATCT |
| | GTCTTAGATGATAACCTGAATGAGAAGATGCTTCAGCTGATATCCCGGTTATGCTGTCTG |
| | TACACGGTACTCTTTGCTACAACAAGAGAAATCCCGAAAATAAGAGGCTTAACTGCAGAA |
| | GAGAAATGTTCAATACTCACTGAGTATTTACTGTCGGATGCTGTGAAACCATTACTTAGC |
| | CCCGATCAAGTGAGCTCTATCATGTCTCCTAACATAATTACATTCCCAGCTAATCTGTAC |
| | TACATGTCTCGGAAGAGCCTCAATTTGATCAGGGAAAGGGAGGACAGGGATACTATCCTG |
| | GCGTTGTTGTTCCCCCAAGAGCCATTATTAGAGTTCCCTTCTGTGCAAGATATTGGTGCT |
| | CGAGTGAAAGATCCATTCACCCGACAACCTGCGGCATTTTTGCAAGAGTTAGATTTGAGT |
| | GCTCCAGCAAGGTATGACGCATTCACACTTAGTCAGATTCATCCTGAACTCACATCTCCA |
| | AATCCGGAGGAAGACTACTTAGTACGATACTTGTTCAGAGGGATAGGGACTGCATCTTCC |
| | TCTTGGTATAAGGCATCTCATCTCCTTTCTGTACCCGAGGTAAGATGTGCAAGACACGGG |
| | AACTCCTTATACTTAGCTGAAGGGAGCGGAGCCATCATGAGTCTTCTCGAACTGCATGTA |
| | CCACATGAAACTATCTATTACAATACGCTCTTTTCAAATGAGATGAACCCCCCGCAACGA |
| | CATTTCGGGCCGACCCCAACTCAGTTTTTGAATTCGGTTGTTTATAGGAATCTACAGGCG |
| | GAGGTAACATGCAAAGATGGATTTGTCCAAGAGTTCCGTCCATTATGGAGAGAAAATACA |
| | GAGGAAAGTGACCTGACCTCAGATAAAGCAGTGGGGTATATTACATCTGCAGTGCCCTAC |
| | AGATCTGTATCATTGCTGCATTGTGACATTGAAATTCCTCCAGGGTCCAATCAAAGCTTA |
| | CTAGATCAACTAGCTATCAATTTATCTCTGATTGCCATGCATTCTGTAAGGGAGGGCGGG |
| | GTAGTAATCATCAAAGTGTTGTATGCAATGGGATACTACTTTCATCTACTCATGAACTTG |
| | TTTGCTCCGTGTTCCACAAAAGGATATATTCTCTCTAATGGTTATGCATGTCGAGGAGAT |
| | ATGGAGTGTTACCTGGTATTTGTCATGGGTTACCTGGGCGGGCCTACATTTGTACATGAG |
| | GTGGTGAGGATGGCAAAAACTCTGGTGCAGCGGCACGGTACGCTCTTGTCTAAATCAGAT |
| | GAGATCACACTGACCAGGTTATTCACCTCACAGCGGCAGCGTGTGACAGACATCCTATCC |
| | AGTCCTTTACCAAGATTAATAAAGTACTTGAGGAAGAATATTGACACTGCGCTGATTGAA |
| | GCCGGGGGACAGCCCGTCCGTCCATTCTGTGCGGAGAGTCTGGTGAGCACGCTAGCGAAC |
| | ATAACTCAGATAACCCAGATTATCGCTAGTCACATTGACACAGTTATCCGGTCTGTGATA |
| | TATATGGAAGCTGAGGGTGATCTCGCTGACACAGTATTTCTATTTACCCCTTACAATCTC |
| | TCTACTGACGGGAAAAAGAGGACATCACTTATACAGTGCACGAGACAGATCCTAGAGGTT |
| | ACAATACTAGGTCTTAGAGTCGAAAATCTCAATAAAATAGGCGATATAATCAGCCTAGTG |
| | CTTAAAGGCATGATCTCCATGGAGGACCTTATCCCACTAAGGACATACTTGAAGCATAGT |
| | ACCTGCCCTAAATATTTGAAGGCTGTCCTAGGTATTACCAAACTCAAAGAAATGTTTACA |
| | GACACTTCTGTATTGTACTTGACTCGTGCTCAACAAAAATTCTACATGAAAACTATAGGC |
| | AATGCAGTCAAAGGATATTACAGTAACTGTGACTCTTAACGAAAATCACATATTAATAGG |
| | CTCCTTTTTTGGCCAATTGTATTCTTGTTGATTTAATCATATTATGTTAGAAAAAAGTTG |
| | AACCCTGACTCCTTAGGACTCGAATTCGAACTCAAATAAATGTCTTAAAAAAAGGTTGCG |
| | CACAATTATTCTTGAGTGTAGTCTCGTCATTCACCAAATCTTTGTTTGGTGGCCGGCATG |
| | GTCCCAGCCTCCTCGCTGGCGCCGGCTGGGCAACATTCCGAGGGGACCGTCCCCTCGGTA |
| | ATGGCGAATGGGACGTCGACTGCTAACAAAGCCCGAAAGGAAGCTGAGTTGGCTGCTGCC |
| | ACCGCTGAGCAATAACTAGCATAACCCCTTGGGGCCTCTAAACGGGTCTTGAGGGGTTTT |
| | TTGCTGAAAGGAGGAACTATAT |
| SEQ ID NO: | MASSGPERAEHQIILPEPHLSSPLVKHKLLYYWKLTGLPLPDECDFDHLILSRQWKKILE |
| 11; Stabilized | SASPDTERMIKLGRAVHQTLNHNSRITGVLHPRCLEQLANIEVPDSTNKFRKIEKKIQIH |
| L protein | NTRYGELFTRLCTHIEKKLLGSSWSNNVPRSEEFSSIRTDPAFWFHSKWSTAKFAWLHIK |
| sequence | QIQRHLMVAAKTRSAANKLVMLTHKVGQVFVTPELVVVTHTNENKFTCLTQELVLMYADM |
| (2204 aa) | MEGRDMVNIISTTAVHLRSLSEKIDDILRLIDALAKDLGNQVYDVVSLMEGFAYGAVQLL |
| | EPSGTFAGDFFAFNLQELKDILIGLLPNDIAESVTHAIATVFSGLEQNQAAEMLCLLRLW |
| | GHPLLESRIAAKAVRSQMCAPKMVDFDMILQVLSFFKGTIINGYRKKNAGVWPRVKVDTI |
| | YGKVIGQLHADSAEISHDIMLREYKSLSALEFEPCIEYDPVTNLSMFLKDKAIAHPNDNW |
| | LASFRRNLLSEDQKKHVKEATSTNRLLIEFLESNDFDPYKEMEYLTTLEYLRDDNVAVSY |
| | SLKEKEVKVNGRIFAKLTKKLRNCQVMAEGILADQIAPFFQGNGVIQDSISLTKSMLAMS |
| | QLSFNSNKKRITDCKERVSSNRNHDPKSKNRRRVATFITTDLQKYCLNWRYQTIKLFAHA |
| | INQLMGLPHFFEWIHLRLMDTTMFVGDPFNPPSDPTDCDLSRVPNDDIYIVSARGGIEGL |
| | CQKLWTMISIAAIQLAAARSHCRVACMVQGDNQVIAVTREVRSDDSPEMVLTQLHQASDN |
| | FFKELIHVNHLIGHNLKDRETIRSDTFFIYSKRIFKDGAILSQVLKNSSKLVLVSGDLSE |
| | NTVMSCANIASTVARLCENGLPKDFCYYLNYIMSCVQTYFDSEFSITNNSHPDLNQSWIE |
| | DISFVHSYVLTPAQLGGLSNLQYSRLYTRNIGDPGTTAFAEIKRLEAVGLLSPNIMTNIL |
| | TRPPGNGDWASLCNDPYSFNFETVASPNIVLKKHTQRVLFETCSNPLLSGVHTEDNEAEE |
| | KALAEFLLNQEVIHPRVAHAIMEASSVGRRKQIQGLVDTTNTVIKIALTRRPLGIKRLMR |
| | IVNYSSMHAMLFRDDVFSSSRSNHPLVSSNMCSLTLADYARNRSWSPLTGGRKILGVSNP |
| | DTIELVEGEILSVSGGCTRCDSGDEQFTWFHLPSNIELTDDTSKNPPMRVPYLGSKTQER |
| | RAASLAKIAHMSPHVKAALRASSVLIWAYGDNEVNWTAALTIAKSRCNVNLEYLRLLSPL |
| | PTAGNLQHRLDDGITQMTFTPASLYRVSPYIHISNDSQRLFTEEGVKEGNVVYQQIMLLG |
| | LSLIESIFPMTTTRTYDEITLHLHSKFSCCIREAPVAVPFELLGVVPELRTVTSNKFMYD |
| | PSPVSEGDFARLDLAIFKSYELNLESYPTIELMNILSISSGKLIGQSVVSYDEDTSIKND |
| | AIIVYDNTRNWISEAQNSDVVRLFEYAALEVLLDCSYQLYYLRVRGLDNIVLYMGDLYKN |
| | MPGILLSNIAATISHPVIHSRLHAVGLVNHDGSHQLADTDFIEMSAKLLVSCTRRVISGL |
| | YSGNKYDLLFPSVLDDNLNEKMLQLISRLCCLYTVLFATTREIPKIRGLTAEEKCSILTE |
| | YLLSDAVKPLLSPDQVSSIMSPNIITFPANLYYMSRKSLNLIREREDRDTILALLFPQEP |
| | LLEFPSVQDIGARVKDPFTRQPAAFLQELDLSAPARYDAFTLSQIHPELTSPNPEEDYLV |
| | RYLFRGIGTASSSWYKASHLLSVPEVRCARHGNSLYLAEGSGAIMSLLELHVPHETIYYN |
| | TLFSNEMNPPQRHFGPTPTQFLNSVVYRNLQAEVTCKDGFVQEFRPLWRENTEESDLTSD |
| | KAVGYITSAVPYRSVSLLHCDIEIPPGSNQSLLDQLAINLSLIAMHSVREGGVVIIKVLY |
| | AMGYYFHLLMNLFAPCSTKGYILSNGYACRGDMECYLVFVMGYLGGPTFVHEVVRMAKTL |
| | VQRHGTLLSKSDEITLTRLFTSQRQRVTDILSSPLPRLIKYLRKNIDTALIEAGGQPVRP |
| | FCAESLVSTLANITQITQIIASHIDTVIRSVIYMEAEGDLADTVFLFTPYNLSTDGKKRT |
| | SLIQCTRQILEVTILGLRVENLNKIGDIISLVLKGMISMEDLIPLRTYLKHSTCPKYLKA |
| | VLGITKLKEMFTDTSVLYLTRAQQKFYMKTIGNAVKGYYSNCDS |
| SEQ ID NO: | MLQLPLTILLSILSAHQSLCLDNSKLIHAGIMSTTEREVNVYAQSITGSIVVRLIPNIPS |
| 12; Chimeric | NHKSCATSQIKLYNDTLTRLLTPIKANLEGLISAVSQDQSQNSGKRKKRFVGAVIGAAAL |
| APMV-5- | GLATAAQVTATVALNQAQENARNILRLKNSIQKTNEAVMELKDAVGQTAVAIDKTQAFIN |
| NDV F gene | NQILPAISNLSCEVLGNKIGVQLSLYLTELTTVFGNQLTNPALTTLSLQALYNLCGDDFN |
| (546 aa) | YLINLLNAKNRNLASLYEANLIQGRITQYDSMNQLLIIQVQIPSISTVSGMRVTELFTLS |
| | VDTPIGEGKALVPKYVLSSGRIMEEVDLSSCAITSTSVFCSSIISRPLPLETINCLNGNV |
| | TQCQFTANTGTLESRYAVIGGLVIANCKAIVCRCLNPPGVIAQNLGLPITIISSNTCQRI |
| | NLEQITLSLGNSILSTYSANLSQVEMNLAPSNPLDISVELNRVNTSLSKVESLIKESNSI |
| | LDSVNPQILNVKTLITYIVLTIISLVFGILSLILACYLMYKQKAQQKTLLWLGNNTLDQM |
| | RATTKM |
| SEQ ID NO: | MDRAVSQVALENDEREAKNTWRLIFRIAILFLTVVTLAISVASLLYSMGASTPSTLISLN |
| 13; Chimeric | NSIITSSNGLKKEILNQNIKEDLIYREVAINIPLTLDRVTVEVGTAVNQITDALRQLQSV |
| NDV-APMV- | NGSAAFASSNSPDYSGGIEHLIFQRNTLINRSVSVSDLIEHPSFIPTPTTQHGCTRIPTF |
| 5 HN gene | HLGTRHWCYSHNIIGQGCADSRASVMYISMGALGVSSLGTPTFTTSAASILSDSLNRKSC |
| (576 aa) | SIVATTEGCDVLCSIVTQTEDQDYADHTPTPMIHGRLWFNGTYTERSLSQSLFLGTWAAQ |
| Underlined | YPAVGSGIMTPGRVIFPFYGGVIPNSPLFLDLERFALFTHNGDLECMNLTQYQKEAIYSA |
| part from | YKPPKIRGSLWAQGFIVCSVGDMGNCSLKVINTSTVMMGAEGRLQLVGDSVMYYQRSSSW |
| NDV; | WPVGILYRLSLVDIIAGDIQVVINSEPLPLSKFPRPTWTPGVCQKPNVCPAVCVTGVYQD |
| remaining | LWAISAGETLSEMTFFGGYLEASTQRKDPWIGVANQYSWFMRRRLFKTSTEAAYSSSTCF |
| part from | RNTRLDRNFCLLVFELTDNLLGDWRIVPLLFELTIV |
| APMV5 | |
| SEQ ID NO: | MLQLPLTILLSILSAHQSLCLDNSKLIHAGIMSTTEREVNVYAQSITGSIVVRLIPNIPS |
| 14; wt APMV- | NHKSCATSQIKLYNDTLTRLLTPIKANLEGLISAVSQDQSQNSGKRKKRFVGAVIGAAAL |
| 5 F | GLATAAQVTATVALNQAQENARNILRLKNSIQKTNEAVMELKDAVGQTAVAIDKTQAFIN |
| (YP_009094158.1; | NQILPAISNLSCEVLGNKIGVQLSLYLTELTTVFGNQLTNPALTTLSLQALYNLCGDDFN |
| 544 | YLINLLNAKNRNLASLYEANLIQGRITQYDSMNQLLIIQVQIPSISTVSGMRVTELFTLS |
| aa) | VDTPIGEGKALVPKYVLSSGRIMEEVDLSSCAITSTSVFCSSIISRPLPLETINCLNGNV |
| | TQCQFTANTGTLESRYAVIGGLVIANCKAIVCRCLNPPGVIAQNLGLPITIISSNTCQRI |
| | NLEQITLSLGNSILSTYSANLSQVEMNLAPSNPLDISVELNRVNTSLSKVESLIKESNSI |
| | LDSVNPQILNVKTVIILAVIIGLIVVWCFILTCLIVRGFMLLVKQQKFKGLSVQNNPYVS |
| | NNSH |
| SEQ ID NO: | MDKSYYIEPEDQRGNSRTWRLLFRLIVLTLLCLIACILVSQLFYPWLPQVLSTLISLNNS |
| 15; wt APMV- | IITSSNGLKKEILNQNIKEDLIYREVAINIPLTLDRVTVEVGTAVNQITDALRQLQSVNG |
| 5 HN (574 aa) | SAAFASSNSPDYSGGIEHLIFQRNTLINRSVSVSDLIEHPSFIPTPTTQHGCTRIPTFHL |
| | GTRHWCYSHNIIGQGCADSRASVMYISMGALGVSSLGTPTFTTSAASILSDSLNRKSCSI |
| | VATTEGCDVLCSIVTQTEDQDYADHTPTPMIHGRLWFNGTYTERSLSQSLFLGTWAAQYP |
| | AVGSGIMTPGRVIFPFYGGVIPNSPLFLDLERFALFTHNGDLECMNLTQYQKEAIYSAYK |
| | PPKIRGSLWAQGFIVCSVGDMGNCSLKVINTSTVMMGAEGRLQLVGDSVMYYQRSSSWWP |
| | VGILYRLSLVDIIAGDIQVVINSEPLPLSKFPRPTWTPGVCQKPNVCPAVCVTGVYQDLW |
| | AISAGETLSEMTFFGGYLEASTQRKDPWIGVANQYSWFMRRRLFKTSTEAAYSSSTCFRN |
| | TRLDRNFCLLVFELTDNLLGDWRIVPLLFELTIV |
| SEQ ID NO: | MASSGPERAEHQIILPEPHLSSPLVKHKLLYYWKLTGLPLPDECDFDHLILSRQWKKILE |
| 16; wild type | SASPDTERMIKLGRAVHQTLNHNSRITGVLHPRCLEQLANIEVPDSTNKFRKIEKKIQIH |
| L protein | NTRYGELFTRLCTHIEKKLLGSSWSNNVPRSEEFSSIRTDPAFWFHSKWSTAKFAWLHIK |
| amino acid | QIQRHLMVAAKTRSAANKLVMLTHKVGQVFVTPELVVVTHTNENKFTCLTQELVLMYADM |
| sequence | MEGRDMVNIISTTAVHLRSLSEKIDDILRLIDALAKDLGNQVYDVVSLMEGFAYGAVQLL |
| (AAC28375.1; | EPSGTFAGDFFAFNLQELKDILIGLLPNDIAESVTHAIATVFSGLEQNQAAEMLCLLRLW |
| 2204 aa) | GHPLLESRIAAKAVRSQMCAPKMVDFDMILQVLSFFKGTIINGYRKKNAGVWPRVKVDTI |
| | YGKVIGQLHADSAEISHDIMLREYKSLSALEFEPCIEYDPVTNLSMFLKDKAIAHPNDNW |
| | LASFRRNLLSEDQKKHVKEATSTNRLLIEFLESNDFDPYKEMEYLTTLEYLRDDNVAVSY |
| | SLKEKEVKVNGRIFAKLTKKLRNCQVMAEGILADQIAPFFQGNGVIQDSISLTKSMLAMS |
| | QLSFNSNKKRITDCKERVSSNRNHDPKSKNRRRVATFITTDLQKYCLNWRYQTIKLFAHA |
| | INQLMGLPHFFEWIHLRLMDTTMFVGDPFNPPSDPTDCDLSRVPNDDIYIVSARGGIEGL |
| | CQKLWTMISIAAIQLAAARSHCRVACMVQGDNQVIAVTREVRSDDSPEMVLTQLHQASDN |
| | FFKELIHVNHLIGHNLKDRETIRSDTFFIYSKRIFKDGAILSQVLKNSSKLVLVSGDLSE |
| | NTVMSCANIASTVARLCENGLPKDFCYYLNYIMSCVQTYFDSEFSITNNSHPDLNQSWIE |
| | DISFVHSYVLTPAQLGGLSNLQYSRLYTRNIGDPGTTAFAEIKRLEAVGLLSPNIMTNIL |
| | TRPPGNGDWASLCNDPYSFNFETVASPNIVLKKHTQRVLFETCSNPLLSGVHTEDNEAEE |
| | KALAEFLLNQEVIHPRVAHAIMEASSVGRRKQIQGLVDTTNTVIKIALTRRPLGIKRLMR |
| | IVNYSSMHAMLFRDDVFSSSRSNHPLVSSNMCSLTLADYARNRSWSPLTGGRKILGVSNP |
| | DTIELVEGEILSVSGGCTRCDSGDEQFTWFHLPSNIELTDDTSKNPPMRVPYLGSKTQER |
| | RAASLAKIAHMSPHVKAALRASSVLIWAYGDNEVNWTAALTIAKSRCNVNLEYLRLLSPL |
| | PTAGNLQHRLDDGITQMTFTPASLYRCHLTFTYPMILKGCSLKKESKRGMWFTNRVMLLG |
| | LSLIESIFPMTTTRTYDEITLHLHSKFSCCIREAPVAVPFELLGVVPELRTVTSNKFMYD |
| | PSPVSEGDFARLDLAIFKSYELNLESYPTIELMNILSISSGKLIGQSVVSYDEDTSIKND |
| | AIIVYDNTRNWISEAQNSDVVRLFEYAALEVLLDCSYQLYYLRVRGLDNIVLYMGDLYKN |
| | MPGILLSNIAATISHPVIHSRLHAVGLVNHDGSHQLADTDFIEMSAKLLVSCTRRVISGL |
| | YSGNKYDLLFPSVLDDNLNEKMLQLISRLCCLYTVLFATTREIPKIRGLTAEEKCSILTE |
| | YLLSDAVKPLLSPDQVSSIMSPNIITFPANLYYMSRKSLNLIREREDRDTILALLFPQEP |
| | LLEFPSVQDIGARVKDPFTRQPAAFLQELDLSAPARYDAFTLSQIHPELTSPNPEEDYLV |
| | RYLFRGIGTASSSWYKASHLLSVPEVRCARHGNSLYLAEGSGAIMSLLELHVPHETIYYN |
| | TLFSNEMNPPQRHFGPTPTQFLNSVVYRNLQAEVTCKDGFVQEFRPLWRENTEESDLTSD |
| | KAVGYITSAVPYRSVSLLHCDIEIPPGSNQSLLDQLAINLSLIAMHSVREGGVVIIKVLY |
| | AMGYYFHLLMNLFAPCSTKGYILSNGYACRGDMECYLVFVMGYLGGPTFVHEVVRMAKTL |
| | VQRHGTLLSKSDEITLTRLFTSQRQRVTDILSSPLPRLIKYLRKNIDTALIEAGGQPVRP |
| | FCAESLVSTLANITQITQIIASHIDTVIRSVIYMEAEGDLADTVFLFTPYNLSTDGKKRT |
| | SLIQCTRQILEVTILGLRVENLNKIGDIISLVLKGMISMEDLIPLRTYLKHSTCPKYLKA |
| | VLGITKLKEMFTDTSVLYLTRAQQKFYMKTIGNAVKGYYSNCDS |
| SEQ ID NO: | ATGTTCGTGTTCCTGGTCCTGCTGCCACTGGTAAGCTCCCAATGTGTAAACTTAACCACA |
| 17; COVID19 | AGAACCCAGCTCCCACCTGCCTACACCAACAGCTTCACCAGAGGCGTTTATTACCCCGAC |
| S gene | AAGGTATTCCGGTCTTCTGTTCTGCACTCTACCCAGGACCTGTTTCTGCCCTTTTTCAGC |
| sequence | AACGTGACATGGTTCCACGCCATCCACGTGTCTGGCACAAACGGCACCAAGCGGTTTGAT |
| | AATCCTGTGCTCCCTTTCAATGACGGCGTGTACTTCGCCTCTACTGAGAAGAGCAACATC |
| | ATCCGGGGCTGGATCTTTGGCACAACACTGGACTCTAAAACCCAGAGCCTGCTGATCGTG |
| | AACAACGCCACCAACGTGGTGATTAAGGTGTGCGAGTTCCAGTTCTGCAATGACCCTTTC |
| | CTCGGCGTGTACTACCACAAGAACAACAAAAGTTGGATGGAAAGCGAATTCAGGGTGTAC |
| | TCAAGCGCCAACAACTGTACCTTCGAGTACGTGAGCCAGCCTTTCCTGATGGACCTAGAA |
| | GGTAAGCAGGGCAATTTCAAGAACCTCAGAGAGTTCGTGTTCAAGAATATTGACGGCTAC |
| | TTCAAAATCTACAGCAAGCACACCCCAATCAACCTGGTGCGGGACCTGCCCCAGGGCTTT |
| | AGCGCGCTGGAGCCTCTGGTGGACCTGCCTATCGGCATCAACATCACCCGGTTCCAGACA |
| | CTGCTGGCTCTGCATAGAAGCTACCTGACACCTGGCGACAGTTCTTCTGGCTGGACAGCC |
| | GGCGCCGCCGCCTACTACGTGGGCTACCTGCAGCCTAGAACATTCCTGCTGAAATACAAC |
| | GAGAACGGCACGATCACAGACGCCGTGGACTGCGCCCTGGATCCCCTGTCTGAGACAAAG |
| | TGCACCCTGAAGTCTTTCACCGTGGAGAAGGGCATCTACCAGACCTCCAACTTCAGAGTG |
| | CAGCCTACCGAATCCATCGTGCGCTTTCCCAACATCACCAACCTGTGCCCCTTCGGCGAG |
| | GTCTTTAATGCCACGAGATTCGCCAGCGTGTATGCCTGGAACAGAAAGAGAATCAGCAAC |
| | TGCGTGGCCGACTACAGCGTGCTGTACAACAGCGCCTCTTTCAGCACATTTAAGTGCTAC |
| | GGAGTGTCTCCTACCAAACTCAACGATCTGTGCTTCACGAACGTGTATGCCGACAGCTTC |
| | GTGATCCGAGGAGATGAGGTGCGGCAGATCGCTCCAGGACAGACAGGCAAGATCGCCGAC |
| | TACAACTACAAGCTGCCCGACGACTTTACCGGCTGCGTGATCGCTTGGAACAGCAATAAC |
| | CTGGACTCAAAGGTTGGAGGAAACTACAACTACCTGTACAGACTGTTCAGAAAGTCCAAC |
| | CTGAAGCCCTTCGAGAGAGACATCTCTACAGAAATCTACCAGGCCGGCAGCACCCCATGT |
| | AACGGCGTGGAAGGCTTCAACTGCTACTTCCCTCTGCAGTCTTATGGCTTCCAGCCCACA |
| | AACGGAGTGGGCTATCAGCCTTACCGCGTGGTTGTCCTGAGCTTTGAGCTGCTGCATGCC |
| | CCTGCTACGGTGTGTGGACCTAAGAAGTCCACCAACCTGGTGAAGAACAAGTGTGTGAAC |
| | TTCAACTTCAACGGCCTGACCGGCACCGGGGTGCTGACAGAGTCTAACAAGAAATTCCTG |
| | CCATTCCAGCAATTCGGCCGGGACATCGCCGACACCACCGACGCCGTGCGGGATCCTCAG |
| | ACCCTCGAAATCCTGGACATCACCCCCTGTAGCTTCGGCGGCGTGAGCGTGATCACCCCT |
| | GGCACAAACACCAGCAATCAAGTGGCTGTCCTGTACCAGGATGTCAATTGCACAGAAGTG |
| | CCTGTGGCCATCCACGCCGATCAGCTGACCCCCACCTGGCGGGTGTACTCGACAGGAAGC |
| | AACGTGTTTCAAACAAGAGCCGGCTGCCTGATCGGGGCCGAGCACGTGAACAATTCCTAC |
| | GAGTGCGACATCCCCATCGGCGCCGGCATCTGTGCCTCTTACCAGACACAGACCAATTCC |
| | CCTCGTAGAGCCAGATCCGTGGCCAGCCAGAGCATCATCGCCTACACCATGAGCCTGGGC |
| | GCCGAAAACAGCGTTGCATATTCCAACAACAGCATCGCCATCCCTACCAACTTCACCATC |
| | AGCGTGACCACAGAAATCCTGCCTGTGTCCATGACCAAGACAAGCGTTGATTGCACCATG |
| | TACATCTGCGGCGATAGCACAGAGTGCAGCAATCTGCTGCTGCAGTACGGTAGCTTCTGC |
| | ACCCAGCTGAATAGAGCCCTGACCGGCATCGCTGTGGAACAGGACAAAAACACCCAGGAG |
| | GTCTTCGCCCAGGTGAAGCAAATCTACAAGACCCCTCCAATCAAGGACTTCGGAGGCTTT |
| | AACTTTAGCCAGATCCTGCCTGATCCCTCCAAGCCTAGCAAACGGAGTTTCATCGAGGAC |
| | CTGCTCTTCAACAAGGTGACCCTGGCTGACGCCGGCTTCATTAAGCAGTACGGCGATTGC |
| | CTCGGCGACATCGCTGCAAGAGACCTGATCTGCGCCCAGAAGTTCAACGGCCTGACCGTG |
| | CTGCCTCCTCTCCTGACAGACGAGATGATCGCCCAGTACACCTCTGCCCTTCTGGCTGGC |
| | ACCATCACCAGCGGATGGACCTTTGGAGCCGGAGCCGCCCTGCAGATCCCTTTCGCTATG |
| | CAGATGGCCTACAGATTCAACGGGATCGGAGTGACCCAAAACGTGCTGTATGAAAACCAG |
| | AAACTGATCGCCAATCAGTTTAACAGCGCCATCGGCAAAATCCAGGATAGCCTGTCCAGC |
| | ACCGCCAGCGCCCTCGGCAAGCTGCAAGATGTGGTGAATCAAAATGCCCAAGCCCTGAAC |
| | ACACTGGTGAAGCAGCTGAGCAGCAACTTCGGCGCCATCAGCAGCGTGCTGAACGACATC |
| | CTGAGCAGACTGGACAAGGTGGAAGCCGAGGTGCAGATCGACAGACTGATCACAGGCAGA |
| | CTGCAGTCCCTGCAGACCTACGTGACCCAGCAGTTGATTAGAGCCGCTGAGATTAGAGCC |
| | AGTGCCAACCTGGCTGCCACAAAGATGTCAGAATGCGTGCTGGGCCAGAGCAAGAGAGTG |
| | GACTTCTGCGGCAAAGGCTACCACCTGATGAGCTTTCCTCAGTCTGCACCCCACGGCGTG |
| | GTGTTTCTCCACGTGACATACGTGCCCGCGCAAGAAAAGAACTTTACAACCGCCCCAGCG |
| | ATCTGCCACGACGGCAAGGCCCACTTCCCTCGGGAGGGTGTGTTCGTGAGCAATGGAACA |
| | CACTGGTTCGTCACCCAGCGGAACTTCTACGAGCCTCAGATCATTACCACCGACAACACC |
| | TTCGTGAGCGGCAACTGTGACGTCGTTATCGGCATCGTGAACAATACCGTGTACGACCCC |
| | CTGCAGCCTGAGCTGGATAGCTTCAAAGAGGAACTGGACAAGTACTTCAAGAACCACACA |
| | AGCCCCGACGTGGACCTAGGCGACATCTCTGGAATCAACGCCAGCGTGGTGAACATCCAA |
| | AAGGAAATCGACAGACTGAACGAGGTGGCCAAGAATCTGAATGAAAGCCTGATCGATCTG |
| | CAGGAGCTGGGCAAGTACGAGCAGTACATCAAATGGCCTTGGTACATCTGGCTGGGCTTC |
| | ATCGCTGGTCTGATCGCTATCGTGATGGTGACCATTATGCTGTGCTGCATGACCTCCTGC |
| | TGCTCTTGCCTGAAGGGCTGTTGTTCTTGCGGCTCTTGCTGCTGATGA |
| SEQ ID NO: | TAATACGACTCACTATAGGGACCAAACAGAGAATCCGTGAGTTACGATAAAAGGCGAAGG |
| 18; NDV- | AGCAATTGAAGTCGCACGGGTAGAAGGTGTGAATCTCGAGTGCGAGCCCGAAGCACAAAC |
| HexaPro S | TCGAGAAAGCCTTCTGCCAACATGTCTTCCGTATTTGATGAGTACGAACAGCTCCTCGCG |
| Molecular | GCTCAGACTCGCCCCAATGGAGCTCATGGAGGGGGAGAAAAAGGGAGTACCTTAAAAGTA |
| Clone | GACGTCCCGGTATTCACTCTTAACAGTGATGACCCAGAAGATAGATGGAGCTTTGTGGTA |
| AF077761.1_ | TTCTGCCTCCGGATTGCTGTTAGCGAAGATGCCAACAAACCACTCAGGCAAGGTGCTCTC |
| LaSota_Kan | ATATCTCTTTTATGCTCCCACTCACAGGTAATGAGGAACCATGTTGCCATTGCAGGGAAA |
| R (with | CAGAATGAAGCCACATTGGCCGTGCTTGAGATTGATGGCTTTGCCAACGGCACGCCCCAG |
| stabilizing | TTCAACAATAGGAGTGGAGTGTCTGAAGAGAGAGCACAGAGATTTGCGATGATAGCAGGA |
| sequence in | TCTCTCCCTCGGGCATGCAGCAACGGAACCCCGTTCGTCACAGCCGGGGCAGAAGATGAT |
| L) | GCACCAGAAGACATCACCGATACCCTGGAGAGGATCCTCTCTATCCAGGCTCAAGTATGG |
| | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCTCTAGATTAGAAAAAATA |
| | CGGGTAGAACCGCCACCATGTTCGTGTTCCTGGTCCTGCTGCCACTGGTAAGCTCCCAAT |
| | GTGTAAACTTAACCACAAGAACCCAGCTCCCACCTGCCTACACCAACAGCTTCACCAGAG |
| | GCGTTTATTACCCCGACAAGGTATTCCGGTCTTCTGTTCTGCACTCTACCCAGGACCTGT |
| | TTCTGCCCTTTTTCAGCAACGTGACATGGTTCCACGCCATCCACGTGTCTGGCACAAACG |
| | GCACCAAGCGGTTTGATAATCCTGTGCTCCCTTTCAATGACGGCGTGTACTTCGCCTCTA |
| | CTGAGAAGAGCAACATCATCCGGGGCTGGATCTTTGGCACAACACTGGACTCTAAAACCC |
| | AGAGCCTGCTGATCGTGAACAACGCCACCAACGTGGTGATTAAGGTGTGCGAGTTCCAGT |
| | TCTGCAATGACCCTTTCCTCGGCGTGTACTACCACAAGAACAACAAAAGTTGGATGGAAA |
| | GCGAATTCAGGGTGTACTCAAGCGCCAACAACTGTACCTTCGAGTACGTGAGCCAGCCTT |
| | TCCTGATGGACCTAGAAGGTAAGCAGGGCAATTTCAAGAACCTCAGAGAGTTCGTGTTCA |
| | AGAATATTGACGGCTACTTCAAAATCTACAGCAAGCACACCCCAATCAACCTGGTGCGGG |
| | ACCTGCCCCAGGGCTTTAGCGCGCTGGAGCCTCTGGTGGACCTGCCTATCGGCATCAACA |
| | TCACCCGGTTCCAGACACTGCTGGCTCTGCATAGAAGCTACCTGACACCTGGCGACAGTT |
| | CTTCTGGCTGGACAGCCGGCGCCGCCGCCTACTACGTGGGCTACCTGCAGCCTAGAACAT |
| | TCCTGCTGAAATACAACGAGAACGGCACGATCACAGACGCCGTGGACTGCGCCCTGGATC |
| | CCCTGTCTGAGACAAAGTGCACCCTGAAGTCTTTCACCGTGGAGAAGGGCATCTACCAGA |
| | CCTCCAACTTCAGAGTGCAGCCTACCGAATCCATCGTGCGCTTTCCCAACATCACCAACC |
| | TGTGCCCCTTCGGCGAGGTCTTTAATGCCACGAGATTCGCCAGCGTGTATGCCTGGAACA |
| | GAAAGAGAATCAGCAACTGCGTGGCCGACTACAGCGTGCTGTACAACAGCGCCTCTTTCA |
| | GCACATTTAAGTGCTACGGAGTGTCTCCTACCAAACTCAACGATCTGTGCTTCACGAACG |
| | TGTATGCCGACAGCTTCGTGATCCGAGGAGATGAGGTGCGGCAGATCGCTCCAGGACAGA |
| | CAGGCAAGATCGCCGACTACAACTACAAGCTGCCCGACGACTTTACCGGCTGCGTGATCG |
| | CTTGGAACAGCAATAACCTGGACTCAAAGGTTGGAGGAAACTACAACTACCTGTACAGAC |
| | TGTTCAGAAAGTCCAACCTGAAGCCCTTCGAGAGAGACATCTCTACAGAAATCTACCAGG |
| | CCGGCAGCACCCCATGTAACGGCGTGGAAGGCTTCAACTGCTACTTCCCTCTGCAGTCTT |
| | ATGGCTTCCAGCCCACAAACGGAGTGGGCTATCAGCCTTACCGCGTGGTTGTCCTGAGCT |
| | TTGAGCTGCTGCATGCCCCTGCTACGGTGTGTGGACCTAAGAAGTCCACCAACCTGGTGA |
| | AGAACAAGTGTGTGAACTTCAACTTCAACGGCCTGACCGGCACCGGGGTGCTGACAGAGT |
| | CTAACAAGAAATTCCTGCCATTCCAGCAATTCGGCCGGGACATCGCCGACACCACCGACG |
| | CCGTGCGGGATCCTCAGACCCTCGAAATCCTGGACATCACCCCCTGTAGCTTCGGCGGCG |
| | TGAGCGTGATCACCCCTGGCACAAACACCAGCAATCAAGTGGCTGTCCTGTACCAGGATG |
| | TCAATTGCACAGAAGTGCCTGTGGCCATCCACGCCGATCAGCTGACCCCCACCTGGCGGG |
| | TGTACTCGACAGGAAGCAACGTGTTTCAAACAAGAGCCGGCTGCCTGATCGGGGCCGAGC |
| | ACGTGAACAATTCCTACGAGTGCGACATCCCCATCGGCGCCGGCATCTGTGCCTCTTACC |
| | AGACACAGACCAATTCCCCTggtagtgcaagtTCCGTGGCCAGCCAGAGCATCATCGCCT |
| | ACACCATGAGCCTGGGCGCCGAAAACAGCGTTGCATATTCCAACAACAGCATCGCCATCC |
| | CTACCAACTTCACCATCAGCGTGACCACAGAAATCCTGCCTGTGTCCATGACCAAGACAA |
| | GCGTTGATTGCACCATGTACATCTGCGGCGATAGCACAGAGTGCAGCAATCTGCTGCTGC |
| | AGTACGGTAGCTTCTGCACCCAGCTGAATAGAGCCCTGACCGGCATCGCTGTGGAACAGG |
| | ACAAAAACACCCAGGAGGTCTTCGCCCAGGTGAAGCAAATCTACAAGACCCCTCCAATCA |
| | AGGACTTCGGAGGCTTTAACTTTAGCCAGATCCTGCCTGATCCCTCCAAGCCTAGCAAAC |
| | GGAGTcctATCGAGGACCTGCTCTTCAACAAGGTGACCCTGGCTGACGCCGGCTTCATTA |
| | AGCAGTACGGCGATTGCCTCGGCGACATCGCTGCAAGAGACCTGATCTGCGCCCAGAAGT |
| | TCAACGGCCTGACCGTGCTGCCTCCTCTCCTGACAGACGAGATGATCGCCCAGTACACCT |
| | CTGCCCTTCTGGCTGGCACCATCACCAGCGGATGGACCTTTGGAGCCGGAcctGCCCTGC |
| | AGATCCCTTTCcctATGCAGATGGCCTACAGATTCAACGGGATCGGAGTGACCCAAAACG |
| | TGCTGTATGAAAACCAGAAACTGATCGCCAATCAGTTTAACAGCGCCATCGGCAAAATCC |
| | AGGATAGCCTGTCCAGCACCccaAGCGCCCTCGGCAAGCTGCAAGATGTGGTGAATCAAA |
| | ATGCCCAAGCCCTGAACACACTGGTGAAGCAGCTGAGCAGCAACTTCGGCGCCATCAGCA |
| | GCGTGCTGAACGACATCCTGAGCAGACTGGACccacctGAAGCCGAGGTGCAGATCGACA |
| | GACTGATCACAGGCAGACTGCAGTCCCTGCAGACCTACGTGACCCAGCAGTTGATTAGAG |
| | CCGCTGAGATTAGAGCCAGTGCCAACCTGGCTGCCACAAAGATGTCAGAATGCGTGCTGG |
| | GCCAGAGCAAGAGAGTGGACTTCTGCGGCAAAGGCTACCACCTGATGAGCTTTCCTCAGT |
| | CTGCACCCCACGGCGTGGTGTTTCTCCACGTGACATACGTGCCCGCGCAAGAAAAGAACT |
| | TTACAACCGCCCCAGCGATCTGCCACGACGGCAAGGCCCACTTCCCTCGGGAGGGTGTGT |
| | TCGTGAGCAATGGAACACACTGGTTCGTCACCCAGCGGAACTTCTACGAGCCTCAGATCA |
| | TTACCACCGACAACACCTTCGTGAGCGGCAACTGTGACGTCGTTATCGGCATCGTGAACA |
| | ATACCGTGTACGACCCCCTGCAGCCTGAGCTGGATAGCTTCAAAGAGGAACTGGACAAGT |
| | ACTTCAAGAACCACACAAGCCCCGACGTGGACCTAGgcgacATCTCTGGAATCAACGCCA |
| | GCGTGGTGAACATCCAAAAGGAAATCGACAGACTGAACGAGGTGGCCAAGAATCTGAATG |
| | AAAGCCTGATCGATCTGCAGGAGCTGGGCAAGTACGAGCAGTACATCAAATGGCCTTGGT |
| | ACATCTGGCTGGGCTTCATCGCTGGTCTGATCGCTATCGTGATGGTGACCATTATGCTGT |
| | GCTGCATGACCTCCTGCTGCTCTTGCCTGAAGGGCTGTTGTTCTTGCGGCTCTTGCTGCA |
| | AGTTCGACGAGGATGACTCTGAACCTGTTCTGAAGGGCgtgaagctgcactacacctgat |
| | aaacgcgtACCCAAGGTCCAACTCTCCAAGCGGCAATCCTCTCTCGCTTCCTCAGCCCCA |
| | CTGAATGGTCGCGTAACCGTAATTAATCTAGCTACATTTAAGATTAAGAAAAAATACGGG |
| | TAGAATTGGAGTGCCCCAATTGTGCCAAGATGGACTCATCTAGGACAATTGGGCTGTACT |
| | TTGATTCTGCCCATTCTTCTAGCAACCTGTTAGCATTTCCGATCGTCCTACAAGGCACAG |
| | GAGATGGGAAGAAGCAAATCGCCCCGCAATATAGGATCCAGCGCCTTGACTTGTGGACTG |
| | ATAGTAAGGAGGACTCAGTATTCATCACCACCTATGGATTCATCTTTCAAGTTGGGAATG |
| | AAGAAGCCACTGTCGGCATGATCGATGATAAACCCAAGCGCGAGTTACTTTCCGCTGCGA |
| | TGCTCTGCCTAGGAAGCGTCCCAAATACCGGAGACCTTATTGAGCTGGCAAGGGCCTGTC |
| | TCACTATGATAGTCACATGCAAGAAGAGTGCAACTAATACTGAGAGAATGGTTTTCTGAG |
| | TAGTGCAGGCACCCCAAGTGCTGCAAAGCTGTAGGGTTGTGGCAAACAAATACTCATCAG |
| | TGAATGCAGTCAAGCACGTGAAAGCGCCAGAGAAGATTCCCGGGAGTGGAACCCTAGAAT |
| | ACAAGGTGAACTTTGTCTCCTTGACTGTGGTACCGAAGAAGGATGTCTACAAGATCCCAG |
| | CTGCAGTATTGAAGGTTTCTGGCTCGAGTCTGTACAATCTTGCGCTCAATGTCACTATTA |
| | ATGTGGAGGTAGACCCGAGGAGTCCTTTGGTTAAATCTTTGTCTAAGTCTGACAGCGGAT |
| | ACTATGCTAACCTCTTCTTGCATATTGGACTTATGACCACCGTAGATAGGAAGGGGAAGA |
| | AAGTGACATTTGACAAGCTGGAAAAGAAAATAAGGAGCCTTGATCTATCTGTCGGGCTCA |
| | GTGATGTGCTCGGGCCTTCCGTGTTGGTAAAAGCAAGAGGTGCACGGACTAAGCTTTTGG |
| | CACCTTTCTTCTCTAGCAGTGGGACAGCCTGCTATCCCATAGCAAATGCTTCTCCTCAGG |
| | TGGCCAAGATACTCTGGAGTCAAACCGCGTGCCTGCGGAGCGTTAAAATCATTATCCAAG |
| | CAGGTACCCAACGCGCTGTCGCAGTGACCGCCGACCACGAGGTTACCTCTACTAAGCTGG |
| | AGAAGGGGCACACCCTTGCCAAATACAATCCTTTTAAGAAATAAGCTGCGTCTCTGAGAT |
| | TGCGCTCCGCCCACTCACCCAGATCATCATGACACAAAAAACTAATCTGTCTTGATTATT |
| | TACAGTTAGTTTACCTGTCTATCAAGTTAGAAAAAACACGGGTAGAAGATTCTGGATCCC |
| | GGTTGGCGCCCTCCAGGTGCAAGttaattaaATGGGCTCCAGACCTTCTACCAAGAACCC |
| | AGCACCTATGATGCTGACTATCCGGGTTGCGCTGGTACTGAGTTGCATCTGTCCGGCAAA |
| | CTCCATTGATGGCAGGCCTCTTGCAGCTGCAGGAATTGTGGTTACAGGAGACAAAGCCGT |
| | CAACATATACACCTCATCCCAGACAGGATCAATCATAGTTAAGCTCCTCCCGAATCTGCC |
| | CAAGGATAAGGAGGCATGTGCGAAAGCCCCCTTGGATGCATACAACAGGACATTGACCAC |
| | TTTGCTCACCCCCCTTGGTGACTCTATCCGTAGGATACAAGAGTCTGTGACTACATCTGG |
| | AGGGGGGAGACAGGGGCGCCTTATAGGCGCCATTATTGGCGGTGTGGCTCTTGGGGTTGC |
| | AACTGCCGCACAAATAACAGCGGCCGCAGCTCTGATACAAGCCAAACAAAATGCTGCCAA |
| | CATCCTCCGACTTAAAGAGAGCATTGCCGCAACCAATGAGGCTGTGCATGAGGTCACTGA |
| | CGGATTATCGCAACTAGCAGTGGCAGTTGGGAAGATGCAGCAGTTTGTTAATGACCAATT |
| | TAATAAAACAGCTCAGGAATTAGACTGCATCAAAATTGCACAGCAAGTTGGTGTAGAGCT |
| | CAACCTGTACCTAACCGAATTGACTACAGTATTCGGACCACAAATCACTTCACCTGCTTT |
| | AAACAAGCTGACTATTCAGGCACTTTACAATCTAGCTGGTGGAAATATGGATTACTTATT |
| | GACTAAGTTAGGTGTAGGGAACAATCAACTCAGCTCATTAATCGGTAGCGGCTTAATCAC |
| | tGGcAACCCTATTCTATACGACTCACAGACTCAACTCTTGGGTATACAGGTAACTgcaCC |
| | TTCAGTCGGGAACCTAAATAATATGCGTGCCACCTACTTGGAAACCTTATCCGTAAGCAC |
| | AACCAGGGGATTTGCCTCGGCACTTGTCCCCAAAGTGGTGACACAGGTCGGTTCTGTGAT |
| | AGAAGAACTTGACACCTCATACTGTATAGAAACTGACTTAGATTTATATTGTACAAGAAT |
| | AGTAACGTTCCCTATGTCCCCTGGTATTTATTCCTGCTTGAGCGGCAATACGTCGGCCTG |
| | TATGTACTCAAAGACCGAAGGCGCACTTACTACACCATACATGACTATCAAAGGTTCAGT |
| | CATCGCCAACTGCAAGATGACAACATGTAGATGTGTAAACCCCCCGGGTATCATATCGCA |
| | AAACTATGGAGAAGCCGTGTCTCTAATAGATAAACAATCATGCAATGTTTTATCCTTAGG |
| | CGGGATAACTTTAAGGCTCAGTGGGGAATTCGATGTAACTTATCAGAAGAATATCTCAAT |
| | ACAAGATTCTCAAGTAATAATAACAGGCAATCTTGATATCTCAACTGAGCTTGGGAATGT |
| | CAACAACTCGATCAGTAATGCTTTGAATAAGTTAGAGGAAAGCAACAGAAAACTAGACAA |
| | AGTCAATGTCAAACTGACTAGCACATCTGCTCTCATTACgTATATCGTTTTGACTATCAT |
| | ATCTCTTGTTTTTGGTATACTTAGCCTGATTCTAGCATGCTACCTAATGTACAAGCAAAA |
| | GGCGCAACAAAAGACCTTATTATGGCTTGGGAATAATACaCTcGATCAGATGAGAGCCAC |
| | TACAAAAATGTGAACACAGATGAGGAACGAAGGTTTCCCTAATAGTAATTTGTGTGAAAG |
| | TTCTGGTAGTCTGTCAGTTCAGAGAGTTAAGAAAAAACTACCGGTTGTAGATGACCAAAG |
| | GACGATATACGGGTAGAACGGTAAGAGAGGCCGCCCCTCAATTGCGAGCCAGGCTTCACA |
| | ACCTCCGTTCTACCGCTTCACCGACAACAGTCCTCAATCATGGACCGCGCCGTTAGCCAA |
| | GTTGCGTTAGAGAATGATGAAAGAGAGGCAAAAAATACATGGCGCTTGATATTCCGGATT |
| | GCAATCTTATTCTTAACAGTAGTGACCTTGGCTATATCTGTAGCCTCCCTTTTATATAGC |
| | ATGGGGGCTAGCACACCTAGCGATCTTGTAGGCATACCGACTAGGATTTCCAGGGCAGAA |
| | GAAAAGATTACATCTACACTTGGTTCCAATCAAGATGTAGTAGATAGGATATATAAGCAA |
| | GTGGCCCTTGAGTCTCCGTTGGCATTGTTAAATACTGAGACCACAATTATGAACGCAATA |
| | ACATCTCTCTCTTATCAGATTAATGGAGCTGCAAACAACAGTGGGTGGGGGGCACCTATC |
| | CATGACCCAGATTATATAGGGGGGATAGGCAAAGAACTCATTGTAGATGATGCTAGTGAT |
| | GTCACATCATTCTATCCCTCTGCATTTCAAGAACATCTGAATTTTATCCCGGCGCCTACT |
| | ACAGGATCAGGTTGCACTCGAATACCCTCATTTGACATGAGTGCTACCCATTACTGCTAC |
| | ACCCATAATGTAATATTGTCTGGATGCAGAGATCACTCACATTCATATCAGTATTTAGCA |
| | CTTGGTGTGCTCCGGACATCTGCAACAGGGAGGGTATTCTTTTCTACTCTGCGTTCCATC |
| | AACCTGGACGACACCCAAAATCGGAAGTCTTGCAGTGTGAGTGCAACTCCCCTGGGTTGT |
| | GATATGCTGTGCTCGAAAGTCACGGAGACAGAGGAAGAAGATTATAACTCAGCTGTCCCT |
| | ACGCGGATGGTACATGGGAGGTTAGGGTTCGACGGCCAGTACCACGAAAAGGACCTAGAT |
| | GTCACAACATTATTCGGGGACTGGGTGGCCAACTACCCAGGAGTAGGGGGTGGATCTTTT |
| | ATTGACAGCCGCGTATGGTTCTCAGTCTACGGAGGGTTAAAACCCAATTCACCCAGTGAC |
| | ACTGTACAGGAAGGGAAATATGTGATATACAAGCGATACAATGACACATGCCCAGATGAG |
| | CAAGACTACCAGATTCGAATGGCCAAGTCTTCGTATAAGCCTGGACGGTTTGGTGGGAAA |
| | CGCATACAGCAGGCTATCTTATCTATCAAGGTGTCAACATCCTTAGGCGAAGACCCGGTA |
| | CTGACTGTACCGCCCAACACAGTCACACTCATGGGGGCCGAAGGCAGAATTCTCACAGTA |
| | GGGACATCTCATTTCTTGTATCAACGAGGGTCATCATACTTCTCTCCCGCGTTATTATAT |
| | CCTATGACAGTCAGCAACAAAACAGCCACTCTTCATAGTCCTTATACATTCAATGCCTTC |
| | ACTCGGCCAGGTAGTATCCCTTGCCAGGCTTCAGCAAGATGCCCCAACTCGTGTGTTACT |
| | GGAGTCTATACAGATCCATATCCCCTAATCTTCTATAGAAACCACACCTTGCGAGGGGTA |
| | TTCGGGACAATGCTTGATGGTGTACAAGCAAGACTTAACCCTGCGTCTGCAGTATTCGAT |
| | AGCACATCCCGCAGTCGCATTACTCGAGTGAGTTCAAGCAGTACCAAAGCAGCATACACA |
| | ACATCAACTTGTTTTAAAGTGGTCAAGACTAATAAGACCTATTGTCTCAGCATTGCTGAA |
| | ATATCTAATACTCTCTTCGGAGAATTCAGAATCGTCCCGTTACTAGTTGAGATCCTCAAA |
| | GATGACGGGGTTAGAGAAGCCAGGTCTGGCTAGggcgcgccTTGAGTCAATTATAAAGGA |
| | GTTGGAAAGATGGCATTGTATCACCTATCTTCTGCGACATCAAGAATCAAACCGAATGCC |
| | GGCGCGTGCTCGAATTCCATGTTGCCAGTTGACCACAATCAGCCAGTGCTCATGCGATCA |
| | GATTAAGCCTTGTCATTAATCTCTTGATTAAGAAAAAATGTAAGTGGCAATGAGATACAA |
| | GGCAAAACAGCTCATGGTAAATAATACGGGTAGGACATGGCGAGCTCCGGTCCTGAAAGG |
| | GCAGAGCATCAGATTATCCTACCAGAGCCACACCTGTCTTCACCATTGGTCAAGCACAAA |
| | CTACTCTATTACTGGAAATTAACTGGGCTACCGCTTCCTGATGAATGTGACTTCGACCAC |
| | CTCATTCTCAGCCGACAATGGAAAAAAATACTTGAATCGGCCTCTCCTGATACTGAGAGA |
| | ATGATAAAACTCGGAAGGGCAGTACACCAAACTCTTAACCACAATTCCAGAATAACCGGA |
| | GTGCTCCACCCCAGGTGTTTAGAACAACTGGCTAATATTGAGGTCCCAGATTCAACCAAC |
| | AAATTTCGGAAGATTGAGAAGAAGATCCAAATTCACAACACGAGATATGGAGAACTGTTC |
| | ACAAGGCTGTGTACGCATATAGAGAAGAAACTGCTGGGGTCATCTTGGTCTAACAATGTC |
| | CCCCGGTCAGAGGAGTTCAGCAGCATTCGTACGGATCCGGCATTCTGGTTTCACTCAAAA |
| | TGGTCCACAGCCAAGTTTGCATGGCTCCATATAAAACAGATCCAGAGGCATCTGATGGTG |
| | GCAGCTAAGACAAGGTCTGCGGCCAACAAATTGGTGATGCTAACCCATAAGGTAGGCCAA |
| | GTCTTTGTCACTCCTGAACTTGTCGTTGTGACGCATACGAATGAGAACAAGTTCACATGT |
| | CTTACCCAGGAACTTGTATTGATGTATGCAGATATGATGGAGGGCAGAGATATGGTCAAC |
| | ATAATATCAACCACGGCGGTGCATCTCAGAAGCTTATCAGAGAAAATTGATGACATTTTG |
| | CGGTTAATAGACGCTCTGGCAAAAGACTTGGGTAATCAAGTCTACGATGTTGTATCACTA |
| | ATGGAGGGATTTGCATACGGAGCTGTCCAGCTACTCGAGCCGTCAGGTACATTTGCAGGA |
| | GATTTCTTCGCATTCAACCTGCAGGAGCTTAAAGACATTCTAATTGGCCTCCTCCCCAAT |
| | GATATAGCAGAATCCGTGACTCATGCAATCGCTACTGTATTCTCTGGTTTAGAACAGAAT |
| | CAAGCAGCTGAGATGTTGTGTCTGTTGCGTCTGTGGGGTCACCCACTGCTTGAGTCCCGT |
| | ATTGCAGCAAAGGCAGTCAGGAGCCAAATGTGCGCACCGAAAATGGTAGACTTTGATATG |
| | ATCCTTCAGGTACTGTCTTTCTTCAAGGGAACAATCATCAACGGGTACAGAAAGAAGAAT |
| | GCAGGTGTGTGGCCGCGAGTCAAAGTGGATACAATATATGGGAAGGTCATTGGGCAACTA |
| | CATGCAGATTCAGCAGAGATTTCACACGATATCATGTTGAGAGAGTATAAGAGTTTATCT |
| | GCACTTGAATTTGAGCCATGTATAGAATATGACCCTGTCACCAACCTGAGCATGTTCCTA |
| | AAAGACAAGGCAATCGCACACCCCAACGATAATTGGCTTGCCTCGTTTAGGCGGAACCTT |
| | CTCTCCGAAGACCAGAAGAAACATGTAAAAGAAGCAACTTCGACTAATCGCCTCTTGATA |
| | GAGTTTTTAGAGTCAAATGATTTTGATCCATATAAAGAGATGGAATATCTGACGACCCTT |
| | GAGTACCTTAGAGATGACAATGTGGCAGTATCATACTCGCTCAAGGAGAAGGAAGTGAAA |
| | GTTAATGGACGGATCTTCGCTAAGCTGACAAAGAAGTTAAGGAACTGTCAGGTGATGGCG |
| | GAAGGGATCCTAGCCGATCAGATTGCACCTTTCTTTCAGGGAAATGGAGTCATTCAGGAT |
| | AGCATATCCTTGACCAAGAGTATGCTAGCGATGAGTCAACTGTCTTTTAACAGCAATAAG |
| | AAACGTATCACTGACTGTAAAGAAAGAGTATCTTCAAACCGCAATCATGATCCGAAAAGC |
| | AAGAACCGTCGGAGAGTTGCAACCTTCATAACAACTGACCTGCAAAAGTACTGTCTTAAT |
| | TGGAGATATCAGACAATCAAATTGTTCGCTCATGCCATCAATCAGTTGATGGGCCTACCT |
| | CACTTCTTCGAATGGATTCACCTAAGACTGATGGACACTACGATGTTCGTAGGAGACCCT |
| | TTCAATCCTCCAAGTGACCCTACTGACTGTGACCTCTCAAGAGTCCCTAATGATGACATA |
| | TATATTGTCAGTGCCAGAGGGGGTATCGAAGGATTATGCCAGAAGCTATGGACAATGATC |
| | TCAATTGCTGCAATCCAACTTGCTGCAGCTAGATCGCATTGTCGTGTTGCCTGTATGGTA |
| | CAGGGTGATAATCAAGTAATAGCAGTAACGAGAGAGGTAAGATCAGACGACTCTCCGGAG |
| | ATGGTGTTGACACAGTTGCATCAAGCCAGTGATAATTTCTTCAAGGAATTAATTCATGTC |
| | AATCATTTGATTGGCCATAATTTGAAGGATCGTGAAACCATCAGGTCAGACACATTCTTC |
| | ATATACAGCAAACGAATCTTCAAAGATGGAGCAATCCTCAGTCAAGTCCTCAAAAATTCA |
| | TCTAAATTAGTGCTAGTGTCAGGTGATCTCAGTGAAAACACCGTAATGTCCTGTGCCAAC |
| | ATTGCCTCTACTGTAGCACGGCTATGCGAGAACGGGCTTCCCAAAGACTTCTGTTACTAT |
| | TTAAACTATATAATGAGTTGTGTGCAGACATACTTTGACTCTGAGTTCTCCATCACCAAC |
| | AATTCGCACCCCGATCTTAATCAGTCGTGGATTGAGGACATCTCTTTTGTGCACTCATAT |
| | GTTCTGACTCCTGCCCAATTAGGGGGACTGAGTAACCTTCAATACTCAAGGCTCTACACT |
| | AGAAATATCGGTGACCCGGGGACTACTGCTTTTGCAGAGATCAAGCGACTAGAAGCAGTG |
| | GGATTACTGAGTCCTAACATTATGACTAATATCTTAACTAGGCCGCCTGGGAATGGAGAT |
| | TGGGCCAGTCTGTGCAACGACCCATACTCTTTCAATTTTGAGACTGTTGCAAGCCCAAAT |
| | ATTGTTCTTAAGAAACATACGCAAAGAGTCCTATTTGAAACTTGTTCAAATCCCTTATTG |
| | TCTGGAGTGCACACAGAGGATAATGAGGCAGAAGAGAAGGCATTGGCTGAATTCTTGCTT |
| | AATCAAGAGGTGATTCATCCCCGCGTTGCGCATGCCATCATGGAGGCAAGCTCTGTAGGT |
| | AGGAGAAAGCAAATTCAAGGGCTTGTTGACACAACAAACACCGTAATTAAGATTGCGCTT |
| | ACTAGGAGGCCATTAGGCATCAAGAGGCTGATGCGGATAGTCAATTATTCTAGCATGCAT |
| | GCAATGCTGTTTAGAGACGATGTTTTTTCCTCCAGTAGATCCAACCACCCCTTAGTCTCT |
| | TCTAATATGTGTTCTCTGACACTGGCAGACTATGCACGGAATAGAAGCTGGTCACCTTTG |
| | ACGGGAGGCAGGAAAATACTGGGTGTATCTAATCCTGATACGATAGAACTCGTAGAGGGT |
| | GAGATTCTTAGTGTAAGCGGAGGGTGTACAAGATGTGACAGCGGAGATGAACAATTTACT |
| | TGGTTCCATCTTCCAAGCAATATAGAATTGACCGATGACACCAGCAAGAATCCTCCGATG |
| | AGGGTACCATATCTCGGGTCAAAGACACAGGAGAGGAGAGCTGCCTCACTTGCAAAAATA |
| | GCTCATATGTCGCCACATGTAAAGGCTGCCCTAAGGGCATCATCCGTGTTGATCTGGGCT |
| | TATGGGGATAATGAAGTAAATTGGACTGCTGCTCTTACGATTGCAAAATCTCGGTGTAAT |
| | GTAAACTTAGAGTATCTTCGGTTACTGTCCCCTTTACCCACGGCTGGGAATCTTCAACAT |
| | AGACTAGATGATGGTATAACTCAGATGACATTCACCCCTGCATCTCTCTACAGGgtgtca |
| | ccttacattcacatatccaatgattctcaaaggctgttcactgaagaaggagtcaaagag |
| | gggaatgtggtttaccaacagatcATGCTCTTGGGTTTATCTCTAATCGAATCGATCTTT |
| | CCAATGACAACAACCAGGACATATGATGAGATCACACTGCACCTACATAGTAAATTTAGT |
| | TGCTGTATCAGAGAAGCACCTGTTGCGGTTCCTTTCGAGCTACTTGGGGTGGTACCGGAA |
| | CTGAGGACAGTGACCTCAAATAAGTTTATGTATGATCCTAGCCCTGTATCGGAGGGAGAC |
| | TTTGCGAGACTTGACTTAGCTATCTTCAAGAGTTATGAGCTTAATCTGGAGTCATATCCC |
| | ACGATAGAGCTAATGAACATTCTTTCAATATCCAGCGGGAAGTTGATTGGCCAGTCTGTG |
| | GTTTCTTATGATGAAGATACCTCCATAAAGAATGACGCCATAATAGTGTATGACAATACC |
| | CGAAATTGGATCAGTGAAGCTCAGAATTCAGATGTGGTCCGCCTATTTGAATATGCAGCA |
| | CTTGAAGTGCTCCTCGACTGTTCTTACCAACTCTATTACCTGAGAGTAAGAGGCCTAGAC |
| | AATATTGTCTTATATATGGGTGATTTATACAAGAATATGCCAGGAATTCTACTTTCCAAC |
| | ATTGCAGCTACAATATCTCATCCCGTCATTCATTCAAGGTTACATGCAGTGGGCCTGGTC |
| | AACCATGACGGATCACACCAACTTGCAGATACGGATTTTATCGAAATGTCTGCAAAACTA |
| | TTAGTATCTTGCACCCGACGTGTGATCTCCGGCTTATATTCAGGAAATAAGTATGATCTG |
| | CTGTTCCCATCTGTCTTAGATGATAACCTGAATGAGAAGATGCTTCAGCTGATATCCCGG |
| | TTATGCTGTCTGTACACGGTACTCTTTGCTACAACAAGAGAAATCCCGAAAATAAGAGGC |
| | TTAACTGCAGAAGAGAAATGTTCAATACTCACTGAGTATTTACTGTCGGATGCTGTGAAA |
| | CCATTACTTAGCCCCGATCAAGTGAGCTCTATCATGTCTCCTAACATAATTACATTCCCA |
| | GCTAATCTGTACTACATGTCTCGGAAGAGCCTCAATTTGATCAGGGAAAGGGAGGACAGG |
| | GATACTATCCTGGCGTTGTTGTTCCCCCAAGAGCCATTATTAGAGTTCCCTTCTGTGCAA |
| | GATATTGGTGCTCGAGTGAAAGATCCATTCACCCGACAACCTGCGGCATTTTTGCAAGAG |
| | TTAGATTTGAGTGCTCCAGCAAGGTATGACGCATTCACACTTAGTCAGATTCATCCTGAA |
| | CTCACATCTCCAAATCCGGAGGAAGACTACTTAGTACGATACTTGTTCAGAGGGATAGGG |
| | ACTGCATCTTCCTCTTGGTATAAGGCATCTCATCTCCTTTCTGTACCCGAGGTAAGATGT |
| | GCAAGACACGGGAACTCCTTATACTTAGCTGAAGGGAGCGGAGCCATCATGAGTCTTCTC |
| | GAACTGCATGTACCACATGAAACTATCTATTACAATACGCTCTTTTCAAATGAGATGAAC |
| | CCCCCGCAACGACATTTCGGGCCGACCCCAACTCAGTTTTTGAATTCGGTTGTTTATAGG |
| | AATCTACAGGCGGAGGTAACATGCAAAGATGGATTTGTCCAAGAGTTCCGTCCATTATGG |
| | AGAGAAAATACAGAGGAAAGTGACCTGACCTCAGATAAAGCAGTGGGGTATATTACATCT |
| | GCAGTGCCCTACAGATCTGTATCATTGCTGCATTGTGACATTGAAATTCCTCCAGGGTCC |
| | AATCAAAGCTTACTAGATCAACTAGCTATCAATTTATCTCTGATTGCCATGCATTCTGTA |
| | AGGGAGGGCGGGGTAGTAATCATCAAAGTGTTGTATGCAATGGGATACTACTTTCATCTA |
| | CTCATGAACTTGTTTGCTCCGTGTTCCACAAAAGGATATATTCTCTCTAATGGTTATGCA |
| | TGTCGAGGAGATATGGAGTGTTACCTGGTATTTGTCATGGGTTACCTGGGCGGGCCTACA |
| | TTTGTACATGAGGTGGTGAGGATGGCAAAAACTCTGGTGCAGCGGCACGGTACGCTCTTG |
| | TCTAAATCAGATGAGATCACACTGACCAGGTTATTCACCTCACAGCGGCAGCGTGTGACA |
| | GACATCCTATCCAGTCCTTTACCAAGATTAATAAAGTACTTGAGGAAGAATATTGACACT |
| | GCGCTGATTGAAGCCGGGGGACAGCCCGTCCGTCCATTCTGTGCGGAGAGTCTGGTGAGC |
| | ACGCTAGCGAACATAACTCAGATAACCCAGATTATCGCTAGTCACATTGACACAGTTATC |
| | CGGTCTGTGATATATATGGAAGCTGAGGGTGATCTCGCTGACACAGTATTTCTATTTACC |
| | CCTTACAATCTCTCTACTGACGGGAAAAAGAGGACATCACTTATACAGTGCACGAGACAG |
| | ATCCTAGAGGTTACAATACTAGGTCTTAGAGTCGAAAATCTCAATAAAATAGGCGATATA |
| | ATCAGCCTAGTGCTTAAAGGCATGATCTCCATGGAGGACCTTATCCCACTAAGGACATAC |
| | TTGAAGCATAGTACCTGCCCTAAATATTTGAAGGCTGTCCTAGGTATTACCAAACTCAAA |
| | GAAATGTTTACAGACACTTCTGTATTGTACTTGACTCGTGCTCAACAAAAATTCTACATG |
| | AAAACTATAGGCAATGCAGTCAAAGGATATTACAGTAACTGTGACTCTTAACGAAAATCA |
| | CATATTAATAGGCTCCTTTTTTGGCCAATTGTATTCTTGTTGATTTAATCATATTATGTT |
| | AGAAAAAAGTTGAACCCTGACTCCTTAGGACTCGAATTCGAACTCAAATAAATGTCTTAA |
| | AAAAAGGTTGCGCACAATTATTCTTGAGTGTAGTCTCGTCATTCACCAAATCTTTGTTTG |
| | GTGGCCGGCATGGTCCCAGCCTCCTCGCTGGCGCCGGCTGGGCAACATTCCGAGGGGACC |
| | GTCCCCTCGGTAATGGCGAATGGGACGTCGACTGCTAACAAAGCCCGAAAGGAAGCTGAG |
| | TTGGCTGCTGCCACCGCTGAGCAATAACTAGCATAACCCCTTGGGGCCTCTAAACGGGTC |
| | TTGAGGGGTTTTTTGCTGAAAGGAGGAACTATA |
| SEQ ID NO: | TAATACGACTCACTATAGGGACCAAACAGAGAATCCGTGAGTTACGATAAAAGGCGAAGG |
| 19; NDV- | AGCAATTGAAGTCGCACGGGTAGAAGGTGTGAATCTCGAGTGCGAGCCCGAAGCACAAAC |
| FLS-6P | TCGAGAAAGCCTTCTGCCAACATGTCTTCCGTATTTGATGAGTACGAACAGCTCCTCGCG |
| Molecular | GCTCAGACTCGCCCCAATGGAGCTCATGGAGGGGGAGAAAAAGGGAGTACCTTAAAAGTA |
| Clone | GACGTCCCGGTATTCACTCTTAACAGTGATGACCCAGAAGATAGATGGAGCTTTGTGGTA |
| AF077761.1_ | TTCTGCCTCCGGATTGCTGTTAGCGAAGATGCCAACAAACCACTCAGGCAAGGTGCTCTC |
| LaSota_Kan | ATATCTCTTTTATGCTCCCACTCACAGGTAATGAGGAACCATGTTGCCATTGCAGGGAAA |
| R (with | CAGAATGAAGCCACATTGGCCGTGCTTGAGATTGATGGCTTTGCCAACGGCACGCCCCAG |
| stabilizing | TTCAACAATAGGAGTGGAGTGTCTGAAGAGAGAGCACAGAGATTTGCGATGATAGCAGGA |
| sequence in | TCTCTCCCTCGGGCATGCAGCAACGGAACCCCGTTCGTCACAGCCGGGGCAGAAGATGAT |
| L) | GCACCAGAAGACATCACCGATACCCTGGAGAGGATCCTCTCTATCCAGGCTCAAGTATGG |
| | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCTCTAGATTAGAAAAAATA |
| | CGGGTAGAACCGCCACCATGTTCGTGTTCCTGGTCCTGCTGCCACTGGTAAGCTCCCAAT |
| | GTGTAAACTTAACCACAAGAACCCAGCTCCCACCTGCCTACACCAACAGCTTCACCAGAG |
| | GCGTTTATTACCCCGACAAGGTATTCCGGTCTTCTGTTCTGCACTCTACCCAGGACCTGT |
| | TTCTGCCCTTTTTCAGCAACGTGACATGGTTCCACGCCATCCACGTGTCTGGCACAAACG |
| | GCACCAAGCGGTTTGATAATCCTGTGCTCCCTTTCAATGACGGCGTGTACTTCGCCTCTA |
| | CTGAGAAGAGCAACATCATCCGGGGCTGGATCTTTGGCACAACACTGGACTCTAAAACCC |
| | AGAGCCTGCTGATCGTGAACAACGCCACCAACGTGGTGATTAAGGTGTGCGAGTTCCAGT |
| | TCTGCAATGACCCTTTCCTCGGCGTGTACTACCACAAGAACAACAAAAGTTGGATGGAAA |
| | GCGAATTCAGGGTGTACTCAAGCGCCAACAACTGTACCTTCGAGTACGTGAGCCAGCCTT |
| | TCCTGATGGACCTAGAAGGTAAGCAGGGCAATTTCAAGAACCTCAGAGAGTTCGTGTTCA |
| | AGAATATTGACGGCTACTTCAAAATCTACAGCAAGCACACCCCAATCAACCTGGTGCGGG |
| | ACCTGCCCCAGGGCTTTAGCGCGCTGGAGCCTCTGGTGGACCTGCCTATCGGCATCAACA |
| | TCACCCGGTTCCAGACACTGCTGGCTCTGCATAGAAGCTACCTGACACCTGGCGACAGTT |
| | CTTCTGGCTGGACAGCCGGCGCCGCCGCCTACTACGTGGGCTACCTGCAGCCTAGAACAT |
| | TCCTGCTGAAATACAACGAGAACGGCACGATCACAGACGCCGTGGACTGCGCCCTGGATC |
| | CCCTGTCTGAGACAAAGTGCACCCTGAAGTCTTTCACCGTGGAGAAGGGCATCTACCAGA |
| | CCTCCAACTTCAGAGTGCAGCCTACCGAATCCATCGTGCGCTTTCCCAACATCACCAACC |
| | TGTGCCCCTTCGGCGAGGTCTTTAATGCCACGAGATTCGCCAGCGTGTATGCCTGGAACA |
| | GAAAGAGAATCAGCAACTGCGTGGCCGACTACAGCGTGCTGTACAACAGCGCCTCTTTCA |
| | GCACATTTAAGTGCTACGGAGTGTCTCCTACCAAACTCAACGATCTGTGCTTCACGAACG |
| | TGTATGCCGACAGCTTCGTGATCCGAGGAGATGAGGTGCGGCAGATCGCTCCAGGACAGA |
| | CAGGCAAGATCGCCGACTACAACTACAAGCTGCCCGACGACTTTACCGGCTGCGTGATCG |
| | CTTGGAACAGCAATAACCTGGACTCAAAGGTTGGAGGAAACTACAACTACCTGTACAGAC |
| | TGTTCAGAAAGTCCAACCTGAAGCCCTTCGAGAGAGACATCTCTACAGAAATCTACCAGG |
| | CCGGCAGCACCCCATGTAACGGCGTGGAAGGCTTCAACTGCTACTTCCCTCTGCAGTCTT |
| | ATGGCTTCCAGCCCACAAACGGAGTGGGCTATCAGCCTTACCGCGTGGTTGTCCTGAGCT |
| | TTGAGCTGCTGCATGCCCCTGCTACGGTGTGTGGACCTAAGAAGTCCACCAACCTGGTGA |
| | AGAACAAGTGTGTGAACTTCAACTTCAACGGCCTGACCGGCACCGGGGTGCTGACAGAGT |
| | CTAACAAGAAATTCCTGCCATTCCAGCAATTCGGCCGGGACATCGCCGACACCACCGACG |
| | CCGTGCGGGATCCTCAGACCCTCGAAATCCTGGACATCACCCCCTGTAGCTTCGGCGGCG |
| | TGAGCGTGATCACCCCTGGCACAAACACCAGCAATCAAGTGGCTGTCCTGTACCAGGATG |
| | TCAATTGCACAGAAGTGCCTGTGGCCATCCACGCCGATCAGCTGACCCCCACCTGGCGGG |
| | TGTACTCGACAGGAAGCAACGTGTTTCAAACAAGAGCCGGCTGCCTGATCGGGGCCGAGC |
| | ACGTGAACAATTCCTACGAGTGCGACATCCCCATCGGCGCCGGCATCTGTGCCTCTTACC |
| | AGACACAGACCAATTCCCCTCGTAGAGCCAGATCCGTGGCCAGCCAGAGCATCATCGCCT |
| | ACACCATGAGCCTGGGCGCCGAAAACAGCGTTGCATATTCCAACAACAGCATCGCCATCC |
| | CTACCAACTTCACCATCAGCGTGACCACAGAAATCCTGCCTGTGTCCATGACCAAGACAA |
| | GCGTTGATTGCACCATGTACATCTGCGGCGATAGCACAGAGTGCAGCAATCTGCTGCTGC |
| | AGTACGGTAGCTTCTGCACCCAGCTGAATAGAGCCCTGACCGGCATCGCTGTGGAACAGG |
| | ACAAAAACACCCAGGAGGTCTTCGCCCAGGTGAAGCAAATCTACAAGACCCCTCCAATCA |
| | AGGACTTCGGAGGCTTTAACTTTAGCCAGATCCTGCCTGATCCCTCCAAGCCTAGCAAAC |
| | GGAGTcctATCGAGGACCTGCTCTTCAACAAGGTGACCCTGGCTGACGCCGGCTTCATTA |
| | AGCAGTACGGCGATTGCCTCGGCGACATCGCTGCAAGAGACCTGATCTGCGCCCAGAAGT |
| | TCAACGGCCTGACCGTGCTGCCTCCTCTCCTGACAGACGAGATGATCGCCCAGTACACCT |
| | CTGCCCTTCTGGCTGGCACCATCACCAGCGGATGGACCTTTGGAGCCGGAcctGCCCTGC |
| | AGATCCCTTTCcctATGCAGATGGCCTACAGATTCAACGGGATCGGAGTGACCCAAAACG |
| | TGCTGTATGAAAACCAGAAACTGATCGCCAATCAGTTTAACAGCGCCATCGGCAAAATCC |
| | AGGATAGCCTGTCCAGCACCccaAGCGCCCTCGGCAAGCTGCAAGATGTGGTGAATCAAA |
| | ATGCCCAAGCCCTGAACACACTGGTGAAGCAGCTGAGCAGCAACTTCGGCGCCATCAGCA |
| | GCGTGCTGAACGACATCCTGAGCAGACTGGACccacctGAAGCCGAGGTGCAGATCGACA |
| | GACTGATCACAGGCAGACTGCAGTCCCTGCAGACCTACGTGACCCAGCAGTTGATTAGAG |
| | CCGCTGAGATTAGAGCCAGTGCCAACCTGGCTGCCACAAAGATGTCAGAATGCGTGCTGG |
| | GCCAGAGCAAGAGAGTGGACTTCTGCGGCAAAGGCTACCACCTGATGAGCTTTCCTCAGT |
| | CTGCACCCCACGGCGTGGTGTTTCTCCACGTGACATACGTGCCCGCGCAAGAAAAGAACT |
| | TTACAACCGCCCCAGCGATCTGCCACGACGGCAAGGCCCACTTCCCTCGGGAGGGTGTGT |
| | TCGTGAGCAATGGAACACACTGGTTCGTCACCCAGCGGAACTTCTACGAGCCTCAGATCA |
| | TTACCACCGACAACACCTTCGTGAGCGGCAACTGTGACGTCGTTATCGGCATCGTGAACA |
| | ATACCGTGTACGACCCCCTGCAGCCTGAGCTGGATAGCTTCAAAGAGGAACTGGACAAGT |
| | ACTTCAAGAACCACACAAGCCCCGACGTGGACCTAGGCGACATCTCTGGAATCAACGCCA |
| | GCGTGGTGAACATCCAAAAGGAAATCGACAGACTGAACGAGGTGGCCAAGAATCTGAATG |
| | AAAGCCTGATCGATCTGCAGGAGCTGGGCAAGTACGAGCAGTACATCAAATGGCCTTGGT |
| | ACATCTGGCTGGGCTTCATCGCTGGTCTGATCGCTATCGTGATGGTGACCATTATGCTGT |
| | GCTGCATGACCTCCTGCTGCTCTTGCCTGAAGGGCTGTTGTTCTTGCGGCTCTTGCTGCA |
| | AGTTCGACGAGGATGACTCTGAACCTGTTCTGAAGGGCgtgaagctgcactacacctgat |
| | aaacgcgtACCCAAGGTCCAACTCTCCAAGCGGCAATCCTCTCTCGCTTCCTCAGCCCCA |
| | CTGAATGGTCGCGTAACCGTAATTAATCTAGCTACATTTAAGATTAAGAAAAAATACGGG |
| | TAGAATTGGAGTGCCCCAATTGTGCCAAGATGGACTCATCTAGGACAATTGGGCTGTACT |
| | TTGATTCTGCCCATTCTTCTAGCAACCTGTTAGCATTTCCGATCGTCCTACAAGGCACAG |
| | GAGATGGGAAGAAGCAAATCGCCCCGCAATATAGGATCCAGCGCCTTGACTTGTGGACTG |
| | ATAGTAAGGAGGACTCAGTATTCATCACCACCTATGGATTCATCTTTCAAGTTGGGAATG |
| | AAGAAGCCACTGTCGGCATGATCGATGATAAACCCAAGCGCGAGTTACTTTCCGCTGCGA |
| | TGCTCTGCCTAGGAAGCGTCCCAAATACCGGAGACCTTATTGAGCTGGCAAGGGCCTGTC |
| | TCACTATGATAGTCACATGCAAGAAGAGTGCAACTAATACTGAGAGAATGGTTTTCTCAG |
| | TAGTGCAGGCACCCCAAGTGCTGCAAAGCTGTAGGGTTGTGGCAAACAAATACTCATCAG |
| | TGAATGCAGTCAAGCACGTGAAAGCGCCAGAGAAGATTCCCGGGAGTGGAACCCTAGAAT |
| | ACAAGGTGAACTTTGTCTCCTTGACTGTGGTACCGAAGAAGGATGTCTACAAGATCCCAG |
| | CTGCAGTATTGAAGGTTTCTGGCTCGAGTCTGTACAATCTTGCGCTCAATGTCACTATTA |
| | ATGTGGAGGTAGACCCGAGGAGTCCTTTGGTTAAATCTTTGTCTAAGTCTGACAGCGGAT |
| | ACTATGCTAACCTCTTCTTGCATATTGGACTTATGACCACCGTAGATAGGAAGGGGAAGA |
| | AAGTGACATTTGACAAGCTGGAAAAGAAAATAAGGAGCCTTGATCTATCTGTCGGGCTCA |
| | GTGATGTGCTCGGGCCTTCCGTGTTGGTAAAAGCAAGAGGTGCACGGACTAAGCTTTTGG |
| | CACCTTTCTTCTCTAGCAGTGGGACAGCCTGCTATCCCATAGCAAATGCTTCTCCTCAGG |
| | TGGCCAAGATACTCTGGAGTCAAACCGCGTGCCTGCGGAGCGTTAAAATCATTATCCAAG |
| | CAGGTACCCAACGCGCTGTCGCAGTGACCGCCGACCACGAGGTTACCTCTACTAAGCTGG |
| | AGAAGGGGCACACCCTTGCCAAATACAATCCTTTTAAGAAATAAGCTGCGTCTCTGAGAT |
| | TGCGCTCCGCCCACTCACCCAGATCATCATGACACAAAAAACTAATCTGTCTTGATTATT |
| | TACAGTTAGTTTACCTGTCTATCAAGTTAGAAAAAACACGGGTAGAAGATTCTGGATCCC |
| | GGTTGGCGCCCTCCAGGTGCAAGttaattaaATGGGCTCCAGACCTTCTACCAAGAACCC |
| | AGCACCTATGATGCTGACTATCCGGGTTGCGCTGGTACTGAGTTGCATCTGTCCGGCAAA |
| | CTCCATTGATGGCAGGCCTCTTGCAGCTGCAGGAATTGTGGTTACAGGAGACAAAGCCGT |
| | CAACATATACACCTCATCCCAGACAGGATCAATCATAGTTAAGCTCCTCCCGAATCTGCC |
| | CAAGGATAAGGAGGCATGTGCGAAAGCCCCCTTGGATGCATACAACAGGACATTGACCAC |
| | TTTGCTCACCCCCCTTGGTGACTCTATCCGTAGGATACAAGAGTCTGTGACTACATCTGG |
| | AGGGGGGAGACAGGGGCGCCTTATAGGCGCCATTATTGGCGGTGTGGCTCTTGGGGTTGC |
| | AACTGCCGCACAAATAACAGCGGCCGCAGCTCTGATACAAGCCAAACAAAATGCTGCCAA |
| | CATCCTCCGACTTAAAGAGAGCATTGCCGCAACCAATGAGGCTGTGCATGAGGTCACTGA |
| | CGGATTATCGCAACTAGCAGTGGCAGTTGGGAAGATGCAGCAGTTTGTTAATGACCAATT |
| | TAATAAAACAGCTCAGGAATTAGACTGCATCAAAATTGCACAGCAAGTTGGTGTAGAGCT |
| | CAACCTGTACCTAACCGAATTGACTACAGTATTCGGACCACAAATCACTTCACCTGCTTT |
| | AAACAAGCTGACTATTCAGGCACTTTACAATCTAGCTGGTGGAAATATGGATTACTTATT |
| | GACTAAGTTAGGTGTAGGGAACAATCAACTCAGCTCATTAATCGGTAGCGGCTTAATCAC |
| | tGGcAACCCTATTCTATACGACTCACAGACTCAACTCTTGGGTATACAGGTAACTgcaCC |
| | TTCAGTCGGGAACCTAAATAATATGCGTGCCACCTACTTGGAAACCTTATCCGTAAGCAC |
| | AACCAGGGGATTTGCCTCGGCACTTGTCCCCAAAGTGGTGACACAGGTCGGTTCTGTGAT |
| | AGAAGAACTTGACACCTCATACTGTATAGAAACTGACTTAGATTTATATTGTACAAGAAT |
| | AGTAACGTTCCCTATGTCCCCTGGTATTTATTCCTGCTTGAGCGGCAATACGTCGGCCTG |
| | TATGTACTCAAAGACCGAAGGCGCACTTACTACACCATACATGACTATCAAAGGTTCAGT |
| | CATCGCCAACTGCAAGATGACAACATGTAGATGTGTAAACCCCCCGGGTATCATATCGCA |
| | AAACTATGGAGAAGCCGTGTCTCTAATAGATAAACAATCATGCAATGTTTTATCCTTAGG |
| | CGGGATAACTTTAAGGCTCAGTGGGGAATTCGATGTAACTTATCAGAAGAATATCTCAAT |
| | ACAAGATTGTCAAGTAATAATAACAGGCAATCTTGATATCTCAACTGAGGTTGGGAATGT |
| | CAACAACTCGATCAGTAATGCTTTGAATAAGTTAGAGGAAAGCAACAGAAAACTAGACAA |
| | AGTCAATGTCAAACTGACTAGCACATCTGCTCTCATTACgTATATCGTTTTGACTATCAT |
| | ATCTCTTGTTTTTGGTATACTTAGCCTGATTCTAGCATGCTACCTAATGTACAAGCAAAA |
| | GGCGCAACAAAAGACCTTATTATGGCTTGGGAATAATACaCTcGATCAGATGAGAGCCAC |
| | TACAAAAATGTGAACACAGATGAGGAACGAAGGTTTCCCTAATAGTAATTTGTGTGAAAG |
| | TTCTGGTAGTCTGTCAGTTCAGAGAGTTAAGAAAAAACTACCGGTTGTAGATGACCAAAG |
| | GACGATATACGGGTAGAACGGTAAGAGAGGCCGCCCCTCAATTGCGAGCCAGGCTTCACA |
| | ACCTCCGTTCTACCGCTTCACCGACAACAGTCCTCAATCATGGACCGCGCCGTTAGCCAA |
| | GTTGCGTTAGAGAATGATGAAAGAGAGGCAAAAAATACATGGCGCTTGATATTCCGGATT |
| | GCAATCTTATTCTTAACAGTAGTGACCTTGGCTATATCTGTAGCCTCCCTTTTATATAGC |
| | ATGGGGGCTAGCACACCTAGCGATCTTGTAGGCATACCGACTAGGATTTCCAGGGCAGAA |
| | GAAAAGATTACATCTACACTTGGTTCCAATCAAGATGTAGTAGATAGGATATATAAGCAA |
| | GTGGCCCTTGAGTCTCCGTTGGCATTGTTAAATACTGAGACCACAATTATGAACGCAATA |
| | ACATCTCTCTCTTATCAGATTAATGGAGCTGCAAACAACAGTGGGTGGGGGGCACCTATC |
| | CATGACCCAGATTATATAGGGGGGATAGGCAAAGAACTCATTGTAGATGATGCTAGTGAT |
| | GTCACATCATTCTATCCCTCTGCATTTCAAGAACATCTGAATTTTATCCCGGCGCCTACT |
| | ACAGGATCAGGTTGCACTCGAATACCCTCATTTGACATGAGTGCTACCCATTACTGCTAC |
| | ACCCATAATGTAATATTGTCTGGATGCAGAGATCACTCACATTCATATCAGTATTTAGCA |
| | CTTGGTGTGCTCCGGACATCTGCAACAGGGAGGGTATTCTTTTCTACTCTGCGTTCCATC |
| | AACCTGGACGACACCCAAAATCGGAAGTCTTGCAGTGTGAGTGCAACTCCCCTGGGTTGT |
| | GATATGCTGTGCTCGAAAGTCACGGAGACAGAGGAAGAAGATTATAACTCAGCTGTCCCT |
| | ACGCGGATGGTACATGGGAGGTTAGGGTTCGACGGCCAGTACCACGAAAAGGACCTAGAT |
| | GTCACAACATTATTCGGGGACTGGGTGGCCAACTACCCAGGAGTAGGGGGTGGATCTTTT |
| | ATTGACAGCCGCGTATGGTTCTCAGTCTACGGAGGGTTAAAACCCAATTCACCCAGTGAC |
| | ACTGTACAGGAAGGGAAATATGTGATATACAAGCGATACAATGACACATGCCCAGATGAG |
| | CAAGACTACCAGATTCGAATGGCCAAGTCTTCGTATAAGCCTGGACGGTTTGGTGGGAAA |
| | CGCATACAGCAGGCTATCTTATCTATCAAGGTGTCAACATCCTTAGGCGAAGACCCGGTA |
| | CTGACTGTACCGCCCAACACAGTCACACTCATGGGGGCCGAAGGCAGAATTCTCACAGTA |
| | GGGACATCTCATTTCTTGTATCAACGAGGGTCATCATACTTCTCTCCCGCGTTATTATAT |
| | CCTATGACAGTCAGCAACAAAACAGCCACTCTTCATAGTCCTTATACATTCAATGCCTTC |
| | ACTCGGCCAGGTAGTATCCCTTGCCAGGCTTCAGCAAGATGCCCCAACTCGTGTGTTACT |
| | GGAGTCTATACAGATCCATATCCCCTAATCTTCTATAGAAACCACACCTTGCGAGGGGTA |
| | TTCGGGACAATGCTTGATGGTGTACAAGCAAGACTTAACCCTGCGTCTGCAGTATTCGAT |
| | AGCACATCCCGCAGTCGCATTACTCGAGTGAGTTCAAGCAGTACCAAAGCAGCATACACA |
| | ACATCAACTTGTTTTAAAGTGGTCAAGACTAATAAGACCTATTGTCTCAGCATTGCTGAA |
| | ATATCTAATACTCTCTTCGGAGAATTCAGAATCGTCCCGTTACTAGTTGAGATCCTCAAA |
| | GATGACGGGGTTAGAGAAGCCAGGTCTGGCTAGggcgcgccTTGAGTCAATTATAAAGGA |
| | GTTGGAAAGATGGCATTGTATCACCTATCTTCTGCGACATCAAGAATCAAACCGAATGCC |
| | GGCGCGTGCTCGAATTCCATGTTGCCAGTTGACCACAATCAGCCAGTGCTCATGCGATCA |
| | GATTAAGCCTTGTCATTAATCTCTTGATTAAGAAAAAATGTAAGTGGCAATGAGATACAA |
| | GGCAAAACAGCTCATGGTAAATAATACGGGTAGGACATGGCGAGCTCCGGTCCTGAAAGG |
| | GCAGAGCATCAGATTATCCTACCAGAGCCACACCTGTCTTCACCATTGGTCAAGCACAAA |
| | CTACTCTATTACTGGAAATTAACTGGGCTACCGCTTCCTGATGAATGTGACTTCGACCAC |
| | CTCATTCTCAGCCGACAATGGAAAAAAATACTTGAATCGGCCTCTCCTGATACTGAGAGA |
| | ATGATAAAACTCGGAAGGGCAGTACACCAAACTCTTAACCACAATTCCAGAATAACCGGA |
| | GTGCTCCACCCCAGGTGTTTAGAACAACTGGCTAATATTGAGGTCCCAGATTCAACCAAC |
| | AAATTTCGGAAGATTGAGAAGAAGATCCAAATTCACAACACGAGATATGGAGAACTGTTC |
| | ACAAGGCTGTGTACGCATATAGAGAAGAAACTGCTGGGGTCATCTTGGTCTAACAATGTC |
| | CCCCGGTCAGAGGAGTTCAGCAGCATTCGTACGGATCCGGCATTCTGGTTTCACTCAAAA |
| | TGGTCCACAGCCAAGTTTGCATGGCTCCATATAAAACAGATCCAGAGGCATCTGATGGTG |
| | GCAGCTAAGACAAGGTCTGCGGCCAACAAATTGGTGATGCTAACCCATAAGGTAGGCCAA |
| | GTCTTTGTCACTCCTGAACTTGTCGTTGTGACGCATACGAATGAGAACAAGTTCACATGT |
| | CTTACCCAGGAACTTGTATTGATGTATGCAGATATGATGGAGGGCAGAGATATGGTCAAC |
| | ATAATATCAACCACGGCGGTGCATCTCAGAAGCTTATCAGAGAAAATTGATGACATTTTG |
| | CGGTTAATAGACGCTCTGGCAAAAGACTTGGGTAATCAAGTCTACGATGTTGTATCACTA |
| | ATGGAGGGATTTGCATACGGAGCTGTCCAGCTACTCGAGCCGTCAGGTACATTTGCAGGA |
| | GATTTCTTCGCATTCAACCTGCAGGAGCTTAAAGACATTCTAATTGGCCTCCTCCCCAAT |
| | GATATAGCAGAATCCGTGACTCATGCAATCGCTACTGTATTCTCTGGTTTAGAACAGAAT |
| | CAAGCAGCTGAGATGTTGTGTCTGTTGCGTCTGTGGGGTCACCCACTGCTTGAGTCCCGT |
| | ATTGCAGCAAAGGCAGTCAGGAGCCAAATGTGCGCACCGAAAATGGTAGACTTTGATATG |
| | ATCCTTCAGGTACTGTCTTTCTTCAAGGGAACAATCATCAACGGGTACAGAAAGAAGAAT |
| | GCAGGTGTGTGGCCGCGAGTCAAAGTGGATACAATATATGGGAAGGTCATTGGGCAACTA |
| | CATGCAGATTCAGCAGAGATTTCACACGATATCATGTTGAGAGAGTATAAGAGTTTATCT |
| | GCACTTGAATTTGAGCCATGTATAGAATATGACCCTGTCACCAACCTGAGCATGTTCCTA |
| | AAAGACAAGGCAATCGCACACCCCAACGATAATTGGCTTGCCTCGTTTAGGCGGAACCTT |
| | CTCTCCGAAGACCAGAAGAAACATGTAAAAGAAGCAACTTCGACTAATCGCCTCTTGATA |
| | GAGTTTTTAGAGTCAAATGATTTTGATCCATATAAAGAGATGGAATATCTGACGACCCTT |
| | GAGTACCTTAGAGATGACAATGTGGCAGTATCATACTCGCTCAAGGAGAAGGAAGTGAAA |
| | GTTAATGGACGGATCTTCGCTAAGCTGACAAAGAAGTTAAGGAACTGTCAGGTGATGGCG |
| | GAAGGGATCCTAGCCGATCAGATTGCACCTTTCTTTCAGGGAAATGGAGTCATTCAGGAT |
| | AGCATATCCTTGACCAAGAGTATGCTAGCGATGAGTCAACTGTCTTTTAACAGCAATAAG |
| | AAACGTATCACTGACTGTAAAGAAAGAGTATCTTCAAACCGCAATCATGATCCGAAAAGC |
| | AAGAACCGTCGGAGAGTTGCAACCTTCATAACAACTGACCTGCAAAAGTACTGTCTTAAT |
| | TGGAGATATCAGACAATCAAATTGTTCGCTCATGCCATCAATCAGTTGATGGGCCTACCT |
| | CACTTCTTCGAATGGATTCACCTAAGACTGATGGACACTACGATGTTCGTAGGAGACCCT |
| | TTCAATCCTCCAAGTGACCCTACTGACTGTGACCTCTCAAGAGTCCCTAATGATGACATA |
| | TATATTGTCAGTGCCAGAGGGGGTATCGAAGGATTATGCCAGAAGCTATGGACAATGATC |
| | TCAATTGCTGCAATCCAACTTGCTGCAGCTAGATCGCATTGTCGTGTTGCCTGTATGGTA |
| | CAGGGTGATAATCAAGTAATAGCAGTAACGAGAGAGGTAAGATCAGACGACTCTCCGGAG |
| | ATGGTGTTGACACAGTTGCATCAAGCCAGTGATAATTTCTTCAAGGAATTAATTCATGTC |
| | AATCATTTGATTGGCCATAATTTGAAGGATCGTGAAACCATCAGGTCAGACACATTCTTC |
| | ATATACAGCAAACGAATCTTCAAAGATGGAGCAATCCTCAGTCAAGTCCTCAAAAATTCA |
| | TCTAAATTAGTGCTAGTGTCAGGTGATCTCAGTGAAAACACCGTAATGTCCTGTGCCAAC |
| | ATTGCCTCTACTGTAGCACGGCTATGCGAGAACGGGCTTCCCAAAGACTTCTGTTACTAT |
| | TTAAACTATATAATGAGTTGTGTGCAGACATACTTTGACTCTGAGTTCTCCATCACCAAC |
| | AATTCGCACCCCGATCTTAATCAGTCGTGGATTGAGGACATCTCTTTTGTGCACTCATAT |
| | GTTCTGACTCCTGCCCAATTAGGGGGACTGAGTAACCTTCAATACTCAAGGCTCTACACT |
| | AGAAATATCGGTGACCCGGGGACTACTGCTTTTGCAGAGATCAAGCGACTAGAAGCAGTG |
| | GGATTACTGAGTCCTAACATTATGACTAATATCTTAACTAGGCCGCCTGGGAATGGAGAT |
| | TGGGCCAGTCTGTGCAACGACCCATACTCTTTCAATTTTGAGACTGTTGCAAGCCCAAAT |
| | ATTGTTCTTAAGAAACATACGCAAAGAGTCCTATTTGAAACTTGTTCAAATCCCTTATTG |
| | TCTGGAGTGCACACAGAGGATAATGAGGCAGAAGAGAAGGCATTGGCTGAATTCTTGCTT |
| | AATCAAGAGGTGATTCATCCCCGCGTTGCGCATGCCATCATGGAGGCAAGCTCTGTAGGT |
| | AGGAGAAAGCAAATTCAAGGGCTTGTTGACACAACAAACACCGTAATTAAGATTGCGCTT |
| | ACTAGGAGGCCATTAGGCATCAAGAGGCTGATGCGGATAGTCAATTATTCTAGCATGCAT |
| | GCAATGCTGTTTAGAGACGATGTTTTTTCCTCCAGTAGATCCAACCACCCCTTAGTCTCT |
| | TCTAATATGTGTTCTCTGACACTGGCAGACTATGCACGGAATAGAAGCTGGTCACCTTTG |
| | ACGGGAGGCAGGAAAATACTGGGTGTATCTAATCCTGATACGATAGAACTCGTAGAGGGT |
| | GAGATTCTTAGTGTAAGCGGAGGGTGTACAAGATGTGACAGCGGAGATGAACAATTTACT |
| | TGGTTCCATCTTCCAAGCAATATAGAATTGACCGATGACACCAGCAAGAATCCTCCGATG |
| | AGGGTACCATATCTCGGGTCAAAGACACAGGAGAGGAGAGCTGCCTCACTTGCAAAAATA |
| | GCTCATATGTCGCCACATGTAAAGGCTGCCCTAAGGGCATCATCCGTGTTGATCTGGGCT |
| | TATGGGGATAATGAAGTAAATTGGACTGCTGCTCTTACGATTGCAAAATCTCGGTGTAAT |
| | GTAAACTTAGAGTATCTTCGGTTACTGTCCCCTTTACCCACGGCTGGGAATCTTCAACAT |
| | AGACTAGATGATGGTATAACTCAGATGACATTCACCCCTGCATCTCTCTACAGGgtgtca |
| | ccttacattcacatatccaatgattctcaaaggctgttcactgaagaaggagtcaaagag |
| | gggaatgtggtttaccaacagatcATGCTCTTGGGTTTATCTCTAATCGAATCGATCTTT |
| | CCAATGACAACAACCAGGACATATGATGAGATCACACTGCACCTAGATAGTAAATTTAGT |
| | TGCTGTATCAGAGAAGCACCTGTTGCGGTTCCTTTCGAGCTACTTGGGGTGGTACCGGAA |
| | CTGAGGACAGTGACCTCAAATAAGTTTATGTATGATCCTAGCCCTGTATCGGAGGGAGAC |
| | TTTGCGAGACTTGACTTAGCTATCTTCAAGAGTTATGAGCTTAATCTGGAGTCATATCCC |
| | ACGATAGAGCTAATGAACATTCTTTCAATATCCAGCGGGAAGTTGATTGGCCAGTCTGTG |
| | GTTTCTTATGATGAAGATACCTCCATAAAGAATGACGCCATAATAGTGTATGACAATACC |
| | CGAAATTGGATCAGTGAAGCTCAGAATTCAGATGTGGTCCGCCTATTTGAATATGCAGCA |
| | CTTGAAGTGCTCCTCGACTGTTCTTACCAACTCTATTACCTGAGAGTAAGAGGCCTAGAC |
| | AATATTGTCTTATATATGGGTGATTTATACAAGAATATGCCAGGAATTCTACTTTCCAAC |
| | ATTGCAGCTACAATATCTCATCCCGTCATTCATTCAAGGTTACATGCAGTGGGCCTGGTC |
| | AACCATGACGGATCACACCAACTTGCAGATACGGATTTTATCGAAATGTCTGCAAAACTA |
| | TTAGTATCTTGCACCCGACGTGTGATCTCCGGCTTATATTCAGGAAATAAGTATGATCTG |
| | CTGTTCCCATCTGTCTTAGATGATAACCTGAATGAGAAGATGCTTCAGCTGATATCCCGG |
| | TTATGCTGTCTGTACACGGTACTCTTTGCTACAACAAGAGAAATCCCGAAAATAAGAGGC |
| | TTAACTGCAGAAGAGAAATGTTCAATACTCACTGAGTATTTACTGTCGGATGCTGTGAAA |
| | CCATTACTTAGCCCCGATCAAGTGAGCTCTATCATGTCTCCTAACATAATTACATTCCCA |
| | GCTAATCTGTACTACATGTCTCGGAAGAGCCTCAATTTGATCAGGGAAAGGGAGGACAGG |
| | GATACTATCCTGGCGTTGTTGTTCCCCCAAGAGCCATTATTAGAGTTCCCTTCTGTGCAA |
| | GATATTGGTGCTCGAGTGAAAGATCCATTCACCCGACAACCTGCGGCATTTTTGCAAGAG |
| | TTAGATTTGAGTGCTCCAGCAAGGTATGACGCATTCACACTTAGTCAGATTCATCCTGAA |
| | CTCACATCTCCAAATCCGGAGGAAGACTACTTAGTACGATACTTGTTCAGAGGGATAGGG |
| | ACTGCATCTTCCTCTTGGTATAAGGCATCTCATCTCCTTTCTGTACCCGAGGTAAGATGT |
| | GCAAGACACGGGAACTCCTTATACTTAGCTGAAGGGAGCGGAGCCATCATGAGTCTTCTC |
| | GAACTGCATGTACCACATGAAACTATCTATTACAATACGCTCTTTTCAAATGAGATGAAC |
| | CCCCCGCAACGACATTTCGGGCCGACCCCAACTCAGTTTTTGAATTCGGTTGTTTATAGG |
| | AATCTACAGGCGGAGGTAACATGCAAAGATGGATTTGTCCAAGAGTTCCGTCCATTATGG |
| | AGAGAAAATACAGAGGAAAGTGACCTGACCTCAGATAAAGCAGTGGGGTATATTACATCT |
| | GCAGTGCCCTACAGATCTGTATCATTGCTGCATTGTGACATTGAAATTCCTCCAGGGTCC |
| | AATCAAAGCTTACTAGATCAACTAGCTATCAATTTATCTCTGATTGCCATGCATTCTGTA |
| | AGGGAGGGCGGGGTAGTAATCATCAAAGTGTTGTATGCAATGGGATACTACTTTCATCTA |
| | CTCATGAACTTGTTTGCTCCGTGTTCCACAAAAGGATATATTCTCTCTAATGGTTATGCA |
| | TGTCGAGGAGATATGGAGTGTTACCTGGTATTTGTCATGGGTTACCTGGGCGGGCCTACA |
| | TTTGTACATGAGGTGGTGAGGATGGCAAAAACTCTGGTGCAGCGGCACGGTACGCTCTTG |
| | TCTAAATCAGATGAGATCACACTGACCAGGTTATTCACCTCACAGCGGCAGCGTGTGACA |
| | GACATCCTATCCAGTCCTTTACCAAGATTAATAAAGTACTTGAGGAAGAATATTGACACT |
| | GCGCTGATTGAAGCCGGGGGACAGCCCGTCCGTCCATTCTGTGCGGAGAGTCTGGTGAGC |
| | ACGCTAGCGAACATAACTCAGATAACCCAGATTATCGCTAGTCACATTGACACAGTTATC |
| | CGGTCTGTGATATATATGGAAGCTGAGGGTGATCTCGCTGACACAGTATTTCTATTTACC |
| | CCTTACAATCTCTCTACTGACGGGAAAAAGAGGACATCACTTATACAGTGCACGAGACAG |
| | ATCCTAGAGGTTACAATACTAGGTCTTAGAGTCGAAAATCTCAATAAAATAGGCGATATA |
| | ATCAGCCTAGTGCTTAAAGGCATGATCTCCATGGAGGACCTTATCCCACTAAGGACATAC |
| | TTGAAGCATAGTACCTGCCCTAAATATTTGAAGGCTGTCCTAGGTATTACCAAACTCAAA |
| | GAAATGTTTACAGACACTTCTGTATTGTACTTGACTCGTGCTCAACAAAAATTCTACATG |
| | AAAACTATAGGCAATGCAGTCAAAGGATATTACAGTAACTGTGACTCTTAACGAAAATCA |
| | CATATTAATAGGCTCCTTTTTTGGCCAATTGTATTCTTGTTGATTTAATCATATTATGTT |
| | AGAAAAAAGTTGAACCCTGACTCCTTAGGACTCGAATTCGAACTCAAATAAATGTCTTAA |
| | AAAAAGGTTGCGCACAATTATTCTTGAGTGTAGTCTCGTCATTCACCAAATCTTTGTTTG |
| | GTGGCCGGCATGGTCCCAGCCTCCTCGCTGGCGCCGGCTGGGCAACATTCCGAGGGGACC |
| | GTCCCCTCGGTAATGGCGAATGGGACGTCGACTGCTAACAAAGCCCGAAAGGAAGCTGAG |
| | TTGGCTGCTGCCACCGCTGAGCAATAACTAGCATAACCCCTTGGGGCCTCTAAACGGGTC |
| | TTGAGGGGTTTTTTGCTGAAAGGAGGAACTATAT |
| SEQ ID NO: | VSPYIHISNDSQRLFTEEGVKEGNVVYQQI |
| 20; amino | |
| acid | |
| sequence for | |
| stabilizing | |
| segment in L | |
| protein | |
| SEQ ID NO: | GCACCGAGTTCCCCCTCTAGATTAGAAAAAATACGGGTAGAACCGCCAC |
| 21; forward | |
| primer for | |
| expressing | |
| spike protein | |
| SEQ ID NO: | GTTGGACCTTGGGTACGCGTTTATCAGGTGTAGTGCAGCTTCAC |
| 22; reverse | |
| primer for | |
| expressing | |
| spike protein | |
| SEQ ID NO: | TAATACGACTCACTATAGGGACCAAACAGAGAATCCGTGAGTTACGATAAAAGGCGAAGG |
| 23; NDV- | AGCAATTGAAGTCGCACGGGTAGAAGGTGTGAATCTCGAGTGCGAGCCCGAAGCACAAAC |
| GFP-F3 aa | TCGAGAAAGCCTTCTGCCAACATGTCTTCCGTATTTGATGAGTACGAACAGCTCCTCGCG |
| Molecular | GCTCAGACTCGCCCCAATGGAGCTCATGGAGGGGGAGAAAAAGGGAGTACCTTAAAAGTA |
| Clone | GACGTCCCGGTATTCACTCTTAACAGTGATGACCCAGAAGATAGATGGAGCTTTGTGGTA |
| AF077761.1_ | TTCTGCCTCCGGATTGCTGTTAGCGAAGATGCCAACAAACCACTCAGGCAAGGTGCTCTC |
| LaSota_Kan | ATATCTCTTTTATGCTCCCACTCACAGGTAATGAGGAACCATGTTGCCATTGCAGGGAAA |
| R (with | CAGAATGAAGCCACATTGGCCGTGCTTGAGATTGATGGCTTTGCCAACGGCACGCCCCAG |
| stabilizing | TTCAACAATAGGAGTGGAGTGTCTGAAGAGAGAGCACAGAGATTTGCGATGATAGCAGGA |
| sequence in | TCTCTCCCTCGGGCATGCAGCAACGGAACCCCGTTCGTCACAGCCGGGGCAGAAGATGAT |
| L) | GCACCAGAAGACATCACCGATACCCTGGAGAGGATCCTCTCTATCCAGGCTCAAGTATGG |
| (mesogenic) | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaTTAGAAAAAATA |
| | CGGGTAGAACCGCCACCATGGTGAGCAAGGGCGAGGAGCTGTTCACCGGGGTGGTGCCCA |
| | TCCTGGTCGAGCTGGACGGCGACGTAAACGGCCACAAGTTCAGCGTGTCCGGCGAGGGCG |
| | AGGGCGATGCCACCTACGGCAAGCTGACCCTGAAGTTCATCTGCACCACCGGCAAGCTGC |
| | CCGTGCCCTGGCCCACCCTCGTGACCACCCTGACCTACGGCGTGCAGTGCTTCAGCCGCT |
| | ACCCCGACCACATGAAGCAGCACGACTTCTTCAAGTCCGCCATGCCCGAAGGCTACGTCC |
| | AGGAGCGCACCATCTTCTTCAAGGACGACGGCAACTACAAGACCCGCGCCGAGGTGAAGT |
| | TCGAGGGCGACACCCTGGTGAACCGCATCGAGCTGAAGGGCATCGACTTCAAGGAGGACG |
| | GCAACATCCTGGGGCACAAGCTGGAGTACAACTACAACAGCCACAACGTCTATATCATGG |
| | CCGACAAGCAGAAGAACGGCATCAAGGTGAACTTCAAGATCCGCCACAACATCGAGGACG |
| | GCAGCGTGCAGCTCGCCGACCACTACCAGCAGAACACCCCCATCGGCGACGGCCCCGTGC |
| | TGCTGCCCGACAACCACTACCTGAGCACCCAGTCCGCCCTGAGCAAAGACCCCAACGAGA |
| | AGCGCGATCACATGGTCCTGCTGGAGTTCGTGACCGCCGCCGGGATCACTCTCGGCATGG |
| | ACGAGCTGTACAAGTaATaaacgcgtACCCAAGGTCCAACTCTCCAAGCGGCAATCCTCT |
| | CTCGCTTCCTCAGCCCCACTGAATGGTCGCGTAACCGTAATTAATCTAGCTACATTTAAG |
| | ATTAAGAAAAAATACGGGTAGAATTGGAGTGCCCCAATTGTGCCAAGATGGACTCATCTA |
| | GGACAATTGGGCTGTACTTTGATTCTGCCCATTCTTCTAGCAACCTGTTAGCATTTCCGA |
| | TCGTCCTACAAGGCACAGGAGATGGGAAGAAGCAAATCGCCCCGCAATATAGGATCCAGC |
| | GCCTTGACTTGTGGACTGATAGTAAGGAGGACTCAGTATTCATCACCACCTATGGATTCA |
| | TCTTTCAAGTTGGGAATGAAGAAGCCACTGTCGGCATGATCGATGATAAACCCAAGCGCG |
| | AGTTACTTTCCGCTGCGATGCTCTGCCTAGGAAGCGTCCCAAATACCGGAGACCTTATTG |
| | AGCTGGCAAGGGCCTGTCTCACTATGATAGTCACATGCAAGAAGAGTGCAACTAATACTG |
| | AGAGAATGGTTTTCTCAGTAGTGCAGGCACCCCAAGTGCTGCAAAGCTGTAGGGTTGTGG |
| | CAAACAAATACTCATCAGTGAATGCAGTCAAGCACGTGAAAGCGCCAGAGAAGATTCCCG |
| | GGAGTGGAACCCTAGAATACAAGGTGAACTTTGTCTCCTTGACTGTGGTACCGAAGAAGG |
| | ATGTCTACAAGATCCCAGCTGCAGTATTGAAGGTTTCTGGCTCGAGTCTGTACAATCTTG |
| | CGCTCAATGTCACTATTAATGTGGAGGTAGACCCGAGGAGTCCTTTGGTTAAATCTTTGT |
| | CTAAGTCTGACAGCGGATACTATGCTAACCTCTTCTTGCATATTGGACTTATGACCACCG |
| | TAGATAGGAAGGGGAAGAAAGTGACATTTGACAAGCTGGAAAAGAAAATAAGGAGCCTTG |
| | ATCTATCTGTCGGGCTCAGTGATGTGCTCGGGCCTTCCGTGTTGGTAAAAGCAAGAGGTG |
| | CACGGACTAAGCTTTTGGCACCTTTCTTCTCTAGCAGTGGGACAGCCTGCTATCCCATAG |
| | CAAATGCTTCTCCTCAGGTGGCCAAGATACTCTGGAGTCAAACCGCGTGCCTGCGGAGCG |
| | TTAAAATCATTATCCAAGCAGGTACCCAACGCGCTGTCGCAGTGACCGCCGACCACGAGG |
| | TTACCTCTACTAAGCTGGAGAAGGGGCACACCCTTGCCAAATACAATCCTTTTAAGAAAT |
| | AAGCTGCGTCTCTGAGATTGCGCTCCGCCCACTCACCCAGATCATCATGACACAAAAAAC |
| | TAATCTGTCTTGATTATTTACAGTTAGTTTACCTGTCTATCAAGTTAGAAAAAACACGGG |
| | TAGAAGATTCTGGATCCCGGTTGGCGCCCTCCAGGTGCAAGttaattaaATGGGCTCCAG |
| | ACCTTCTACCAAGAACCCAGCACCTATGATGCTGACTATCCGGGTTGCGCTGGTACTGAG |
| | TTGCATCTGTCCGGCAAACTCCATTGATGGCAGGCCTCTTGCAGCTGCAGGAATTGTGGT |
| | TAGAGGAGACAAAGCCGTCAACATATACACCTCATCCCAGACAGGATCAATCATAGTTAA |
| | GCTCCTCCCGAATCTGCCCAAGGATAAGGAGGCATGTGCGAAAGCCCCCTTGGATGCATA |
| | CAACAGGACATTGACCACTTTGCTCACCCCCCTTGGTGACTCTATCCGTAGGATACAAGA |
| | GTCTGTGACTACATCTGGAGGGCGGAGACAGAGGCGCTTTATAGGCGCCATTATTGGCGG |
| | TGTGGCTCTTGGGGTTGCAACTGCCGCACAAATAACAGCGGCCGCAGCTCTGATACAAGC |
| | CAAACAAAATGCTGCCAACATCCTCCGACTTAAAGAGAGCATTGCCGCAACCAATGAGGC |
| | TGTGCATGAGGTCACTGACGGATTATCGCAACTAGCAGTGGCAGTTGGGAAGATGCAGCA |
| | GTTTGTTAATGACCAATTTAATAAAACAGCTCAGGAATTAGACTGCATCAAAATTGCACA |
| | GCAAGTTGGTGTAGAGCTCAACCTGTACCTAACCGAATTGACTACAGTATTCGGACCACA |
| | AATCACTTCACCTGCTTTAAACAAGCTGACTATTCAGGCACTTTACAATCTAGCTGGTGG |
| | AAATATGGATTACTTATTGACTAAGTTAGGTGTAGGGAACAATCAACTCAGCTCATTAAT |
| | CGGTAGCGGCTTAATCACtGGcAACCCTATTCTATACGACTCACAGACTCAACTCTTGGG |
| | TATACAGGTAACTgcaCCTTCAGTCGGGAACCTAAATAATATGCGTGCCACCTACTTGGA |
| | AACCTTATCCGTAAGCACAACCAGGGGATTTGCCTCGGCACTTGTCCCCAAAGTGGTGAC |
| | ACAGGTCGGTTCTGTGATAGAAGAACTTGACACCTCATACTGTATAGAAACTGACTTAGA |
| | TTTATATTGTACAAGAATAGTAACGTTCCCTATGTCCCCTGGTATTTATTCCTGCTTGAG |
| | CGGCAATACGTCGGCCTGTATGTACTCAAAGACCGAAGGCGCACTTACTACACCATACAT |
| | GACTATCAAAGGTTCAGTCATCGCCAACTGCAAGATGACAACATGTAGATGTGTAAACCC |
| | CCCGGGTATCATATCGCAAAACTATGGAGAAGCCGTGTCTCTAATAGATAAACAATCATG |
| | CAATGTTTTATCCTTAGGCGGGATAACTTTAAGGCTCAGTGGGGAATTCGATGTAACTTA |
| | TCAGAAGAATATCTCAATACAAGATTCTCAAGTAATAATAACAGGCAATCTTGATATCTC |
| | AACTGAGCTTGGGAATGTCAACAACTCGATCAGTAATGCTTTGAATAAGTTAGAGGAAAG |
| | CAACAGAAAACTAGACAAAGTCAATGTCAAACTGACTAGCACATCTGCTCTCATTACgTA |
| | TATCGTTTTGACTATCATATCTCTTGTTTTTGGTATACTTAGCCTGATTCTAGCATGCTA |
| | CCTAATGTACAAGCAAAAGGCGCAACAAAAGACCTTATTATGGCTTGGGAATAATACaCT |
| | cGATCAGATGAGAGCCACTACAAAAATGTGAACACAGATGAGGAACGAAGGTTTCCCTAA |
| | TAGTAATTTGTGTGAAAGTTCTGGTAGTCTGTCAGTTCAGAGAGTTAAGAAAAAACTACC |
| | GGTTGTAGATGACCAAAGGACGATATACGGGTAGAACGGTAAGAGAGGCCGCCCCTCAAT |
| | TGCGAGCCAGGCTTCACAACCTCCGTTCTACCGCTTCACCGACAACAGTCCTCAATCATG |
| | GACCGCGCCGTTAGCCAAGTTGCGTTAGAGAATGATGAAAGAGAGGCAAAAAATACATGG |
| | CGCTTGATATTCCGGATTGCAATCTTATTCTTAACAGTAGTGACCTTGGCTATATCTGTA |
| | GCCTCCCTTTTATATAGCATGGGGGCTAGCACACCTAGCGATCTTGTAGGCATACCGACT |
| | AGGATTTCCAGGGCAGAAGAAAAGATTACATCTACACTTGGTTCCAATCAAGATGTAGTA |
| | GATAGGATATATAAGCAAGTGGCCCTTGAGTCTCCGTTGGCATTGTTAAATACTGAGACC |
| | ACAATTATGAACGCAATAACATCTCTCTCTTATCAGATTAATGGAGCTGCAAACAACAGT |
| | GGGTGGGGGGCACCTATCCATGACCCAGATTATATAGGGGGGATAGGCAAAGAACTCATT |
| | GTAGATGATGCTAGTGATGTCACATCATTCTATCCCTCTGCATTTCAAGAACATCTGAAT |
| | TTTATCCCGGCGCCTACTACAGGATCAGGTTGCACTCGAATACCCTCATTTGACATGAGT |
| | GCTACCCATTACTGCTACACCCATAATGTAATATTGTCTGGATGCAGAGATCACTCACAT |
| | TCATATCAGTATTTAGCACTTGGTGTGCTCCGGACATCTGCAACAGGGAGGGTATTCTTT |
| | TCTACTCTGCGTTCCATCAACCTGGACGACACCCAAAATCGGAAGTCTTGCAGTGTGAGT |
| | GCAACTCCCCTGGGTTGTGATATGCTGTGCTCGAAAGTCACGGAGACAGAGGAAGAAGAT |
| | TATAACTCAGCTGTCCCTACGCGGATGGTACATGGGAGGTTAGGGTTCGACGGCCAGTAC |
| | CACGAAAAGGACCTAGATGTCACAACATTATTCGGGGACTGGGTGGCCAACTACCCAGGA |
| | GTAGGGGGTGGATCTTTTATTGACAGCCGCGTATGGTTCTCAGTCTACGGAGGGTTAAAA |
| | CCCAATTCACCCAGTGACACTGTACAGGAAGGGAAATATGTGATATACAAGCGATACAAT |
| | GACACATGCCCAGATGAGCAAGACTACCAGATTCGAATGGCCAAGTCTTCGTATAAGCCT |
| | GGACGGTTTGGTGGGAAACGCATACAGCAGGCTATCTTATCTATCAAGGTGTCAACATCC |
| | TTAGGCGAAGACCCGGTACTGACTGTACCGCCCAACACAGTCACACTCATGGGGGCCGAA |
| | GGCAGAATTCTCACAGTAGGGACATCTCATTTCTTGTATCAACGAGGGTCATCATACTTC |
| | TCTCCCGCGTTATTATATCCTATGACAGTCAGCAACAAAACAGCCACTCTTCATAGTCCT |
| | TATACATTCAATGCCTTCACTCGGCCAGGTAGTATCCCTTGCCAGGCTTCAGCAAGATGC |
| | CCCAACTCGTGTGTTACTGGAGTCTATACAGATCCATATCCCCTAATCTTCTATAGAAAC |
| | CACACCTTGCGAGGGGTATTCGGGACAATGCTTGATGGTGTACAAGCAAGACTTAACCCT |
| | GCGTCTGCAGTATTCGATAGCACATCCCGCAGTCGCATTACTCGAGTGAGTTCAAGCAGT |
| | ACCAAAGCAGCATACACAACATCAACTTGTTTTAAAGTGGTCAAGACTAATAAGACCTAT |
| | TGTCTCAGCATTGCTGAAATATCTAATACTCTCTTCGGAGAATTCAGAATCGTCCCGTTA |
| | CTAGTTGAGATCCTCAAAGATGACGGGGTTAGAGAAGCCAGGTCTGGCTAGggcgcgccT |
| | TGAGTCAATTATAAAGGAGTTGGAAAGATGGCATTGTATCACCTATCTTCTGCGACATCA |
| | AGAATCAAACCGAATGCCGGCGCGTGCTCGAATTCCATGTTGCCAGTTGACCACAATCAG |
| | CCAGTGCTCATGCGATCAGATTAAGCCTTGTCATTAATCTCTTGATTAAGAAAAAATGTA |
| | AGTGGCAATGAGATACAAGGCAAAACAGCTCATGGTAAATAATACGGGTAGGACATGGCG |
| | AGCTCCGGTCCTGAAAGGGCAGAGCATCAGATTATCCTACCAGAGCCACACCTGTCTTCA |
| | CCATTGGTCAAGCACAAACTACTCTATTACTGGAAATTAACTGGGCTACCGCTTCCTGAT |
| | GAATGTGACTTCGACCACCTCATTCTCAGCCGACAATGGAAAAAAATACTTGAATCGGCC |
| | TCTCCTGATACTGAGAGAATGATAAAACTCGGAAGGGCAGTACACCAAACTCTTAACCAC |
| | AATTCCAGAATAACCGGAGTGCTCCACCCCAGGTGTTTAGAACAACTGGCTAATATTGAG |
| | GTCCCAGATTCAACCAACAAATTTCGGAAGATTGAGAAGAAGATCCAAATTCACAACACG |
| | AGATATGGAGAACTGTTCACAAGGCTGTGTACGCATATAGAGAAGAAACTGCTGGGGTCA |
| | TCTTGGTCTAACAATGTCCCCCGGTCAGAGGAGTTCAGCAGCATTCGTACGGATCCGGCA |
| | TTCTGGTTTCACTCAAAATGGTCCACAGCCAAGTTTGCATGGCTCCATATAAAACAGATC |
| | CAGAGGCATCTGATGGTGGCAGCTAAGACAAGGTCTGCGGCCAACAAATTGGTGATGCTA |
| | ACCCATAAGGTAGGCCAAGTCTTTGTCACTCCTGAACTTGTCGTTGTGACGCATACGAAT |
| | GAGAACAAGTTCACATGTCTTACCCAGGAACTTGTATTGATGTATGCAGATATGATGGAG |
| | GGCAGAGATATGGTCAACATAATATCAACCACGGCGGTGCATCTCAGAAGCTTATCAGAG |
| | AAAATTGATGACATTTTGCGGTTAATAGACGCTCTGGCAAAAGACTTGGGTAATCAAGTC |
| | TACGATGTTGTATCACTAATGGAGGGATTTGCATACGGAGCTGTCCAGCTACTCGAGCCG |
| | TCAGGTACATTTGCAGGAGATTTCTTCGCATTCAACCTGCAGGAGCTTAAAGACATTCTA |
| | ATTGGCCTCCTCCCCAATGATATAGCAGAATCCGTGACTCATGCAATCGCTACTGTATTC |
| | TCTGGTTTAGAACAGAATCAAGCAGCTGAGATGTTGTGTCTGTTGCGTCTGTGGGGTCAC |
| | CCACTGCTTGAGTCCCGTATTGCAGCAAAGGCAGTCAGGAGCCAAATGTGCGCACCGAAA |
| | ATGGTAGACTTTGATATGATCCTTCAGGTACTGTCTTTCTTCAAGGGAACAATCATCAAC |
| | GGGTACAGAAAGAAGAATGCAGGTGTGTGGCCGCGAGTCAAAGTGGATACAATATATGGG |
| | AAGGTCATTGGGCAACTACATGCAGATTCAGCAGAGATTTCACACGATATCATGTTGAGA |
| | GAGTATAAGAGTTTATCTGCACTTGAATTTGAGCCATGTATAGAATATGACCCTGTCACC |
| | AACCTGAGCATGTTCCTAAAAGACAAGGCAATCGCACACCCCAACGATAATTGGCTTGCC |
| | TCGTTTAGGCGGAACCTTCTCTCCGAAGACCAGAAGAAACATGTAAAAGAAGCAACTTCG |
| | ACTAATCGCCTCTTGATAGAGTTTTTAGAGTCAAATGATTTTGATCCATATAAAGAGATG |
| | GAATATCTGACGACCCTTGAGTACCTTAGAGATGACAATGTGGCAGTATCATACTCGCTC |
| | AAGGAGAAGGAAGTGAAAGTTAATGGACGGATCTTCGCTAAGCTGACAAAGAAGTTAAGG |
| | AACTGTCAGGTGATGGCGGAAGGGATCCTAGCCGATCAGATTGCACCTTTCTTTCAGGGA |
| | AATGGAGTCATTCAGGATAGCATATCCTTGACCAAGAGTATGCTAGCGATGAGTCAACTG |
| | TCTTTTAACAGCAATAAGAAACGTATCACTGACTGTAAAGAAAGAGTATCTTCAAACCGC |
| | AATCATGATCCGAAAAGCAAGAACCGTCGGAGAGTTGCAACCTTCATAACAACTGACCTG |
| | CAAAAGTACTGTCTTAATTGGAGATATCAGACAATCAAATTGTTCGCTCATGCCATCAAT |
| | CAGTTGATGGGCCTACCTCACTTCTTCGAATGGATTCACCTAAGACTGATGGACACTACG |
| | ATGTTCGTAGGAGACCCTTTCAATCCTCCAAGTGACCCTACTGACTGTGACCTCTCAAGA |
| | GTCCCTAATGATGACATATATATTGTCAGTGCCAGAGGGGGTATCGAAGGATTATGCCAG |
| | AAGCTATGGACAATGATCTCAATTGCTGCAATCCAACTTGCTGCAGCTAGATCGCATTGT |
| | CGTGTTGCCTGTATGGTACAGGGTGATAATCAAGTAATAGCAGTAACGAGAGAGGTAAGA |
| | TCAGACGACTCTCCGGAGATGGTGTTGACACAGTTGCATCAAGCCAGTGATAATTTCTTC |
| | AAGGAATTAATTCATGTCAATCATTTGATTGGCCATAATTTGAAGGATCGTGAAACCATC |
| | AGGTCAGACACATTCTTCATATACAGCAAACGAATCTTCAAAGATGGAGCAATCCTCAGT |
| | CAAGTCCTCAAAAATTCATCTAAATTAGTGCTAGTGTCAGGTGATCTCAGTGAAAACACC |
| | GTAATGTCCTGTGCCAACATTGCCTCTACTGTAGCACGGCTATGCGAGAACGGGCTTCCC |
| | AAAGACTTCTGTTACTATTTAAACTATATAATGAGTTGTGTGCAGACATACTTTGACTCT |
| | GAGTTCTCCATCACCAACAATTCGCACCCCGATCTTAATCAGTCGTGGATTGAGGACATC |
| | TCTTTTGTGCACTCATATGTTCTGACTCCTGCCCAATTAGGGGGACTGAGTAACCTTCAA |
| | TACTCAAGGCTCTACACTAGAAATATCGGTGACCCGGGGACTACTGCTTTTGCAGAGATC |
| | AAGCGACTAGAAGCAGTGGGATTACTGAGTCCTAACATTATGACTAATATCTTAACTAGG |
| | CCGCCTGGGAATGGAGATTGGGCCAGTCTGTGCAACGACCCATACTCTTTCAATTTTGAG |
| | ACTGTTGCAAGCCCAAATATTGTTCTTAAGAAACATACGCAAAGAGTCCTATTTGAAACT |
| | TGTTCAAATCCCTTATTGTCTGGAGTGCACACAGAGGATAATGAGGCAGAAGAGAAGGCA |
| | TTGGCTGAATTCTTGCTTAATCAAGAGGTGATTCATCCCCGCGTTGCGCATGCCATCATG |
| | GAGGCAAGCTCTGTAGGTAGGAGAAAGCAAATTCAAGGGCTTGTTGACACAACAAACACC |
| | GTAATTAAGATTGCGCTTACTAGGAGGCCATTAGGCATCAAGAGGCTGATGCGGATAGTC |
| | AATTATTCTAGCATGCATGCAATGCTGTTTAGAGACGATGTTTTTTCCTCCAGTAGATCC |
| | AACCACCCCTTAGTCTCTTCTAATATGTGTTCTCTGACACTGGCAGACTATGCACGGAAT |
| | AGAAGCTGGTCACCTTTGACGGGAGGCAGGAAAATACTGGGTGTATCTAATCCTGATACG |
| | ATAGAACTCGTAGAGGGTGAGATTCTTAGTGTAAGCGGAGGGTGTACAAGATGTGACAGC |
| | GGAGATGAACAATTTACTTGGTTCCATCTTCCAAGCAATATAGAATTGACCGATGACACC |
| | AGCAAGAATCCTCCGATGAGGGTACCATATCTCGGGTCAAAGACACAGGAGAGGAGAGCT |
| | GCCTCACTTGCAAAAATAGCTCATATGTCGCCACATGTAAAGGCTGCCCTAAGGGCATCA |
| | TCCGTGTTGATCTGGGCTTATGGGGATAATGAAGTAAATTGGACTGCTGCTCTTACGATT |
| | GCAAAATCTCGGTGTAATGTAAACTTAGAGTATCTTCGGTTACTGTCCCCTTTACCCACG |
| | GCTGGGAATCTTCAACATAGACTAGATGATGGTATAACTCAGATGACATTCACCCCTGCA |
| | TCTCTCTACAGGgtgtcaccttacattcacatatccaatgattctcaaaggctgttcact |
| | gaagaaggagtcaaagaggggaatgtggtttaccaacagatcATGCTCTTGGGTTTATCT |
| | CTAATCGAATCGATCTTTCCAATGACAACAACCAGGACATATGATGAGATCACACTGCAC |
| | CTACATAGTAAATTTAGTTGCTGTATCAGAGAAGCACCTGTTGCGGTTCCTTTCGAGCTA |
| | CTTGGGGTGGTACCGGAACTGAGGACAGTGACCTCAAATAAGTTTATGTATGATCCTAGC |
| | CCTGTATCGGAGGGAGACTTTGCGAGACTTGACTTAGCTATCTTCAAGAGTTATGAGCTT |
| | AATCTGGAGTCATATCCCACGATAGAGCTAATGAACATTCTTTCAATATCCAGCGGGAAG |
| | TTGATTGGCCAGTCTGTGGTTTCTTATGATGAAGATACCTCCATAAAGAATGACGCCATA |
| | ATAGTGTATGACAATACCCGAAATTGGATCAGTGAAGCTCAGAATTCAGATGTGGTCCGC |
| | CTATTTGAATATGCAGCACTTGAAGTGCTCCTCGACTGTTCTTACCAACTCTATTACCTG |
| | AGAGTAAGAGGCCTAGACAATATTGTCTTATATATGGGTGATTTATACAAGAATATGCCA |
| | GGAATTCTACTTTCCAACATTGCAGCTACAATATCTCATCCCGTCATTCATTCAAGGTTA |
| | CATGCAGTGGGCCTGGTCAACCATGACGGATCACACCAACTTGCAGATACGGATTTTATC |
| | GAAATGTCTGCAAAACTATTAGTATCTTGCACCCGACGTGTGATCTCCGGCTTATATTCA |
| | GGAAATAAGTATGATCTGCTGTTCCCATCTGTCTTAGATGATAACCTGAATGAGAAGATG |
| | CTTCAGCTGATATCCCGGTTATGCTGTCTGTACACGGTACTCTTTGCTACAACAAGAGAA |
| | ATCCCGAAAATAAGAGGCTTAACTGCAGAAGAGAAATGTTCAATACTCACTGAGTATTTA |
| | CTGTCGGATGCTGTGAAACCATTACTTAGCCCCGATCAAGTGAGCTCTATCATGTCTCCT |
| | AACATAATTACATTCCCAGCTAATCTGTACTACATGTCTCGGAAGAGCCTCAATTTGATC |
| | AGGGAAAGGGAGGACAGGGATACTATCCTGGCGTTGTTGTTCCCCCAAGAGCCATTATTA |
| | GAGTTCCCTTCTGTGCAAGATATTGGTGCTCGAGTGAAAGATCCATTCACCCGACAACCT |
| | GCGGCATTTTTGCAAGAGTTAGATTTGAGTGCTCCAGCAAGGTATGACGCATTCACACTT |
| | AGTCAGATTCATCCTGAACTCACATCTCCAAATCCGGAGGAAGACTACTTAGTACGATAC |
| | TTGTTCAGAGGGATAGGGACTGCATCTTCCTCTTGGTATAAGGCATCTCATCTCCTTTCT |
| | GTACCCGAGGTAAGATGTGCAAGACACGGGAACTCCTTATACTTAGCTGAAGGGAGCGGA |
| | GCCATCATGAGTCTTCTCGAACTGCATGTACCACATGAAACTATCTATTACAATACGCTC |
| | TTTTCAAATGAGATGAACCCCCCGCAACGACATTTCGGGCCGACCCCAACTCAGTTTTTG |
| | AATTCGGTTGTTTATAGGAATCTACAGGCGGAGGTAACATGCAAAGATGGATTTGTCCAA |
| | GAGTTCCGTCCATTATGGAGAGAAAATACAGAGGAAAGTGACCTGACCTCAGATAAAGCA |
| | GTGGGGTATATTACATCTGCAGTGCCCTACAGATCTGTATCATTGCTGCATTGTGACATT |
| | GAAATTCCTCCAGGGTCCAATCAAAGCTTACTAGATCAACTAGCTATCAATTTATCTCTG |
| | ATTGCCATGCATTCTGTAAGGGAGGGCGGGGTAGTAATCATCAAAGTGTTGTATGCAATG |
| | GGATACTACTTTCATCTACTCATGAACTTGTTTGCTCCGTGTTCCACAAAAGGATATATT |
| | CTCTCTAATGGTTATGCATGTCGAGGAGATATGGAGTGTTACCTGGTATTTGTCATGGGT |
| | TACCTGGGCGGGCCTACATTTGTACATGAGGTGGTGAGGATGGCAAAAACTCTGGTGCAG |
| | CGGCACGGTACGCTCTTGTCTAAATCAGATGAGATCACACTGACCAGGTTATTCACCTCA |
| | CAGCGGCAGCGTGTGACAGACATCCTATCCAGTCCTTTACCAAGATTAATAAAGTACTTG |
| | AGGAAGAATATTGACACTGCGCTGATTGAAGCCGGGGGACAGCCCGTCCGTCCATTCTGT |
| | GCGGAGAGTCTGGTGAGCACGCTAGCGAACATAACTCAGATAACCCAGATTATCGCTAGT |
| | CACATTGACACAGTTATCCGGTCTGTGATATATATGGAAGCTGAGGGTGATCTCGCTGAC |
| | ACAGTATTTCTATTTACCCCTTACAATCTCTCTACTGACGGGAAAAAGAGGACATCACTT |
| | ATACAGTGCACGAGACAGATCCTAGAGGTTACAATACTAGGTCTTAGAGTCGAAAATCTC |
| | AATAAAATAGGCGATATAATCAGCCTAGTGCTTAAAGGCATGATCTCCATGGAGGACCTT |
| | ATCCCACTAAGGACATACTTGAAGCATAGTACCTGCCCTAAATATTTGAAGGCTGTCCTA |
| | GGTATTACCAAACTCAAAGAAATGTTTACAGACACTTCTGTATTGTACTTGACTCGTGCT |
| | CAACAAAAATTCTACATGAAAACTATAGGCAATGCAGTCAAAGGATATTACAGTAACTGT |
| | GACTCTTAACGAAAATCACATATTAATAGGCTCCTTTTTTGGCCAATTGTATTCTTGTTG |
| | ATTTAATCATATTATGTTAGAAAAAAGTTGAACCCTGACTCCTTAGGACTCGAATTCGAA |
| | CTCAAATAAATGTCTTAAAAAAAGGTTGCGCACAATTATTCTTGAGTGTAGTCTCGTCAT |
| | TCACCAAATCTTTGTTTGGTGGCCGGCATGGTCCCAGCCTCCTCGCTGGCGCCGGCTGGG |
| | CAACATTCCGAGGGGACCGTCCCCTCGGTAATGGCGAATGGGACGTCGACTGCTAACAAA |
| | GCCCGAAAGGAAGCTGAGTTGGCTGCTGCCACCGCTGAGCAATAACTAGCATAACCCCTT |
| | GGGGCCTCTAAACGGGTCTTGAGGGGTTTTTTGCTGAAAGGAGGAACTATA |
| SEQ ID NO: | GTTGGACCTTGGGTACGCGTTTATCATCAGCAGCAAGAGCCGCAAGAACAAC |
| 24; reverse | |
| primer for | |
| truncated | |
| form of the | |
| spike protein | |
| (SΔ19) | |
| SEQ ID NO: | GGGAGACAGGGGCGCC |
| 25; | |
| lentogenic | |
| nucleic acid | |
| sequence | |
| SEQ ID NO: | GRQGRL |
| 26; | |
| lentogenic | |
| amino acid | |
| sequence | |
| SEQ ID NO: | TAATACGACTCACTATAGGGACCAAACAGAGAATCCGTGAGTTACGATAAAAGGCGAAGG |
| 27; NDV- | AGCAATTGAAGTCGCACGGGTAGAAGGTGTGAATCTCGAGTGCGAGCCCGAAGCACAAAC |
| F3 aa | TCGAGAAAGCCTTCTGCCAACATGTCTTCCGTATTTGATGAGTACGAACAGCTCCTCGCG |
| Molecular | GCTCAGACTCGCCCCAATGGAGCTCATGGAGGGGGAGAAAAAGGGAGTACCTTAAAAGTA |
| Clone | GACGTCCCGGTATTCACTCTTAACAGTGATGACCCAGAAGATAGATGGAGCTTTGTGGTA |
| AF077761.1_ | TTCTGCCTCCGGATTGCTGTTAGCGAAGATGCCAACAAACCACTCAGGCAAGGTGCTCTC |
| LaSota_Kan | ATATCTCTTTTATGCTCCCACTCACAGGTAATGAGGAACCATGTTGCCATTGCAGGGAAA |
| R (with | CAGAATGAAGCCACATTGGCCGTGCTTGAGATTGATGGCTTTGCCAACGGCACGCCCCAG |
| stabilizing | TTCAACAATAGGAGTGGAGTGTCTGAAGAGAGAGCACAGAGATTTGCGATGATAGCAGGA |
| sequence in | TCTCTCCCTCGGGCATGCAGCAACGGAACCCCGTTCGTCACAGCCGGGGCAGAAGATGAT |
| L) | GCACCAGAAGACATCACCGATACCCTGGAGAGGATCCTCTCTATCCAGGCTCAAGTATGG |
| (mesogenic) | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaggacgcgtACCC |
| | AAGGTCCAACTCTCCAAGCGGCAATCCTCTCTCGCTTCCTCAGCCCCACTGAATGGTCGC |
| | GTAACCGTAATTAATCTAGCTACATTTAAGATTAAGAAAAAATACGGGTAGAATTGGAGT |
| | GCCCCAATTGTGCCAAGATGGACTCATCTAGGACAATTGGGCTGTACTTTGATTCTGCCC |
| | ATTCTTCTAGCAACCTGTTAGCATTTCCGATCGTCCTACAAGGCACAGGAGATGGGAAGA |
| | AGCAAATCGCCCCGCAATATAGGATCCAGCGCCTTGACTTGTGGACTGATAGTAAGGAGG |
| | ACTCAGTATTCATCACCACCTATGGATTCATCTTTCAAGTTGGGAATGAAGAAGCCACTG |
| | TCGGCATGATCGATGATAAACCCAAGCGCGAGTTACTTTCCGCTGCGATGCTCTGCCTAG |
| | GAAGCGTCCCAAATACCGGAGACCTTATTGAGCTGGCAAGGGCCTGTCTCACTATGATAG |
| | TCACATGCAAGAAGAGTGCAACTAATACTGAGAGAATGGTTTTGTCAGTAGTGCAGGCAC |
| | CCCAAGTGCTGCAAAGCTGTAGGGTTGTGGCAAACAAATACTCATCAGTGAATGCAGTCA |
| | AGCACGTGAAAGCGCCAGAGAAGATTCCCGGGAGTGGAACCCTAGAATACAAGGTGAACT |
| | TTGTCTCCTTGACTGTGGTACCGAAGAAGGATGTCTACAAGATCCCAGCTGCAGTATTGA |
| | AGGTTTCTGGCTCGAGTCTGTACAATCTTGCGCTCAATGTCACTATTAATGTGGAGGTAG |
| | ACCCGAGGAGTCCTTTGGTTAAATCTTTGTCTAAGTCTGACAGCGGATACTATGCTAACC |
| | TCTTCTTGCATATTGGACTTATGACCACCGTAGATAGGAAGGGGAAGAAAGTGACATTTG |
| | ACAAGCTGGAAAAGAAAATAAGGAGCCTTGATCTATCTGTCGGGCTCAGTGATGTGCTCG |
| | GGCCTTCCGTGTTGGTAAAAGCAAGAGGTGCACGGACTAAGCTTTTGGCACCTTTCTTCT |
| | CTAGCAGTGGGACAGCCTGCTATCCCATAGCAAATGCTTCTCCTCAGGTGGCCAAGATAC |
| | TCTGGAGTCAAACCGCGTGCCTGCGGAGCGTTAAAATCATTATCCAAGCAGGTACCCAAC |
| | GCGCTGTCGCAGTGACCGCCGACCACGAGGTTACCTCTACTAAGCTGGAGAAGGGGCACA |
| | CCCTTGCCAAATACAATCCTTTTAAGAAATAAGCTGCGTCTCTGAGATTGCGCTCCGCCC |
| | ACTCACCCAGATCATCATGACACAAAAAACTAATCTGTCTTGATTATTTACAGTTAGTTT |
| | ACCTGTCTATCAAGTTAGAAAAAACACGGGTAGAAGATTCTGGATCCCGGTTGGCGCCCT |
| | CCAGGTGCAAGttaattaaATGGGCTCCAGACCTTCTACCAAGAACCCAGCACCTATGAT |
| | GCTGACTATCCGGGTTGCGCTGGTACTGAGTTGCATCTGTCCGGCAAACTCCATTGATGG |
| | CAGGCCTCTTGCAGCTGCAGGAATTGTGGTTACAGGAGACAAAGCCGTCAACATATACAC |
| | CTCATCCCAGACAGGATCAATCATAGTTAAGCTCCTCCCGAATCTGCCCAAGGATAAGGA |
| | GGCATGTGCGAAAGCCCCCTTGGATGCATACAACAGGACATTGACCACTTTGCTCACCCC |
| | CCTTGGTGACTCTATCCGTAGGATACAAGAGTCTGTGACTACATCTGGAGGGCGGAGACA |
| | GAGGCGCTTTATAGGCGCCATTATTGGCGGTGTGGCTCTTGGGGTTGCAACTGCCGCACA |
| | AATAACAGCGGCCGCAGCTCTGATACAAGCCAAACAAAATGCTGCCAACATCCTCCGACT |
| | TAAAGAGAGCATTGCCGCAACCAATGAGGCTGTGCATGAGGTCACTGACGGATTATCGCA |
| | ACTAGCAGTGGCAGTTGGGAAGATGCAGCAGTTTGTTAATGACCAATTTAATAAAACAGC |
| | TCAGGAATTAGACTGCATCAAAATTGCACAGCAAGTTGGTGTAGAGCTCAACCTGTACCT |
| | AACCGAATTGACTACAGTATTCGGACCACAAATCACTTCACCTGCTTTAAACAAGCTGAC |
| | TATTCAGGCACTTTACAATCTAGCTGGTGGAAATATGGATTACTTATTGACTAAGTTAGG |
| | TGTAGGGAACAATCAACTCAGCTCATTAATCGGTAGCGGCTTAATCACtGGCAACCCTAT |
| | TCTATACGACTCACAGACTCAACTCTTGGGTATACAGGTAACTgCaCCTTCAGTCGGGAA |
| | CCTAAATAATATGCGTGCCACCTACTTGGAAACCTTATCCGTAAGCACAACCAGGGGATT |
| | TGCCTCGGCACTTGTCCCCAAAGTGGTGACACAGGTCGGTTCTGTGATAGAAGAACTTGA |
| | CACCTCATACTGTATAGAAACTGACTTAGATTTATATTGTACAAGAATAGTAACGTTCCC |
| | TATGTCCCCTGGTATTTATTCCTGCTTGAGCGGCAATACGTCGGCCTGTATGTACTCAAA |
| | GACCGAAGGCGCACTTACTACACCATACATGACTATCAAAGGTTCAGTCATCGCCAACTG |
| | CAAGATGACAACATGTAGATGTGTAAACCCCCCGGGTATCATATCGCAAAACTATGGAGA |
| | AGCCGTGTCTCTAATAGATAAACAATCATGCAATGTTTTATCCTTAGGCGGGATAACTTT |
| | AAGGCTCAGTGGGGAATTCGATGTAACTTATCAGAAGAATATCTCAATACAAGATTCTCA |
| | AGTAATAATAACAGGCAATCTTGATATCTCAACTGAGCTTGGGAATGTCAACAACTCGAT |
| | CAGTAATGCTTTGAATAAGTTAGAGGAAAGCAACAGAAAACTAGACAAAGTCAATGTCAA |
| | ACTGACTAGCACATCTGCTCTCATTACgTATATCGTTTTGACTATCATATCTCTTGTTTT |
| | TGGTATACTTAGCCTGATTCTAGCATGCTACCTAATGTACAAGCAAAAGGCGCAACAAAA |
| | GACCTTATTATGGCTTGGGAATAATACaCTcGATCAGATGAGAGCCACTACAAAAATGTG |
| | AACACAGATGAGGAACGAAGGTTTCCCTAATAGTAATTTGTGTGAAAGTTCTGGTAGTCT |
| | GTCAGTTCAGAGAGTTAAGAAAAAACTACCGGTTGTAGATGACCAAAGGACGATATACGG |
| | GTAGAACGGTAAGAGAGGCCGCCCCTCAATTGCGAGCCAGGCTTCACAACCTCCGTTCTA |
| | CCGCTTCACCGACAACAGTCCTCAATCATGGACCGCGCCGTTAGCCAAGTTGCGTTAGAG |
| | AATGATGAAAGAGAGGCAAAAAATACATGGCGCTTGATATTCCGGATTGCAATCTTATTC |
| | TTAACAGTAGTGACCTTGGCTATATCTGTAGCCTCCCTTTTATATAGCATGGGGGCTAGC |
| | ACACCTAGCGATCTTGTAGGCATACCGACTAGGATTTCCAGGGCAGAAGAAAAGATTACA |
| | TCTACACTTGGTTCCAATCAAGATGTAGTAGATAGGATATATAAGCAAGTGGCCCTTGAG |
| | TCTCCGTTGGCATTGTTAAATACTGAGACCACAATTATGAACGCAATAACATCTCTCTCT |
| | TATCAGATTAATGGAGCTGCAAACAACAGTGGGTGGGGGGCACCTATCCATGACCCAGAT |
| | TATATAGGGGGGATAGGCAAAGAACTCATTGTAGATGATGCTAGTGATGTCACATCATTC |
| | TATCCCTCTGCATTTCAAGAACATCTGAATTTTATCCCGGCGCCTACTACAGGATCAGGT |
| | TGCACTCGAATACCCTCATTTGACATGAGTGCTACCCATTACTGCTACACCCATAATGTA |
| | ATATTGTCTGGATGCAGAGATCACTCACATTCATATCAGTATTTAGCACTTGGTGTGCTC |
| | CGGACATCTGCAACAGGGAGGGTATTCTTTTCTACTCTGCGTTCCATCAACCTGGACGAC |
| | ACCCAAAATCGGAAGTCTTGCAGTGTGAGTGCAACTCCCCTGGGTTGTGATATGCTGTGC |
| | TCGAAAGTCACGGAGACAGAGGAAGAAGATTATAACTCAGCTGTCCCTACGCGGATGGTA |
| | CATGGGAGGTTAGGGTTCGACGGCCAGTACCACGAAAAGGACCTAGATGTCACAACATTA |
| | TTCGGGGACTGGGTGGCCAACTACCCAGGAGTAGGGGGTGGATCTTTTATTGACAGCCGC |
| | GTATGGTTCTCAGTCTACGGAGGGTTAAAACCCAATTCACCCAGTGACACTGTACAGGAA |
| | GGGAAATATGTGATATACAAGCGATACAATGACACATGCCCAGATGAGCAAGACTACCAG |
| | ATTCGAATGGCCAAGTCTTCGTATAAGCCTGGACGGTTTGGTGGGAAACGCATACAGCAG |
| | GCTATCTTATCTATCAAGGTGTCAACATCCTTAGGCGAAGACCCGGTACTGACTGTACCG |
| | CCCAACACAGTCACACTCATGGGGGCCGAAGGCAGAATTCTCACAGTAGGGACATCTCAT |
| | TTCTTGTATCAACGAGGGTCATCATACTTCTCTCCCGCGTTATTATATCCTATGACAGTC |
| | AGCAACAAAACAGCCACTCTTCATAGTCCTTATACATTCAATGCCTTCACTCGGCCAGGT |
| | AGTATCCCTTGCCAGGCTTCAGCAAGATGCCCCAACTCGTGTGTTACTGGAGTCTATACA |
| | GATCCATATCCCCTAATCTTCTATAGAAACCACACCTTGCGAGGGGTATTCGGGACAATG |
| | CTTGATGGTGTACAAGCAAGACTTAACCCTGCGTCTGCAGTATTCGATAGCACATCCCGC |
| | AGTCGCATTACTCGAGTGAGTTCAAGCAGTACCAAAGCAGCATACACAACATCAACTTGT |
| | TTTAAAGTGGTCAAGACTAATAAGACCTATTGTCTCAGCATTGCTGAAATATCTAATACT |
| | CTCTTCGGAGAATTCAGAATCGTCCCGTTACTAGTTGAGATCCTCAAAGATGACGGGGTT |
| | AGAGAAGCCAGGTCTGGCTAGggcgcgccTTGAGTCAATTATAAAGGAGTTGGAAAGATG |
| | GCATTGTATCACCTATCTTCTGCGACATCAAGAATCAAACCGAATGCCGGCGCGTGCTCG |
| | AATTCCATGTTGCCAGTTGACCACAATCAGCCAGTGCTCATGCGATCAGATTAAGCCTTG |
| | TCATTAATCTCTTGATTAAGAAAAAATGTAAGTGGCAATGAGATACAAGGCAAAACAGCT |
| | CATGGTAAATAATACGGGTAGGACATGGCGAGCTCCGGTCCTGAAAGGGCAGAGCATCAG |
| | ATTATCCTACCAGAGCCACACCTGTCTTCACCATTGGTCAAGCACAAACTACTCTATTAC |
| | TGGAAATTAACTGGGCTACCGCTTCCTGATGAATGTGACTTCGACCACCTCATTCTCAGC |
| | CGACAATGGAAAAAAATACTTGAATCGGCCTCTCCTGATACTGAGAGAATGATAAAACTC |
| | GGAAGGGCAGTACACCAAACTCTTAACCACAATTCCAGAATAACCGGAGTGCTCCACCCC |
| | AGGTGTTTAGAACAACTGGCTAATATTGAGGTCCCAGATTCAACCAACAAATTTCGGAAG |
| | ATTGAGAAGAAGATCCAAATTCACAACACGAGATATGGAGAACTGTTCACAAGGCTGTGT |
| | ACGCATATAGAGAAGAAACTGCTGGGGTCATCTTGGTCTAACAATGTCCCCCGGTCAGAG |
| | GAGTTCAGCAGCATTCGTACGGATCCGGCATTCTGGTTTCACTCAAAATGGTCCACAGCC |
| | AAGTTTGCATGGCTCCATATAAAACAGATCCAGAGGCATCTGATGGTGGCAGCTAAGACA |
| | AGGTCTGCGGCCAACAAATTGGTGATGCTAACCCATAAGGTAGGCCAAGTCTTTGTCACT |
| | CCTGAACTTGTCGTTGTGACGCATACGAATGAGAACAAGTTCACATGTCTTACCCAGGAA |
| | CTTGTATTGATGTATGCAGATATGATGGAGGGCAGAGATATGGTCAACATAATATCAACC |
| | ACGGCGGTGCATCTCAGAAGCTTATCAGAGAAAATTGATGACATTTTGCGGTTAATAGAC |
| | GCTCTGGCAAAAGACTTGGGTAATCAAGTCTACGATGTTGTATCACTAATGGAGGGATTT |
| | GCATACGGAGCTGTCCAGCTACTCGAGCCGTCAGGTACATTTGCAGGAGATTTCTTCGCA |
| | TTCAACCTGCAGGAGCTTAAAGACATTCTAATTGGCCTCCTCCCCAATGATATAGCAGAA |
| | TCCGTGACTCATGCAATCGCTACTGTATTCTCTGGTTTAGAACAGAATCAAGCAGCTGAG |
| | ATGTTGTGTCTGTTGCGTCTGTGGGGTCACCCACTGCTTGAGTCCCGTATTGCAGCAAAG |
| | GCAGTCAGGAGCCAAATGTGCGCACCGAAAATGGTAGACTTTGATATGATCCTTCAGGTA |
| | CTGTCTTTCTTCAAGGGAACAATCATCAACGGGTACAGAAAGAAGAATGCAGGTGTGTGG |
| | CCGCGAGTCAAAGTGGATACAATATATGGGAAGGTCATTGGGCAACTACATGCAGATTCA |
| | GCAGAGATTTCACACGATATCATGTTGAGAGAGTATAAGAGTTTATCTGCACTTGAATTT |
| | GAGCCATGTATAGAATATGACCCTGTCACCAACCTGAGCATGTTCCTAAAAGACAAGGCA |
| | ATCGCACACCCCAACGATAATTGGCTTGCCTCGTTTAGGCGGAACCTTCTCTCCGAAGAC |
| | CAGAAGAAACATGTAAAAGAAGCAACTTCGACTAATCGCCTCTTGATAGAGTTTTTAGAG |
| | TCAAATGATTTTGATCCATATAAAGAGATGGAATATCTGACGACCCTTGAGTACCTTAGA |
| | GATGACAATGTGGCAGTATCATACTCGCTCAAGGAGAAGGAAGTGAAAGTTAATGGACGG |
| | ATCTTCGCTAAGCTGACAAAGAAGTTAAGGAACTGTCAGGTGATGGCGGAAGGGATCCTA |
| | GCCGATCAGATTGCACCTTTCTTTCAGGGAAATGGAGTCATTCAGGATAGCATATCCTTG |
| | ACCAAGAGTATGCTAGCGATGAGTCAACTGTCTTTTAACAGCAATAAGAAACGTATCACT |
| | GACTGTAAAGAAAGAGTATCTTCAAACCGCAATCATGATCCGAAAAGCAAGAACCGTCGG |
| | AGAGTTGCAACCTTCATAACAACTGACCTGCAAAAGTACTGTCTTAATTGGAGATATCAG |
| | ACAATCAAATTGTTCGCTCATGCCATCAATCAGTTGATGGGCCTACCTCACTTCTTCGAA |
| | TGGATTCACCTAAGACTGATGGACACTACGATGTTCGTAGGAGACCCTTTCAATCCTCCA |
| | AGTGACCCTACTGACTGTGACCTCTCAAGAGTCCCTAATGATGACATATATATTGTCAGT |
| | GCCAGAGGGGGTATCGAAGGATTATGCCAGAAGCTATGGACAATGATCTCAATTGCTGCA |
| | ATCCAACTTGCTGCAGCTAGATCGCATTGTCGTGTTGCCTGTATGGTACAGGGTGATAAT |
| | CAAGTAATAGCAGTAACGAGAGAGGTAAGATCAGACGACTCTCCGGAGATGGTGTTGACA |
| | CAGTTGCATCAAGCCAGTGATAATTTCTTCAAGGAATTAATTCATGTCAATCATTTGATT |
| | GGCCATAATTTGAAGGATCGTGAAACCATCAGGTCAGACACATTCTTCATATACAGCAAA |
| | CGAATCTTCAAAGATGGAGCAATCCTCAGTCAAGTCCTCAAAAATTCATCTAAATTAGTG |
| | CTAGTGTCAGGTGATCTCAGTGAAAACACCGTAATGTCCTGTGCCAACATTGCCTCTACT |
| | GTAGCACGGCTATGCGAGAACGGGCTTCCCAAAGACTTCTGTTACTATTTAAACTATATA |
| | ATGAGTTGTGTGCAGACATACTTTGACTCTGAGTTCTCCATCACCAACAATTCGCACCCC |
| | GATCTTAATCAGTCGTGGATTGAGGACATCTCTTTTGTGCACTCATATGTTCTGACTCCT |
| | GCCCAATTAGGGGGACTGAGTAACCTTCAATACTCAAGGCTCTACACTAGAAATATCGGT |
| | GACCCGGGGACTACTGCTTTTGCAGAGATCAAGCGACTAGAAGCAGTGGGATTACTGAGT |
| | CCTAACATTATGACTAATATCTTAACTAGGCCGCCTGGGAATGGAGATTGGGCCAGTCTG |
| | TGCAACGACCCATACTCTTTCAATTTTGAGACTGTTGCAAGCCCAAATATTGTTCTTAAG |
| | AAACATACGCAAAGAGTCCTATTTGAAACTTGTTCAAATCCCTTATTGTCTGGAGTGCAC |
| | ACAGAGGATAATGAGGCAGAAGAGAAGGCATTGGCTGAATTCTTGCTTAATCAAGAGGTG |
| | ATTCATCCCCGCGTTGCGCATGCCATCATGGAGGCAAGCTCTGTAGGTAGGAGAAAGCAA |
| | ATTCAAGGGCTTGTTGACACAACAAACACCGTAATTAAGATTGCGCTTACTAGGAGGCCA |
| | TTAGGCATCAAGAGGCTGATGCGGATAGTCAATTATTCTAGCATGCATGCAATGCTGTTT |
| | AGAGACGATGTTTTTTCCTCCAGTAGATCCAACCACCCCTTAGTCTCTTCTAATATGTGT |
| | TCTCTGACACTGGCAGACTATGCACGGAATAGAAGCTGGTCACCTTTGACGGGAGGCAGG |
| | AAAATACTGGGTGTATCTAATCCTGATACGATAGAACTCGTAGAGGGTGAGATTCTTAGT |
| | GTAAGCGGAGGGTGTACAAGATGTGACAGCGGAGATGAACAATTTACTTGGTTCCATCTT |
| | CCAAGCAATATAGAATTGACCGATGACACCAGCAAGAATCCTCCGATGAGGGTACCATAT |
| | CTCGGGTCAAAGACACAGGAGAGGAGAGCTGCCTCACTTGCAAAAATAGCTCATATGTCG |
| | CCACATGTAAAGGCTGCCCTAAGGGCATCATCCGTGTTGATCTGGGCTTATGGGGATAAT |
| | GAAGTAAATTGGACTGCTGCTCTTACGATTGCAAAATCTCGGTGTAATGTAAACTTAGAG |
| | TATCTTCGGTTACTGTCCCCTTTACCCACGGCTGGGAATCTTCAACATAGACTAGATGAT |
| | GGTATAACTCAGATGACATTCACCCCTGCATCTCTCTACAGGgtgtcaccttacattcac |
| | atatccaatgattctcaaaggctgttcactgaagaaggagtcaaagaggggaatgtggtt |
| | taccaacagatcATGCTCTTGGGTTTATCTCTAATCGAATCGATCTTTCCAATGACAACA |
| | ACCAGGACATATGATGAGATCACACTGCACCTACATAGTAAATTTAGTTGCTGTATCAGA |
| | GAAGCACCTGTTGCGGTTCCTTTCGAGCTACTTGGGGTGGTACCGGAACTGAGGACAGTG |
| | ACCTCAAATAAGTTTATGTATGATCCTAGCCCTGTATCGGAGGGAGACTTTGCGAGACTT |
| | GACTTAGCTATCTTCAAGAGTTATGAGCTTAATCTGGAGTCATATCCCACGATAGAGCTA |
| | ATGAACATTCTTTCAATATCCAGCGGGAAGTTGATTGGCCAGTCTGTGGTTTCTTATGAT |
| | GAAGATACCTCCATAAAGAATGACGCCATAATAGTGTATGACAATACCCGAAATTGGATC |
| | AGTGAAGCTCAGAATTCAGATGTGGTCCGCCTATTTGAATATGCAGCACTTGAAGTGCTC |
| | CTCGACTGTTCTTACCAACTCTATTACCTGAGAGTAAGAGGCCTAGACAATATTGTCTTA |
| | TATATGGGTGATTTATACAAGAATATGCCAGGAATTCTACTTTCCAACATTGCAGCTACA |
| | ATATCTCATCCCGTCATTCATTCAAGGTTACATGCAGTGGGCCTGGTCAACCATGACGGA |
| | TCACACCAACTTGCAGATACGGATTTTATCGAAATGTCTGCAAAACTATTAGTATCTTGC |
| | ACCCGACGTGTGATCTCCGGCTTATATTCAGGAAATAAGTATGATCTGCTGTTCCCATCT |
| | GTCTTAGATGATAACCTGAATGAGAAGATGCTTCAGCTGATATCCCGGTTATGCTGTCTG |
| | TACACGGTACTCTTTGCTACAACAAGAGAAATCCCGAAAATAAGAGGCTTAACTGCAGAA |
| | GAGAAATGTTCAATACTCACTGAGTATTTACTGTCGGATGCTGTGAAACCATTACTTAGC |
| | CCCGATCAAGTGAGCTCTATCATGTCTCCTAACATAATTACATTCCCAGCTAATCTGTAC |
| | TACATGTCTCGGAAGAGCCTCAATTTGATCAGGGAAAGGGAGGACAGGGATACTATCCTG |
| | GCGTTGTTGTTCCCCCAAGAGCCATTATTAGAGTTCCCTTCTGTGCAAGATATTGGTGCT |
| | CGAGTGAAAGATCCATTCACCCGACAACCTGCGGCATTTTTGCAAGAGTTAGATTTGAGT |
| | GCTCCAGCAAGGTATGACGCATTCACACTTAGTCAGATTCATCCTGAACTCACATCTCCA |
| | AATCCGGAGGAAGACTACTTAGTACGATACTTGTTCAGAGGGATAGGGACTGCATCTTCC |
| | TCTTGGTATAAGGCATCTCATCTCCTTTCTGTACCCGAGGTAAGATGTGCAAGACACGGG |
| | AACTCCTTATACTTAGCTGAAGGGAGCGGAGCCATCATGAGTCTTCTCGAACTGCATGTA |
| | CCACATGAAACTATCTATTACAATACGCTCTTTTCAAATGAGATGAACCCCCCGCAACGA |
| | CATTTCGGGCCGACCCCAACTCAGTTTTTGAATTCGGTTGTTTATAGGAATCTACAGGCG |
| | GAGGTAACATGCAAAGATGGATTTGTCCAAGAGTTCCGTCCATTATGGAGAGAAAATACA |
| | GAGGAAAGTGACCTGACCTCAGATAAAGCAGTGGGGTATATTACATCTGCAGTGCCCTAC |
| | AGATCTGTATCATTGCTGCATTGTGACATTGAAATTCCTCCAGGGTCCAATCAAAGCTTA |
| | CTAGATCAACTAGCTATCAATTTATCTCTGATTGCCATGCATTCTGTAAGGGAGGGCGGG |
| | GTAGTAATCATCAAAGTGTTGTATGCAATGGGATACTACTTTCATCTACTCATGAACTTG |
| | TTTGCTCCGTGTTCCACAAAAGGATATATTCTCTCTAATGGTTATGCATGTCGAGGAGAT |
| | ATGGAGTGTTACCTGGTATTTGTCATGGGTTACCTGGGCGGGCCTACATTTGTACATGAG |
| | GTGGTGAGGATGGCAAAAACTCTGGTGCAGCGGCACGGTACGCTCTTGTCTAAATCAGAT |
| | GAGATCACACTGACCAGGTTATTCACCTCACAGCGGCAGCGTGTGACAGACATCCTATCC |
| | AGTCCTTTACCAAGATTAATAAAGTACTTGAGGAAGAATATTGACACTGCGCTGATTGAA |
| | GCCGGGGGACAGCCCGTCCGTCCATTCTGTGCGGAGAGTCTGGTGAGCACGCTAGCGAAC |
| | ATAACTCAGATAACCCAGATTATCGCTAGTCACATTGACACAGTTATCCGGTCTGTGATA |
| | TATATGGAAGCTGAGGGTGATCTCGCTGACACAGTATTTCTATTTACCCCTTACAATCTC |
| | TCTACTGACGGGAAAAAGAGGACATCACTTATACAGTGCACGAGACAGATCCTAGAGGTT |
| | ACAATACTAGGTCTTAGAGTCGAAAATCTCAATAAAATAGGCGATATAATCAGCCTAGTG |
| | CTTAAAGGCATGATCTCCATGGAGGACCTTATCCCACTAAGGACATACTTGAAGCATAGT |
| | ACCTGCCCTAAATATTTGAAGGCTGTCCTAGGTATTACCAAACTCAAAGAAATGTTTACA |
| | GACACTTCTGTATTGTACTTGACTCGTGCTCAACAAAAATTCTACATGAAAACTATAGGC |
| | AATGCAGTCAAAGGATATTACAGTAACTGTGACTCTTAACGAAAATCACATATTAATAGG |
| | CTCCTTTTTTGGCCAATTGTATTCTTGTTGATTTAATCATATTATGTTAGAAAAAAGTTG |
| | AACCCTGACTCCTTAGGACTCGAATTCGAACTCAAATAAATGTCTTAAAAAAAGGTTGCG |
| | CACAATTATTCTTGAGTGTAGTCTCGTCATTCACCAAATCTTTGTTTGGTGGCCGGCATG |
| | GTCCCAGCCTCCTCGCTGGCGCCGGCTGGGCAACATTCCGAGGGGACCGTCCCCTCGGTA |
| | ATGGCGAATGGGACGTCGACTGCTAACAAAGCCCGAAAGGAAGCTGAGTTGGCTGCTGCC |
| | ACCGCTGAGCAATAACTAGCATAACCCCTTGGGGCCTCTAAACGGGTCTTGAGGGGTTTT |
| | TTGCTGAAAGGAGGAACTATA |
| SEQ ID NO: | MGSRPSTKNPAPMMLTIRVALVLSCICPANSIDGRPLAAAGIVVTGDKAVNIYTSSQTGS |
| 28; NDV F | IIVKLLPNLPKDKEACAKAPLDAYNRTLTTLLTPLGDSIRRIQESVTTSGGGRQGRLIGA |
| gene wildtype | IIGGVALGVATAAQITAAAALIQAKQNAANILRLKESIAATNEAVHEVTDGLSQLAVAVG |
| (lentogenic) | KMQQFVNDQFNKTAQELDCIKIAQQVGVELNLYLTELTTVFGPQITSPALNKLTIQALYN |
| (553 aa) | LAGGNMDYLLTKLGVGNNQLSSLIGSGLITGNPILYDSQTQLLGIQVTAPSVGNLNNMRA |
| | TYLETLSVSTTRGFASALVPKVVTQVGSVIEELDTSYCIETDLDLYCTRIVTFPMSPGIY |
| | SCLSGNTSACMYSKTEGALTTPYMTIKGSVIANCKMTTCRCVNPPGIISQNYGEAVSLID |
| | KQSCNVLSLGGITLRLSGEFDVTYQKNISIQDSQVIITGNLDISTELGNVNNSISNALNK |
| | LEESNRKLDKVNVKLTSTSALITYIVLTIISLVFGILSLILACYLMYKQKAQQKTLLWLG |
| | NNTLDQMRATTKM |
| SEQ ID NO: | MFVFLVLLPLVSSQCVNLTTRTQLPPAYTNSFTRGVYYPDKVFRSSVLHSTQDLFLPFFS |
| 29; B117 | NVTWFHAISGTNGTKRFDNPVLPFNDGVYFASTEKSNIIRGWIFGTTLDSKTQSLLIVNN |
| spike protein | ATNVVIKVCEFQFCNDPFLGVYHKNNKSWMESEFRVYSSANNCTFEYVSQPFLMDLEGKQ |
| | GNFKNLREFVFKNIDGYFKIYSKHTPINLVRDLPQGFSALEPLVDLPIGINITRFQTLLA |
| | LHRSYLTPGDSSSGWTAGAAAYYVGYLQPRTFLLKYNENGTITDAVDCALDPLSETKCTL |
| | KSFTVEKGIYQTSNFRVQPTESIVRFPNITNLCPFGEVFNATRFASVYAWNRKRISNCVA |
| | DYSVLYNSASFSTFKCYGVSPTKLNDLCFTNVYADSFVIRGDEVRQIAPGQTGKIADYNY |
| | KLPDDFTGGVIAWNSNNLDSKVGGNYNYLYRLFRKSNLKPFERDISTEIYQAGSTPCNGV |
| | EGFNCYFPLQSYGFQPTYGVGYQPYRVVVLSFELLHAPATVCGPKKSTNLVKNKCVNFNF |
| | NGLTGTGVLTESNKKFLPFQQFGRDIDDTTDAVRDPQTLEILDITPCSFGGVSVITPGTN |
| | TSNQVAVLYQGVNCTEVPVAIHADQLTPTWRVYSTGSNVFQTRAGCLIGAEHVNNSYECD |
| | IPIGAGICASYQTQTNSHRRARSVASQSIIAYTMSLGAENSVAYSNNSIAIPINFTISVT |
| | TEILPVSMTKTSVDCTMYICGDSTECSNLLLQYGSFCTQLNRALTGIAVEQDKNTQEVFA |
| | QVKQIYKTPPIKDFGGFNFSQILPDPSKPSKRSFIEDLLFNKVTLADAGFIKQYGDCLGD |
| | IAARDLICAQKFNGLTVLPPLLTDEMIAQYTSALLAGTITSGWTFGAGAALQIPFAMQMA |
| | YRFNGIGVTQNVLYENQKLIANQFNSAIGKIQDSLSSTASALGKLQDVVNQNAQALNTLV |
| | KQLSSNFGAISSVLNDILARLDKVEAEVQIDRLITGRLQSLQTYVTQQLIRAAEIRASAN |
| | LAATKMSECVLGQSKRVDFCGKGYHLMSFPQSAPHGVVFLHVTYVPAQEKNFTTAPAICH |
| | DGKAHFPREGVFVSNGTHWFVTQRNFYEPQIITTHNTFVSGNCDVVIGIVNNTVYDPLQP |
| | ELDSFKEELDKYFKNHTSPDVDLGDISGINASVVNIQKEIDRLNEVAKNLNESLIDLQEL |
| | GKYEQYIKWPWYIWLGFIAGLIAIVMVTIMLCCMTSCCSCLKGCCSCGSCCKFDEDDSEP |
| | VLKGVKLHYT |
| SEQ ID NO: | MFVFLVLLPLVSSQCVNFTTRTQLPPAYTNSFTRGVYYPDKVFRSSVLHSTQDLFLPFFS |
| 30; B1.351 | NVTWFHAIHVSGTNGTKRFANPVLPFNDGVYFASTEKSNIIRGWIFGTTLDSKTQSLLIV |
| spike protein | NNATNVVIKVCEFQFCNDPFLGVYYHKNNKSWMESEFRVYSSANNCTFEYVSQPFLMDLE |
| | GKQGNFKNLREFVFKNIDGYFKIYSKHTPINLVRGLPQGFSALEPLVDLPIGINITRFQT |
| | LHISYLTPGDSSSGWTAGAAAYYVGYLQPRTFLLKYNENGTITDAVDCALDPLSETKCTL |
| | KSFTVEKGIYQTSNFRVQPTESIVRFPNITNLCPFGEVFNATRFASVYAWNRKRISNCVA |
| | DYSVLYNSASFSTFKCYGVSPTKLNDLCFTNVYADSFVIRGDEVRQIAPGQTGNIADYNY |
| | KLPDDFTGGVIAWNSNNLDSKVGGNYNYLYRLFRKSNLKPFERDISTEIYQAGSTPCNGV |
| | KGFNCYFPLQSYGFQPTYGVGYQPYRVVVLSFELLHAPATVCGPKKSTNLVKNKCVNFNF |
| | NGLTGTGVLTESNKKFLPFQQFGRDIADTTDAVRDPQTLEILDITPCSFGGVSVITPGTN |
| | TSNQVAVLYQGVNCTEVPVAIHADQLTPTWRVYSTGSNVFQTRAGCLIGAEHVNNSYECD |
| | IPIGAGICASYQTQTNSPRRARSVASQSIIAYTMSLGVENSVAYSNNSIAIPTNFTISVT |
| | TEILPVSMTKTSVDCTMYICGDSTECSNLLLQYGSFCTQLNRALTGIAVEQDKNTQEVFA |
| | QVKQIYKTPPIKDFGGFNFSQILPDPSKPSKRSFIEDLLFNKVTLADAGFIKQYGDCLGD |
| | IAARDLICAQKFNGLTVLPPLLTDEMIAQYTSALLAGTITSGWTFGAGAALQIPFAMQMA |
| | YRFNGIGVTQNVLYENQKLIANQFNSAIGKIQDSLSSTASALGKLQDVVNQNAQALNTLV |
| | KQLSSNFGAISSVLNDILSRLDKVEAEVQIDRLITGRLQSLQTYVTQQLIRAAEIRASAN |
| | LAATKMSECVLGQSKRVDFCGKGYHLMSFPQSAPHGVVFLHVTYVPAQEKNFTTAPAICH |
| | DGKAHFPREGVFVSNGTHWFVTQRNFYEPQIITTDNTFVSGNCDVVIGIVNNTVYDPLQP |
| | ELDSFKEELDKYFKNHTSPDVDLGDISGINASVVNIQKEIDRLNEVAKNLNESLIDLQEL |
| | GKYEQYIKWPWYIWLGFIAGLIAIVMVTIMLCCMTSCCSCLKGCCSCGSCCKFDEDDSEP |
| | VLKGVKLHYT |
| SEQ ID NO: | MFVFLVLLPLVSSQCVNFTTRTQLPPAYTNSFTRGVYYPDKVFRSSVLHSTQDLFLPFFS |
| 31; | NVTWFHAIHVSGTNGTKRFANPVLPFNDGVYFASTEKSNIIRGWIFGTTLDSKTQSLLIV |
| B1.351PP | NNATNVVIKVCEFQFCNDPFLGVYYHKNNKSWMESEFRVYSSANNCTFEYVSQPFLMDLE |
| spike protein | GKQGNFKNLREFVFKNIDGYFKIYSKHTPINLVRGLPQGFSALEPLVDLPIGINITRFQT |
| | LHISYLTPGDSSSGWTAGAAAYYVGYLQPRTFLLKYNENGTITDAVDCALDPLSETKCTL |
| | KSFTVEKGIYQTSNFRVQPTESIVRFPNITNLCPFGEVFNATRFASVYAWNRKRISNCVA |
| | DYSVLYNSASFSTFKCYGVSPTKLNDLCFTNVYADSFVIRGDEVRQIAPGQTGNIADYNY |
| | KLPDDFTGGVIAWNSNNLDSKVGGNYNYLYRLFRKSNLKPFERDISTEIYQAGSTPCNGV |
| | KGFNCYFPLQSYGFQPTYGVGYQPYRVVVLSFELLHAPATVCGPKKSTNLVKNKCVNFNF |
| | NGLTGTGVLTESNKKFLPFQQFGRDIADTTDAVRDPQTLEILDITPCSFGGVSVITPGTN |
| | TSNQVAVLYQGVNCTEVPVAIHADQLTPTWRVYSTGSNVFQTRAGCLIGAEHVNNSYECD |
| | IPIGAGICASYQTQTNSPRRARSVASQSIIAYTMSLGVENSVAYSNNSIAIPTNFTISVT |
| | TEILPVSMTKTSVDCTMYICGDSTECSNLLLQYGSFCTQLNRALTGIAVEQDKNTQEVFA |
| | QVKQIYKTPPIKDFGGFNFSQILPDPSKPSKRSFIEDLLFNKVTLADAGFIKQYGDCLGD |
| | IAARDLICAQKFNGLTVLPPLLTDEMIAQYTSALLAGTITSGWTFGAGAALQIPFAMQMA |
| | YRFNGIGVTQNVLYENQKLIANQFNSAIGKIQDSLSSTASALGKLQDVVNQNAQALNTLV |
| | KQLSSNFGAISSVLNDILSRLDPPEAEVQIDRLITGRLQSLQTYVTQQLIRAAEIRASAN |
| | LAATKMSECVLGQSKRVDFCGKGYHLMSFPQSAPHGVVFLHVTYVPAQEKNFTTAPAICH |
| | DGKAHFPREGVFVSNGTHWFVTQRNFYEPQIITTDNTFVSGNCDVVIGIVNNTVYDPLQP |
| | ELDSFKEELDKYFKNHTSPDVDLGDISGINASVVNIQKEIDRLNEVAKNLNESLIDLQEL |
| | GKYEQYIKWPWYIWLGFIAGLIAIVMVTIMLCCMTSCCSCLKGCCSCGSCCKFDEDDSEP |
| | VLKGVKLHYT |
| SEQ ID NO: | ATGGGCTCCAGACCTTCTACCAAGAACCCAGCACCTATGATGCTGACTATCCGGGTTGCG |
| 32; NDV wild | CTGGTACTGAGTTGCATCTGTCCGGCAAACTCCATTGATGGCAGGCCTCTTGCAGCTGCA |
| type F gene | GGAATTGTGGTTACAGGAGACAAAGCCGTCAACATATACACCTCATCCCAGACAGGATCA |
| from | ATCATAGTTAAGCTCCTCCCGAATCTGCCCAAGGATAAGGAGGCATGTGCGAAAGCCCCC |
| accession # | TTGGATGCATACAACAGGACATTGACCACTTTGCTCACCCCCCTTGGTGACTCTATCCGT |
| AF077761.1 | AGGATACAAGAGTCTGTGACTACATCTGGAGGGGGGAGACAGGGGCGCCTTATAGGCGCC |
| | ATTATTGGCGGTGTGGCTCTTGGGGTTGCAACTGCCGCACAAATAACAGCGGCCGCAGCT |
| | CTGATACAAGCCAAACAAAATGCTGCCAACATCCTCCGACTTAAAGAGAGCATTGCCGCA |
| | ACCAATGAGGCTGTGCATGAGGTCACTGACGGATTATCGCAACTAGCAGTGGCAGTTGGG |
| | AAGATGCAGCAGTTTGTTAATGACCAATTTAATAAAACAGCTCAGGAATTAGACTGCATC |
| | AAAATTGCACAGCAAGTTGGTGTAGAGCTCAACCTGTACCTAACCGAATTGACTACAGTA |
| | TTCGGACCACAAATCACTTCACCTGCTTTAAACAAGCTGACTATTCAGGCACTTTACAAT |
| | CTAGCTGGTGGAAATATGGATTACTTATTGACTAAGTTAGGTGTAGGGAACAATCAACTC |
| | AGCTCATTAATCGGTAGCGGCTTAATCACtGGcAACCCTATTCTATACGACTCACAGACT |
| | CAACTCTTGGGTATACAGGTAACTgcaCCTTCAGTCGGGAACCTAAATAATATGCGTGCC |
| | ACCTACTTGGAAACCTTATCCGTAAGCACAACCAGGGGATTTGCCTCGGCACTTGTCCCC |
| | AAAGTGGTGACACAGGTCGGTTGTGTGATAGAAGAACTTGACACCTCATACTGTATAGAA |
| | ACTGACTTAGATTTATATTGTACAAGAATAGTAACGTTCCCTATGTCCCCTGGTATTTAT |
| | TCCTGCTTGAGCGGCAATACGTCGGCCTGTATGTACTCAAAGACCGAAGGCGCACTTACT |
| | ACACCATACATGACTATCAAAGGTTCAGTCATCGCCAACTGCAAGATGACAACATGTAGA |
| | TGTGTAAACCCCCCGGGTATCATATCGCAAAACTATGGAGAAGCCGTGTCTCTAATAGAT |
| | AAACAATCATGCAATGTTTTATCCTTAGGCGGGATAACTTTAAGGCTCAGTGGGGAATTC |
| | GATGTAACTTATCAGAAGAATATCTCAATACAAGATTCTCAAGTAATAATAACAGGCAAT |
| | CTTGATATCTCAACTGAGCTTGGGAATGTCAACAACTCGATCAGTAATGCTTTGAATAAG |
| | TTAGAGGAAAGCAACAGAAAACTAGACAAAGTCAATGTCAAACTGACTAGCACATCTGCT |
| | CTCATTACgTATATCGTTTTGACTATCATATCTCTTGTTTTTGGTATACTTAGCCTGATT |
| | CTAGCATGCTACCTAATGTACAAGCAAAAGGCGCAACAAAAGACCTTATTATGGCTTGGG |
| | AATAATACaCTcGATCAGATGAGAGCCACTACAAAAATGTGA |
| SEQ ID NO: | ATGGACCGCGCCGTTAGCCAAGTTGCGTTAGAGAATGATGAAAGAGAGGCAAAAAATACA |
| 33; NDV wild | TGGCGCTTGATATTCCGGATTGCAATCTTATTCTTAACAGTAGTGACCTTGGCTATATCT |
| type HN gene | GTAGCCTCCCTTTTATATAGCATGGGGGCTAGCACACCTAGCGATCTTGTAGGCATACCG |
| from | ACTAGGATTTCCAGGGCAGAAGAAAAGATTACATCTACACTTGGTTCCAATCAAGATGTA |
| accession # | GTAGATAGGATATATAAGCAAGTGGCCCTTGAGTCTCCGTTGGCATTGTTAAATACTGAG |
| AF077761.1 | ACCACAATTATGAACGCAATAACATCTCTCTCTTATCAGATTAATGGAGCTGCAAACAAC |
| | AGTGGGTGGGGGGCACCTATCCATGACCCAGATTATATAGGGGGGATAGGCAAAGAACTC |
| | ATTGTAGATGATGCTAGTGATGTCACATCATTCTATCCCTCTGCATTTCAAGAACATCTG |
| | AATTTTATCCCGGCGCCTACTACAGGATCAGGTTGCACTCGAATACCCTCATTTGACATG |
| | AGTGCTACCCATTACTGCTACACCCATAATGTAATATTGTCTGGATGCAGAGATCACTCA |
| | CATTCATATCAGTATTTAGCACTTGGTGTGCTCCGGACATCTGCAACAGGGAGGGTATTC |
| | TTTTCTACTCTGCGTTCCATCAACCTGGACGACACCCAAAATCGGAAGTCTTGCAGTGTG |
| | AGTGCAACTCCCCTGGGTTGTGATATGCTGTGCTCGAAAGTCACGGAGACAGAGGAAGAA |
| | GATTATAACTCAGCTGTCCCTACGCGGATGGTACATGGGAGGTTAGGGTTCGACGGCCAG |
| | TACCACGAAAAGGACCTAGATGTCACAACATTATTCGGGGACTGGGTGGCCAACTACCCA |
| | GGAGTAGGGGGTGGATCTTTTATTGACAGCCGCGTATGGTTCTCAGTCTACGGAGGGTTA |
| | AAACCCAATTCACCCAGTGACACTGTACAGGAAGGGAAATATGTGATATACAAGCGATAC |
| | AATGACACATGCCCAGATGAGCAAGACTACCAGATTCGAATGGCCAAGTCTTCGTATAAG |
| | CCTGGACGGTTTGGTGGGAAACGCATACAGCAGGCTATCTTATCTATCAAGGTGTCAACA |
| | TCCTTAGGCGAAGACCCGGTACTGACTGTACCGCCCAACACAGTCACACTCATGGGGGCC |
| | GAAGGCAGAATTCTCACAGTAGGGACATCTCATTTCTTGTATCAACGAGGGTCATCATAC |
| | TTCTCTCCCGCGTTATTATATCCTATGACAGTCAGCAACAAAACAGCCACTCTTCATAGT |
| | CCTTATACATTCAATGCCTTCACTCGGCCAGGTAGTATCCCTTGCCAGGCTTCAGCAAGA |
| | TGCCCCAACTCGTGTGTTACTGGAGTCTATACAGATCCATATCCCCTAATCTTCTATAGA |
| | AACCACACCTTGCGAGGGGTATTCGGGACAATGCTTGATGGTGTACAAGCAAGACTTAAC |
| | CCTGCGTCTGCAGTATTCGATAGCACATCCCGCAGTCGCATTACTCGAGTGAGTTCAAGC |
| | AGTACCAAAGCAGCATACACAACATCAACTTGTTTTAAAGTGGTCAAGACTAATAAGACC |
| | TATTGTCTCAGCATTGCTGAAATATCTAATACTCTCTTCGGAGAATTCAGAATCGTCCCG |
| | TTACTAGTTGAGATCCTCAAAGATGACGGGGTTAGAGAAGCCAGGTCTGGCTAG |
| SEQ ID NO: | MDRAVSQVALENDEREAKNTWRLIFRIAILFLTVVTLAISVASLLYSMGASTPSDLVGIP |
| 34; NDV wild | TRISRAEEKITSTLGSNQDVVDRIYKQVALESPLALLNTETTIMNAITSLSYQINGAANN |
| type HN | SGWGAPIHDPDYIGGIGKELIVDDASDVTSFYPSAFQEHLNFIPAPTTGSGCTRIPSFDM |
| protein | SATHYCYTHNVILSGCRDHSHSYQYLALGVLRTSATGRVFFSTLRSINLDDTQNRKSCSV |
| encoded by | SATPLGCDMLCSKVTETEEEDYNSAVPTRMVHGRLGFDGQYHEKDLDVTTLFGDWVANYP |
| SEQ ID NO: | GVGGGSFIDSRVWFSVYGGLKPNSPSDTVQEGKYVIYKRYNDTCPDEQDYQIRMAKSSYK |
| 33, from | PGRFGGKRIQQAILSIKVSTSLGEDPVLTVPPNTVTLMGAEGRILTVGTSHFLYQRGSSY |
| accession # | FSPALLYPMTVSNKTATLHSPYTFNAFTRPGSIPCQASARCPNSCVTGVYTDPYPLIFYR |
| AF077761.1 | NHTLRGVFGTMLDGVQARLNPASAVFDSTSRSRITRVSSSSTKAAYTTSTCFKVVKTNKT |
| | YCLSIAEISNTLFGEFRIVPLLVEILKDDGVREARSG |
| SEQ ID NO: | GTGTCACCTTACATTCACATATCCAATGATTCTCAAAGGCTGTTCACTGAAGAAGGAGTC |
| 35; encodes | AAAGAGGGGAATGTGGTTTACCAACAGATC |
| the stabilizing | |
| segment in L | |
| protein | |
| SEQ ID NO: | RRQRRF |
| 36; | |
| mesogenic | |
| amino acid | |
| sequence | |
| SEQ ID NO: | ACAGGTACGTTAATAGTTAATAGCGT |
| 37; | |
| E_Sarbeco_ | |
| F1 | |
| SEQ ID NO: | ATATTGCAGCAGTACGCACACA |
| 38; | |
| E_Sarbeco_ | |
| R2 | |
| SEQ ID NO: | FAM-ACACTAGCCATCCTTACTGCGCTTCG-BBQ |
| 39; | |
| E_Sarbeco_ | |
| P1 | |
| SEQ ID NO: | ATGTTCGTGTTCCTGGTCCTGCTGCCACTGGTAAGCTCCCAATGTGTAAACTTAACCACA |
| 40 | AGAACCCAGCTCCCACCTGCCTACACCAACAGCTTCACCAGAGGCGTTTATTACCCCGAC |
| chimeric | AAGGTATTCCGGTCTTCTGTTCTGCACTCTACCCAGGACCTGTTTCTGCCCTTTTTCAGC |
| SARS-CoV-2 | AACGTGACATGGTTCCACGCCATCCACGTGTCTGGCACAAACGGCACCAAGCGGTTTGAT |
| spike gene | AATCCTGTGCTCCCTTTCAATGACGGCGTGTACTTCGCCTCTACTGAGAAGAGCAACATC |
| encoding | ATCCGGGGCTGGATCTTTGGCACAACACTGGACTCTAAAACCCAGAGCCTGCTGATCGTG |
| protein | AACAACGCCACCAACGTGGTGATTAAGGTGTGCGAGTTCCAGTTCTGCAATGACCCTTTC |
| containing | CTCGGCGTGTACTACCACAAGAACAACAAAAGTTGGATGGAAAGCGAATTCAGGGTGTAC |
| the | TCAAGCGCCAACAACTGTACCTTCGAGTACGTGAGCCAGCCTTTCCTGATGGACCTAGAA |
| transmembrane | GGTAAGCAGGGCAATTTCAAGAACCTCAGAGAGTTCGTGTTCAAGAATATTGACGGCTAC |
| (TM) and | TTCAAAATCTACAGCAAGCACACCCCAATCAACCTGGTGCGGGACCTGCCCCAGGGCTTT |
| cytoplasmic | AGCGCGCTGGAGCCTCTGGTGGACCTGCCTATCGGCATCAACATCACCCGGTTCCAGACA |
| (CT) domain | CTGCTGGCTCTGCATAGAAGCTACCTGACACCTGGCGACAGTTCTTCTGGCTGGACAGCC |
| of the NDV F | GGCGCCGCCGCCTACTACGTGGGCTACCTGCAGCCTAGAACATTCCTGCTGAAATACAAC |
| protein | GAGAACGGCACGATCACAGACGCCGTGGACTGCGCCCTGGATCCCCTGTCTGAGACAAAG |
| | TGCACCCTGAAGTCTTTCACCGTGGAGAAGGGCATCTACCAGACCTCCAACTTCAGAGTG |
| | CAGCCTACCGAATCCATCGTGCGCTTTCCCAACATCACCAACCTGTGCCCCTTCGGCGAG |
| | GTCTTTAATGCCACGAGATTCGCCAGCGTGTATGCCTGGAACAGAAAGAGAATCAGCAAC |
| | TGCGTGGCCGACTACAGCGTGCTGTACAACAGCGCCTCTTTCAGCACATTTAAGTGCTAC |
| | GGAGTGTCTCCTACCAAACTCAACGATCTGTGCTTCACGAACGTGTATGCCGACAGCTTC |
| | GTGATCCGAGGAGATGAGGTGCGGCAGATCGCTCCAGGACAGACAGGCAAGATCGCCGAC |
| | TACAACTACAAGCTGCCCGACGACTTTACCGGCTGCGTGATCGCTTGGAACAGCAATAAC |
| | CTGGACTCAAAGGTTGGAGGAAACTACAACTACCTGTACAGACTGTTCAGAAAGTCCAAC |
| | CTGAAGCCCTTCGAGAGAGACATCTCTACAGAAATCTACCAGGCCGGCAGCACCCCATGT |
| | AACGGCGTGGAAGGCTTCAACTGCTACTTCCCTCTGCAGTCTTATGGCTTCCAGCCCACA |
| | AACGGAGTGGGCTATCAGCCTTACCGCGTGGTTGTCCTGAGCTTTGAGCTGCTGCATGCC |
| | CCTGCTACGGTGTGTGGACCTAAGAAGTCCACCAACCTGGTGAAGAACAAGTGTGTGAAC |
| | TTCAACTTCAACGGCCTGACCGGCACCGGGGTGCTGACAGAGTCTAACAAGAAATTCCTG |
| | CCATTCCAGCAATTCGGCCGGGACATCGCCGACACCACCGACGCCGTGCGGGATCCTCAG |
| | ACCCTCGAAATCCTGGACATCACCCCCTGTAGCTTCGGCGGCGTGAGCGTGATCACCCCT |
| | GGCACAAACACCAGCAATCAAGTGGCTGTCCTGTACCAGGATGTCAATTGCACAGAAGTG |
| | CCTGTGGCCATCCACGCCGATCAGCTGACCCCCACCTGGCGGGTGTACTCGACAGGAAGC |
| | AACGTGTTTCAAACAAGAGCCGGCTGCCTGATCGGGGCCGAGCACGTGAACAATTCCTAC |
| | GAGTGCGACATCCCCATCGGCGCCGGCATCTGTGCCTCTTACCAGACACAGACCAATTCC |
| | CCTggtagtgcaagtTCCGTGGCCAGCCAGAGCATCATCGCCTACACCATGAGCCTGGGC |
| | GCCGAAAACAGCGTTGCATATTCCAACAACAGCATCGCCATCCCTACCAACTTCACCATC |
| | AGCGTGACCACAGAAATCCTGCCTGTGTCCATGACCAAGACAAGCGTTGATTGCACCATG |
| | TACATCTGCGGCGATAGCACAGAGTGCAGCAATCTGCTGCTGCAGTACGGTAGCTTCTGC |
| | ACCCAGCTGAATAGAGCCCTGACCGGCATCGCTGTGGAACAGGACAAAAACACCCAGGAG |
| | GTCTTCGCCCAGGTGAAGCAAATCTACAAGACCCCTCCAATCAAGGACTTCGGAGGCTTT |
| | AACTTTAGCCAGATCCTGCCTGATCCCTCCAAGCCTAGCAAACGGAGTcctATCGAGGAC |
| | CTGCTCTTCAACAAGGTGACCCTGGCTGACGCCGGCTTCATTAAGCAGTACGGCGATTGC |
| | CTCGGCGACATCGCTGCAAGAGACCTGATCTGCGCCCAGAAGTTCAACGGCCTGACCGTG |
| | CTGCCTCCTCTCCTGACAGACGAGATGATCGCCCAGTACACCTCTGCCCTTCTGGCTGGC |
| | ACCATCACCAGCGGATGGACCTTTGGAGCCGGAcctGCCCTGCAGATCCCTTTCcctATG |
| | CAGATGGCCTACAGATTCAACGGGATCGGAGTGACCCAAAACGTGCTGTATGAAAACCAG |
| | AAACTGATCGCCAATCAGTTTAACAGCGCCATCGGCAAAATCCAGGATAGCCTGTCCAGC |
| | ACCccaAGCGCCCTCGGCAAGCTGCAAGATGTGGTGAATCAAAATGCCCAAGCCCTGAAC |
| | ACACTGGTGAAGCAGCTGAGCAGCAACTTCGGCGCCATCAGCAGCGTGCTGAACGACATC |
| | CTGAGCAGACTGGACccacctGAAGCCGAGGTGCAGATCGACAGACTGATCACAGGCAGA |
| | CTGCAGTCCCTGCAGACCTACGTGACCCAGCAGTTGATTAGAGCCGCTGAGATTAGAGCC |
| | AGTGCCAACCTGGCTGCCACAAAGATGTCAGAATGCGTGCTGGGCCAGAGCAAGAGAGTG |
| | GACTTCTGCGGCAAAGGCTACCACCTGATGAGCTTTCCTCAGTCTGCACCCCACGGCGTG |
| | GTGTTTCTCCACGTGACATACGTGCCCGCGCAAGAAAAGAACTTTACAACCGCCCCAGCG |
| | ATCTGCCACGACGGCAAGGCCCACTTCCCTCGGGAGGGTGTGTTCGTGAGCAATGGAACA |
| | CACTGGTTCGTCACCCAGCGGAACTTCTACGAGCCTCAGATCATTACCACCGACAACACC |
| | TTCGTGAGCGGCAACTGTGACGTCGTTATCGGCATCGTGAACAATACCGTGTACGACCCC |
| | CTGCAGCCTGAGCTGGATAGCTTCAAAGAGGAACTGGACAAGTACTTCAAGAACCACACA |
| | AGCCCCGACGTGGACCTAGGCGACATCTCTGGAATCAACGCCAGCGTGGTGAACATCCAA |
| | AAGGAAATCGACAGACTGAACGAGGTGGCCAAGAATCTGAATGAAAGCCTGATCGATCTG |
| | CAGGAGCTGGGCAAGTACGAGCAGggtggcggtggctcgCTGATTACCTATATCGTCCTG |
| | ACTATTATCTCCCTGGTGTTTGGCATTCTGTCCCTGATTCTGGCCTGTTACCTGATGTAC |
| | AAGCAGAAGGCCCAGCAGAAGACCCTGCTGTGGCTGGGCAATAATACACTGGATCAGATG |
| | CGGGCTACAACTAAGATGTGA |
| SEQ ID NO: | MFVFLVLLPLVSSQCVNLTTRTQLPPAYTNSFTRGVYYPDKVFRSSVLHSTQDLFLPFFS |
| 41 | NVTWFHAIHVSGTNGTKRFDNPVLPFNDGVYFASTEKSNIIRGWIFGTTLDSKTQSLLIV |
| chimeric | NNATNVVIKVCEFQFCNDPFLGVYYHKNNKSWMESEFRVYSSANNCTFEYVSQPFLMDLE |
| SARS-CoV-2 | GKQGNFKNLREFVFKNIDGYFKIYSKHTPINLVRDLPQGFSALEPLVDLPIGINITRFQT |
| spike protein | LLALHRSYLTPGDSSSGWTAGAAAYYVGYLQPRTFLLKYNENGTITDAVDCALDPLSETK |
| containing | CTLKSFTVEKGIYQTSNFRVQPTESIVRFPNITNLCPFGEVFNATRFASVYAWNRKRISN |
| the | CVADYSVLYNSASFSTFKCYGVSPTKLNDLCFTNVYADSFVIRGDEVRQIAPGQTGKIAD |
| transmembrane | YNYKLPDDFTGCVIAWNSNNLDSKVGGNYNYLYRLFRKSNLKPFERDISTEIYQAGSTPC |
| (TM) and | NGVEGFNCYFPLQSYGFQPTNGVGYQPYRVVVLSFELLHAPATVCGPKKSTNLVKNKCVN |
| cytoplasmic | FNFNGLTGTGVLTESNKKFLPFQQFGRDIADTTDAVRDPQTLEILDITPCSFGGVSVITP |
| (CT) domain | GTNTSNQVAVLYQDVNCTEVPVAIHADQLTPTWRVYSTGSNVFQTRAGCLIGAEHVNNSY |
| of the NDV F | ECDIPIGAGICASYQTQTNSPGSASSVASQSIIAYTMSLGAENSVAYSNNSIAIPTNFTI |
| protein | SVTTEILPVSMTKTSVDCTMYICGDSTECSNLLLQYGSFCTQLNRALTGIAVEQDKNTQE |
| | VFAQVKQIYKTPPIKDFGGFNFSQILPDPSKPSKRSPIEDLLFNKVTLADAGFIKQYGDC |
| | LGDIAARDLICAQKFNGLTVLPPLLTDEMIAQYTSALLAGTITSGWTFGAGPALQIPFPM |
| | QMAYRFNGIGVTQNVLYENQKLIANQFNSAIGKIQDSLSSTPSALGKLQDVVNQNAQALN |
| | TLVKQLSSNFGAISSVLNDILSRLDPPEAEVQIDRLITGRLQSLQTYVTQQLIRAAEIRA |
| | SANLAATKMSECVLGQSKRVDFCGKGYHLMSFPQSAPHGVVFLHVTYVPAQEKNFTTAPA |
| | ICHDGKAHFPREGVFVSNGTHWFVTQRNFYEPQIITTDNTFVSGNCDVVIGIVNNTVYDP |
| | LQPELDSFKEELDKYFKNHTSPDVDLGDISGINASVVNIQKEIDRLNEVAKNLNESLIDL |
| | QELGKYEQGGGGSLITYIVLTIISLVFGILSLILACYLMYKQKAQQKTLLWLGNNTLDQM |
| | RATTKM |
| SEQ ID NO: | TAATACGACTCACTATAGGGACCAAACAGAGAATCCGTGAGTTACGATAAAAGGCGAAGG |
| 42 | AGCAATTGAAGTCGCACGGGTAGAAGGTGTGAATCTCGAGTGCGAGCCCGAAGCACAAAC |
| NDV- | TCGAGAAAGCCTTCTGCCAACATGTCTTCCGTATTTGATGAGTACGAACAGCTCCTCGCG |
| Molecular | GCTCAGACTCGCCCCAATGGAGCTCATGGAGGGGGAGAAAAAGGGAGTACCTTAAAAGTA |
| Clone | GACGTCCCGGTATTCACTCTTAACAGTGATGACCCAGAAGATAGATGGAGCTTTGTGGTA |
| AF077761.1_ | TTCTGCCTCCGGATTGCTGTTAGCGAAGATGCCAACAAACCACTCAGGCAAGGTGCTCTC |
| LaSota_Kan | ATATCTCTTTTATGCTCCCACTCACAGGTAATGAGGAACCATGTTGCCATTGCAGGGAAA |
| R (with | CAGAATGAAGCCACATTGGCCGTGCTTGAGATTGATGGCTTTGCCAACGGCACGCCCCAG |
| stabilizing | TTCAACAATAGGAGTGGAGTGTCTGAAGAGAGAGCACAGAGATTTGCGATGATAGCAGGA |
| sequence in | TCTCTCCCTCGGGCATGCAGCAACGGAACCCCGTTCGTCACAGCCGGGGCAGAAGATGAT |
| L) backbone | GCACCAGAAGACATCACCGATACCCTGGAGAGGATCCTCTCTATCCAGGCTCAAGTATGG |
| | GTCACAGTAGCAAAAGCCATGACTGCGTATGAGACTGCAGATGAGTCGGAAACAAGGCGA |
| | ATCAATAAGTATATGCAGCAAGGCAGGGTCCAAAAGAAATACATCCTCTACCCCGTATGC |
| | AGGAGCACAATCCAACTCACGATCAGACAGTCTCTTGCAGTCCGCATCTTTTTGGTTAGC |
| | GAGCTCAAGAGAGGCCGCAACACGGCAGGTGGTACCTCTACTTATTATAACCTGGTAGGG |
| | GACGTAGACTCATACATCAGGAATACCGGGCTTACTGCATTCTTCTTGACACTCAAGTAC |
| | GGAATCAACACCAAGACATCAGCCCTTGCACTTAGTAGCCTCTCAGGCGACATCCAGAAG |
| | ATGAAGCAGCTCATGCGTTTGTATCGGATGAAAGGAGATAATGCGCCGTACATGACATTA |
| | CTTGGTGATAGTGACCAGATGAGCTTTGCGCCTGCCGAGTATGCACAACTTTACTCCTTT |
| | GCCATGGGTATGGCATCAGTCCTAGATAAAGGTACTGGGAAATACCAATTTGCCAGGGAC |
| | TTTATGAGCACATCATTCTGGAGACTTGGAGTAGAGTACGCTCAGGCTCAGGGAAGTAGC |
| | ATTAACGAGGATATGGCTGCCGAGCTAAAGCTAACCCCAGCAGCAATGAAGGGCCTGGCA |
| | GCTGCTGCCCAACGGGTCTCCGACGATACCAGCAGCATATACATGCCTACTCAACAAGTC |
| | GGAGTCCTCACTGGGCTTAGCGAGGGGGGGTCCCAAGCTCTACAAGGCGGATCGAATAGA |
| | TCGCAAGGGCAACCAGAAGCCGGGGATGGGGAGACCCAATTCCTGGATCTGATGAGAGCG |
| | GTAGCAAATAGCATGAGGGAGGCGCCAAACTCTGCACAGGGCACTCCCCAATCGGGGCCT |
| | CCCCCAACTCCTGGGCCATCCCAAGATAACGACACCGACTGGGGGTATTGATGGACAAAA |
| | CCCAGCCTGCTTCCACAAAAACATCCCAATGCCCTCACCCGTAGTCGACCCCTCGATTTG |
| | CGGCTCTATATGACCACACCCTCAAACAAACATCCCCCTCTTTCCTCCCTCCCCCTGCTG |
| | TACAACTCCGCACGCCCTAGATACCACAGGCACAATGCGGCTCACTAACAATCAAAACAG |
| | AGCCGAGGGAATTAGAAAAAAGTACGGGTAGAAGAGGGATATTCAGAGATCAGGGCAAGT |
| | CTCCCGAGTCTCTGCTCTCTCCTCTACCTGATAGACCAGGACAAACATGGCCACCTTTAC |
| | AGATGCAGAGATCGACGAGCTATTTGAGACAAGTGGAACTGTCATTGACAACATAATTAC |
| | AGCCCAGGGTAAACCAGCAGAGACTGTTGGAAGGAGTGCAATCCCACAAGGCAAGACCAA |
| | GGTGCTGAGCGCAGCATGGGAGAAGCATGGGAGCATCCAGCCACCGGCCAGTCAAGACAA |
| | CCCCGATCGACAGGACAGATCTGACAAACAACCATCCACACCCGAGCAAACGACCCCGCA |
| | TGACAGCCCGCCGGCCACATCCGCCGACCAGCCCCCCACCCAGGCCACAGACGAAGCCGT |
| | CGACACACAGTTCAGGACCGGAGCAAGCAACTCTCTGCTGTTGATGCTTGACAAGCTCAG |
| | CAATAAATCGTCCAATGCTAAAAAGGGCCCATGGTCGAGCCCCCAAGAGGGGAATCACCA |
| | ACGTCCGACTCAACAGCAGGGGAGTCAACCCAGTCGCGGAAACAGTCAGGAAAGACCGCA |
| | GAACCAAGTCAAGGCCGCCCCTGGAAACCAGGGCACAGACGTGAACACAGCATATCATGG |
| | ACAATGGGAGGAGTCACAACTATCAGCTGGTGCAACCCCTCATGCTCTCCGATCAAGGCA |
| | GAGCCAAGACAATACCCTTGTATCTGCGGATCATGTCCAGCCACCTGTAGACTTTGTGCA |
| | AGCGATGATGTCTATGATGGAGGCGATATCACAGAGAGTAAGTAAGGTTGACTATCAGCT |
| | AGATCTTGTCTTGAAACAGACATCCTCCATCCCTATGATGCGGTCCGAAATCCAACAGCT |
| | GAAAACATCTGTTGCAGTCATGGAAGCCAACTTGGGAATGATGAAGATTCTGGATCCCGG |
| | TTGTGCCAACATTTCATCTCTGAGTGATCTACGGGCAGTTGCCCGATCTCACCCGGTTTT |
| | AGTTTCAGGCCCTGGAGACCCCTCTCCCTATGTGACACAAGGAGGCGAAATGGCACTTAA |
| | TAAACTTTCGCAACCAGTGCCACATCCATCTGAATTGATTAAACCCGCCACTGCATGCGG |
| | GCCTGATATAGGAGTGGAAAAGGACACTGTCCGTGCATTGATCATGTCACGCCCAATGCA |
| | CCCGAGTTCTTCAGCCAAGCTCCTAAGCAAGTTAGATGCAGCCGGGTCGATCGAGGAAAT |
| | CAGGAAAATCAAGCGCCTTGCTCTAAATGGCTAATTACTACTGCCACACGTAGCGGGTCC |
| | CTGTCCACTCGGCATCACACGGAATCTGCACCGAGTTCCCCCtctagaTTAGAAAAAATA |
| | CGGGTAGAACCGCCACCacgcgtACCCAAGGTCCAACTCTCCAAGCGGCAATCCTCTCTC |
| | GCTTCCTCAGCCCCACTGAATGGTCGCGTAACCGTAATTAATCTAGCTACATTTAAGATT |
| | AAGAAAAAATACGGGTAGAATTGGAGTGCCCCAATTGTGCCAAGATGGACTCATCTAGGA |
| | CAATTGGGCTGTACTTTGATTCTGCCCATTCTTCTAGCAACCTGTTAGCATTTCCGATCG |
| | TCCTACAAGGCACAGGAGATGGGAAGAAGCAAATCGCCCCGCAATATAGGATCCAGCGCC |
| | TTGACTTGTGGACTGATAGTAAGGAGGACTCAGTATTCATCACCACCTATGGATTCATCT |
| | TTCAAGTTGGGAATGAAGAAGCCACTGTCGGCATGATCGATGATAAACCCAAGCGCGAGT |
| | TACTTTCCGCTGCGATGCTCTGCCTAGGAAGCGTCCCAAATACCGGAGACCTTATTGAGC |
| | TGGCAAGGGCCTGTCTCACTATGATAGTCACATGCAAGAAGAGTGCAACTAATACTGAGA |
| | GAATGGTTTTCTCAGTAGTGCAGGCACCCCAAGTGCTGCAAAGCTGTAGGGTTGTGGCAA |
| | ACAAATACTCATCAGTGAATGCAGTCAAGCACGTGAAAGCGCCAGAGAAGATTCCCGGGA |
| | GTGGAACCCTAGAATACAAGGTGAACTTTGTCTCCTTGACTGTGGTACCGAAGAAGGATG |
| | TCTACAAGATCCCAGCTGCAGTATTGAAGGTTTCTGGCTCGAGTCTGTACAATCTTGCGC |
| | TCAATGTCACTATTAATGTGGAGGTAGACCCGAGGAGTCCTTTGGTTAAATCTTTGTCTA |
| | AGTCTGACAGCGGATACTATGCTAACCTCTTCTTGCATATTGGACTTATGACCACCGTAG |
| | ATAGGAAGGGGAAGAAAGTGACATTTGACAAGCTGGAAAAGAAAATAAGGAGCCTTGATC |
| | TATCTGTCGGGCTCAGTGATGTGCTCGGGCCTTCCGTGTTGGTAAAAGCAAGAGGTGCAC |
| | GGACTAAGCTTTTGGCACCTTTCTTCTCTAGCAGTGGGACAGCCTGCTATCCCATAGCAA |
| | ATGCTTCTCCTCAGGTGGCCAAGATACTCTGGAGTCAAACCGCGTGCCTGCGGAGCGTTA |
| | AAATCATTATCCAAGCAGGTACCCAACGCGCTGTCGCAGTGACCGCCGACCACGAGGTTA |
| | CCTCTACTAAGCTGGAGAAGGGGCACACCCTTGCCAAATACAATCCTTTTAAGAAATAAG |
| | CTGCGTCTCTGAGATTGCGCTCCGCCCACTCACCCAGATCATCATGACACAAAAAACTAA |
| | TCTGTCTTGATTATTTACAGTTAGTTTACCTGTCTATCAAGTTAGAAAAAACACGGGTAG |
| | AAGATTCTGGATCCCGGTTGGCGCCCTCCAGGTGCAAGttaattaaATGGGCTCCAGACC |
| | TTCTACCAAGAACCCAGCACCTATGATGCTGACTATCCGGGTTGCGCTGGTACTGAGTTG |
| | CATCTGTCCGGCAAACTCCATTGATGGCAGGCCTCTTGCAGCTGCAGGAATTGTGGTTAC |
| | AGGAGACAAAGCCGTCAACATATACACCTCATCCCAGACAGGATCAATCATAGTTAAGCT |
| | CCTCCCGAATCTGCCCAAGGATAAGGAGGCATGTGCGAAAGCCCCCTTGGATGCATACAA |
| | CAGGACATTGACCACTTTGCTCACCCCCCTTGGTGACTCTATCCGTAGGATACAAGAGTC |
| | TGTGACTACATCTGGAGGGGGGAGACAGGGGCGCCTTATAGGCGCCATTATTGGCGGTGT |
| | GGCTCTTGGGGTTGCAACTGCCGCACAAATAACAGCGGCCGCAGCTCTGATACAAGCCAA |
| | ACAAAATGCTGCCAACATCCTCCGACTTAAAGAGAGCATTGCCGCAACCAATGAGGCTGT |
| | GCATGAGGTCACTGACGGATTATCGCAACTAGCAGTGGCAGTTGGGAAGATGCAGCAGTT |
| | TGTTAATGACCAATTTAATAAAACAGCTCAGGAATTAGACTGCATCAAAATTGCACAGCA |
| | AGTTGGTGTAGAGCTCAACCTGTACCTAACCGAATTGACTACAGTATTCGGACCACAAAT |
| | CACTTCACCTGCTTTAAACAAGCTGACTATTCAGGCACTTTACAATCTAGCTGGTGGAAA |
| | TATGGATTACTTATTGACTAAGTTAGGTGTAGGGAACAATCAACTCAGCTCATTAATCGG |
| | TAGCGGCTTAATCACtGGcAACCCTATTCTATACGACTCACAGACTCAACTCTTGGGTAT |
| | ACAGGTAACTgcaCCTTCAGTCGGGAACCTAAATAATATGCGTGCCACCTACTTGGAAAC |
| | CTTATCCGTAAGCACAACCAGGGGATTTGCCTCGGCACTTGTCCCCAAAGTGGTGACACA |
| | GGTCGGTTCTGTGATAGAAGAACTTGACACCTCATACTGTATAGAAACTGACTTAGATTT |
| | ATATTGTACAAGAATAGTAACGTTCCCTATGTCCCCTGGTATTTATTCCTGCTTGAGCGG |
| | CAATACGTCGGCCTGTATGTACTCAAAGACCGAAGGCGCACTTACTACACCATACATGAC |
| | TATCAAAGGTTCAGTCATCGCCAACTGCAAGATGACAACATGTAGATGTGTAAACCCCCC |
| | GGGTATCATATCGCAAAACTATGGAGAAGCCGTGTCTCTAATAGATAAACAATCATGCAA |
| | TGTTTTATCCTTAGGCGGGATAACTTTAAGGCTCAGTGGGGAATTCGATGTAACTTATCA |
| | GAAGAATATCTCAATACAAGATTCTCAAGTAATAATAACAGGCAATCTTGATATCTCAAC |
| | TGAGCTTGGGAATGTCAACAACTCGATCAGTAATGCTTTGAATAAGTTAGAGGAAAGCAA |
| | CAGAAAACTAGACAAAGTCAATGTCAAACTGACTAGCACATCTGCTCTCATTACgTATAT |
| | CGTTTTGACTATCATATCTCTTGTTTTTGGTATACTTAGCCTGATTCTAGCATGCTACCT |
| | AATGTACAAGCAAAAGGCGCAACAAAAGACCTTATTATGGCTTGGGAATAATACaCTcGA |
| | TCAGATGAGAGCCACTACAAAAATGTGAACACAGATGAGGAACGAAGGTTTCCCTAATAG |
| | TAATTTGTGTGAAAGTTCTGGTAGTCTGTCAGTTCAGAGAGTTAAGAAAAAACTACCGGT |
| | TGTAGATGACCAAAGGACGATATACGGGTAGAACGGTAAGAGAGGCCGCCCCTCAATTGC |
| | GAGCCAGGCTTCACAACCTCCGTTCTACCGCTTCACCGACAACAGTCCTCAATCATGGAC |
| | CGCGCCGTTAGCCAAGTTGCGTTAGAGAATGATGAAAGAGAGGCAAAAAATACATGGCGC |
| | TTGATATTCCGGATTGCAATCTTATTCTTAACAGTAGTGACCTTGGCTATATCTGTAGCC |
| | TCCCTTTTATATAGCATGGGGGCTAGCACACCTAGCGATCTTGTAGGCATACCGACTAGG |
| | ATTTCCAGGGCAGAAGAAAAGATTACATCTACACTTGGTTCCAATCAAGATGTAGTAGAT |
| | AGGATATATAAGCAAGTGGCCCTTGAGTCTCCGTTGGCATTGTTAAATACTGAGACCACA |
| | ATTATGAACGCAATAACATCTCTCTCTTATCAGATTAATGGAGCTGCAAACAACAGTGGG |
| | TGGGGGGCACCTATCCATGACCCAGATTATATAGGGGGGATAGGCAAAGAACTCATTGTA |
| | GATGATGCTAGTGATGTCACATCATTCTATCCCTCTGCATTTCAAGAACATCTGAATTTT |
| | ATCCCGGCGCCTACTACAGGATCAGGTTGCACTCGAATACCCTCATTTGACATGAGTGCT |
| | ACCCATTACTGCTACACCCATAATGTAATATTGTCTGGATGCAGAGATCACTCACATTCA |
| | TATCAGTATTTAGCACTTGGTGTGCTCCGGACATCTGCAACAGGGAGGGTATTCTTTTCT |
| | ACTCTGCGTTCCATCAACCTGGACGACACCCAAAATCGGAAGTCTTGCAGTGTGAGTGCA |
| | ACTCCCCTGGGTTGTGATATGCTGTGCTCGAAAGTCACGGAGACAGAGGAAGAAGATTAT |
| | AACTCAGCTGTCCCTACGCGGATGGTACATGGGAGGTTAGGGTTCGACGGCCAGTACCAC |
| | GAAAAGGACCTAGATGTCACAACATTATTCGGGGACTGGGTGGCCAACTACCCAGGAGTA |
| | GGGGGTGGATCTTTTATTGACAGCCGCGTATGGTTCTCAGTCTACGGAGGGTTAAAACCC |
| | AATTCACCCAGTGACACTGTACAGGAAGGGAAATATGTGATATACAAGCGATACAATGAC |
| | ACATGCCCAGATGAGCAAGACTACCAGATTCGAATGGCCAAGTCTTCGTATAAGCCTGGA |
| | CGGTTTGGTGGGAAACGCATACAGCAGGCTATCTTATCTATCAAGGTGTCAACATCCTTA |
| | GGCGAAGACCCGGTACTGACTGTACCGCCCAACACAGTCACACTCATGGGGGCCGAAGGC |
| | AGAATTCTCACAGTAGGGACATCTCATTTCTTGTATCAACGAGGGTCATCATACTTCTCT |
| | CCCGCGTTATTATATCCTATGACAGTCAGCAACAAAACAGCCACTCTTCATAGTCCTTAT |
| | ACATTCAATGCCTTCACTCGGCCAGGTAGTATCCCTTGCCAGGCTTCAGCAAGATGCCCC |
| | AACTCGTGTGTTACTGGAGTCTATACAGATCCATATCCCCTAATCTTCTATAGAAACCAC |
| | ACCTTGCGAGGGGTATTCGGGACAATGCTTGATGGTGTACAAGCAAGACTTAACCCTGCG |
| | TCTGCAGTATTCGATAGCACATCCCGCAGTCGCATTACTCGAGTGAGTTCAAGCAGTACC |
| | AAAGCAGCATACACAACATCAACTTGTTTTAAAGTGGTCAAGACTAATAAGACCTATTGT |
| | CTCAGCATTGCTGAAATATCTAATACTCTCTTCGGAGAATTCAGAATCGTCCCGTTACTA |
| | GTTGAGATCCTCAAAGATGACGGGGTTAGAGAAGCCAGGTCTGGCTAGggcgcgccTTGA |
| | GTCAATTATAAAGGAGTTGGAAAGATGGCATTGTATCACCTATCTTCTGCGACATCAAGA |
| | ATCAAACCGAATGCCGGCGCGTGCTCGAATTCCATGTTGCCAGTTGACCACAATCAGCCA |
| | GTGCTCATGCGATCAGATTAAGCCTTGTCATTAATCTCTTGATTAAGAAAAAATGTAAGT |
| | GGCAATGAGATACAAGGCAAAACAGCTCATGGTAAATAATACGGGTAGGACATGGCGAGC |
| | TCCGGTCCTGAAAGGGCAGAGCATCAGATTATCCTACCAGAGCCACACCTGTCTTCACCA |
| | TTGGTCAAGCACAAACTACTCTATTACTGGAAATTAACTGGGCTACCGCTTCCTGATGAA |
| | TGTGACTTCGACCACCTCATTCTCAGCCGACAATGGAAAAAAATACTTGAATCGGCCTCT |
| | CCTGATACTGAGAGAATGATAAAACTCGGAAGGGCAGTACACCAAACTCTTAACCACAAT |
| | TCCAGAATAACCGGAGTGCTCCACCCCAGGTGTTTAGAACAACTGGCTAATATTGAGGTC |
| | CCAGATTCAACCAACAAATTTCGGAAGATTGAGAAGAAGATCCAAATTCACAACACGAGA |
| | TATGGAGAACTGTTCACAAGGCTGTGTACGCATATAGAGAAGAAACTGCTGGGGTCATCT |
| | TGGTCTAACAATGTCCCCCGGTCAGAGGAGTTCAGCAGCATTCGTACGGATCCGGCATTC |
| | TGGTTTCACTCAAAATGGTCCACAGCCAAGTTTGCATGGCTCCATATAAAACAGATCCAG |
| | AGGCATCTGATGGTGGCAGCTAAGACAAGGTCTGCGGCCAACAAATTGGTGATGCTAACC |
| | CATAAGGTAGGCCAAGTCTTTGTCACTCCTGAACTTGTCGTTGTGACGCATACGAATGAG |
| | AACAAGTTCACATGTCTTACCCAGGAACTTGTATTGATGTATGCAGATATGATGGAGGGC |
| | AGAGATATGGTCAACATAATATCAACCACGGCGGTGCATCTCAGAAGCTTATCAGAGAAA |
| | ATTGATGACATTTTGCGGTTAATAGACGCTCTGGCAAAAGACTTGGGTAATCAAGTCTAC |
| | GATGTTGTATCACTAATGGAGGGATTTGCATACGGAGCTGTCCAGCTACTCGAGCCGTCA |
| | GGTACATTTGCAGGAGATTTCTTCGCATTCAACCTGCAGGAGCTTAAAGACATTCTAATT |
| | GGCCTCCTCCCCAATGATATAGCAGAATCCGTGACTCATGCAATCGCTACTGTATTCTCT |
| | GGTTTAGAACAGAATCAAGCAGCTGAGATGTTGTGTCTGTTGCGTCTGTGGGGTCACCCA |
| | CTGCTTGAGTCCCGTATTGCAGCAAAGGCAGTCAGGAGCCAAATGTGCGCACCGAAAATG |
| | GTAGACTTTGATATGATCCTTCAGGTACTGTCTTTCTTCAAGGGAACAATCATCAACGGG |
| | TACAGAAAGAAGAATGCAGGTGTGTGGCCGCGAGTCAAAGTGGATACAATATATGGGAAG |
| | GTCATTGGGCAACTACATGCAGATTCAGCAGAGATTTCACACGATATCATGTTGAGAGAG |
| | TATAAGAGTTTATCTGCACTTGAATTTGAGCCATGTATAGAATATGACCCTGTCACCAAC |
| | CTGAGCATGTTCCTAAAAGACAAGGCAATCGCACACCCCAACGATAATTGGCTTGCCTCG |
| | TTTAGGCGGAACCTTCTCTCCGAAGACCAGAAGAAACATGTAAAAGAAGCAACTTCGACT |
| | AATCGCCTCTTGATAGAGTTTTTAGAGTCAAATGATTTTGATCCATATAAAGAGATGGAA |
| | TATCTGACGACCCTTGAGTACCTTAGAGATGACAATGTGGCAGTATCATACTCGCTCAAG |
| | GAGAAGGAAGTGAAAGTTAATGGACGGATCTTCGCTAAGCTGACAAAGAAGTTAAGGAAC |
| | TGTCAGGTGATGGCGGAAGGGATCCTAGCCGATCAGATTGCACCTTTCTTTCAGGGAAAT |
| | GGAGTCATTCAGGATAGCATATCCTTGACCAAGAGTATGCTAGCGATGAGTCAACTGTCT |
| | TTTAACAGCAATAAGAAACGTATCACTGACTGTAAAGAAAGAGTATCTTCAAACCGCAAT |
| | CATGATCCGAAAAGCAAGAACCGTCGGAGAGTTGCAACCTTCATAACAACTGACCTGCAA |
| | AAGTACTGTCTTAATTGGAGATATCAGACAATCAAATTGTTCGCTCATGCCATCAATCAG |
| | TTGATGGGCCTACCTCACTTCTTCGAATGGATTCACCTAAGACTGATGGACACTACGATG |
| | TTCGTAGGAGACCCTTTCAATCCTCCAAGTGACCCTACTGACTGTGACCTCTCAAGAGTC |
| | CCTAATGATGACATATATATTGTCAGTGCCAGAGGGGGTATCGAAGGATTATGCCAGAAG |
| | CTATGGACAATGATCTCAATTGCTGCAATCCAACTTGCTGCAGCTAGATCGCATTGTCGT |
| | GTTGCCTGTATGGTACAGGGTGATAATCAAGTAATAGCAGTAACGAGAGAGGTAAGATCA |
| | GACGACTCTCCGGAGATGGTGTTGACACAGTTGCATCAAGCCAGTGATAATTTCTTCAAG |
| | GAATTAATTCATGTCAATCATTTGATTGGCCATAATTTGAAGGATCGTGAAACCATCAGG |
| | TCAGACACATTCTTCATATACAGCAAACGAATCTTCAAAGATGGAGCAATCCTCAGTCAA |
| | GTCCTCAAAAATTCATCTAAATTAGTGCTAGTGTCAGGTGATCTCAGTGAAAACACCGTA |
| | ATGTCCTGTGCCAACATTGCCTCTACTGTAGCACGGCTATGCGAGAACGGGCTTCCCAAA |
| | GACTTCTGTTACTATTTAAACTATATAATGAGTTGTGTGCAGACATACTTTGACTCTGAG |
| | TTCTCCATCACCAACAATTCGCACCCCGATCTTAATCAGTCGTGGATTGAGGACATCTCT |
| | TTTGTGCACTCATATGTTCTGACTCCTGCCCAATTAGGGGGACTGAGTAACCTTCAATAC |
| | TCAAGGCTCTACACTAGAAATATCGGTGACCCGGGGACTACTGCTTTTGCAGAGATCAAG |
| | CGACTAGAAGCAGTGGGATTACTGAGTCCTAACATTATGACTAATATCTTAACTAGGCCG |
| | CCTGGGAATGGAGATTGGGCCAGTCTGTGCAACGACCCATACTCTTTCAATTTTGAGACT |
| | GTTGCAAGCCCAAATATTGTTCTTAAGAAACATACGCAAAGAGTCCTATTTGAAACTTGT |
| | TCAAATCCCTTATTGTCTGGAGTGCACACAGAGGATAATGAGGCAGAAGAGAAGGCATTG |
| | GCTGAATTCTTGCTTAATCAAGAGGTGATTCATCCCCGCGTTGCGCATGCCATCATGGAG |
| | GCAAGCTCTGTAGGTAGGAGAAAGCAAATTCAAGGGCTTGTTGACACAACAAACACCGTA |
| | ATTAAGATTGCGCTTACTAGGAGGCCATTAGGCATCAAGAGGCTGATGCGGATAGTCAAT |
| | TATTCTAGCATGCATGCAATGCTGTTTAGAGACGATGTTTTTTCCTCCAGTAGATCCAAC |
| | CACCCCTTAGTCTCTTCTAATATGTGTTCTCTGACACTGGCAGACTATGCACGGAATAGA |
| | AGCTGGTCACCTTTGACGGGAGGCAGGAAAATACTGGGTGTATCTAATCCTGATACGATA |
| | GAACTCGTAGAGGGTGAGATTCTTAGTGTAAGCGGAGGGTGTACAAGATGTGACAGCGGA |
| | GATGAACAATTTACTTGGTTCCATCTTCCAAGCAATATAGAATTGACCGATGACACCAGC |
| | AAGAATCCTCCGATGAGGGTACCATATCTCGGGTCAAAGACACAGGAGAGGAGAGCTGCC |
| | TCACTTGCAAAAATAGCTCATATGTCGCCACATGTAAAGGCTGCCCTAAGGGCATCATCC |
| | GTGTTGATCTGGGCTTATGGGGATAATGAAGTAAATTGGACTGCTGCTCTTACGATTGCA |
| | AAATCTCGGTGTAATGTAAACTTAGAGTATCTTCGGTTACTGTCCCCTTTACCCACGGCT |
| | GGGAATCTTCAACATAGACTAGATGATGGTATAACTCAGATGACATTCACCCCTGCATCT |
| | CTCTACAGGgtgtcaccttacattcacatatccaatgattctcaaaggctgttcactgaa |
| | gaaggagtcaaagaggggaatgtggtttaccaacagatcATGCTCTTGGGTTTATCTCTA |
| | ATCGAATCGATCTTTCCAATGACAACAACCAGGACATATGATGAGATCACACTGCACCTA |
| | CATAGTAAATTTAGTTGCTGTATCAGAGAAGCACCTGTTGCGGTTCCTTTCGAGCTACTT |
| | GGGGTGGTACCGGAACTGAGGACAGTGACCTCAAATAAGTTTATGTATGATCCTAGCCCT |
| | GTATCGGAGGGAGACTTTGCGAGACTTGACTTAGCTATCTTCAAGAGTTATGAGCTTAAT |
| | CTGGAGTCATATCCCACGATAGAGCTAATGAACATTCTTTCAATATCCAGCGGGAAGTTG |
| | ATTGGCCAGTCTGTGGTTTCTTATGATGAAGATACCTCCATAAAGAATGACGCCATAATA |
| | GTGTATGACAATACCCGAAATTGGATCAGTGAAGCTCAGAATTCAGATGTGGTCCGCCTA |
| | TTTGAATATGCAGCACTTGAAGTGCTCCTCGACTGTTCTTACCAACTCTATTACCTGAGA |
| | GTAAGAGGCCTAGACAATATTGTCTTATATATGGGTGATTTATACAAGAATATGCCAGGA |
| | ATTCTACTTTCCAACATTGCAGCTACAATATCTCATCCCGTCATTCATTCAAGGTTACAT |
| | GCAGTGGGCCTGGTCAACCATGACGGATCACACCAACTTGCAGATACGGATTTTATCGAA |
| | ATGTCTGCAAAACTATTAGTATCTTGCACCCGACGTGTGATCTCCGGCTTATATTCAGGA |
| | AATAAGTATGATCTGCTGTTCCCATCTGTCTTAGATGATAACCTGAATGAGAAGATGCTT |
| | CAGCTGATATCCCGGTTATGCTGTCTGTACACGGTACTCTTTGCTACAACAAGAGAAATC |
| | CCGAAAATAAGAGGCTTAACTGCAGAAGAGAAATGTTCAATACTCACTGAGTATTTACTG |
| | TCGGATGCTGTGAAACCATTACTTAGCCCCGATCAAGTGAGCTCTATCATGTCTCCTAAC |
| | ATAATTACATTCCCAGCTAATCTGTACTACATGTCTCGGAAGAGCCTCAATTTGATCAGG |
| | GAAAGGGAGGACAGGGATACTATCCTGGCGTTGTTGTTCCCCCAAGAGCCATTATTAGAG |
| | TTCCCTTCTGTGCAAGATATTGGTGCTCGAGTGAAAGATCCATTCACCCGACAACCTGCG |
| | GCATTTTTGCAAGAGTTAGATTTGAGTGCTCCAGCAAGGTATGACGCATTCACACTTAGT |
| | CAGATTCATCCTGAACTCACATCTCCAAATCCGGAGGAAGACTACTTAGTACGATACTTG |
| | TTCAGAGGGATAGGGACTGCATCTTCCTCTTGGTATAAGGCATCTCATCTCCTTTCTGTA |
| | CCCGAGGTAAGATGTGCAAGACACGGGAACTCCTTATACTTAGCTGAAGGGAGCGGAGCC |
| | ATCATGAGTCTTCTCGAACTGCATGTACCACATGAAACTATCTATTACAATACGCTCTTT |
| | TCAAATGAGATGAACCCCCCGCAACGACATTTCGGGCCGACCCCAACTCAGTTTTTGAAT |
| | TCGGTTGTTTATAGGAATCTACAGGCGGAGGTAACATGCAAAGATGGATTTGTCCAAGAG |
| | TTCCGTCCATTATGGAGAGAAAATACAGAGGAAAGTGACCTGACCTCAGATAAAGCAGTG |
| | GGGTATATTACATCTGCAGTGCCCTACAGATCTGTATCATTGCTGCATTGTGACATTGAA |
| | ATTCCTCCAGGGTCCAATCAAAGCTTACTAGATCAACTAGCTATCAATTTATCTCTGATT |
| | GCCATGCATTCTGTAAGGGAGGGCGGGGTAGTAATCATCAAAGTGTTGTATGCAATGGGA |
| | TACTACTTTCATCTACTCATGAACTTGTTTGCTCCGTGTTCCACAAAAGGATATATTCTC |
| | TCTAATGGTTATGCATGTCGAGGAGATATGGAGTGTTACCTGGTATTTGTCATGGGTTAC |
| | CTGGGCGGGCCTACATTTGTACATGAGGTGGTGAGGATGGCAAAAACTCTGGTGCAGCGG |
| | CACGGTACGCTCTTGTCTAAATCAGATGAGATCACACTGACCAGGTTATTCACCTCACAG |
| | CGGCAGCGTGTGACAGACATCCTATCCAGTCCTTTACCAAGATTAATAAAGTACTTGAGG |
| | AAGAATATTGACACTGCGCTGATTGAAGCCGGGGGACAGCCCGTCCGTCCATTCTGTGCG |
| | GAGAGTCTGGTGAGCACGCTAGCGAACATAACTCAGATAACCCAGATTATCGCTAGTCAC |
| | ATTGACACAGTTATCCGGTCTGTGATATATATGGAAGCTGAGGGTGATCTCGCTGACACA |
| | GTATTTCTATTTACCCCTTACAATCTCTCTACTGACGGGAAAAAGAGGACATCACTTATA |
| | CAGTGCACGAGACAGATCCTAGAGGTTACAATACTAGGTCTTAGAGTCGAAAATCTCAAT |
| | AAAATAGGCGATATAATCAGCCTAGTGCTTAAAGGCATGATCTCCATGGAGGACCTTATC |
| | CCACTAAGGACATACTTGAAGCATAGTACCTGCCCTAAATATTTGAAGGCTGTCCTAGGT |
| | ATTACCAAACTCAAAGAAATGTTTACAGACACTTCTGTATTGTACTTGACTCGTGCTCAA |
| | CAAAAATTCTACATGAAAACTATAGGCAATGCAGTCAAAGGATATTACAGTAACTGTGAG |
| | TCTTAACGAAAATCACATATTAATAGGCTCCTTTTTTGGCCAATTGTATTCTTGTTGATT |
| | TAATCATATTATGTTAGAAAAAAGTTGAACCCTGACTCCTTAGGACTCGAATTCGAACTC |
| | AAATAAATGTCTTAAAAAAAGGTTGCGCACAATTATTCTTGAGTGTAGTCTCGTCATTCA |
| | CCAAATCTTTGTTTGGTGGCCGGCATGGTCCCAGCCTCCTCGCTGGCGCCGGCTGGGCAA |
| | CATTCCGAGGGGACCGTCCCCTCGGTAATGGCGAATGGGACGTCGACTGCTAACAAAGCC |
| | CGAAAGGAAGCTGAGTTGGCTGCTGCCACCGCTGAGCAATAACTAGCATAACCCCTTGGG |
| | GCCTCTAAACGGGTCTTGAGGGGTTTTTTGCTGAAAGGAGGAACTATA |

Inventors have also engineered and rescued a chimeric NDV virus that has the F protein and HN protein from avian paramyxovirus 5 (APMV5) (SEQ ID NO: 9). F protein and HN protein are constituents of the NDV envelope, embedded within the lipid bilayer membrane. The inventors designed and produced this chimeric virus because the APMV5 F gene has a multi-basic cleavage site, which, without wishing to be bound by theory, can be useful for fusion with cells. Since APMV-5 is not pathogenic in chickens, the swapping of portion of APMV5 F protein with NDV F protein would broaden the use of this virus as an oncolytic agent in jurisdictions where there are restrictions imposed on avian pathogens, for example in the US by the authority of USDA/CDC. Specifically, for the NDV-APMV5 F-HN chimeric molecular clone sequence, NDV-APMV5 F is composed mostly of APMV5 but the last 53 amino acids are from NDV. NDV-APMV5 HN is composed mostly of APMV5 but the first 53 amino acids are from NDV.

Accordingly, also provided is an engineered NDV vector comprising a nucleic acid having a nucleic acid sequence encoding a L protein comprising a stabilizing segment, a chimeric F protein, and a chimeric HN protein, wherein the chimeric F protein comprises avian paramyxovirus 5 (APMV5) F protein segment thereof at the N-terminus and an NDV F protein segment at the C-terminus, and wherein the chimeric HN protein comprises an NDV HN protein segment at the N-terminus and an AMPV5 HN protein segment at the C-terminus. In some embodiments, the nucleic acid comprises XbaI and MluI restriction endonuclease sites between nucleic acid sequence encoding phosphoprotein and matrix protein. In some embodiments, the chimeric F protein comprises at the C-terminus 53 amino acid of NDV F protein from amino acid positions 501 to 553 of SEQ ID NO: 28. In some embodiments, the chimeric HN protein comprises at the N-terminus 53 amino acids of NDV HN protein from amino acid positions 1 to 53 of SEQ ID NO: 34. In some embodiments, the stabilizing segment comprises an amino acid sequence as set forth in SEQ ID NO: 20. In some embodiments, the stabilizing segment is encoded by a nucleic acid comprising a nucleic acid sequence as set forth in SEQ ID NO: 35. In some embodiments, the L protein comprises an amino acid sequence having at least 85%, 90%, 95%, 96%, 97%, 98%, 99%, 99.5%, 99.9%, or 100% identity to the amino acid sequence as set forth in SEQ ID NO: 11. In some embodiments, the chimeric F protein comprises an amino acid sequence having at least 85%, 90%, 95%, 96%, 97%, 98%, 99%, 99.5%, 99.9%, or 100% identity to the amino acid sequence of SEQ ID NO: 12. In some embodiments, the chimeric HN protein comprises an amino acid sequence having at least 85%, 90%, 95%, 96%, 97%, 98%, 99%, 99.5%, 99.9%, or 100% identity to the amino acid sequence of SEQ ID NO: 13. In some embodiments, the nucleic acid further comprises at least one heterologous nucleic acid segment encoding a therapeutic agent operably linked to a promoter capable of expressing the segment in a host cell. In some embodiments, the therapeutic agent comprises a SARS-CoV-2 spike protein. In some embodiments, the SARS-CoV-2 spike protein comprises an amino acid sequence having at least 85%, 90%, 95%, 96%, 97%, 98%, 99%, 99.5%, 99.9%, or 100% identity to the amino acid sequence of SEQ ID NO: 6, 7, 29, 30, 31, or 41.

### Methods and Uses

The term “infectious disease”, “transmissible disease” or “communicable disease”, and their derivatives, as used herein, refer to or describe a disease or disorder resulted from an infection, for example, caused by infectious agents including viruses, viroids, prions, bacteria, nematodes such as parasitic roundworms and pinworms, arthropods such as ticks, mites, fleas, and lice, fungi such as ringworm, and other macroparasites such as tapeworms and other helminths. Examples of infectious diseases include viral diseases such as viral hemorrhagic fevers such as Ebola and Marburg virus disease, gastroenteritis, dengue fever, West Nile fever, yellow fever, influenza, respiratory syncytial virus disease, Lassa fever, rabies, smallpox, cowpox, horsepox, monkeypox, Hantavirus pulmonary syndrome, Hendra virus disease, human immunodeficiency virus infection and acquired immunodeficiency disease syndrome, Hepatitis, Zika fever, Severe Acute Respiratory Syndrome (SARS), Middle East Respiratory Syndrome (MERS), Coronavirus disease 2019 (COVID-19), infectious bronchitis, infectious laryngotracheitis, Rift Valley fever, porcine epidemic diarrhea, porcine transmissible gastroenteritis, swine acute diarrhea syndrome, feline infectious peritonitis, African swine fever, classical swine fever, and bacterial diseases including drug resistant bacterial diseases such as tuberculosis and methicillin-resistant Staphylococcus aureus infection, and drug resistant parasitic diseases such as malaria. In an embodiment of this disclosure, the infectious disease is a viral disease or a bacterial disease. In an embodiment, the viral disease is viral hemorrhagic fever, gastroenteritis, dengue fever, West Nile fever, yellow fever, influenza, respiratory syncytial virus disease, Lassa fever, rabies, smallpox, cowpox, horsepox, monkeypox, Hantavirus pulmonary syndrome, Hendra virus disease, human immunodeficiency virus infection and acquired immunodeficiency disease syndrome, Hepatitis, Zika fever, SARS, MERS, COVID-19, infectious bronchitis, infectious laryngotracheitis, Rift Valley fever, porcine epidemic diarrhea, porcine transmissible gastroenteritis, swine acute diarrhea syndrome, feline infectious peritonitis, African swine fever, or classical swine fever. In an embodiment, the viral hemorrhagic fever is Ebola or Marburg virus disease. In an embodiment, the bacterial disease is a drug resistant bacterial disease. In an embodiment, the drug resistant bacterial disease is tuberculosis, methicillin-resistant Staphylococcus aureus infection, or a drug resistant parasitic disease. In an embodiment, the drug resistant parasitic disease is malaria. In an embodiment, the infectious disease is COVID-19.

The term “cancer” and its derivates, as used herein, refers to a group of diseases comprising cells having abnormal cell growth and metastasized or the potential to metastasize, i.e. invade or spread to other parts of the body. For example, cancer includes but not limited to pancreatic cancer, kidney cancer such as renal cell carcinoma, urogenital cancer such as urothelial carcinomas, melanoma, prostate carcinoma, lung carcinomas such as non-small cell carcinoma, small cell carcinoma, neuroendocrine carcinoma, or carcinoid tumor, breast carcinomas such as ductal carcinoma, lobular carcinoma, or mixed ductal and lobular carcinoma, thyroid carcinomas such as papillary thyroid carcinoma, follicular carcinoma, or medullary carcinoma, brain cancers such as meningioma, astrocytoma, glioblastoma, cerebellum tumors, or medulloblastoma, ovarian carcinomas such as serous, mucinous, or endometrioid types carcinomas, cervical cancers such as squamous cell carcinoma in situ, invasive squamous cell carcinoma, or endocervical adenocarcinoma, uterine endometrial carcinoma such as endometrioid or serous and mucinous types carcinomas, primary peritoneal carcinoma, mesothelioma such as pleura or peritoneum mesothelioma, eye cancer such as retinoblastoma, muscle cancer such as rhabdosarcoma or leiomyosarcoma, lymphomas, esophageal cancer such as adenocarcinoma or squamous cell carcinoma, gastric cancers such as gastric adenocarcinoma or gastrointestinal stroma tumour (GIST), liver cancers such as hepatocellular carcinoma or bile duct cancer, small intestinal tumors such as small intestinal stromal tumor or carcinoid tumor, colon cancer such as adenocarcinoma of the colon, colon high grade dysplasia, or colon carcinoid tumor, testicular cancer, skin cancers such as melanoma or squamous cell carcinoma, or adrenal carcinoma.

The term “treating” and its derivatives, as used herein, refers to improving the condition associated with a disease, such as reducing or alleviating symptoms associated with the condition or improving the prognosis or survival of the subject. The term “preventing” and its derivatives, as used herein, refer to averting or delaying the onset of the disease, such as inhibiting or avoiding the advent of the disease, or vaccinated against the disease, or the lessening of symptoms upon onset of the disease, in the subject. The term “prophylactic” shall have a corresponding meaning.

The term “subject” as used herein refers to any member of the animal kingdom, optionally a mammal, optionally a human. In an embodiment, the subject is a mammal. In an embodiment, the subject is a human, a non-human primate, a rodent, a feline, a canine, an ovine, a bovine, a porcine, a caprine, an equine, a lupine, a vulpine, or a mustelid. In an embodiment, the subject is human. In an embodiment, the Mustela is a weasel, a polecat, stoats, a ferret or a mink. In an embodiment, the subject is a mink.

Accordingly, the present disclosure provides a method of treating or preventing a disease in a subject, comprising administering an engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, and wherein the nucleic acid comprises or further comprises at least one heterologous nucleic acid segment encoding a therapeutic agent operably linked to a promoter capable of expressing the segment in a host cell. In an embodiment, the nucleic acid comprises a nucleic acid sequence encoding an L protein having a stabilizing segment. In an embodiment, the host cell is selected from the group consisting of a human, primate, murine, feline, canine, ovine, bovine, porcine, caprine, equine, lupine, vulpine, and Mustela host cell. In a further embodiment, the promoter is capable of expressing the at least one heterologous nucleic acid segment encoding the therapeutic agent in muscle, airway, or lung cells. In an embodiment, the therapeutic agent is any therapeutic agent as described herein. In an embodiment, the disease is any disease described herein.

The engineered NDV vector of the present disclosure is also useful for eliciting an immune response. According, also provided is a method for eliciting an immune response in a subject comprising administering an engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises XbaI and MluI restriction endonuclease sites between nucleic acid sequence encoding phosphoprotein and matrix protein. In an embodiment, the nucleic acid comprises or further comprises at least one heterologous nucleic acid segment encoding a therapeutic agent operably linked to a promoter capable of expressing the segment in a host cell. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 2, 3, 4, 18, 19, 23, 27, or 42. In an embodiment, the immunogenic agent activates B-cells, CD4+ T-cells and/or CD8+ T-cells.

Also provided is use of an engineered NDV vector for eliciting an immune response in a subject, wherein the engineered NDV vector comprises a nucleic acid having a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the at least one heterologous nucleic acid segment encodes a therapeutic agent operably linked to a promoter capable of expressing the segment in a host cell. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 2, 3, 4, 18, or 19. In an embodiment, the immunogenic agent activates B-cells, CD4+ T-cells and/or CD8+ T-cells.

Further provided is use of an engineered NDV vector in the manufacture of a medicament for eliciting an immune response in a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the at least one heterologous nucleic acid segment encodes a therapeutic agent operably linked to a promoter capable of expressing the segment in a host cell. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 2, 3, 4, 18, or 19. In an embodiment, the immunogenic agent activates B-cells, CD4+ T-cells and/or CD8+ T-cells.

Even further provided is an engineered NDV vector for use in eliciting an immune response, wherein the engineered NDV vector comprises a nucleic acid having a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment. wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the at least one heterologous nucleic acid segment encodes a therapeutic agent operably linked to a promoter capable of expressing the segment in a host cell. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence any one of SEQ ID NO: 2, 3, 4, 18, or 19. In an embodiment, the immunogenic agent activates B-cells, CD4+ T-cells and/or CD8+ T-cells.

The ability of the engineered NDV vector of the present disclosure to activate an immune response is useful for its use as a vaccine or an immunogenic composition. Accordingly, also provided is a method for vaccination, the method comprises administering a vaccine comprising an engineered NDV vector having a nucleic acid comprises a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment. wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the at least one heterologous nucleic acid segment encoding a therapeutic agent operably linked to a promoter capable of expressing the segment in a host cell. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 2, 3, 4, 18, or 19. In an embodiment, the immunogenic agent activates B-cells, CD4+ T-cells and/or CD8+ T-cells.

Also provided is use of a vaccine comprising an engineered NDV vector for vaccinating a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the at least one heterologous nucleic acid segment encoding a therapeutic agent operably linked to a promoter capable of expressing the segment in a host cell. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 2, 3, 4, 18, or 19. In an embodiment, the immunogenic agent activates B-cells, CD4+ T-cells and/or CD8+ T-cells.

Further provided is use of a vaccine comprising an engineered NDV vector in the manufacture of a medicament for vaccinating a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the at least one heterologous nucleic acid segment encodes a therapeutic agent operably linked to a promoter capable of expressing the segment in a host cell. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 2, 3, 4, 18, or 19. In an embodiment, the immunogenic agent activates B-cells, CD4+ T-cells and/or CD8+ T-cells.

Even further provided is a vaccine comprising an engineered NDV vector for use in vaccinating a subject, wherein the engineered NDV vector comprises a nucleic acid having a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the at least one heterologous nucleic acid segment encodes a therapeutic agent operably linked to a promoter capable of expressing the segment in a host cell. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 2, 3, 4, 18, 19, 23, 27, or 42. In an embodiment, the immunogenic agent activates B-cells, CD4+ T-cells and/or CD8+ T-cells.

Also provided is a method for administering an immunogenic composition in a subject, the method comprises administering an immunogenic composition comprising an engineered NDV vector having a nucleic acid comprises a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 2, 3, 4, 18, or 19. In an embodiment, the immunogenic agent activates B-cells, CD4+ T-cells and/or CD8+ T-cells.

Also provided is use of an immunogenic composition comprising an engineered NDV vector for eliciting an immune response in a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 2, 3, 4, 18, 19, 23, 27, or 42. In an embodiment, the immunogenic agent activates B-cells, CD4+ T-cells and/or CD8+ T-cells.

Further provided is use of an immunogenic composition comprising an engineered NDV vector in the manufacture of a medicament for eliciting an immune response in a subject, wherein the engineered NDV vector comprises a nucleic acid having a nucleic acid sequence that is at least 75%, 80%, 85%, 90%, 95%, 96%, 97%, 98%, 99%, 99.5%, 99.9% A or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 2, 3, 4, 18, or 19. In an embodiment, the immunogenic agent activates B-cells, CD4+ T-cells and/or CD8+ T-cells.

Even further provided is an immunogenic composition comprising an engineered NDV vector for use in eliciting an immune response in a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a therapeutic agent operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the therapeutic agent is an immunogenic agent. In an embodiment, the immunogenic agent is SARS-CoV-2 spike protein or fragment thereof. In an embodiment, the nucleic acid comprises a nucleic acid sequence that is at least 75%, 80%, 85%, 90%, 95%, 96%, 97%, 98%, 99%, 99.5%, 99.9% A or 100% identical to the nucleic acid sequence any one of SEQ ID NO: 2, 3, 4, 18, 19, 23, 27, or 42. In an embodiment, the immunogenic agent activates B-cells, CD4+ T-cells and/or CD8+ T-cells.

The engineered NDV vector can function as a delivery vehicle that delivers heterologous nucleic acid segment (“payloads”) encoding a therapeutic agent for treating or preventing a disease such as an infectious. In one embodiment, the infectious disease is selected from the group consisting of viral diseases such as viral hemorrhagic fevers, Ebola, Marburg virus disease, gastroenteritis, dengue fever, West Nile fever, yellow fever, influenza, respiratory syncytial virus disease, Lassa fever, rabies, smallpox, cowpox, horsepox, monkeypox, Hantavirus pulmonary syndrome, Hendra virus disease, human immunodeficiency virus disease and acquired immunodeficiency disease syndrome, Hepatitis, Zika fever, optionally Ebola or Marburg virus disease, Severe Acute Respiratory Syndrome (SARS), Middle East Respiratory Syndrome (MERS), Coronavirus disease 2019 (COVID-19), and bacterial diseases including drug resistant bacterial diseases such as tuberculosis and methicillin-resistant Staphylococcus aureus infection, and drug resistant parasitic diseases such as malaria. In an embodiment, the infectious disease is COVID-19.

The immune response can be independent of expression of a therapeutic agent such as an immunogenic agent. For example, the engineered NDV vector disclosed herein can activate NK cells in a subject bearing tumour. In some embodiments, the immune response comprises activation of NK cells. In some embodiments, the activation of NK cells comprises production of CD69, PD-L1, Granzyme B and/or IFNgamma. Such an immune response is useful for the treatment of, for example, cancer, such that the engineered NDV vector of the present disclosure is also useful as an anti-cancer agent. According, also provided is a method of treating cancer in a subject, comprising administering an engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1, 5, 9, or 10.

Also provided is use of an engineered NDV vector for treating cancer in a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1, 5, 9, or 10.

Further provided is use of an engineered NDV vector in the manufacture of a medicament for treating cancer in a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1, 5, 9, or 10.

Even further provided is an engineered NDV vector for use in treating cancer in a subject, wherein the engineered NDV vector comprises a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1, 5, 9, or 10.

In some embodiments, the engineered NDV vector comprises a nucleic acid having a nucleic acid sequence that is at least 75%, 80%, 85%, 90%, 95%, 96%, 97%, 98%, 99%, 99.5%, 99.9% A or 100% identical to the nucleic acid sequence of SEQ ID NO: 9, 10, 23, or 27. In some embodiments, the cancer is pancreatic cancer, kidney cancer such as renal cell carcinoma, urogenital cancer such as urothelial carcinomas, melanoma, prostate carcinoma, lung carcinomas such as non-small cell carcinoma, small cell carcinoma, neuroendocrine carcinoma, or carcinoid tumor, breast carcinomas such as ductal carcinoma, lobular carcinoma, or mixed ductal and lobular carcinoma, thyroid carcinomas such as papillary thyroid carcinoma, follicular carcinoma, or medullary carcinoma, brain cancers such as meningioma, astrocytoma, glioblastoma, cerebellum tumors, or medulloblastoma, ovarian carcinomas such as serous, mucinous, or endometrioid types carcinomas, cervical cancers such as squamous cell carcinoma in situ, invasive squamous cell carcinoma, or endocervical adenocarcinoma, uterine endometrial carcinoma such as endometrioid or serous and mucinous types carcinomas, primary peritoneal carcinoma, mesothelioma such as pleura or peritoneum mesothelioma, eye cancer such as retinoblastoma, muscle cancer such as rhabdosarcoma or leiomyosarcoma, lymphomas, esophageal cancer such as adenocarcinoma or squamous cell carcinoma, gastric cancers such as gastric adenocarcinoma or gastrointestinal stroma tumour (GIST), liver cancers such as hepatocellular carcinoma or bile duct cancer, small intestinal tumors such as small intestinal stromal tumor or carcinoid tumor, colon cancer such as adenocarcinoma of the colon, colon high grade dysplasia, or colon carcinoid tumor, testicular cancer, skin cancers such as melanoma or squamous cell carcinoma, or adrenal carcinoma. In an embodiment, the cancer is an ovarian cancer.

The use or administration of an engineered NDV vector to a subject comprises ingestion, instillation such as intranasally, inhalation such as via aerosol, or injection. The route of injection includes but is not limited to intradermal, subcutaneous, intramuscular, intravenous, intraosseous, intraperitoneal, intrathecal, epidural, intracardiac, intraarticular, intracavernous, intravitreal, intracerebral, intracerebroventricular, intratracheal or intraportal. In an embodiment, the engineered NDV vector is administered or used intravenously, intranasally, intratracheal, intramuscularly, or via aerosol. In an embodiment, the engineered NDV vector is administered or used intranasally. In an embodiment, the engineered NDV vector is administered or used intramuscularly. In an embodiment, the engineered NDV vector is delivered to muscle, airway, or lung cells or tissues.

The present disclosure further provides a method of producing a protein in vivo in a subject, comprising delivering or introducing into the subject an engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a protein operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence of SEQ ID NO: 9, 10, 23, or 27.

In addition, the present disclosure provides a method of producing at least one protein in vitro in a host cell, comprising introducing into the host cell an engineered NDV vector comprising a nucleic acid having a nucleic acid sequence that is at least 75%, 80%, 85%, 90%, 95%, 96%, 97%, 98%, 99%, 99.5%, 99.9% or 100% identical to the nucleic acid sequence of any one of SEQ ID NO: 1-5, 9, 10, 18, 19, 23, 27, or 42, wherein the nucleic acid comprises a nucleic acid sequence encoding an L protein having a stabilizing segment, wherein the nucleic acid comprises or further comprises at least one heterologous nucleic acid segment encoding a protein operably linked to a promoter capable of expressing the segment in a host cell, and wherein the nucleic acid comprises XbaI and MluI restriction endonuclease sites between nucleic acid sequence encoding phosphoprotein and matrix protein. In an embodiment, the protein is any protein described herein. The skilled person can readily recognize the suitable production or manufacturing methods for producing proteins such as therapeutic agents using the engineered NDV vector as described herein. In an embodiment, the nucleic acid comprises a nucleic acid sequence that is at least 75%, 80%, 85%, 90%, 95%, 96%, 97%, 98%, 99%, 99.5%, 99.9% or 100% identical to the nucleic acid sequence of SEQ ID NO: 9, 10, 23, or 27.

Also provided is a method for selecting a stable engineered NDV vector genome. Inventors have developed a visual screening tool for selecting stable engineered clones based on their growth pattern on Luria-Bertani (LB) plates. When cloning transgenes (e.g. viral antigen for vaccine purposes) into the NDV genome and screening for colonies that contain the full-length NDV genome plasmid with the correct insert, the transformed bacteria often grow as both large and small colonies. The large colonies are visible after 16 hours whereas the smaller colonies need to grow for at least 24 hours before they are large enough to inoculate a liquid culture. The large colonies often contain mutated NDV genome plasm ids, whereas the small colonies invariably contain stable NDV clones and are thus selected for growth in liquid culture. Accordingly, also provided is a method for selecting an engineered NDV vector genome comprising a stabilizing segment in L gene, the method comprises:
a) growing bacterial cells comprising an engineered NDV vector genome plasmid in growth medium broth;
b) growing the bacterial cells on an agar-growth medium, wherein the agar-growth medium comprises a selection agent;
c) identifying small bacterial cell colonies having about 0.5 mm to about 1 mm in diameter after at least 24 hours of growth;
d) repeating step a) to step c) two to nine times to enrich for small bacterial cell colonies; and
e) isolating the engineered NDV vector genome from the small bacterial cell colonies,
wherein the small bacterial cells colonies comprise stable engineered NDV vector genome having the stabilizing segment in L gene.

In an embodiment, the growth medium broth is a Luria Bertani (LB) broth. In an embodiment, the agar-growth medium is agar-Luria Bertani (LB). In an embodiment, the selection agent is an antibiotic. In an embodiment, the antibiotic is kanamycin. In an embodiment, the stabilizing segment comprises an amino acid sequence as set forth in SEQ ID NO: 20. In an embodiment, the stabilizing segment is encoded by a nucleic acid comprising a nucleic acid sequence as set forth in SEQ ID NO: 35. In an embodiment, the stable engineered NDV vector genome encodes a full-length L protein (SEQ ID NO: 11). In an embodiment, the bacterial cells are E. coli. In an embodiment, the E. coli is an E. coli strain Stellar, NEBStable, or GT116.

The following non-limiting Examples are illustrative of the present disclosure:

### Example 1A. Development of NDV-FLS and NDV-A19S Immunogens Using Engineered Newcastle Disease Virus Vectors Expressing SARS-CoV-2 Spike Proteins

### Materials and Methods

### Engineered NDV Vector

The full-length cDNA genome of lentogenic NDV LaSota strain was synthetically designed based on accession AF077761.1 to contain a GFP reporter gene and essential NDV-specific RNA transcriptional signals, flanked by a 5′ XbaI site and a 3′ MluI site at position 3143 nucleotide between the P and M genes. Unique restriction sites between the P gene and the M gene were chosen because transgenes expressed between these sites are highly expressed and these restriction sites do not interfere with the stability of the recombinant virus. A leucine to alanine mutation at position 289 was also introduced into the Fusion gene. To construct recombinant NDV expressing SARS-CoV-2 Spike protein, forward 5′GCACCGAGTTCCCCCTCTAGATTAGAAAAAATACGG GTAGAACCGCCAC-3′ (SEQ ID NO: 21) and reverse 5′GTTGGACCTTGGGTAC GCGTTTATCAGGTGTAGTGCAGCTTCAC-3′ (SEQ ID NO: 22) primers were used to amplify human codon optimized SARS-CoV-2 full length spike protein. Additionally, a 19 amino acid truncated form of the Spike protein (SΔ19) was amplified using the above forward primer (SEQ ID NO: 21) and a reverse 5′G TTGGACCTTGGGTACGCGTTTATCATCAGCAGCAAGAGCCGCAAGAACAAC-3′ (SEQ ID NO: 24). Infusion Cloning™ was used to insert transgenes into the NDV backbone according to the manufacturer's protocol (Takara Bio USA), with the 5′ end of the primer including 15 bp of homology with each end of the linearized vector including the XbaI or MluI sites. Viruses were rescued from cDNA, amplified and purified using methods described previously (Santry, L. A. et al., 2017) and confirmed by RT-PCR and sequencing.

### DF-1 Infection Protocol

DF-1 cells (ATCC CRL-12203) were seeded into 6-well plates at 1.5×10⁶ cells/well in 1 mL of DMEM supplemented with 2% bovine calf serum (BCS) and 5% allantoic fluid. After adherence, the cells were infected with either NDV-FLS, -Δ19S or -GFP at MOI of 1 and 10 in replicate plates. The plates were incubated at 37° C. One day post infection, the replicate plates were observed under an inverted phase contrast microscope to examine and document cytopathic effect (CPE) with photographs. Subsequently, one set of replicate plates was collected for protein extraction and Western blot analysis, and the second set of replicate plates was used for immunofluorescence assay (IFA).

### Immunofluorescence Assay

Approximately 1 day post infection, old media were removed and cells were rinsed twice with phosphate-buffered saline (PBS). Cells were then fixed in 4% paraformaldehyde (PFA) for 15 minutes at room temperature (RT). After fixation, cells were washed three times with PBS-T (PBS-1% tween) for 5 minutes each. The cells were then permeabilized in 0.1% NP-40 for 10 minutes at RT followed by three washes with PBS-T for 5 minutes each. Subsequently, cells were blocked in blocking buffer [5% (v/v) normal goat serum in PBS-T] either for one hour at RT or overnight at 4° C. After blocking, cells were incubated in primary mouse anti-NDV (NBP2-11633; Novus Biologicals) diluted 1:2000 in blocking buffer for one hour at RT (or overnight at 4° C.). Following the primary antibody incubation, cells were washed three times with PBS-T for 5 minutes each and then incubated with secondary goat-anti-mouse-488 (Invitrogen, ThermoFisher) diluted in 1:1000 in PBS-T for one hour at RT in the dark. Following secondary antibody incubation, cells were once more washed 3 times with PBS-T for 5 minutes each. After the final wash was removed, PBS-T was added to keep cells submerged under solution, and cells were imaged using an Axio observer inverted fluorescent microscope.

### SDS-PAGE (Denaturing) and Western Blot Analysis

Infected DF-1 cells were washed with PBS and lysed in radioimmunoprecipitation assay (RIPA) buffer (50 mM Tris-HCl pH 8, 150 mM NaCl, 1% NP-40, 0.5% sodium deoxycholate, 0.1% sodium dodecyl sulfate, 1× protease inhibitor cocktail) for 30 min on ice. Following lysis, cell lysates were centrifuged at 10,000×g for 15 min at 4° C. The supernatants were transferred to a new collection tube and debris was discarded. Protein amount in the supernatants were quantified using the Pierce BCA Protein Assay Kit (ThermoFisher) according to the manufacturer's instructions. For SDS-PAGE, cell lysates (mixed with 6× loading dye containing and 30% β-mercaptoethanol) were heated at 95° C. for 10 min to denature proteins, followed by cooling on ice. Protein, with amounts ranging from 5 μg to 70 μg depending on experiment, were loaded into wells of 4% stacking/12% resolving gels. The same protein amount of each sample was loaded within each experiment. Proteins were resolved at 120 V for 1.5 h in running buffer (0.025 mM Tris-base, 0.192 M glycine, 0.1% SDS), followed by semi-dry transfer to a 0.2 μm PVDF membrane for 30 min using the BioRad Trans-Blot Turbo Transfer System and BioRad proprietary buffer (BioRad Trans-Blot Turbo RTA Mini PVDF Transfer Kit). Following transfer, the rest of the protocol was performed as previously described (Pham P H et al., 2020). All wash steps were performed with PBS-T. The primary antibodies were either the mouse anti-NDV antibody (dilution: 1:5000; NBP2-11633; Novus Biologicals), rabbit anti-SARS spike protein antibody (dilution: 1:1000; NB100-56578; Novus Biologicals), or mouse anti-beta actin antibody (diluted 1:1000; MA5-15739; ThermoFisher). Primary antibodies were incubated overnight at 4° C. The secondary antibodies were either goat anti-rabbit or goat anti-mouse IgG conjugated to horseradish peroxidase (diluted 1:2000; ThermoFisher). Secondary antibodies were incubated for 1 to 3 h at RT. Protein was detected using the Pierce SuperSignal West Pico PLUS Chemiluminescent Substrate (ThermoFisher) and a BioRad ChemiDoc MP Imaging System (BioRad Image Lab 6.0.1. software).

### Determination of Mean Death Time (MDT)

The MDT was determined for three viruses: NDV-FLS, -SΔ19, and -GFP. The virus stocks were equalized to the starting titre of 6.14×10⁶ FFU/mL. Each virus was diluted in a 10-fold 1 mL serial dilution series from 10⁻¹ to 10⁻⁸ in PBS. To determine the MDT, virus dilutions from 10⁻⁴ to 10⁻⁸ were chosen to be inoculated into SPF eggs (Canadian Food Inspection Agency) at 9 to 11 days of embryonation. For each of the three viruses, a total of 50 eggs were used for two replicate MDT experiments (25 eggs per replicate), which were done in the same day but separated by 3 to 4 hours between replicates. Of the 25 eggs in each replicate MDT experiment, five replicate eggs received 100 μL of 10⁻⁴ diluted virus, five received 100 μL of 10⁻⁵ diluted virus, five received 100 μL of 10⁻⁸ diluted virus, five received 100 μL of 10⁻⁷ diluted virus and five received 100 μL of 10⁻⁸ diluted virus. For the entire MDT experiment involving all three viruses, a total of 150 eggs were used. After virus inoculation, the eggs were incubated for up to 7 days and checked and scored twice daily for embryo mortality. Allantoic fluid was collected from dead embryos to check for presence of NDV by hemagglutination assay (HA). If no MDT was reached by the end of the experiment (7 days post inoculation), then HA was performed on allantoic fluid collected from eggs inoculated with the virus dilution containing the highest virus amount (10⁻⁴) to confirm presence of NDV in eggs containing embryos that did not die (as defined by the AVIS Consortium, see http://www.fao.org/ag/againfo/programmes/en/empres/gemp/avis/A160-newcastle/mod0/0344-mdt-tests.html).

### Hemagglutination Assay (HA)

For the HA, allantoic fluid (from eggs inoculated with NDV) was diluted in a 2-fold 100 μL serial dilution series from 2⁻¹ (e.g. 50 μL of allantoic fluid and 50 μL of PBS) to 2⁻⁷ in PBS, in duplicate wells of a 96-well V-bottom plates. At the last dilution of 2⁻⁷, after mixing, 50 μL of the mixture was discarded, leaving 50 μL remaining in these wells and the wells of the other dilutions. The above procedure was repeated for PBS alone and for allantoic fluid from uninfected control eggs; these served as negative controls for the HA. Once serial dilution was completed, 50 μL of 1% chicken red blood cells (diluted in PBS) was added to each well. The plates were incubated at RT for 45 min followed by scoring of the plates and documentation by photographs.

### Rescue of SARS-CoV-2 Spike Protein Pseudotyped Lentiviral Particles

HEK 293T (human kidney cells, ATCC CRL-11268) cells grown in DMEM with 10% FBS and 1% penicillin/streptomycin were seeded in a 10 cm cell culture dish so that they would be 60-70% confluent the following day. 16-24 h post-seeding, cells were transfected using PolyJet™ Reagent (SignaGen Laboratories) in a 1:1 ratio of reagent-to-DNA with 6.7 μg of each of the following plasm ids: pSin-EF1α-luciferase, psPAX2 (Didier Trono; Addgene plasmid #12260; http://n2t.net/addgene:12260; RRID:Addgene_12260), and pCASI-SARS-CoV-2-Spike-Δ19. The following day the media was changed to fresh complete media. Starting at 48 hours post-media change, lentivirus was collected twice per day by changing media and replacing with complete media. Lentivirus was collected until 96 hours post-media change for a total 5 collections. Lentivirus collections were pooled, filtered through a 0.45 μm PES filter and frozen as aliquots at −80° C.

### Assessment of Luciferase Activity

1.25×10⁴ HEK293T-hACE2 cells (Dr. Paul Spagnuolo, University of Guelph) were seeded per well in a 96-well plate and left to adhere overnight. The following day, media was removed and replaced with 40 μL of fresh complete media. Cells were then transduced with 60 μL of lentivirus, along with polybrene at a final concentration of 8 μg/mL. 60 hours post-transduction, luciferase activity was measured using the Pierce™ Firefly Luciferase Glow Assay Kit (Thermo Scientific) as per manufacturer's instructions. Luciferase readings were measured in white plates using an Enspire® Multimode Plate Reader (Perkin Elmer).

### Statistical Analysis

All results were analyzed and plotted using GraphPad Prism 8 Software. Statistical significance was assessed using Mann-Whitney test, one-way analysis of variance (ANOVA), two-way ANOVA where appropriate.

### Results

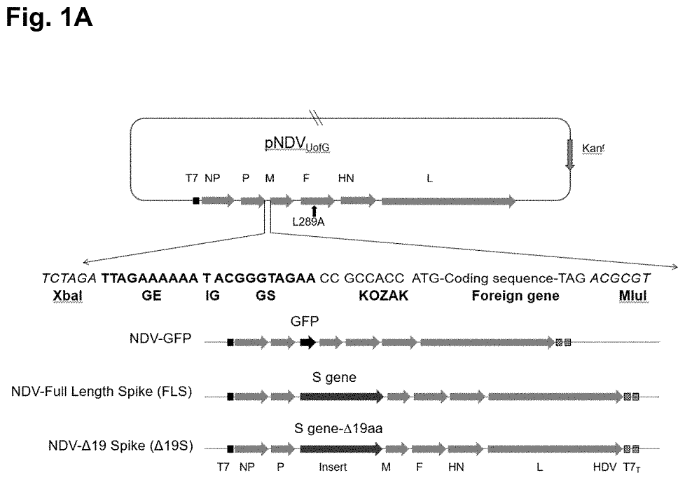

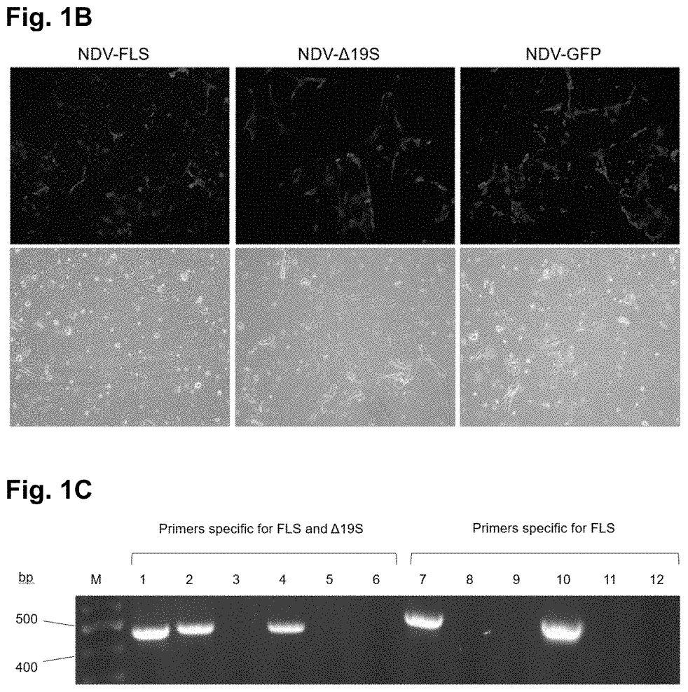

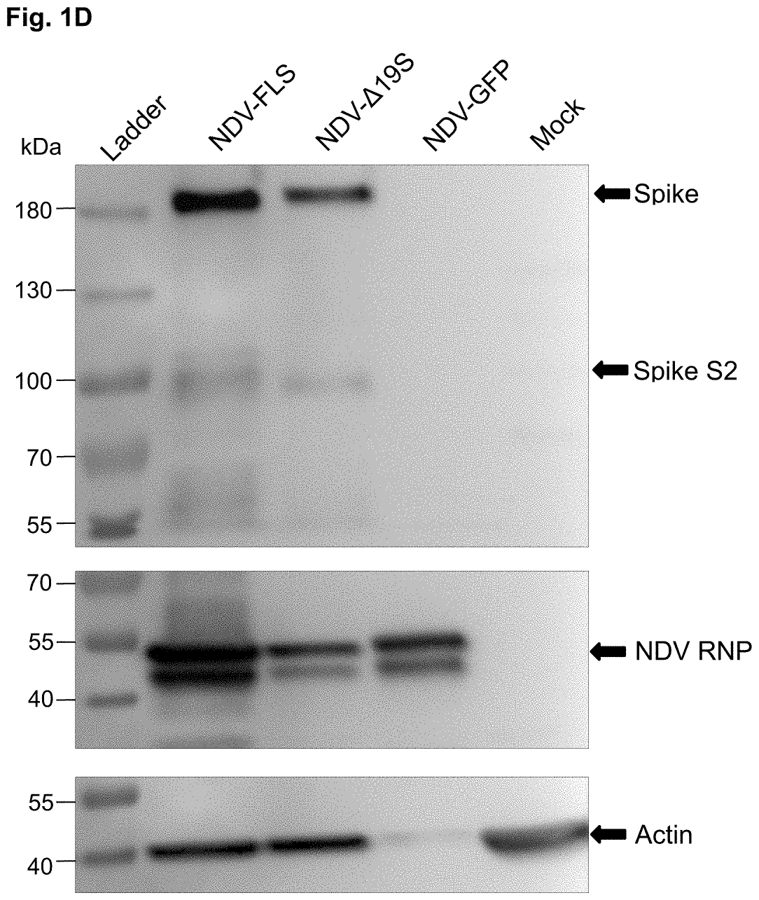

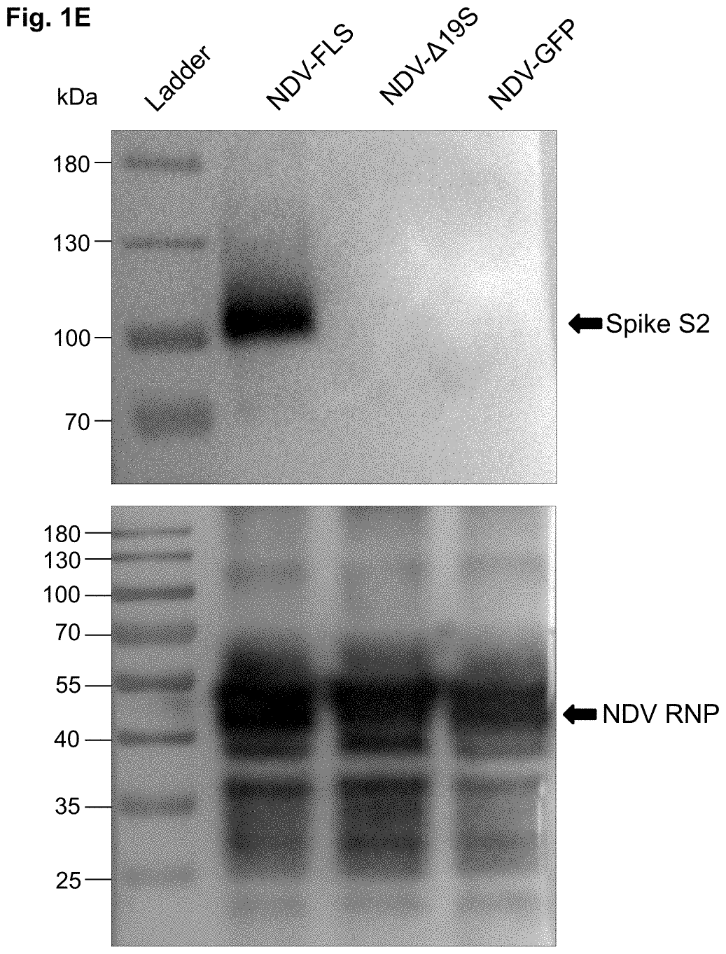

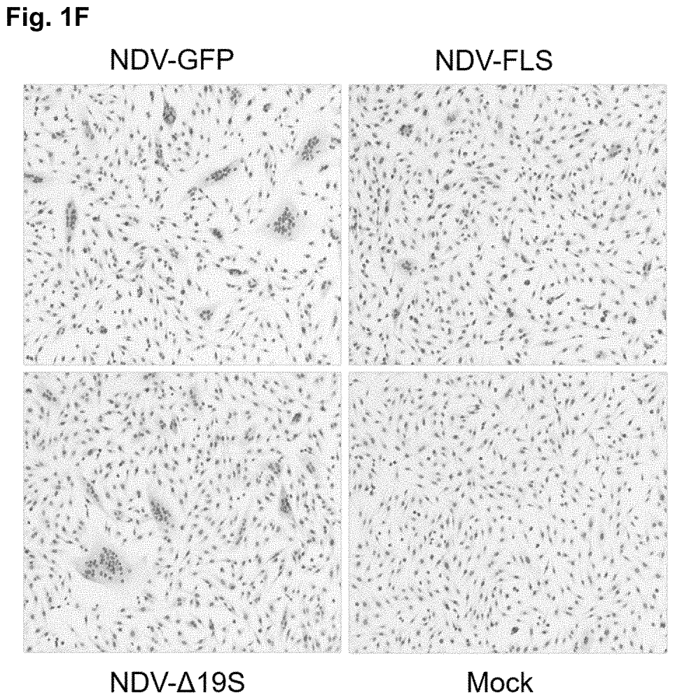

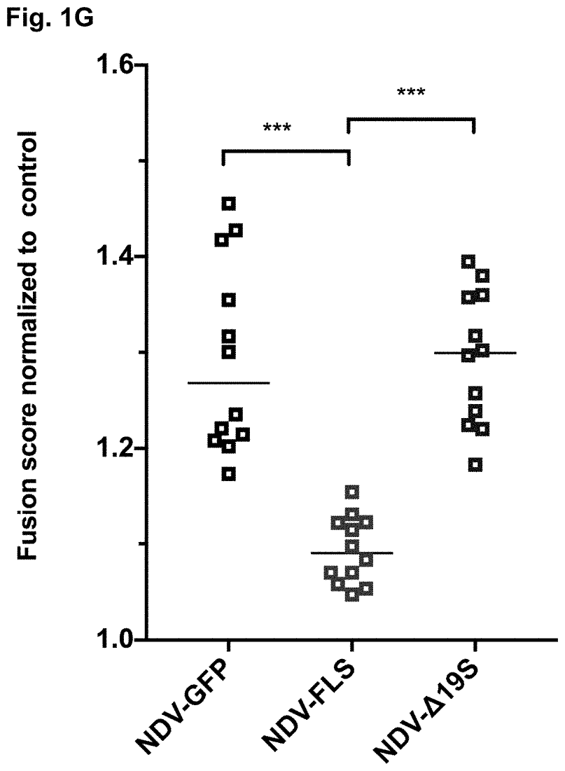

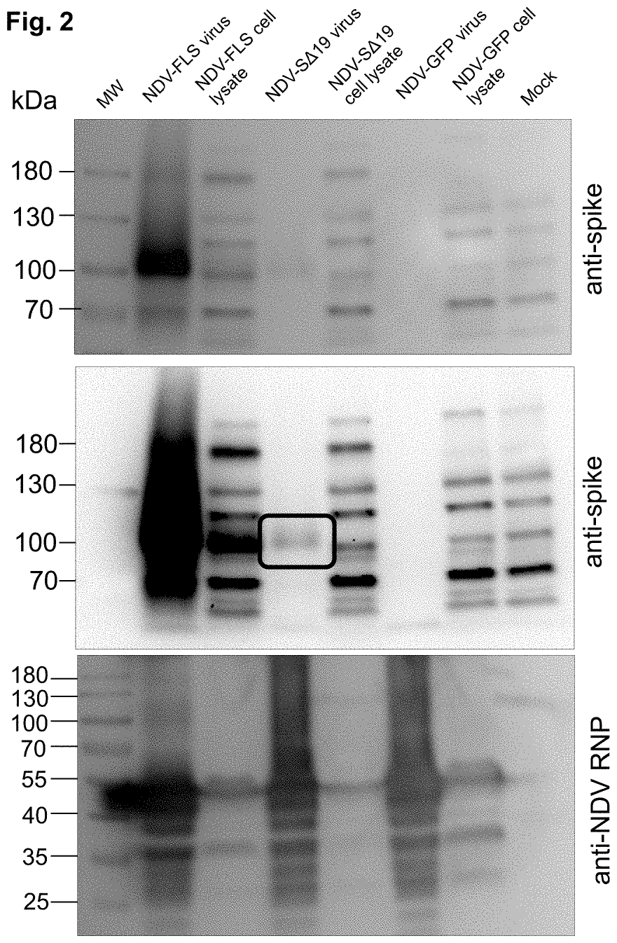

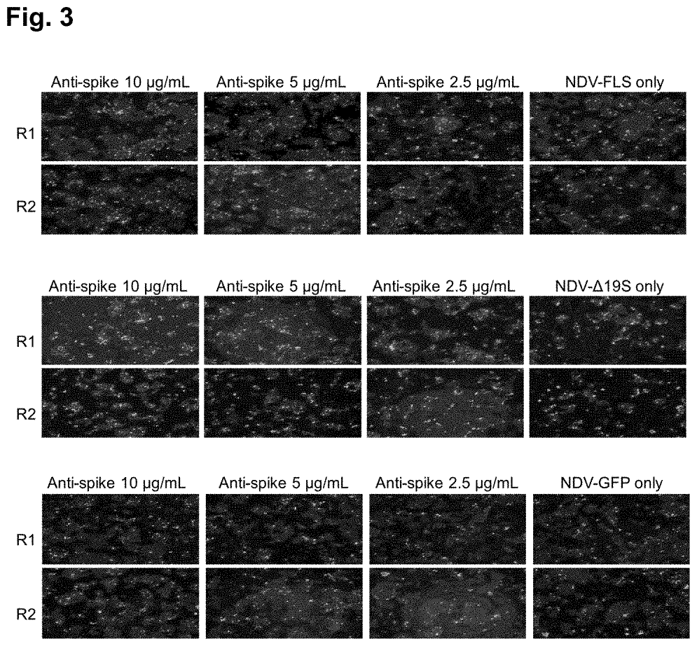

A fully synthetic molecular clone was engineered from lentogenic NDV (LaSota strain, Genbank accession AF077761.1) encoding a T7 promoter followed by three non-templated G's, unique XbaI and MluI restriction sites between the phosphoprotein (P) and the matrix (M) genes to facilitate transgene insertion, and a T7 terminator sequence. Also, an L289A mutation in the fusion (F) gene was also incorporated for enhanced fusion (Sergei, T. A et al 2000), and a self-cleaving hepatitis delta virus (HDV) ribozyme sequence was added to ensure adherence to the “rule of six” by self-cleaving immediately at the end of the viral antigenomic transcript (Kolakofsky, D., et al., 1998) (FIG. 1A). Engineered NDV vectors expressing the full length human codon optimized SARS CoV-2 spike protein (NDV-FLS), spike protein with 19 amino acids deleted from its C-terminus (NDV-Δ19S), which has been shown to promote more efficient incorporation of spike protein into lentiviral (Johnson, M. C., et al 2020) and VSV (Fukushi, S., et al 2005) particles, and GFP (NDV-GFP), between the P and M genes (FIG. 1A). Recombinant viruses were initially verified by immunofluorescence analysis of ribonucleoprotein (RNP) complex expression in NDV-FLS, NDV-119S and NDV-GFP infected DF-1 cells (FIG. 1B) and by RT-PCR confirmation of spike gene insertion (FIG. 1C). Western blot analysis of whole cell lysates from DF-1 cells infected with NDV-FLS or NDV-Δ19S showed robust expression of the full length spike protein, and in the case of NDV-FLS infected cells, weak expression of the cleaved S1 receptor-binding subunit (FIG. 1D). To investigate whether the spike protein expressed from NDV would be incorporated into the NDV virion, virus purified by gradient ultracentrifugation was subjected to Western blot analysis. As shown in FIG. 1E, spike protein was incorporated into the virion of the NDV-FLS virus; however, spike protein lacking 19 amino acids from C-terminus was poorly incorporated into the NDV virion, and was only visible after over-exposure of the Western blot (FIG. 2). Next, the spike protein was incorporated into the NDV virion to determine whether it would increase NDV infectivity in HEK 293T cells over-expressing human angiotensin-converting enzyme 2 (ACE2), the receptor for SARS-CoV-2. Using a 119S pseudotyped lentivirus neutralization assay, it was shown that neutralizing antibodies against SARS-CoV-2 spike do not affect NDV-FLS or NDV-Δ19S infection (FIG. 3) indicating that incorporation of spike protein on the surface of the NDV virion does not alter infectivity or tropism of the vaccine.

To investigate whether expressing the SARS-CoV-2 spike protein, which retains its multi-basic cleavage site, would impact the fusogenic properties of NDV, DF-1 cells were infected with NDV-FLS, NDV-Δ19S or NDV-GFP and the number of multinucleated syncytia quantified. As shown in FIG. 1F, all three viruses formed syncytia in the presence of trypsin. This shows that NDV expressing the spike protein is not more fusogenic than the parental NDV-GFP, suggesting that the spike protein, which has a multi-basic cleavage site, is not enhancing the fusogenicity of the NDV-FLS vaccine. However, NDV-expressing the FLS formed significantly smaller sized syncytia compared to either NDV-Δ19S or NDV-GFP (FIG. 1G).

Finally, to confirm that engineering NDV to express FLS, Δ19S or GFP does not alter pathogenicity of NDV in its host species, mean death time (MDT) in embryonated chicken eggs was determined. All viruses had an MDT>110 hours and thus retained their lentogenic phenotype.

Taken together, these data demonstrate that NDV can be engineered to express the SARS-CoV-2 spike protein without altering the safety profile of this viral vector. Moreover, the full length spike protein is incorporated into the NDV virion more efficiently than the Δ19 truncated version. Inventors have herein provided engineered synthetic molecular clones that are advantageous over other molecular clones of NDV in that, for example, unique restriction sites introduced allow for efficient insertion of transgenes between the P and M genes in an orientation dependent manner as well as allow for the exchange of the F and HN genes, for example, with those from other paramyxoviruses.

### Example 1B: Engineered Chimeric NDV Vector

Inventors have also engineered and rescued a chimeric NDV virus that has the F protein and HN protein from avian paramyxovirus 5 (APMV5) (SEQ ID NO. 4). F protein and HN protein are constituents of the NDV envelope, embedded within the lipid bilayer membrane. The inventors designed and produced this chimeric virus because the APMV5 F gene also has a multi-basic cleavage site, which, without wishing to be bound by theory, can be useful for fusion with cells. Since APMV-5 is not pathogenic in chickens the swapping of portion of NDV F protein with APMV5 F protein would broaden the use of this virus as an oncolytic agent in jurisdictions where there are restrictions imposed on avian pathogens, for example in the US by the authority of USDA/CDC. Specifically, for the NDV-APMV5 F-HN chimeric molecular clone sequence, NDV-APMV5 F is composed mostly of APMV5 but the last 53 amino acid are from NDV. NDV-APMV5 HN is composed mostly of APMV5 but the first 53 amino acids are from NDV.

### Example 1C: Screening Tool and Method for Selecting Stable Engineered NDV Clones

Inventors have also developed a visual screening tool for selecting positive, stable engineered clones based on their growth pattern on Luria-Bertani (LB) plates. Normally, molecular clone of NDV is unstable in most strains of E coli (e.g. Stellar, DH5alpha, GT116) in so for a large portion of the polymerase gene (L) would be deleted resulting in the growth of large and small colonies. The large colonies invariably possessed deletions in the L gene. However, inventors showed that selection of small colonies (about 0.5 mm to about 1 mm in diameter after 24 h of growth) followed by multiple rounds of growth in LB broth followed by selection of small colonies on LB-Kanamycin plates resulted in selection of bacteria that formed small colonies and harbored stable molecular clones of NDV.

### Example 2: Lyophilized NDV-FLS Retains its Infectivity

### Materials and Methods

Triplicate samples of freshly harvested allantoic fluid containing NDV-FLS were aliquoted into 15 mL conical tubes in 1 mL volumes. Aliquots were either left untreated or adjusted to a final concentration of 5% sucrose, 5% sucrose/5% Iodixanol or mixed 1:1 with a solution containing 10% Lactose, 2% peptone, 10 mM Tris-HCl, pH 7.6. Using a LABCONCO Freeze Dry system Freezone®4.5, samples were immediately lyophilized at 44×10-3 MBAR and −52° C. for 16 hours. Lyophilized samples were stored at 4° C. for 48 hours before being resuspended in 1 mL 5% sucrose/PBS and titered. Three 1 mL aliquots of allantoic fluid containing NDV-FLS were adjusted to 5% sucrose and frozen at −80° C. before titering. An additional three 1 mL aliquots were used to titer NDV-FLS in allantoic fluid immediately following harvest from eggs. All samples were titered by TCID50 on DF-1 cells as described above.

### Results

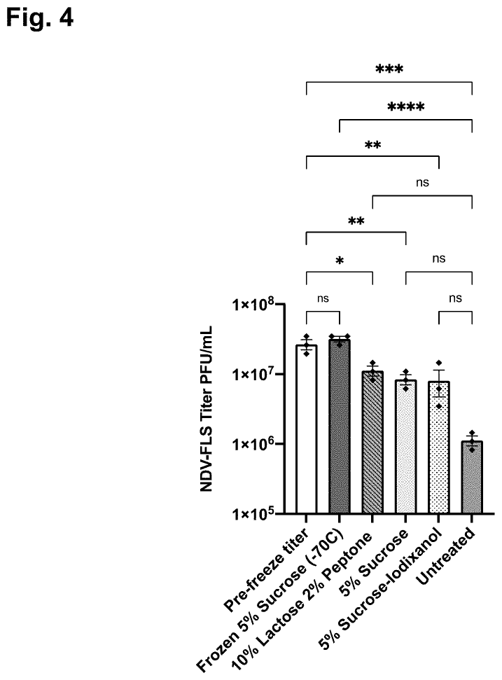

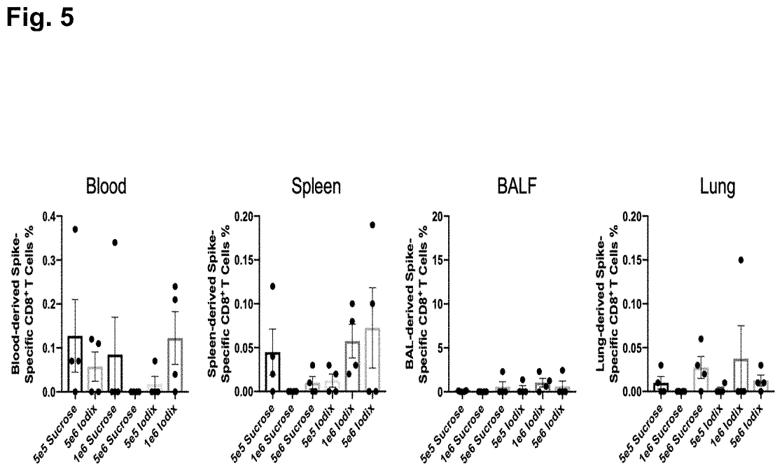

Inventors demonstrated that NDV-FLS can be lyophilized to simplify storage and distribution requirements, without significant negative effects. Aliquots of NDV-FLS were brought to a final concentration of 5% sucrose, 5% sucrose/5% Iodixanol or mixed 1:1 with a solution containing 10% lactose, 2% peptone, 10 mM Tris-HCl, pH 7.6 and lyophilized for 16 h at −52° C. Two days later, samples were reconstituted and virus titer determined as shown in FIG. 4. There was a ˜2-fold loss of infectivity when NDV-FLS is lyophilized in 10% lactose, 2% peptone, 10 mM Tris-HCl, pH 7.6 compared to virus frozen at −70° C.; however, given the convenience and greatly simplified storage and transportation requirements of a lyophilized vaccine, this reduction in infectivity is an acceptable tradeoff.

### Example 3: Engineered NDV Vector as a Vaccine for COVID-19 in Mice

### Methods and Materials

### T Cell Responses

Male Balb/c mice were administered intranasally various doses of a vaccine comprising NDV that expresses the spike protein from SARS-CoV-2 (NDV-FLS). After 32 days, mice were boosted with the same dose of vaccine via the same route of administration. Five days after boost, the mice were euthanized and spike protein-specific CD8+ T cell and CD4+ T cell responses were quantified in the blood, spleen, bronchoalveolar fluid, and lung.

### Intranasal Vs Intramuscular Administration

Male C57BL/6 or Balb/c mice were vaccinated either intranasally or intramuscularly with 5×10⁶ PFU NDV-FLS. At day 10 post-vaccine administration, a subset (n=4) of mice were terminally bled and the spike protein specific CD8+ and CD4+ T cell responses quantified. Mice were non-terminally bled prior to being boosted on day 28 with the same dose of vaccine, and then bled again on days 5 and 10 post-boost, and spike protein specific CD8+ and CD4+ T cell responses quantified. In addition, at 10 days post-boost, bronchoalveolar lavage fluid was collected and measured for SARS-CoV-2 spike protein-specific IgA antibodies.

### Results

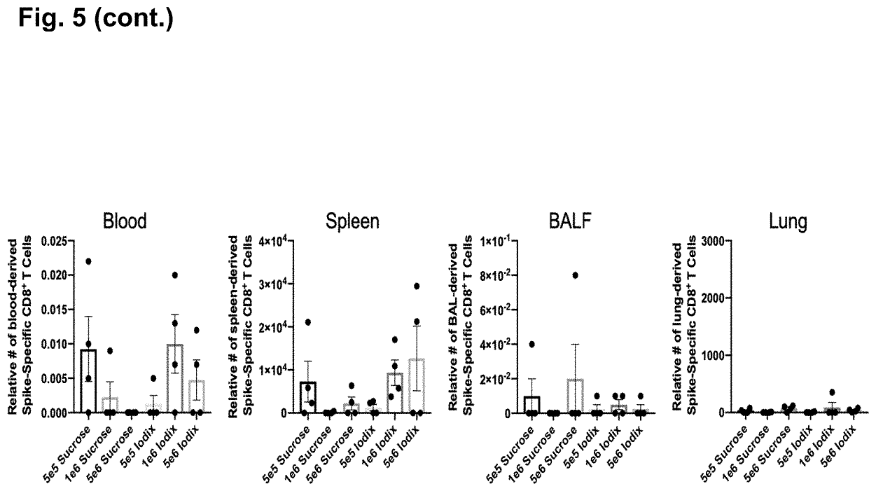

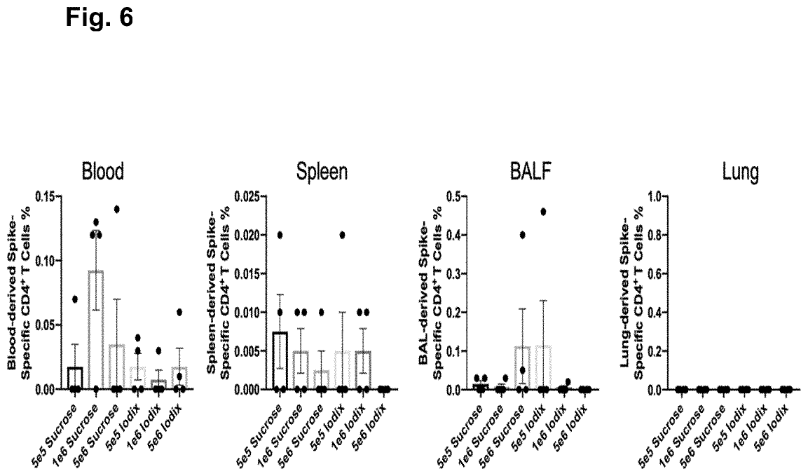

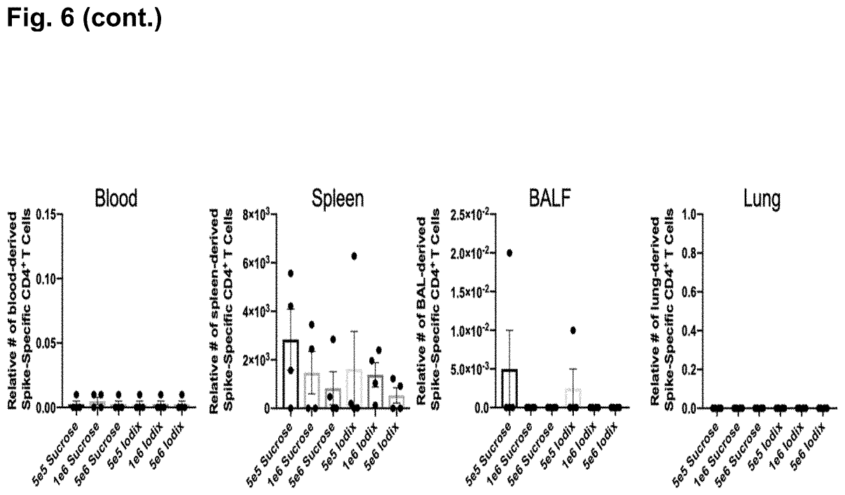

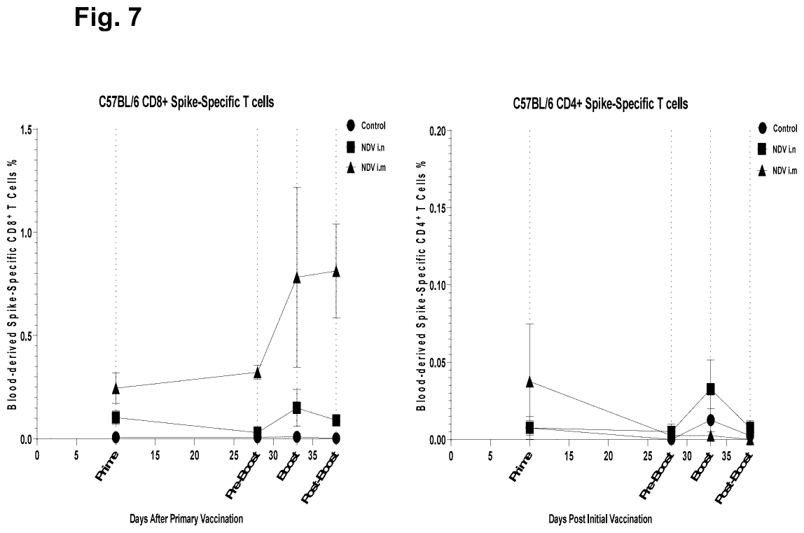

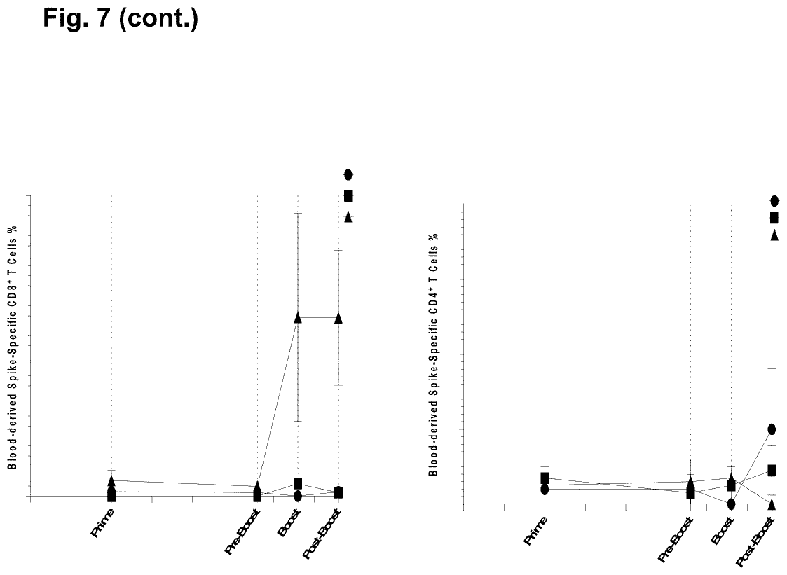

Inventors show that administration of engineered NDV vector expressing SARS-CoV-2 spike protein to mice elicits humoral and cellular responses. SARS-CoV-2 spike protein-specific CD8+ T cell and CD4+ T cell responses were detected quantified and are shown in FIG. 5 and FIG. 6, respectively. SARS-CoV-2 spike protein specific CD8+ and CD4+ T cell responses after intranasal or intramuscular administration were detected, quantified and compared, as shown in FIG. 7. As well, robust anti-spike IgA antibodies were detected in the Balb/c strain of mice after intranasal delivery of the NDV-FLS spike using a primer (5×10⁶ PFU) boost (5×10⁶ PFU) regimen (see Table 2).

TABLE 2
Spike-specific IgA antibodies in bronchoalveolar lavage fluid
IgA

| Treatment | Dilution | OD1 | Dilution | OD1 | Dilution | OD1 | Dilution | OD1 | Dilution | OD1 | Dilution | OD1 |
|---|---|---|---|---|---|---|---|---|---|---|---|---|
| NDV-FLS I.N C57BL6 | 1:5 | 0.063 | | 0 | | | | | | | | |
| NDV-FLS I.N C57BL6 | 1:5 | 0.113 | | 0 | | | | | | | | |
| NDV-FLS I.N C57BL6 | 1:5 | 0.101 | | 0 | | | | | | | | |
| NDV-FLS I.N C57BL6 | 1:5 | 0.125 | 1:10 | 0.045 | | | | | | | | |
| NDV-FLS I.N BalbC | 1:5 | 0.124 | 1:10 | 0.074 | 1:20 | 0.041 | | | | | | |
| NDV-FLS I.N BalbC | 1:5 | 0.51 | 1:10 | 0.173 | 1:20 | 0.206 | 1:40 | 0.015 | | | | |
| NDV-FLS I.N BalbC | 1:5 | 0.236 | 1:10 | 0.142 | 1:20 | 0.09 | 1:40 | 0.075 | 1:80 | 0.083 | 1:160 | 0.036 |
| NDV-FLS I.N BalbC | 1:5 | 0.012 | 1:10 | 0 | 1:20 | 0.712 | | | | | | |
| NDV-FLS I.M C57BL6 | 1:5 | 0.064 | | 0 | | | | | | | | |
| NDV-FLS I.M C57BL6 | 1:5 | 0.134 | 1:10 | 0.028 | 1:20 | 0.006 | 1:40 | 0.344 | | | | |
| NDV-FLS I.M C57BL6 | 1:5 | 0 | | 0 | | | | | | | | |
| NDV-FLS I.M C57BL6 | 1:5 | 0.047 | | 0 | | | | | | | | |
| NDV-FLS I.M BalbC | 1:5 | 0 | | 0 | | | | | | | | |
| NDV-FLS I.M BalbC | 1:5 | 0 | | 0 | | | | | | | | |
| NDV-FLS I.M BalbC | 1:5 | 0 | | 0 | | | | | | | | |

Thus, inventors have demonstrated that the engineered NDV vector molecular clone designed to express the SARS-CoV-2 spike protein (NDV-FLS) leads to the production of spike protein-specific serum IgG and mucosal IgA antibodies as well as spike protein-specific T cells responses in mice administered with the NDV-FLS vaccine intranasally.

### Example 4: Engineered NDV Vector Kills Tumor Cells In Vitro

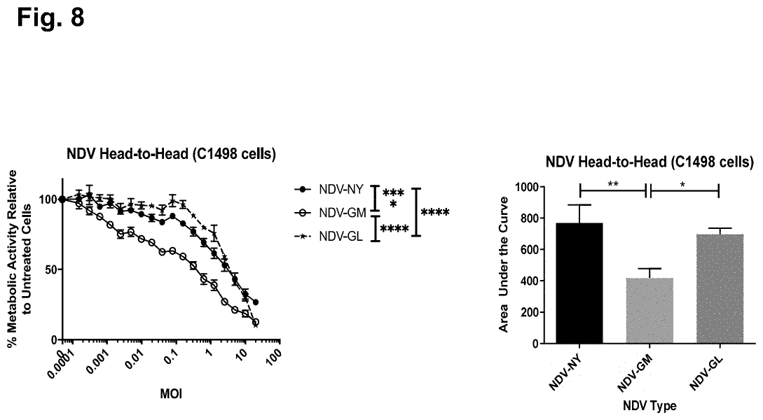

The ability of engineered NDV vector of this disclosure in killing tumor cells was tested in vitro using cells from murine acute myeloid leukemia (AML) C1498 cell line. Cultured C1498 cells were treated with NDV-GFP-NY (Park M-S et al, PNAS 2006; Gao Q et al, J Virol 2008), mesogenic NDV-GFP-GM (which has a 3 amino acid change in the F gene that makes it mesogenic (i.e. fusogenic), i.e. from GRQGRL to RRQRRF at amino acid positions 112, 115, and 117 in reference SEQ ID NO: 28, or lentogenic NDV-GFP-GL at varying MOI, and metabolic activity relative to untreated cells were measured by resazurin (cell proliferation) assay (FIG. 8, left panel). The area under the curve in FIG. 8, left panel was plotted in the graph on the right panel. These results show that mesogenic NDV-GFP-GM was significantly better than NDV-GFP-NY and lentogenic NDV-GFP-GL at killing C1498 cells in vitro.

### Example 5: Engineered NDV Vector Stimulates NK Cells in Ovarian Tumor

### Bearing Mice

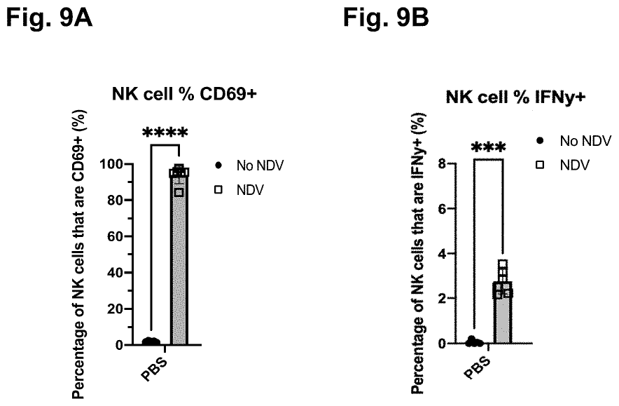

The ability of engineered NDV vector to stimulate the immune system was tested in a model of ovarian tumor bearing mice (Russell et al., 2015). These tumor bearing mice were injected with phosphate-buffered saline mock control, adeno-associated virus (AAV) expressing thrombospondin-1 type I repeats (3TSR), AAV expressing Fc3TSR, or AAV expressing bevacizumab, in the absence or presence of engineered NDV-GFP-GM vector. The 3TSR is a glycoprotein with potent anti-angiogenic factor, which is used in cancer treatment; Fc3TSR is a stabilized form of this glycoprotein. Bevacizumab is a recombinant antibody targeting the vascular endothelial growth factor (VEGF), a pro-angiogenic protein. In this Example, 3TSR, Fc3TSR and bevazicumab were expressed by an adeno-associated virus, and used in combination with NDV-GFP delivered intravenously. Blood was obtained from the mice via retro-orbital bleeds 36 hours post NDV-GFP infection. Red blood cells were lysed, and remaining cells were stained via flow cytometry to analyze for markers indicative of immune stimulation. Over 90% NK cells were detected to express the early activation marker CD69 (FIG. 9A) and over 20% NK cells were PD-L1+ in all groups injected with the engineered NDV-GFP vector, but there was negligible detection in its absence. Granzyme B+ and IFNy+NK cells were also detected in the engineered NDV-GFP vector group but not in its absence (FIG. 9B). Together, these results demonstrated that NDV-GFP leads to the potent stimulation of NK cells in ovarian tumor bearing mice. NDV of the present disclosure is useful as an oncolytic agent.

### Example 6: NDV-Prefusion Stabilized SARS-CoV-2 Spike (NDV-PFS) Protects Against SARS-CoV-2 in Hamsters

### Prefusion Stabilized SARS-CoV-2 Spike (PFS) Expression

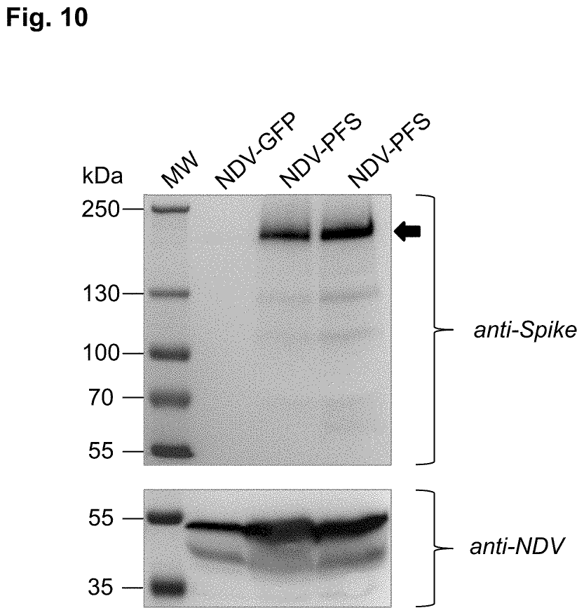

Expression of prefusion stabilized SARS-CoV-2 spike (PFS; SEQ ID NO: 41) in the allantoic fluid of embryonated eggs inoculated with NDV-PFS (SEQ ID NO: 4) was determined by Western immunoblotting. A 6% SDS-PAGE gel and rabbit anti-SARS-CoV-2 S1 (dilution: 1:1000; PA5-81795; ThermoFisher) was used for detection of SARS-CoV-2 spike (FIG. 10; black arrow). A 10% SDS-PAGE gel and mouse anti-NDV ribonucleoprotein (dilution: 1:5000; NBP2-11633; Novus Biologicals) was used for detection of NDV. 20 μL of allantoic fluid was loaded in for samples. NDV-GFP was loaded as a control. MW used was the PageRuler™ Plus Prestained Protein Ladder (Thermo Scientific). These results showed robust expression of SARS-CoV-2 S1 from embryonated eggs inoculated with NDV-PFS, indicating the ability of this NDV platform for delivering a payload such as SARS-CoV-2 S1.

Protection from Weight Loss in NDV-COVID-19 Vaccinated Hamsters Challenged with SARS-CoV-2

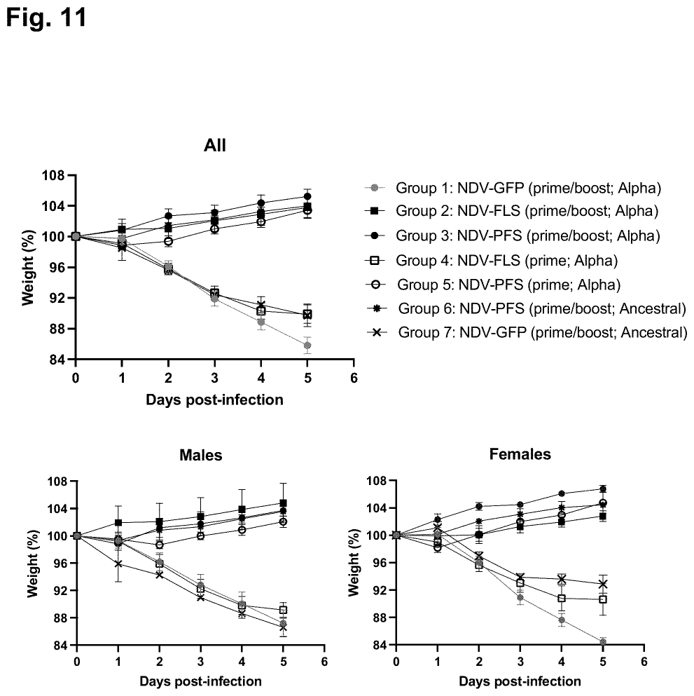

The inventors next determined the effects of NDV-PFS vaccination on hamsters challenged with SARS-CoV-2. Groups of eight Syrian Golden hamsters (four male and four female, four to six weeks of age; Charles River) were anaesthetized with inhalation isoflurane and administered 1E7 PFU/animal of recombinant NDV-GFP, NDV-FLS, or NDV-PFS via the intranasal (IN) route. For IN vaccinations, anaesthetized hamsters were scruffed and vaccines were delivered in a 100 μL volume (q.s. with PBS) through the nares (50 μL per nare). Animals had their mouths held closed to ensure inhalation through the nose. For the prime/boost groups, 28 days following the initial vaccine administration, hamsters were administered a second dose of the homologous vaccine (1E7 PFU/animal by IN route). At 28 days post-prime or 28 days post-prime/boost, hamsters were moved into a CL-3 facility, anaesthetized with inhaled isoflurane and infected SARS-CoV-2 via the same IN method described above. Challenge dose: Alpha variant @ 8.5E4 PFU/animal by IN, Ancestral (Wuhan) @ 1E5 PFU/animal by IN. After recovery from anesthetic hamsters were monitored daily throughout the course of infection. FIG. 11 shows graphs of results of body weights of hamsters, which were recorded daily (error bars represent mean+/−SEM). These results showed that NDV-COVID-19 vaccination, in particular NDV-PFS vaccination, provided protection from weight loss in hamsters challenged with SARS-CoV-2, whether with the alpha variant or the ancestral strain.

### Reduced SARS-CoV-2 Viral RNA Copies in the Lung and Nasal Turbinates of Vaccinated and Challenged Syrian Hamsters

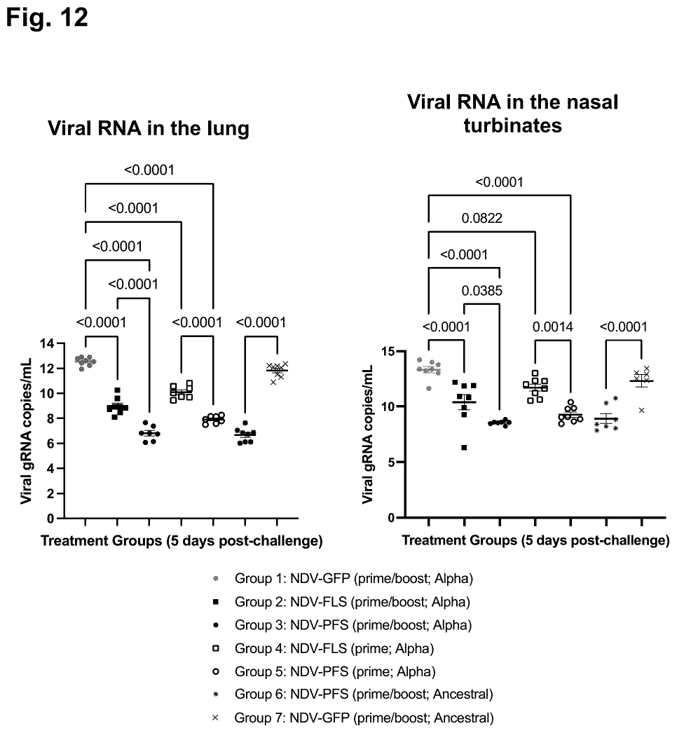

The effects of NDV-COVID-19 vaccination on SARS-CoV-2 viral RNA copies in the lung and nasal turbinates in hamsters were determined. The hamsters were vaccinated and challenged as above, and at 5 days post challenge with Alpha variant @ 8.5E4 PFU/animal by IN or Ancestral (Wuhan) @ 1E5 PFU/animal by IN, vaccinated hamsters were euthanized and viral RNA copies in the lung and nasal turbinates quantified by qRT-PCR. RNA was extracted with the QIAamp Viral RNA Mini kit (Qiagen) and reverse transcribed and amplified using the primers reported by the WHO and include E_Sarbeco_F1 (5′-ACAGGTACGTTAATAGTTAATAGCGT-3′; SEQ ID NO: 37) and E_Sarbeco_R2 (5′-ATATTGCAGCAGTA CGCACACA-3′; SEQ ID NO: 38) and probe E_Sarbeco_P1 (5′-FAM-ACACTAGCCATCCTTACTGCGCTTCG-BBQ-3′; SEQ ID NO: 39). A standard curve produced with synthesized target DNA was run with every plate and used for the interpolation of viral genome copy numbers. FIG. 12 shows graphs of viral RNA levels reported as genome copy number (error bars represent mean+/−SEM). Differences in the magnitude of virus copy number were assessed by Kruskall-Wallis test with Dunn's test for multiple comparisons. These results showed that NDV-COVID-19 vaccination, in particular NDV-PFS vaccination, reduced SARS-CoV-2 viral RNA copies in the lung and nasal turbinates of hamsters challenged with SARS-CoV-2, whether with the alpha variant or the ancestral strain.

### Reduced Infectious SARS-CoV-2 in the Lung and Nasal Turbinates of Vaccinated and Challenged Syrian Hamsters

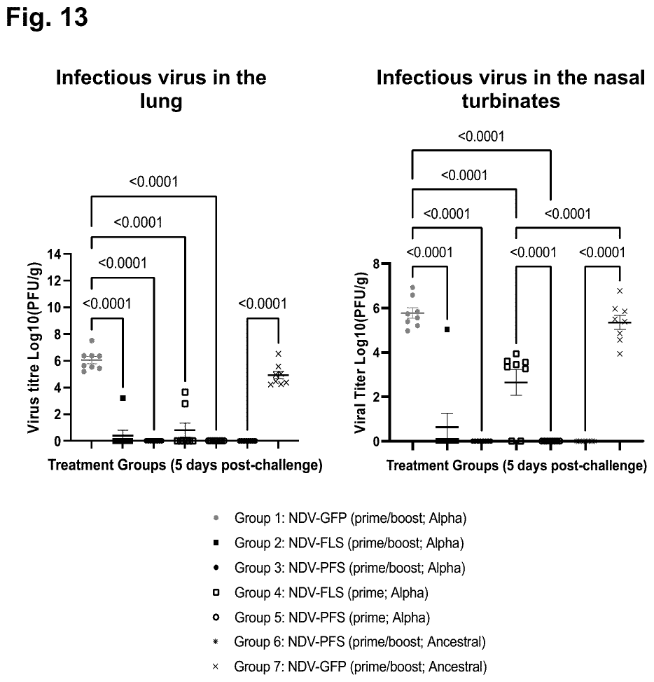

The effects of NDV-COVID-19 vaccination on infectious SARS-CoV-2 in the lung and nasal turbinates in hamsters were determined. The hamsters were vaccinated and challenged as above, and at 5 days post challenge with Alpha variant @ 8.5E4 PFU/animal by IN or Ancestral (Wuhan) @ 1E5 PFU/animal by IN, vaccinated hamsters were euthanized and infectious titers of SARS-CoV-2 in the lung and nasal turbinates determined. For infectious virus assays, thawed tissue samples were weighed and placed in 1 mL of minimum essential medium supplemented with 1% heat-inactivated fetal bovine serum (FBS) and 1×L-glutamine, then homogenized in a Bead Ruptor Elite Bead Mill Homogenizer (Omni International) at 4 m/s for 30 seconds then clarified by centrifugation at 1,500×g for 10 minutes. Samples were serially diluted 10-fold in media and dilutions were then added to 96-well plates of 95% confluent Vero cells containing 50 μL of the same medium in replicates of three and incubated for five days at 37° C. with 5% CO₂. FIG. 13 shows graphs of results from plates that were scored for the presence of cytopathic effect on day five after infection, and the titers were calculated using the Reed-Muench method, converted to PFU after multiplying by 0.69 and reported as PFU/g of tissue. These results showed that NDV-COVID-19 vaccination, in particular NDV-PFS vaccination, reduced infectious SARS-CoV-2 in the lung and nasal turbinates of hamsters challenged with SARS-CoV-2, whether with the alpha variant or the ancestral strain. Together, these results showed that NDV-COVID-19 of the present disclosure, including NDV-PFS, is a useful platform for vaccine against COVID-19.

While the present disclosure has been described with reference to what are presently considered to be the preferred example, it is to be understood that the disclosure is not limited to the disclosed example. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Bukreyev, A., et al., Recombinant newcastle disease virus expressing a foreign viral antigen is attenuated and highly immunogenic in primates. J Virol, 2005. 79(21): p. 13275-84.
Csatary, L. K., et al., Attenuated veterinary virus vaccine for the treatment of cancer. Cancer Detect Prev, 1993. 17(6): p. 619-27.
DiNapoli, J. M., et al., Respiratory tract immunization of non-human primates with a Newcastle disease virus-vectored vaccine candidate against Ebola virus elicits a neutralizing antibody response. Vaccine, 2010. 29(1): p. 17-25.
DiNapoli, J. M., et al., Immunization of primates with a Newcastle disease virus-vectored vaccine via the respiratory tract induces a high titer of serum neutralizing antibodies against highly pathogenic avian influenza virus. J Virol, 2007. 81(21): p. 11560-8.
Fukushi, S., et al., Vesicular stomatitis virus pseudotyped with severe acute respiratory syndrome coronavirus spike protein. J Gen Virol, 2005. 86(Pt 8): p. 2269-2274.
Gao Q et al, Expression of transgenes from newcastle disease virus with a segmented genome. J Virol 2008 March; 82(6):2692-2698.
Hsieh, C-L, et al. Structure-based design of prefusion-stabilized SARS-CoV-2 spikes. Science 369.6510 (2020): 1501-1505.
Kolakofsky, D., et al., Paramyxovirus RNA synthesis and the requirement for hexamer genome length: the rule of six revisited. J Virol, 1998. 72(2): p. 891-9.
Johnson, M. C., et al., Optimized Pseudotyping Conditions for the SARS-COV-2 Spike Glycoprotein. J Virol, 2020. 94(21).
Park, M-S, et al., Engineered viral vaccine constructs with dual specificity: avian influenza and Newcastle disease. Proceedings of the National Academy of Sciences 103.21 (2006): 8203-8208.
Pecora, A. L., et al., Phase I trial of intravenous administration of PV701, an oncolytic virus, in patients with advanced solid cancers. J Clin Oncol, 2002. 20(9): p. 2251-66.
Peiris, M. and G. M. Leung, What can we expect from first-generation COVID-19 vaccines? Lancet, 2020. 396(10261): p. 1467-1469.
Pham P H, et al., Isolation of Ontario aquatic bird bornavirus 1 and characterization of its replication in immortalized avian cell lines. Virol J. 2020 Jan. 31; 17(1):16. doi: 10.1186/s12985-020-1286-6. PMID: 32005267;
Russell S et al. Combined therapy with thrombospondin-1 type I repeats (3TSR) and chemotherapy induces regression and significantly improves survival in a preclinical model of advanced stage epithelial ovarian cancer. FASEB J. 2015 February; 29(2):576-88.
Santry, L. A., et al., Production and Purification of High-Titer Newcastle Disease Virus for Use in Preclinical Mouse Models of Cancer. Mol Ther Methods Clin Dev. 2017 Oct. 16; 9:181-191. doi: 10.1016/j.omtm.2017.10.004.
Sergei, T. A., L. W. McGinnes, and T. G. Morrison, A single amino acid change in the Newcastle disease virus fusion protein alters the requirement for HN protein in fusion. J Virol, 2000. 74(11): p. 5101-7.
Wheelock, E. F. and J. H. Dingle, Observations on the Repeated Administration of Viruses to a Patient with Acute Leukemia. A Preliminary Report. N Engl J

Med, 1964. 271: p. 645-51.

1. An engineered Newcastle Disease Virus (NDV) vector comprising a nucleic acid having a nucleic acid sequence that is at least 95% or 100% identical to the nucleic acid sequence of any one of SEQ ID NO: 1-5, 9, 10, 18, 19, 23, 27, or 42, wherein the nucleic acid comprises or further comprises at least one heterologous nucleic acid segment encoding a therapeutic agent operably linked to a promoter capable of expressing the segment in a host cell, wherein the nucleic acid comprises a nucleic acid sequence encoding an L protein having a stabilizing segment, and wherein the nucleic acid comprises XbaI and MluI restriction endonuclease sites between nucleic acid sequence encoding phosphoprotein and matrix protein.

2. The engineered NDV vector claim 1, comprising a nucleic acid having a nucleic acid sequence that is at least 95% or 100% identical to the nucleic acid sequence of SEQ ID NO: 9, 10, 23, or 27.

3. The engineered NDV vector of claim 1, wherein the therapeutic agent comprises a SARS-CoV-2 spike protein.

4. The engineered NDV vector of claim 3, wherein the SARS-CoV-2 spike protein comprises an amino acid sequence having at least 95% identity to the amino acid sequence of SEQ ID NO: 6, 7, 29, 30, 31, or 41.

5. The engineered NDV vector of claim 1, comprising a nucleic acid having a nucleic acid sequence a chimeric F protein, and a chimeric HN protein, wherein the chimeric F protein comprises avian paramyxovirus 5 (APMV5) F protein segment thereof at the N-terminus and an NDV F protein segment at the C-terminus, and wherein the chimeric HN protein comprises an NDV HN protein segment at the N-terminus and an AMPV5 HN protein segment at the C-terminus.

6. An engineered Newcastle Disease Virus (NDV) vector comprising a nucleic acid having a nucleic acid sequence encoding an L protein having a stabilizing segment, a chimeric F protein, and a chimeric HN protein, wherein the chimeric F protein comprises avian paramyxovirus 5 (APMV5) F protein segment thereof at the N-terminus and an NDV F protein segment at the C-terminus, and wherein the chimeric HN protein comprises an NDV HN protein segment at the N-terminus and an AMPV5 HN protein segment at the C-terminus.

7. The engineered NDV vector of claim 6, wherein the nucleic acid comprises XbaI and MluI restriction endonuclease sites between nucleic acid sequence encoding phosphoprotein and matrix protein.

8. The engineered NDV vector of claim 6, wherein the stabilizing segment comprises an amino acid sequence as set forth in SEQ ID NO: 20, or comprises an amino acid sequence encoded by a nucleic acid comprising a nucleic acid sequence as set forth in SEQ ID NO: 35.

9. The engineered NDV vector of claim 6, wherein the chimeric F protein comprises at the C-terminus 53 amino acid of NDV F protein from amino acid positions 501 to 553 of SEQ ID NO: 28, or the chimeric HN protein comprises at the N-terminus 53 amino acids of NDV HN protein from amino acid positions 1 to 53 of SEQ ID NO: 34.

10. The engineered NDV vector of claim 6, wherein the L protein comprises an amino acid sequence having at least 95% identity to the amino acid sequence as set forth in SEQ ID NO: 11, the chimeric F protein comprises an amino acid sequence having at least 95% identity to the amino acid sequence of SEQ ID NO: 12, and/or the chimeric HN protein comprises an amino acid sequence having at least 95% identity to the amino acid sequence of SEQ ID NO: 13.

11. The engineered NDV vector of claim 6, wherein the NDV vector is lentogenic, and wherein the nucleic acid comprises a nucleic acid sequence of SEQ ID NO: 25.

12. The engineered NDV vector of claim 6, wherein the nucleic acid further comprises at least one heterologous nucleic acid segment encoding a therapeutic agent operably linked to a promoter capable of expressing the segment in a host cell.

13. The engineered NDV vector of claim 6, wherein the therapeutic agent comprises a SARS-CoV-2 spike protein.

14. The engineered NDV vector of claim 13, wherein the SARS-CoV-2 spike protein comprises an amino acid sequence having at least 95% identity to the amino acid sequence of SEQ ID NO: 6, 7, 29, 30, 31, or 41.

15. An immunogenic composition, oncolytic agent, or vaccine comprising the engineered NDV vector of claim 6.

16. A method for treating a disease, comprising administering to a subject the engineered NDV vector of claim 6.

17. A method of eliciting an immune response, comprising administering to a subject the engineered NDV vector of claim 6.

18. A method of treating cancer, comprising administering to a subject the engineered NDV vector of claim 1, wherein the NDV vector comprises a nucleic acid having a nucleic acid sequence that is at least 95% or 100% identical to the nucleic acid sequence of any one of SEQ ID NO: 1, 5, 9, 10, 23, or 27.

19. A method for selecting an engineered NDV vector genome comprising a stabilizing segment in L gene, the method comprises:
a) growing bacterial cells comprising an engineered NDV vector genome in a growth medium broth;
b) growing the bacterial cells on an agar-growth medium, wherein the agar-growth medium comprises a selection agent;
c) identifying small bacterial cell colonies having about 0.5 mm to about 1 mm in diameter after at least 24 hours of growth;
d) repeating step a) to step c) two to nine times to enrich for small bacterial cell colonies; and
e) isolating the engineered NDV vector genome from the small bacterial cells colonies,
wherein the small bacterial cells colonies comprise stable engineered NDV vector genome having the stabilizing segment in L gene.


SEQUENCE LISTING (42 sequences)

SEQ ID NO: 1 - DNA, 16192 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagattagaaaaaatacgggtagaaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatat

SEQ ID NO: 2 - DNA, 19293 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagattagaaaaaatacgggtagaaccgccaccatgttcgtgttcctggtcctgctgccactggtaagctcccaatgtgtaaacttaaccacaagaacccagctcccacctgcctacaccaacagcttcaccagaggcgtttattaccccgacaaggtattccggtcttctgttctgcactctacccaggacctgtttctgccctttttcagcaacgtgacatggttccacgccatccacgtgtctggcacaaacggcaccaagcggtttgataatcctgtgctccctttcaatgacggcgtgtacttcgcctctactgagaagagcaacatcatccggggctggatctttggcacaacactggactctaaaacccagagcctgctgatcgtgaacaacgccaccaacgtggtgattaaggtgtgcgagttccagttctgcaatgaccctttcctcggcgtgtactaccacaagaacaacaaaagttggatggaaagcgaattcagggtgtactcaagcgccaacaactgtaccttcgagtacgtgagccagcctttcctgatggacctagaaggtaagcagggcaatttcaagaacctcagagagttcgtgttcaagaatattgacggctacttcaaaatctacagcaagcacaccccaatcaacctggtgcgggacctgccccagggctttagcgcgctggagcctctggtggacctgcctatcggcatcaacatcacccggttccagacactgctggctctgcatagaagctacctgacacctggcgacagttcttctggctggacagccggcgccgccgcctactacgtgggctacctgcagcctagaacattcctgctgaaatacaacgagaacggcacgatcacagacgccgtggactgcgccctggatcccctgtctgagacaaagtgcaccctgaagtctttcaccgtggagaagggcatctaccagacctccaacttcagagtgcagcctaccgaatccatcgtgcgctttcccaacatcaccaacctgtgccccttcggcgaggtctttaatgccacgagattcgccagcgtgtatgcctggaacagaaagagaatcagcaactgcgtggccgactacagcgtgctgtacaacagcgcctctttcagcacatttaagtgctacggagtgtctcctaccaaactcaacgatctgtgcttcacgaacgtgtatgccgacagcttcgtgatccgaggagatgaggtgcggcagatcgctccaggacagacaggcaagatcgccgactacaactacaagctgcccgacgactttaccggctgcgtgatcgcttggaacagcaataacctggactcaaaggttggaggaaactacaactacctgtacagactgttcagaaagtccaacctgaagcccttcgagagagacatctctacagaaatctaccaggccggcagcaccccatgtaacggcgtggaaggcttcaactgctacttccctctgcagtcttatggcttccagcccacaaacggagtgggctatcagccttaccgcgtggttgtcctgagctttgagctgctgcatgcccctgctacggtgtgtggacctaagaagtccaccaacctggtgaagaacaagtgtgtgaacttcaacttcaacggcctgaccggcaccggggtgctgacagagtctaacaagaaattcctgccattccagcaattcggccgggacatcgccgacaccaccgacgccgtgcgggatcctcagaccctcgaaatcctggacatcaccccctgtagcttcggcggcgtgagcgtgatcacccctggcacaaacaccagcaatcaagtggctgtcctgtaccaggatgtcaattgcacagaagtgcctgtggccatccacgccgatcagctgacccccacctggcgggtgtactcgacaggaagcaacgtgtttcaaacaagagccggctgcctgatcggggccgagcacgtgaacaattcctacgagtgcgacatccccatcggcgccggcatctgtgcctcttaccagacacagaccaattcccctcgtagagccagatccgtggccagccagagcatcatcgcctacaccatgagcctgggcgccgaaaacagcgttgcatattccaacaacagcatcgccatccctaccaacttcaccatcagcgtgaccacagaaatcctgcctgtgtccatgaccaagacaagcgttgattgcaccatgtacatctgcggcgatagcacagagtgcagcaatctgctgctgcagtacggtagcttctgcacccagctgaatagagccctgaccggcatcgctgtggaacaggacaaaaacacccaggaggtcttcgcccaggtgaagcaaatctacaagacccctccaatcaaggacttcggaggctttaactttagccagatcctgcctgatccctccaagcctagcaaacggagtttcatcgaggacctgctcttcaacaaggtgaccctggctgacgccggcttcattaagcagtacggcgattgcctcggcgacatcgctgcaagagacctgatctgcgcccagaagttcaacggcctgaccgtgctgcctcctctcctgacagacgagatgatcgcccagtacacctctgcccttctggctggcaccatcaccagcggatggacctttggagccggagccgccctgcagatccctttcgctatgcagatggcctacagattcaacgggatcggagtgacccaaaacgtgctgtatgaaaaccagaaactgatcgccaatcagtttaacagcgccatcggcaaaatccaggatagcctgtccagcaccgccagcgccctcggcaagctgcaagatgtggtgaatcaaaatgcccaagccctgaacacactggtgaagcagctgagcagcaacttcggcgccatcagcagcgtgctgaacgacatcctgagcagactggacaaggtggaagccgaggtgcagatcgacagactgatcacaggcagactgcagtccctgcagacctacgtgacccagcagttgattagagccgctgagattagagccagtgccaacctggctgccacaaagatgtcagaatgcgtgctgggccagagcaagagagtggacttctgcggcaaaggctaccacctgatgagctttcctcagtctgcaccccacggcgtggtgtttctccacgtgacatacgtgcccgcgcaagaaaagaactttacaaccgccccagcgatctgccacgacggcaaggcccacttccctcgggagggtgtgttcgtgagcaatggaacacactggttcgtcacccagcggaacttctacgagcctcagatcattaccaccgacaacaccttcgtgagcggcaactgtgacgtcgttatcggcatcgtgaacaataccgtgtacgaccccctgcagcctgagctggatagcttcaaagaggaactggacaagtacttcaagaaccacacaagccccgacgtggacctaggcgacatctctggaatcaacgccagcgtggtgaacatccaaaaggaaatcgacagactgaacgaggtggccaagaatctgaatgaaagcctgatcgatctgcaggagctgggcaagtacgagcagtacatcaaatggccttggtacatctggctgggcttcatcgctggtctgatcgctatcgtgatggtgaccattatgctgtgctgcatgacctcctgctgctcttgcctgaagggctgttgttcttgcggctcttgctgcaagttcgacgaggatgactctgaacctgttctgaagggcgtgaagctgcactacacctgataaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactata

SEQ ID NO: 3 - DNA, 19240 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagattagaaaaaatacgggtagaaccgccaccatgttcgtgttcctggtcctgctgccactggtaagctcccaatgtgtaaacttaaccacaagaacccagctcccacctgcctacaccaacagcttcaccagaggcgtttattaccccgacaaggtattccggtcttctgttctgcactctacccaggacctgtttctgccctttttcagcaacgtgacatggttccacgccatccacgtgtctggcacaaacggcaccaagcggtttgataatcctgtgctccctttcaatgacggcgtgtacttcgcctctactgagaagagcaacatcatccggggctggatctttggcacaacactggactctaaaacccagagcctgctgatcgtgaacaacgccaccaacgtggtgattaaggtgtgcgagttccagttctgcaatgaccctttcctcggcgtgtactaccacaagaacaacaaaagttggatggaaagcgaattcagggtgtactcaagcgccaacaactgtaccttcgagtacgtgagccagcctttcctgatggacctagaaggtaagcagggcaatttcaagaacctcagagagttcgtgttcaagaatattgacggctacttcaaaatctacagcaagcacaccccaatcaacctggtgcgggacctgccccagggctttagcgcgctggagcctctggtggacctgcctatcggcatcaacatcacccggttccagacactgctggctctgcatagaagctacctgacacctggcgacagttcttctggctggacagccggcgccgccgcctactacgtgggctacctgcagcctagaacattcctgctgaaatacaacgagaacggcacgatcacagacgccgtggactgcgccctggatcccctgtctgagacaaagtgcaccctgaagtctttcaccgtggagaagggcatctaccagacctccaacttcagagtgcagcctaccgaatccatcgtgcgctttcccaacatcaccaacctgtgccccttcggcgaggtctttaatgccacgagattcgccagcgtgtatgcctggaacagaaagagaatcagcaactgcgtggccgactacagcgtgctgtacaacagcgcctctttcagcacatttaagtgctacggagtgtctcctaccaaactcaacgatctgtgcttcacgaacgtgtatgccgacagcttcgtgatccgaggagatgaggtgcggcagatcgctccaggacagacaggcaagatcgccgactacaactacaagctgcccgacgactttaccggctgcgtgatcgcttggaacagcaataacctggactcaaaggttggaggaaactacaactacctgtacagactgttcagaaagtccaacctgaagcccttcgagagagacatctctacagaaatctaccaggccggcagcaccccatgtaacggcgtggaaggcttcaactgctacttccctctgcagtcttatggcttccagcccacaaacggagtgggctatcagccttaccgcgtggttgtcctgagctttgagctgctgcatgcccctgctacggtgtgtggacctaagaagtccaccaacctggtgaagaacaagtgtgtgaacttcaacttcaacggcctgaccggcaccggggtgctgacagagtctaacaagaaattcctgccattccagcaattcggccgggacatcgccgacaccaccgacgccgtgcgggatcctcagaccctcgaaatcctggacatcaccccctgtagcttcggcggcgtgagcgtgatcacccctggcacaaacaccagcaatcaagtggctgtcctgtaccaggatgtcaattgcacagaagtgcctgtggccatccacgccgatcagctgacccccacctggcgggtgtactcgacaggaagcaacgtgtttcaaacaagagccggctgcctgatcggggccgagcacgtgaacaattcctacgagtgcgacatccccatcggcgccggcatctgtgcctcttaccagacacagaccaattcccctcgtagagccagatccgtggccagccagagcatcatcgcctacaccatgagcctgggcgccgaaaacagcgttgcatattccaacaacagcatcgccatccctaccaacttcaccatcagcgtgaccacagaaatcctgcctgtgtccatgaccaagacaagcgttgattgcaccatgtacatctgcggcgatagcacagagtgcagcaatctgctgctgcagtacggtagcttctgcacccagctgaatagagccctgaccggcatcgctgtggaacaggacaaaaacacccaggaggtcttcgcccaggtgaagcaaatctacaagacccctccaatcaaggacttcggaggctttaactttagccagatcctgcctgatccctccaagcctagcaaacggagtttcatcgaggacctgctcttcaacaaggtgaccctggctgacgccggcttcattaagcagtacggcgattgcctcggcgacatcgctgcaagagacctgatctgcgcccagaagttcaacggcctgaccgtgctgcctcctctcctgacagacgagatgatcgcccagtacacctctgcccttctggctggcaccatcaccagcggatggacctttggagccggagccgccctgcagatccctttcgctatgcagatggcctacagattcaacgggatcggagtgacccaaaacgtgctgtatgaaaaccagaaactgatcgccaatcagtttaacagcgccatcggcaaaatccaggatagcctgtccagcaccgccagcgccctcggcaagctgcaagatgtggtgaatcaaaatgcccaagccctgaacacactggtgaagcagctgagcagcaacttcggcgccatcagcagcgtgctgaacgacatcctgagcagactggacaaggtggaagccgaggtgcagatcgacagactgatcacaggcagactgcagtccctgcagacctacgtgacccagcagttgattagagccgctgagattagagccagtgccaacctggctgccacaaagatgtcagaatgcgtgctgggccagagcaagagagtggacttctgcggcaaaggctaccacctgatgagctttcctcagtctgcaccccacggcgtggtgtttctccacgtgacatacgtgcccgcgcaagaaaagaactttacaaccgccccagcgatctgccacgacggcaaggcccacttccctcgggagggtgtgttcgtgagcaatggaacacactggttcgtcacccagcggaacttctacgagcctcagatcattaccaccgacaacaccttcgtgagcggcaactgtgacgtcgttatcggcatcgtgaacaataccgtgtacgaccccctgcagcctgagctggatagcttcaaagaggaactggacaagtacttcaagaaccacacaagccccgacgtggacctaggcgacatctctggaatcaacgccagcgtggtgaacatccaaaaggaaatcgacagactgaacgaggtggccaagaatctgaatgaaagcctgatcgatctgcaggagctgggcaagtacgagcagtacatcaaatggccttggtacatctggctgggcttcatcgctggtctgatcgctatcgtgatggtgaccattatgctgtgctgcatgacctcctgctgctcttgcctgaagggctgttgttcttgcggctcttgctgctgatgataaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatat

SEQ ID NO: 4 - DNA, 19269 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagattagaaaaaatacgggtagaaccgccaccatgttcgtgttcctggtcctgctgccactggtaagctcccaatgtgtaaacttaaccacaagaacccagctcccacctgcctacaccaacagcttcaccagaggcgtttattaccccgacaaggtattccggtcttctgttctgcactctacccaggacctgtttctgccctttttcagcaacgtgacatggttccacgccatccacgtgtctggcacaaacggcaccaagcggtttgataatcctgtgctccctttcaatgacggcgtgtacttcgcctctactgagaagagcaacatcatccggggctggatctttggcacaacactggactctaaaacccagagcctgctgatcgtgaacaacgccaccaacgtggtgattaaggtgtgcgagttccagttctgcaatgaccctttcctcggcgtgtactaccacaagaacaacaaaagttggatggaaagcgaattcagggtgtactcaagcgccaacaactgtaccttcgagtacgtgagccagcctttcctgatggacctagaaggtaagcagggcaatttcaagaacctcagagagttcgtgttcaagaatattgacggctacttcaaaatctacagcaagcacaccccaatcaacctggtgcgggacctgccccagggctttagcgcgctggagcctctggtggacctgcctatcggcatcaacatcacccggttccagacactgctggctctgcatagaagctacctgacacctggcgacagttcttctggctggacagccggcgccgccgcctactacgtgggctacctgcagcctagaacattcctgctgaaatacaacgagaacggcacgatcacagacgccgtggactgcgccctggatcccctgtctgagacaaagtgcaccctgaagtctttcaccgtggagaagggcatctaccagacctccaacttcagagtgcagcctaccgaatccatcgtgcgctttcccaacatcaccaacctgtgccccttcggcgaggtctttaatgccacgagattcgccagcgtgtatgcctggaacagaaagagaatcagcaactgcgtggccgactacagcgtgctgtacaacagcgcctctttcagcacatttaagtgctacggagtgtctcctaccaaactcaacgatctgtgcttcacgaacgtgtatgccgacagcttcgtgatccgaggagatgaggtgcggcagatcgctccaggacagacaggcaagatcgccgactacaactacaagctgcccgacgactttaccggctgcgtgatcgcttggaacagcaataacctggactcaaaggttggaggaaactacaactacctgtacagactgttcagaaagtccaacctgaagcccttcgagagagacatctctacagaaatctaccaggccggcagcaccccatgtaacggcgtggaaggcttcaactgctacttccctctgcagtcttatggcttccagcccacaaacggagtgggctatcagccttaccgcgtggttgtcctgagctttgagctgctgcatgcccctgctacggtgtgtggacctaagaagtccaccaacctggtgaagaacaagtgtgtgaacttcaacttcaacggcctgaccggcaccggggtgctgacagagtctaacaagaaattcctgccattccagcaattcggccgggacatcgccgacaccaccgacgccgtgcgggatcctcagaccctcgaaatcctggacatcaccccctgtagcttcggcggcgtgagcgtgatcacccctggcacaaacaccagcaatcaagtggctgtcctgtaccaggatgtcaattgcacagaagtgcctgtggccatccacgccgatcagctgacccccacctggcgggtgtactcgacaggaagcaacgtgtttcaaacaagagccggctgcctgatcggggccgagcacgtgaacaattcctacgagtgcgacatccccatcggcgccggcatctgtgcctcttaccagacacagaccaattcccctggtagtgcaagttccgtggccagccagagcatcatcgcctacaccatgagcctgggcgccgaaaacagcgttgcatattccaacaacagcatcgccatccctaccaacttcaccatcagcgtgaccacagaaatcctgcctgtgtccatgaccaagacaagcgttgattgcaccatgtacatctgcggcgatagcacagagtgcagcaatctgctgctgcagtacggtagcttctgcacccagctgaatagagccctgaccggcatcgctgtggaacaggacaaaaacacccaggaggtcttcgcccaggtgaagcaaatctacaagacccctccaatcaaggacttcggaggctttaactttagccagatcctgcctgatccctccaagcctagcaaacggagtcctatcgaggacctgctcttcaacaaggtgaccctggctgacgccggcttcattaagcagtacggcgattgcctcggcgacatcgctgcaagagacctgatctgcgcccagaagttcaacggcctgaccgtgctgcctcctctcctgacagacgagatgatcgcccagtacacctctgcccttctggctggcaccatcaccagcggatggacctttggagccggacctgccctgcagatccctttccctatgcagatggcctacagattcaacgggatcggagtgacccaaaacgtgctgtatgaaaaccagaaactgatcgccaatcagtttaacagcgccatcggcaaaatccaggatagcctgtccagcaccccaagcgccctcggcaagctgcaagatgtggtgaatcaaaatgcccaagccctgaacacactggtgaagcagctgagcagcaacttcggcgccatcagcagcgtgctgaacgacatcctgagcagactggacccacctgaagccgaggtgcagatcgacagactgatcacaggcagactgcagtccctgcagacctacgtgacccagcagttgattagagccgctgagattagagccagtgccaacctggctgccacaaagatgtcagaatgcgtgctgggccagagcaagagagtggacttctgcggcaaaggctaccacctgatgagctttcctcagtctgcaccccacggcgtggtgtttctccacgtgacatacgtgcccgcgcaagaaaagaactttacaaccgccccagcgatctgccacgacggcaaggcccacttccctcgggagggtgtgttcgtgagcaatggaacacactggttcgtcacccagcggaacttctacgagcctcagatcattaccaccgacaacaccttcgtgagcggcaactgtgacgtcgttatcggcatcgtgaacaataccgtgtacgaccccctgcagcctgagctggatagcttcaaagaggaactggacaagtacttcaagaaccacacaagccccgacgtggacctaggcgacatctctggaatcaacgccagcgtggtgaacatccaaaaggaaatcgacagactgaacgaggtggccaagaatctgaatgaaagcctgatcgatctgcaggagctgggcaagtacgagcagggtggcggtggctcgctgattacctatatcgtcctgactattatctccctggtgtttggcattctgtccctgattctggcctgttacctgatgtacaagcagaaggcccagcagaagaccctgctgtggctgggcaataatacactggatcagatgcgggctacaactaagatgtgaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactata

SEQ ID NO: 5 - DNA, 16168 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagattagaaaaaatacgggtagaaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgttacaacttcccttgaccattcttcttagcattcttagtgctcaccagtcgctttgtctagacaacagtaagctcattcatgcaggaatcatgagtactactgagagagaagttaatgtttatgcacaatctattactgggtcaatagtggtgagattgattccaaatatcccaagtaaccataaatcttgtgcaactagccaaatcaaattatacaatgacacgttaacaagattgttgaccccaattaaagctaatctagaaggacttattagtgctgtttctcaggaccaatcgcagaattctgggaagagaaagaagcgttttgtaggcgcagtaattggagcagctgcccttggtttggcaactgctgcacaggtgactgccactgtagcattaaatcaagcgcaagaaaacgctcggaatatcctaaggcttaaaaactcgattcagaagacaaacgaggcggtgatggaacttaaagatgctgtgggccaaacagcagtagctattgacaaaactcaggccttcataaataatcaaatcttgcctgcaatttcaaatctctcatgtgaggtcctagggaataaaattggggtccaattatctttgtaccttactgaattaacaacagtattcggcaaccaactgacaaacccagcccttaccacactgtcattacaagccttgtacaatctttgtggagatgacttcaattacttaatcaacctattaaatgcaaaaaatcgtaacttagcctcactttatgaagcaaacctaattcaggggagaattactcaatatgactcaatgaatcagttattaattattcaggtacaaataccaagcatctccacagtgtcaggaatgagggtcacagaattgttcacacttagtgttgacacacctataggagagggaaaggccctagtaccaaaatatgtcctatcctcagggagaataatggaagaggttgacctaagcagttgcgctataacatcaacatcagttttctgttcctctatcatctctagaccccttccacttgaaacaataaattgcctgaatgggaatgttacacagtgtcaatttaccgccaacacaggaacccttgaatcgagatacgctgttataggaggattggtgattgctaactgtaaggctatagtatgcaggtgcctaaatccaccaggtgtcattgcgcaaaatcttggcttaccaattacaatcatctcatccaatacttgtcagcgaattaatttagaacaaatcactttgtctcttgggaacagcatattatctacatacagtgccaatttatcccaagttgagatgaatttagctccatcaaatcctctggatatctcagttgaattgaatcgagtcaacaccagtctctctaaagtggaatctctaataaaagaaagcaatagtatcctggactcagtaaaccctcaaattttaaatgtcaagacactcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacaccttccactctgatcagcctaaataactcaattatcacaagcagcaatggtctcaaaaaggaaatcctgaaccagaacataaaagaggacctcatatatagagaagttgctataaatatacctttaacattagatagggttactgttgaggtagggactgcagtaaaccagattactgatgcactcaggcaactccagtcagttaatggatctgctgcattcgcctcatcaaactctcctgattatagtgggggaatagaacacctgattttccaaaggaatacgcttattaatcgctcagtgagtgtctcagatttaatagaacaccccagtttcataccaactcctactacacagcatggttgtaccagaatccccacattccacctaggaactcgccactggtgctatagtcacaatataataggtcagggatgtgctgattctagagctagtgtgatgtatatttcaatgggagcactgggtgtcagttcattgggaaccccgaccttcacaacatctgctgcatcaatattatctgatagcctcaatcggaagagttgcagtatagtagcaacaactgagggttgtgacgtactctgcagtatagttacacaaacagaagaccaagattatgctgatcacactcctactccaatgatacatggtagattatggtttaatggcacatacacagagagatccttatcccagagtttattccttggaacatgggctgcgcaatatccggctgtaggatctggtataatgacacctgggcgagttatattccctttctatggaggtgtgatccctaactctcctctcttcttggatctcgaaagattcgctttattcacacataatggagacttagaatgcatgaacttaacacaatatcagaaagaagcaatttactctgcatataagcctcccaagattagaggatcactgtgggcacaaggcttcatagtatgttcagtaggagacatggggaattgctctcttaaagtgatcaatacaagcacagttatgatgggtgcagaaggtcggctacaattagttggggactccgttatgtactatcagagatcatcatcctggtggcctgtaggaattctttatcggttgagtcttgtagacatcatcgccggagatatacaggtcgtcataaacagtgaaccactccctctgagcaagttcccgcggccaacctggactccaggagtgtgtcaaaaaccaaatgtatgccctgcagtttgtgtaactggggtctatcaagacctttgggcaatttccgcaggggagacactatctgaaatgacattctttggaggatatttagaggcatccacccaacgaaaagatccatggataggcgttgctaatcaatatagttggttcatgagaagaagattattcaagacaagcactgaagctgcatattcgtcatcaacgtgttttaggaacactagactggatcgaaatttctgcctattagtctttgaattaactgataacttacttggagactggagaattgtccccctcttatttgaattaaccatcgtataaggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatat

SEQ ID NO: 6 - protein, 1273 aa, SARS Coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1273 aa, SARS Coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - DNA, 3825 bp, Artificial
  Synthetic Construct
atgttcgtgttcctggtcctgctgccactggtaagctcccaatgtgtaaacttaaccacaagaacccagctcccacctgcctacaccaacagcttcaccagaggcgtttattaccccgacaaggtattccggtcttctgttctgcactctacccaggacctgtttctgccctttttcagcaacgtgacatggttccacgccatccacgtgtctggcacaaacggcaccaagcggtttgataatcctgtgctccctttcaatgacggcgtgtacttcgcctctactgagaagagcaacatcatccggggctggatctttggcacaacactggactctaaaacccagagcctgctgatcgtgaacaacgccaccaacgtggtgattaaggtgtgcgagttccagttctgcaatgaccctttcctcggcgtgtactaccacaagaacaacaaaagttggatggaaagcgaattcagggtgtactcaagcgccaacaactgtaccttcgagtacgtgagccagcctttcctgatggacctagaaggtaagcagggcaatttcaagaacctcagagagttcgtgttcaagaatattgacggctacttcaaaatctacagcaagcacaccccaatcaacctggtgcgggacctgccccagggctttagcgcgctggagcctctggtggacctgcctatcggcatcaacatcacccggttccagacactgctggctctgcatagaagctacctgacacctggcgacagttcttctggctggacagccggcgccgccgcctactacgtgggctacctgcagcctagaacattcctgctgaaatacaacgagaacggcacgatcacagacgccgtggactgcgccctggatcccctgtctgagacaaagtgcaccctgaagtctttcaccgtggagaagggcatctaccagacctccaacttcagagtgcagcctaccgaatccatcgtgcgctttcccaacatcaccaacctgtgccccttcggcgaggtctttaatgccacgagattcgccagcgtgtatgcctggaacagaaagagaatcagcaactgcgtggccgactacagcgtgctgtacaacagcgcctctttcagcacatttaagtgctacggagtgtctcctaccaaactcaacgatctgtgcttcacgaacgtgtatgccgacagcttcgtgatccgaggagatgaggtgcggcagatcgctccaggacagacaggcaagatcgccgactacaactacaagctgcccgacgactttaccggctgcgtgatcgcttggaacagcaataacctggactcaaaggttggaggaaactacaactacctgtacagactgttcagaaagtccaacctgaagcccttcgagagagacatctctacagaaatctaccaggccggcagcaccccatgtaacggcgtggaaggcttcaactgctacttccctctgcagtcttatggcttccagcccacaaacggagtgggctatcagccttaccgcgtggttgtcctgagctttgagctgctgcatgcccctgctacggtgtgtggacctaagaagtccaccaacctggtgaagaacaagtgtgtgaacttcaacttcaacggcctgaccggcaccggggtgctgacagagtctaacaagaaattcctgccattccagcaattcggccgggacatcgccgacaccaccgacgccgtgcgggatcctcagaccctcgaaatcctggacatcaccccctgtagcttcggcggcgtgagcgtgatcacccctggcacaaacaccagcaatcaagtggctgtcctgtaccaggatgtcaattgcacagaagtgcctgtggccatccacgccgatcagctgacccccacctggcgggtgtactcgacaggaagcaacgtgtttcaaacaagagccggctgcctgatcggggccgagcacgtgaacaattcctacgagtgcgacatccccatcggcgccggcatctgtgcctcttaccagacacagaccaattcccctcgtagagccagatccgtggccagccagagcatcatcgcctacaccatgagcctgggcgccgaaaacagcgttgcatattccaacaacagcatcgccatccctaccaacttcaccatcagcgtgaccacagaaatcctgcctgtgtccatgaccaagacaagcgttgattgcaccatgtacatctgcggcgatagcacagagtgcagcaatctgctgctgcagtacggtagcttctgcacccagctgaatagagccctgaccggcatcgctgtggaacaggacaaaaacacccaggaggtcttcgcccaggtgaagcaaatctacaagacccctccaatcaaggacttcggaggctttaactttagccagatcctgcctgatccctccaagcctagcaaacggagtttcatcgaggacctgctcttcaacaaggtgaccctggctgacgccggcttcattaagcagtacggcgattgcctcggcgacatcgctgcaagagacctgatctgcgcccagaagttcaacggcctgaccgtgctgcctcctctcctgacagacgagatgatcgcccagtacacctctgcccttctggctggcaccatcaccagcggatggacctttggagccggagccgccctgcagatccctttcgctatgcagatggcctacagattcaacgggatcggagtgacccaaaacgtgctgtatgaaaaccagaaactgatcgccaatcagtttaacagcgccatcggcaaaatccaggatagcctgtccagcaccgccagcgccctcggcaagctgcaagatgtggtgaatcaaaatgcccaagccctgaacacactggtgaagcagctgagcagcaacttcggcgccatcagcagcgtgctgaacgacatcctgagcagactggacaaggtggaagccgaggtgcagatcgacagactgatcacaggcagactgcagtccctgcagacctacgtgacccagcagttgattagagccgctgagattagagccagtgccaacctggctgccacaaagatgtcagaatgcgtgctgggccagagcaagagagtggacttctgcggcaaaggctaccacctgatgagctttcctcagtctgcaccccacggcgtggtgtttctccacgtgacatacgtgcccgcgcaagaaaagaactttacaaccgccccagcgatctgccacgacggcaaggcccacttccctcgggagggtgtgttcgtgagcaatggaacacactggttcgtcacccagcggaacttctacgagcctcagatcattaccaccgacaacaccttcgtgagcggcaactgtgacgtcgttatcggcatcgtgaacaataccgtgtacgaccccctgcagcctgagctggatagcttcaaagaggaactggacaagtacttcaagaaccacacaagccccgacgtggacctaggcgacatctctggaatcaacgccagcgtggtgaacatccaaaaggaaatcgacagactgaacgaggtggccaagaatctgaatgaaagcctgatcgatctgcaggagctgggcaagtacgagcagtacatcaaatggccttggtacatctggctgggcttcatcgctggtctgatcgctatcgtgatggtgaccattatgctgtgctgcatgacctcctgctgctcttgcctgaagggctgttgttcttgcggctcttgctgcaagttcgacgaggatgactctgaacctgttctgaagggcgtgaagctgcactacacctgataa

SEQ ID NO: 9 - DNA, 15418 bp, Artificial
  Synthetic construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagaaaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgttacaacttcccttgaccattcttcttagcattcttagtgctcaccagtcgctttgtctagacaacagtaagctcattcatgcaggaatcatgagtactactgagagagaagttaatgtttatgcacaatctattactgggtcaatagtggtgagattgattccaaatatcccaagtaaccataaatcttgtgcaactagccaaatcaaattatacaatgacacgttaacaagattgttgaccccaattaaagctaatctagaaggacttattagtgctgtttctcaggaccaatcgcagaattctgggaagagaaagaagcgttttgtaggcgcagtaattggagcagctgcccttggtttggcaactgctgcacaggtgactgccactgtagcattaaatcaagcgcaagaaaacgctcggaatatcctaaggcttaaaaactcgattcagaagacaaacgaggcggtgatggaacttaaagatgctgtgggccaaacagcagtagctattgacaaaactcaggccttcataaataatcaaatcttgcctgcaatttcaaatctctcatgtgaggtcctagggaataaaattggggtccaattatctttgtaccttactgaattaacaacagtattcggcaaccaactgacaaacccagcccttaccacactgtcattacaagccttgtacaatctttgtggagatgacttcaattacttaatcaacctattaaatgcaaaaaatcgtaacttagcctcactttatgaagcaaacctaattcaggggagaattactcaatatgactcaatgaatcagttattaattattcaggtacaaataccaagcatctccacagtgtcaggaatgagggtcacagaattgttcacacttagtgttgacacacctataggagagggaaaggccctagtaccaaaatatgtcctatcctcagggagaataatggaagaggttgacctaagcagttgcgctataacatcaacatcagttttctgttcctctatcatctctagaccccttccacttgaaacaataaattgcctgaatgggaatgttacacagtgtcaatttaccgccaacacaggaacccttgaatcgagatacgctgttataggaggattggtgattgctaactgtaaggctatagtatgcaggtgcctaaatccaccaggtgtcattgcgcaaaatcttggcttaccaattacaatcatctcatccaatacttgtcagcgaattaatttagaacaaatcactttgtctcttgggaacagcatattatctacatacagtgccaatttatcccaagttgagatgaatttagctccatcaaatcctctggatatctcagttgaattgaatcgagtcaacaccagtctctctaaagtggaatctctaataaaagaaagcaatagtatcctggactcagtaaaccctcaaattttaaatgtcaagacactcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacaccttccactctgatcagcctaaataactcaattatcacaagcagcaatggtctcaaaaaggaaatcctgaaccagaacataaaagaggacctcatatatagagaagttgctataaatatacctttaacattagatagggttactgttgaggtagggactgcagtaaaccagattactgatgcactcaggcaactccagtcagttaatggatctgctgcattcgcctcatcaaactctcctgattatagtgggggaatagaacacctgattttccaaaggaatacgcttattaatcgctcagtgagtgtctcagatttaatagaacaccccagtttcataccaactcctactacacagcatggttgtaccagaatccccacattccacctaggaactcgccactggtgctatagtcacaatataataggtcagggatgtgctgattctagagctagtgtgatgtatatttcaatgggagcactgggtgtcagttcattgggaaccccgaccttcacaacatctgctgcatcaatattatctgatagcctcaatcggaagagttgcagtatagtagcaacaactgagggttgtgacgtactctgcagtatagttacacaaacagaagaccaagattatgctgatcacactcctactccaatgatacatggtagattatggtttaatggcacatacacagagagatccttatcccagagtttattccttggaacatgggctgcgcaatatccggctgtaggatctggtataatgacacctgggcgagttatattccctttctatggaggtgtgatccctaactctcctctcttcttggatctcgaaagattcgctttattcacacataatggagacttagaatgcatgaacttaacacaatatcagaaagaagcaatttactctgcatataagcctcccaagattagaggatcactgtgggcacaaggcttcatagtatgttcagtaggagacatggggaattgctctcttaaagtgatcaatacaagcacagttatgatgggtgcagaaggtcggctacaattagttggggactccgttatgtactatcagagatcatcatcctggtggcctgtaggaattctttatcggttgagtcttgtagacatcatcgccggagatatacaggtcgtcataaacagtgaaccactccctctgagcaagttcccgcggccaacctggactccaggagtgtgtcaaaaaccaaatgtatgccctgcagtttgtgtaactggggtctatcaagacctttgggcaatttccgcaggggagacactatctgaaatgacattctttggaggatatttagaggcatccacccaacgaaaagatccatggataggcgttgctaatcaatatagttggttcatgagaagaagattattcaagacaagcactgaagctgcatattcgtcatcaacgtgttttaggaacactagactggatcgaaatttctgcctattagtctttgaattaactgataacttacttggagactggagaattgtccccctcttatttgaattaaccatcgtataaggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatat

SEQ ID NO: 10 - DNA, 15442 bp, Avian orthoavulavirus 1
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagaaaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatat

SEQ ID NO: 11 - protein, 2204 aa, Avian orthoavulavirus 1
MASSGPERAEHQIILPEPHLSSPLVKHKLLYYWKLTGLPLPDECDFDHLILSRQWKKILESASPDTERMIKLGRAVHQTLNHNSRITGVLHPRCLEQLANIEVPDSTNKFRKIEKKIQIHNTRYGELFTRLCTHIEKKLLGSSWSNNVPRSEEFSSIRTDPAFWFHSKWSTAKFAWLHIKQIQRHLMVAAKTRSAANKLVMLTHKVGQVFVTPELVVVTHTNENKFTCLTQELVLMYADMMEGRDMVNIISTTAVHLRSLSEKIDDILRLIDALAKDLGNQVYDVVSLMEGFAYGAVQLLEPSGTFAGDFFAFNLQELKDILIGLLPNDIAESVTHAIATVFSGLEQNQAAEMLCLLRLWGHPLLESRIAAKAVRSQMCAPKMVDFDMILQVLSFFKGTIINGYRKKNAGVWPRVKVDTIYGKVIGQLHADSAEISHDIMLREYKSLSALEFEPCIEYDPVTNLSMFLKDKAIAHPNDNWLASFRRNLLSEDQKKHVKEATSTNRLLIEFLESNDFDPYKEMEYLTTLEYLRDDNVAVSYSLKEKEVKVNGRIFAKLTKKLRNCQVMAEGILADQIAPFFQGNGVIQDSISLTKSMLAMSQLSFNSNKKRITDCKERVSSNRNHDPKSKNRRRVATFITTDLQKYCLNWRYQTIKLFAHAINQLMGLPHFFEWIHLRLMDTTMFVGDPFNPPSDPTDCDLSRVPNDDIYIVSARGGIEGLCQKLWTMISIAAIQLAAARSHCRVACMVQGDNQVIAVTREVRSDDSPEMVLTQLHQASDNFFKELIHVNHLIGHNLKDRETIRSDTFFIYSKRIFKDGAILSQVLKNSSKLVLVSGDLSENTVMSCANIASTVARLCENGLPKDFCYYLNYIMSCVQTYFDSEFSITNNSHPDLNQSWIEDISFVHSYVLTPAQLGGLSNLQYSRLYTRNIGDPGTTAFAEIKRLEAVGLLSPNIMTNILTRPPGNGDWASLCNDPYSFNFETVASPNIVLKKHTQRVLFETCSNPLLSGVHTEDNEAEEKALAEFLLNQEVIHPRVAHAIMEASSVGRRKQIQGLVDTTNTVIKIALTRRPLGIKRLMRIVNYSSMHAMLFRDDVFSSSRSNHPLVSSNMCSLTLADYARNRSWSPLTGGRKILGVSNPDTIELVEGEILSVSGGCTRCDSGDEQFTWFHLPSNIELTDDTSKNPPMRVPYLGSKTQERRAASLAKIAHMSPHVKAALRASSVLIWAYGDNEVNWTAALTIAKSRCNVNLEYLRLLSPLPTAGNLQHRLDDGITQMTFTPASLYRVSPYIHISNDSQRLFTEEGVKEGNVVYQQIMLLGLSLIESIFPMTTTRTYDEITLHLHSKFSCCIREAPVAVPFELLGVVPELRTVTSNKFMYDPSPVSEGDFARLDLAIFKSYELNLESYPTIELMNILSISSGKLIGQSVVSYDEDTSIKNDAIIVYDNTRNWISEAQNSDVVRLFEYAALEVLLDCSYQLYYLRVRGLDNIVLYMGDLYKNMPGILLSNIAATISHPVIHSRLHAVGLVNHDGSHQLADTDFIEMSAKLLVSCTRRVISGLYSGNKYDLLFPSVLDDNLNEKMLQLISRLCCLYTVLFATTREIPKIRGLTAEEKCSILTEYLLSDAVKPLLSPDQVSSIMSPNIITFPANLYYMSRKSLNLIREREDRDTILALLFPQEPLLEFPSVQDIGARVKDPFTRQPAAFLQELDLSAPARYDAFTLSQIHPELTSPNPEEDYLVRYLFRGIGTASSSWYKASHLLSVPEVRCARHGNSLYLAEGSGAIMSLLELHVPHETIYYNTLFSNEMNPPQRHFGPTPTQFLNSVVYRNLQAEVTCKDGFVQEFRPLWRENTEESDLTSDKAVGYITSAVPYRSVSLLHCDIEIPPGSNQSLLDQLAINLSLIAMHSVREGGVVIIKVLYAMGYYFHLLMNLFAPCSTKGYILSNGYACRGDMECYLVFVMGYLGGPTFVHEVVRMAKTLVQRHGTLLSKSDEITLTRLFTSQRQRVTDILSSPLPRLIKYLRKNIDTALIEAGGQPVRPFCAESLVSTLANITQITQIIASHIDTVIRSVIYMEAEGDLADTVFLFTPYNLSTDGKKRTSLIQCTRQILEVTILGLRVENLNKIGDIISLVLKGMISMEDLIPLRTYLKHSTCPKYLKAVLGITKLKEMFTDTSVLYLTRAQQKFYMKTIGNAVKGYYSNCDS

SEQ ID NO: 12 - protein, 546 aa, Artificial
  Synthetic construct
MLQLPLTILLSILSAHQSLCLDNSKLIHAGIMSTTEREVNVYAQSITGSIVVRLIPNIPSNHKSCATSQIKLYNDTLTRLLTPIKANLEGLISAVSQDQSQNSGKRKKRFVGAVIGAAALGLATAAQVTATVALNQAQENARNILRLKNSIQKTNEAVMELKDAVGQTAVAIDKTQAFINNQILPAISNLSCEVLGNKIGVQLSLYLTELTTVFGNQLTNPALTTLSLQALYNLCGDDFNYLINLLNAKNRNLASLYEANLIQGRITQYDSMNQLLIIQVQIPSISTVSGMRVTELFTLSVDTPIGEGKALVPKYVLSSGRIMEEVDLSSCAITSTSVFCSSIISRPLPLETINCLNGNVTQCQFTANTGTLESRYAVIGGLVIANCKAIVCRCLNPPGVIAQNLGLPITIISSNTCQRINLEQITLSLGNSILSTYSANLSQVEMNLAPSNPLDISVELNRVNTSLSKVESLIKESNSILDSVNPQILNVKTLITYIVLTIISLVFGILSLILACYLMYKQKAQQKTLLWLGNNTLDQMRATTKM

SEQ ID NO: 13 - protein, 576 aa, Artificial
  Synthetic Construct
MDRAVSQVALENDEREAKNTWRLIFRIAILFLTVVTLAISVASLLYSMGASTPSTLISLNNSIITSSNGLKKEILNQNIKEDLIYREVAINIPLTLDRVTVEVGTAVNQITDALRQLQSVNGSAAFASSNSPDYSGGIEHLIFQRNTLINRSVSVSDLIEHPSFIPTPTTQHGCTRIPTFHLGTRHWCYSHNIIGQGCADSRASVMYISMGALGVSSLGTPTFTTSAASILSDSLNRKSCSIVATTEGCDVLCSIVTQTEDQDYADHTPTPMIHGRLWFNGTYTERSLSQSLFLGTWAAQYPAVGSGIMTPGRVIFPFYGGVIPNSPLFLDLERFALFTHNGDLECMNLTQYQKEAIYSAYKPPKIRGSLWAQGFIVCSVGDMGNCSLKVINTSTVMMGAEGRLQLVGDSVMYYQRSSSWWPVGILYRLSLVDIIAGDIQVVINSEPLPLSKFPRPTWTPGVCQKPNVCPAVCVTGVYQDLWAISAGETLSEMTFFGGYLEASTQRKDPWIGVANQYSWFMRRRLFKTSTEAAYSSSTCFRNTRLDRNFCLLVFELTDNLLGDWRIVPLLFELTIV

SEQ ID NO: 14 - protein, 544 aa, Avian paramyxovirus serotype 5
MLQLPLTILLSILSAHQSLCLDNSKLIHAGIMSTTEREVNVYAQSITGSIVVRLIPNIPSNHKSCATSQIKLYNDTLTRLLTPIKANLEGLISAVSQDQSQNSGKRKKRFVGAVIGAAALGLATAAQVTATVALNQAQENARNILRLKNSIQKTNEAVMELKDAVGQTAVAIDKTQAFINNQILPAISNLSCEVLGNKIGVQLSLYLTELTTVFGNQLTNPALTTLSLQALYNLCGDDFNYLINLLNAKNRNLASLYEANLIQGRITQYDSMNQLLIIQVQIPSISTVSGMRVTELFTLSVDTPIGEGKALVPKYVLSSGRIMEEVDLSSCAITSTSVFCSSIISRPLPLETINCLNGNVTQCQFTANTGTLESRYAVIGGLVIANCKAIVCRCLNPPGVIAQNLGLPITIISSNTCQRINLEQITLSLGNSILSTYSANLSQVEMNLAPSNPLDISVELNRVNTSLSKVESLIKESNSILDSVNPQILNVKTVIILAVIIGLIVVWCFILTCLIVRGFMLLVKQQKFKGLSVQNNPYVSNNSH

SEQ ID NO: 15 - protein, 574 aa, Avian paramyxovirus serotype 5
MDKSYYIEPEDQRGNSRTWRLLFRLIVLTLLCLIACILVSQLFYPWLPQVLSTLISLNNSIITSSNGLKKEILNQNIKEDLIYREVAINIPLTLDRVTVEVGTAVNQITDALRQLQSVNGSAAFASSNSPDYSGGIEHLIFQRNTLINRSVSVSDLIEHPSFIPTPTTQHGCTRIPTFHLGTRHWCYSHNIIGQGCADSRASVMYISMGALGVSSLGTPTFTTSAASILSDSLNRKSCSIVATTEGCDVLCSIVTQTEDQDYADHTPTPMIHGRLWFNGTYTERSLSQSLFLGTWAAQYPAVGSGIMTPGRVIFPFYGGVIPNSPLFLDLERFALFTHNGDLECMNLTQYQKEAIYSAYKPPKIRGSLWAQGFIVCSVGDMGNCSLKVINTSTVMMGAEGRLQLVGDSVMYYQRSSSWWPVGILYRLSLVDIIAGDIQVVINSEPLPLSKFPRPTWTPGVCQKPNVCPAVCVTGVYQDLWAISAGETLSEMTFFGGYLEASTQRKDPWIGVANQYSWFMRRRLFKTSTEAAYSSSTCFRNTRLDRNFCLLVFELTDNLLGDWRIVPLLFELTIV

SEQ ID NO: 16 - protein, 2204 aa, Avian orthoavulavirus 1
MASSGPERAEHQIILPEPHLSSPLVKHKLLYYWKLTGLPLPDECDFDHLILSRQWKKILESASPDTERMIKLGRAVHQTLNHNSRITGVLHPRCLEQLANIEVPDSTNKFRKIEKKIQIHNTRYGELFTRLCTHIEKKLLGSSWSNNVPRSEEFSSIRTDPAFWFHSKWSTAKFAWLHIKQIQRHLMVAAKTRSAANKLVMLTHKVGQVFVTPELVVVTHTNENKFTCLTQELVLMYADMMEGRDMVNIISTTAVHLRSLSEKIDDILRLIDALAKDLGNQVYDVVSLMEGFAYGAVQLLEPSGTFAGDFFAFNLQELKDILIGLLPNDIAESVTHAIATVFSGLEQNQAAEMLCLLRLWGHPLLESRIAAKAVRSQMCAPKMVDFDMILQVLSFFKGTIINGYRKKNAGVWPRVKVDTIYGKVIGQLHADSAEISHDIMLREYKSLSALEFEPCIEYDPVTNLSMFLKDKAIAHPNDNWLASFRRNLLSEDQKKHVKEATSTNRLLIEFLESNDFDPYKEMEYLTTLEYLRDDNVAVSYSLKEKEVKVNGRIFAKLTKKLRNCQVMAEGILADQIAPFFQGNGVIQDSISLTKSMLAMSQLSFNSNKKRITDCKERVSSNRNHDPKSKNRRRVATFITTDLQKYCLNWRYQTIKLFAHAINQLMGLPHFFEWIHLRLMDTTMFVGDPFNPPSDPTDCDLSRVPNDDIYIVSARGGIEGLCQKLWTMISIAAIQLAAARSHCRVACMVQGDNQVIAVTREVRSDDSPEMVLTQLHQASDNFFKELIHVNHLIGHNLKDRETIRSDTFFIYSKRIFKDGAILSQVLKNSSKLVLVSGDLSENTVMSCANIASTVARLCENGLPKDFCYYLNYIMSCVQTYFDSEFSITNNSHPDLNQSWIEDISFVHSYVLTPAQLGGLSNLQYSRLYTRNIGDPGTTAFAEIKRLEAVGLLSPNIMTNILTRPPGNGDWASLCNDPYSFNFETVASPNIVLKKHTQRVLFETCSNPLLSGVHTEDNEAEEKALAEFLLNQEVIHPRVAHAIMEASSVGRRKQIQGLVDTTNTVIKIALTRRPLGIKRLMRIVNYSSMHAMLFRDDVFSSSRSNHPLVSSNMCSLTLADYARNRSWSPLTGGRKILGVSNPDTIELVEGEILSVSGGCTRCDSGDEQFTWFHLPSNIELTDDTSKNPPMRVPYLGSKTQERRAASLAKIAHMSPHVKAALRASSVLIWAYGDNEVNWTAALTIAKSRCNVNLEYLRLLSPLPTAGNLQHRLDDGITQMTFTPASLYRCHLTFTYPMILKGCSLKKESKRGMWFTNRVMLLGLSLIESIFPMTTTRTYDEITLHLHSKFSCCIREAPVAVPFELLGVVPELRTVTSNKFMYDPSPVSEGDFARLDLAIFKSYELNLESYPTIELMNILSISSGKLIGQSVVSYDEDTSIKNDAIIVYDNTRNWISEAQNSDVVRLFEYAALEVLLDCSYQLYYLRVRGLDNIVLYMGDLYKNMPGILLSNIAATISHPVIHSRLHAVGLVNHDGSHQLADTDFIEMSAKLLVSCTRRVISGLYSGNKYDLLFPSVLDDNLNEKMLQLISRLCCLYTVLFATTREIPKIRGLTAEEKCSILTEYLLSDAVKPLLSPDQVSSIMSPNIITFPANLYYMSRKSLNLIREREDRDTILALLFPQEPLLEFPSVQDIGARVKDPFTRQPAAFLQELDLSAPARYDAFTLSQIHPELTSPNPEEDYLVRYLFRGIGTASSSWYKASHLLSVPEVRCARHGNSLYLAEGSGAIMSLLELHVPHETIYYNTLFSNEMNPPQRHFGPTPTQFLNSVVYRNLQAEVTCKDGFVQEFRPLWRENTEESDLTSDKAVGYITSAVPYRSVSLLHCDIEIPPGSNQSLLDQLAINLSLIAMHSVREGGVVIIKVLYAMGYYFHLLMNLFAPCSTKGYILSNGYACRGDMECYLVFVMGYLGGPTFVHEVVRMAKTLVQRHGTLLSKSDEITLTRLFTSQRQRVTDILSSPLPRLIKYLRKNIDTALIEAGGQPVRPFCAESLVSTLANITQITQIIASHIDTVIRSVIYMEAEGDLADTVFLFTPYNLSTDGKKRTSLIQCTRQILEVTILGLRVENLNKIGDIISLVLKGMISMEDLIPLRTYLKHSTCPKYLKAVLGITKLKEMFTDTSVLYLTRAQQKFYMKTIGNAVKGYYSNCDS

SEQ ID NO: 17 - DNA, 3768 bp, SARS Coronavirus 2
atgttcgtgttcctggtcctgctgccactggtaagctcccaatgtgtaaacttaaccacaagaacccagctcccacctgcctacaccaacagcttcaccagaggcgtttattaccccgacaaggtattccggtcttctgttctgcactctacccaggacctgtttctgccctttttcagcaacgtgacatggttccacgccatccacgtgtctggcacaaacggcaccaagcggtttgataatcctgtgctccctttcaatgacggcgtgtacttcgcctctactgagaagagcaacatcatccggggctggatctttggcacaacactggactctaaaacccagagcctgctgatcgtgaacaacgccaccaacgtggtgattaaggtgtgcgagttccagttctgcaatgaccctttcctcggcgtgtactaccacaagaacaacaaaagttggatggaaagcgaattcagggtgtactcaagcgccaacaactgtaccttcgagtacgtgagccagcctttcctgatggacctagaaggtaagcagggcaatttcaagaacctcagagagttcgtgttcaagaatattgacggctacttcaaaatctacagcaagcacaccccaatcaacctggtgcgggacctgccccagggctttagcgcgctggagcctctggtggacctgcctatcggcatcaacatcacccggttccagacactgctggctctgcatagaagctacctgacacctggcgacagttcttctggctggacagccggcgccgccgcctactacgtgggctacctgcagcctagaacattcctgctgaaatacaacgagaacggcacgatcacagacgccgtggactgcgccctggatcccctgtctgagacaaagtgcaccctgaagtctttcaccgtggagaagggcatctaccagacctccaacttcagagtgcagcctaccgaatccatcgtgcgctttcccaacatcaccaacctgtgccccttcggcgaggtctttaatgccacgagattcgccagcgtgtatgcctggaacagaaagagaatcagcaactgcgtggccgactacagcgtgctgtacaacagcgcctctttcagcacatttaagtgctacggagtgtctcctaccaaactcaacgatctgtgcttcacgaacgtgtatgccgacagcttcgtgatccgaggagatgaggtgcggcagatcgctccaggacagacaggcaagatcgccgactacaactacaagctgcccgacgactttaccggctgcgtgatcgcttggaacagcaataacctggactcaaaggttggaggaaactacaactacctgtacagactgttcagaaagtccaacctgaagcccttcgagagagacatctctacagaaatctaccaggccggcagcaccccatgtaacggcgtggaaggcttcaactgctacttccctctgcagtcttatggcttccagcccacaaacggagtgggctatcagccttaccgcgtggttgtcctgagctttgagctgctgcatgcccctgctacggtgtgtggacctaagaagtccaccaacctggtgaagaacaagtgtgtgaacttcaacttcaacggcctgaccggcaccggggtgctgacagagtctaacaagaaattcctgccattccagcaattcggccgggacatcgccgacaccaccgacgccgtgcgggatcctcagaccctcgaaatcctggacatcaccccctgtagcttcggcggcgtgagcgtgatcacccctggcacaaacaccagcaatcaagtggctgtcctgtaccaggatgtcaattgcacagaagtgcctgtggccatccacgccgatcagctgacccccacctggcgggtgtactcgacaggaagcaacgtgtttcaaacaagagccggctgcctgatcggggccgagcacgtgaacaattcctacgagtgcgacatccccatcggcgccggcatctgtgcctcttaccagacacagaccaattcccctcgtagagccagatccgtggccagccagagcatcatcgcctacaccatgagcctgggcgccgaaaacagcgttgcatattccaacaacagcatcgccatccctaccaacttcaccatcagcgtgaccacagaaatcctgcctgtgtccatgaccaagacaagcgttgattgcaccatgtacatctgcggcgatagcacagagtgcagcaatctgctgctgcagtacggtagcttctgcacccagctgaatagagccctgaccggcatcgctgtggaacaggacaaaaacacccaggaggtcttcgcccaggtgaagcaaatctacaagacccctccaatcaaggacttcggaggctttaactttagccagatcctgcctgatccctccaagcctagcaaacggagtttcatcgaggacctgctcttcaacaaggtgaccctggctgacgccggcttcattaagcagtacggcgattgcctcggcgacatcgctgcaagagacctgatctgcgcccagaagttcaacggcctgaccgtgctgcctcctctcctgacagacgagatgatcgcccagtacacctctgcccttctggctggcaccatcaccagcggatggacctttggagccggagccgccctgcagatccctttcgctatgcagatggcctacagattcaacgggatcggagtgacccaaaacgtgctgtatgaaaaccagaaactgatcgccaatcagtttaacagcgccatcggcaaaatccaggatagcctgtccagcaccgccagcgccctcggcaagctgcaagatgtggtgaatcaaaatgcccaagccctgaacacactggtgaagcagctgagcagcaacttcggcgccatcagcagcgtgctgaacgacatcctgagcagactggacaaggtggaagccgaggtgcagatcgacagactgatcacaggcagactgcagtccctgcagacctacgtgacccagcagttgattagagccgctgagattagagccagtgccaacctggctgccacaaagatgtcagaatgcgtgctgggccagagcaagagagtggacttctgcggcaaaggctaccacctgatgagctttcctcagtctgcaccccacggcgtggtgtttctccacgtgacatacgtgcccgcgcaagaaaagaactttacaaccgccccagcgatctgccacgacggcaaggcccacttccctcgggagggtgtgttcgtgagcaatggaacacactggttcgtcacccagcggaacttctacgagcctcagatcattaccaccgacaacaccttcgtgagcggcaactgtgacgtcgttatcggcatcgtgaacaataccgtgtacgaccccctgcagcctgagctggatagcttcaaagaggaactggacaagtacttcaagaaccacacaagccccgacgtggacctaggcgacatctctggaatcaacgccagcgtggtgaacatccaaaaggaaatcgacagactgaacgaggtggccaagaatctgaatgaaagcctgatcgatctgcaggagctgggcaagtacgagcagtacatcaaatggccttggtacatctggctgggcttcatcgctggtctgatcgctatcgtgatggtgaccattatgctgtgctgcatgacctcctgctgctcttgcctgaagggctgttgttcttgcggctcttgctgctgatga

SEQ ID NO: 18 - DNA, 19293 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagattagaaaaaatacgggtagaaccgccaccatgttcgtgttcctggtcctgctgccactggtaagctcccaatgtgtaaacttaaccacaagaacccagctcccacctgcctacaccaacagcttcaccagaggcgtttattaccccgacaaggtattccggtcttctgttctgcactctacccaggacctgtttctgccctttttcagcaacgtgacatggttccacgccatccacgtgtctggcacaaacggcaccaagcggtttgataatcctgtgctccctttcaatgacggcgtgtacttcgcctctactgagaagagcaacatcatccggggctggatctttggcacaacactggactctaaaacccagagcctgctgatcgtgaacaacgccaccaacgtggtgattaaggtgtgcgagttccagttctgcaatgaccctttcctcggcgtgtactaccacaagaacaacaaaagttggatggaaagcgaattcagggtgtactcaagcgccaacaactgtaccttcgagtacgtgagccagcctttcctgatggacctagaaggtaagcagggcaatttcaagaacctcagagagttcgtgttcaagaatattgacggctacttcaaaatctacagcaagcacaccccaatcaacctggtgcgggacctgccccagggctttagcgcgctggagcctctggtggacctgcctatcggcatcaacatcacccggttccagacactgctggctctgcatagaagctacctgacacctggcgacagttcttctggctggacagccggcgccgccgcctactacgtgggctacctgcagcctagaacattcctgctgaaatacaacgagaacggcacgatcacagacgccgtggactgcgccctggatcccctgtctgagacaaagtgcaccctgaagtctttcaccgtggagaagggcatctaccagacctccaacttcagagtgcagcctaccgaatccatcgtgcgctttcccaacatcaccaacctgtgccccttcggcgaggtctttaatgccacgagattcgccagcgtgtatgcctggaacagaaagagaatcagcaactgcgtggccgactacagcgtgctgtacaacagcgcctctttcagcacatttaagtgctacggagtgtctcctaccaaactcaacgatctgtgcttcacgaacgtgtatgccgacagcttcgtgatccgaggagatgaggtgcggcagatcgctccaggacagacaggcaagatcgccgactacaactacaagctgcccgacgactttaccggctgcgtgatcgcttggaacagcaataacctggactcaaaggttggaggaaactacaactacctgtacagactgttcagaaagtccaacctgaagcccttcgagagagacatctctacagaaatctaccaggccggcagcaccccatgtaacggcgtggaaggcttcaactgctacttccctctgcagtcttatggcttccagcccacaaacggagtgggctatcagccttaccgcgtggttgtcctgagctttgagctgctgcatgcccctgctacggtgtgtggacctaagaagtccaccaacctggtgaagaacaagtgtgtgaacttcaacttcaacggcctgaccggcaccggggtgctgacagagtctaacaagaaattcctgccattccagcaattcggccgggacatcgccgacaccaccgacgccgtgcgggatcctcagaccctcgaaatcctggacatcaccccctgtagcttcggcggcgtgagcgtgatcacccctggcacaaacaccagcaatcaagtggctgtcctgtaccaggatgtcaattgcacagaagtgcctgtggccatccacgccgatcagctgacccccacctggcgggtgtactcgacaggaagcaacgtgtttcaaacaagagccggctgcctgatcggggccgagcacgtgaacaattcctacgagtgcgacatccccatcggcgccggcatctgtgcctcttaccagacacagaccaattcccctggtagtgcaagttccgtggccagccagagcatcatcgcctacaccatgagcctgggcgccgaaaacagcgttgcatattccaacaacagcatcgccatccctaccaacttcaccatcagcgtgaccacagaaatcctgcctgtgtccatgaccaagacaagcgttgattgcaccatgtacatctgcggcgatagcacagagtgcagcaatctgctgctgcagtacggtagcttctgcacccagctgaatagagccctgaccggcatcgctgtggaacaggacaaaaacacccaggaggtcttcgcccaggtgaagcaaatctacaagacccctccaatcaaggacttcggaggctttaactttagccagatcctgcctgatccctccaagcctagcaaacggagtcctatcgaggacctgctcttcaacaaggtgaccctggctgacgccggcttcattaagcagtacggcgattgcctcggcgacatcgctgcaagagacctgatctgcgcccagaagttcaacggcctgaccgtgctgcctcctctcctgacagacgagatgatcgcccagtacacctctgcccttctggctggcaccatcaccagcggatggacctttggagccggacctgccctgcagatccctttccctatgcagatggcctacagattcaacgggatcggagtgacccaaaacgtgctgtatgaaaaccagaaactgatcgccaatcagtttaacagcgccatcggcaaaatccaggatagcctgtccagcaccccaagcgccctcggcaagctgcaagatgtggtgaatcaaaatgcccaagccctgaacacactggtgaagcagctgagcagcaacttcggcgccatcagcagcgtgctgaacgacatcctgagcagactggacccacctgaagccgaggtgcagatcgacagactgatcacaggcagactgcagtccctgcagacctacgtgacccagcagttgattagagccgctgagattagagccagtgccaacctggctgccacaaagatgtcagaatgcgtgctgggccagagcaagagagtggacttctgcggcaaaggctaccacctgatgagctttcctcagtctgcaccccacggcgtggtgtttctccacgtgacatacgtgcccgcgcaagaaaagaactttacaaccgccccagcgatctgccacgacggcaaggcccacttccctcgggagggtgtgttcgtgagcaatggaacacactggttcgtcacccagcggaacttctacgagcctcagatcattaccaccgacaacaccttcgtgagcggcaactgtgacgtcgttatcggcatcgtgaacaataccgtgtacgaccccctgcagcctgagctggatagcttcaaagaggaactggacaagtacttcaagaaccacacaagccccgacgtggacctaggcgacatctctggaatcaacgccagcgtggtgaacatccaaaaggaaatcgacagactgaacgaggtggccaagaatctgaatgaaagcctgatcgatctgcaggagctgggcaagtacgagcagtacatcaaatggccttggtacatctggctgggcttcatcgctggtctgatcgctatcgtgatggtgaccattatgctgtgctgcatgacctcctgctgctcttgcctgaagggctgttgttcttgcggctcttgctgcaagttcgacgaggatgactctgaacctgttctgaagggcgtgaagctgcactacacctgataaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactata

SEQ ID NO: 19 - DNA, 19294 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagattagaaaaaatacgggtagaaccgccaccatgttcgtgttcctggtcctgctgccactggtaagctcccaatgtgtaaacttaaccacaagaacccagctcccacctgcctacaccaacagcttcaccagaggcgtttattaccccgacaaggtattccggtcttctgttctgcactctacccaggacctgtttctgccctttttcagcaacgtgacatggttccacgccatccacgtgtctggcacaaacggcaccaagcggtttgataatcctgtgctccctttcaatgacggcgtgtacttcgcctctactgagaagagcaacatcatccggggctggatctttggcacaacactggactctaaaacccagagcctgctgatcgtgaacaacgccaccaacgtggtgattaaggtgtgcgagttccagttctgcaatgaccctttcctcggcgtgtactaccacaagaacaacaaaagttggatggaaagcgaattcagggtgtactcaagcgccaacaactgtaccttcgagtacgtgagccagcctttcctgatggacctagaaggtaagcagggcaatttcaagaacctcagagagttcgtgttcaagaatattgacggctacttcaaaatctacagcaagcacaccccaatcaacctggtgcgggacctgccccagggctttagcgcgctggagcctctggtggacctgcctatcggcatcaacatcacccggttccagacactgctggctctgcatagaagctacctgacacctggcgacagttcttctggctggacagccggcgccgccgcctactacgtgggctacctgcagcctagaacattcctgctgaaatacaacgagaacggcacgatcacagacgccgtggactgcgccctggatcccctgtctgagacaaagtgcaccctgaagtctttcaccgtggagaagggcatctaccagacctccaacttcagagtgcagcctaccgaatccatcgtgcgctttcccaacatcaccaacctgtgccccttcggcgaggtctttaatgccacgagattcgccagcgtgtatgcctggaacagaaagagaatcagcaactgcgtggccgactacagcgtgctgtacaacagcgcctctttcagcacatttaagtgctacggagtgtctcctaccaaactcaacgatctgtgcttcacgaacgtgtatgccgacagcttcgtgatccgaggagatgaggtgcggcagatcgctccaggacagacaggcaagatcgccgactacaactacaagctgcccgacgactttaccggctgcgtgatcgcttggaacagcaataacctggactcaaaggttggaggaaactacaactacctgtacagactgttcagaaagtccaacctgaagcccttcgagagagacatctctacagaaatctaccaggccggcagcaccccatgtaacggcgtggaaggcttcaactgctacttccctctgcagtcttatggcttccagcccacaaacggagtgggctatcagccttaccgcgtggttgtcctgagctttgagctgctgcatgcccctgctacggtgtgtggacctaagaagtccaccaacctggtgaagaacaagtgtgtgaacttcaacttcaacggcctgaccggcaccggggtgctgacagagtctaacaagaaattcctgccattccagcaattcggccgggacatcgccgacaccaccgacgccgtgcgggatcctcagaccctcgaaatcctggacatcaccccctgtagcttcggcggcgtgagcgtgatcacccctggcacaaacaccagcaatcaagtggctgtcctgtaccaggatgtcaattgcacagaagtgcctgtggccatccacgccgatcagctgacccccacctggcgggtgtactcgacaggaagcaacgtgtttcaaacaagagccggctgcctgatcggggccgagcacgtgaacaattcctacgagtgcgacatccccatcggcgccggcatctgtgcctcttaccagacacagaccaattcccctcgtagagccagatccgtggccagccagagcatcatcgcctacaccatgagcctgggcgccgaaaacagcgttgcatattccaacaacagcatcgccatccctaccaacttcaccatcagcgtgaccacagaaatcctgcctgtgtccatgaccaagacaagcgttgattgcaccatgtacatctgcggcgatagcacagagtgcagcaatctgctgctgcagtacggtagcttctgcacccagctgaatagagccctgaccggcatcgctgtggaacaggacaaaaacacccaggaggtcttcgcccaggtgaagcaaatctacaagacccctccaatcaaggacttcggaggctttaactttagccagatcctgcctgatccctccaagcctagcaaacggagtcctatcgaggacctgctcttcaacaaggtgaccctggctgacgccggcttcattaagcagtacggcgattgcctcggcgacatcgctgcaagagacctgatctgcgcccagaagttcaacggcctgaccgtgctgcctcctctcctgacagacgagatgatcgcccagtacacctctgcccttctggctggcaccatcaccagcggatggacctttggagccggacctgccctgcagatccctttccctatgcagatggcctacagattcaacgggatcggagtgacccaaaacgtgctgtatgaaaaccagaaactgatcgccaatcagtttaacagcgccatcggcaaaatccaggatagcctgtccagcaccccaagcgccctcggcaagctgcaagatgtggtgaatcaaaatgcccaagccctgaacacactggtgaagcagctgagcagcaacttcggcgccatcagcagcgtgctgaacgacatcctgagcagactggacccacctgaagccgaggtgcagatcgacagactgatcacaggcagactgcagtccctgcagacctacgtgacccagcagttgattagagccgctgagattagagccagtgccaacctggctgccacaaagatgtcagaatgcgtgctgggccagagcaagagagtggacttctgcggcaaaggctaccacctgatgagctttcctcagtctgcaccccacggcgtggtgtttctccacgtgacatacgtgcccgcgcaagaaaagaactttacaaccgccccagcgatctgccacgacggcaaggcccacttccctcgggagggtgtgttcgtgagcaatggaacacactggttcgtcacccagcggaacttctacgagcctcagatcattaccaccgacaacaccttcgtgagcggcaactgtgacgtcgttatcggcatcgtgaacaataccgtgtacgaccccctgcagcctgagctggatagcttcaaagaggaactggacaagtacttcaagaaccacacaagccccgacgtggacctaggcgacatctctggaatcaacgccagcgtggtgaacatccaaaaggaaatcgacagactgaacgaggtggccaagaatctgaatgaaagcctgatcgatctgcaggagctgggcaagtacgagcagtacatcaaatggccttggtacatctggctgggcttcatcgctggtctgatcgctatcgtgatggtgaccattatgctgtgctgcatgacctcctgctgctcttgcctgaagggctgttgttcttgcggctcttgctgcaagttcgacgaggatgactctgaacctgttctgaagggcgtgaagctgcactacacctgataaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactatat

SEQ ID NO: 20 - protein, 30 aa, Avian orthoavulavirus 1
VSPYIHISNDSQRLFTEEGVKEGNVVYQQI

SEQ ID NO: 21 - DNA, 49 bp, Artificial
  Synthetic Consstruct
gcaccgagttccccctctagattagaaaaaatacgggtagaaccgccac

SEQ ID NO: 22 - DNA, 44 bp, Artificial
  Synthetic Construct
gttggaccttgggtacgcgtttatcaggtgtagtgcagcttcac

SEQ ID NO: 23 - DNA, 16191 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagattagaaaaaatacgggtagaaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataaacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactata

SEQ ID NO: 24 - DNA, 52 bp, Artificial
  Synthetic Construct
gttggaccttgggtacgcgtttatcatcagcagcaagagccgcaagaacaac

SEQ ID NO: 25 - DNA, 16 bp, Avian orthoavulavirus 1
gggagacaggggcgcc

SEQ ID NO: 26 - protein, 6 aa, Avian orthoavulavirus 1
GRQGRL

SEQ ID NO: 27 - DNA, 15441 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagaggacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactata

SEQ ID NO: 28 - protein, 553 aa, Avian orthoavulavirus 1
MGSRPSTKNPAPMMLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGVGNNQLSSLIGSGLITGNPILYDSQTQLLGIQVTAPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 29 - protein, 1270 aa, SARS Coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 30 - protein, 1270 aa, SARS Coronavirus 2
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - protein, 1270 aa, SARS Coronavirus 2
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 32 - DNA, 1662 bp, Avian orthoavulavirus 1
atgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtga

SEQ ID NO: 33 - DNA, 1734 bp, Avian orthoavulavirus 1
atggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctag

SEQ ID NO: 34 - protein, 577 aa, Avian orthoavulavirus 1
MDRAVSQVALENDEREAKNTWRLIFRIAILFLTVVTLAISVASLLYSMGASTPSDLVGIPTRISRAEEKITSTLGSNQDVVDRIYKQVALESPLALLNTETTIMNAITSLSYQINGAANNSGWGAPIHDPDYIGGIGKELIVDDASDVTSFYPSAFQEHLNFIPAPTTGSGCTRIPSFDMSATHYCYTHNVILSGCRDHSHSYQYLALGVLRTSATGRVFFSTLRSINLDDTQNRKSCSVSATPLGCDMLCSKVTETEEEDYNSAVPTRMVHGRLGFDGQYHEKDLDVTTLFGDWVANYPGVGGGSFIDSRVWFSVYGGLKPNSPSDTVQEGKYVIYKRYNDTCPDEQDYQIRMAKSSYKPGRFGGKRIQQAILSIKVSTSLGEDPVLTVPPNTVTLMGAEGRILTVGTSHFLYQRGSSYFSPALLYPMTVSNKTATLHSPYTFNAFTRPGSIPCQASARCPNSCVTGVYTDPYPLIFYRNHTLRGVFGTMLDGVQARLNPASAVFDSTSRSRITRVSSSSTKAAYTTSTCFKVVKTNKTYCLSIAEISNTLFGEFRIVPLLVEILKDDGVREARSG

SEQ ID NO: 35 - DNA, 90 bp, Avian orthoavulavirus 1
gtgtcaccttacattcacatatccaatgattctcaaaggctgttcactgaagaaggagtcaaagaggggaatgtggtttaccaacagatc

SEQ ID NO: 36 - protein, 6 aa, Avian orthoavulavirus 1
RRQRRF

SEQ ID NO: 37 - DNA, 26 bp, Artificial
  Synthetic Construct
acaggtacgttaatagttaatagcgt

SEQ ID NO: 38 - DNA, 22 bp, Artificial
  Synthetic Construct
atattgcagcagtacgcacaca

SEQ ID NO: 39 - DNA, 26 bp, Artificial
  Synthetic Construct
  misc_feature (1)..(1): n is 6-carboxyfluorescein-adenosine
  misc_feature (26)..(26): n is guanosine-blackberry quencher
ncactagccatccttactgcgcttcn

SEQ ID NO: 40 - DNA, 3801 bp, Artificial
  Synthetic Construct
atgttcgtgttcctggtcctgctgccactggtaagctcccaatgtgtaaacttaaccacaagaacccagctcccacctgcctacaccaacagcttcaccagaggcgtttattaccccgacaaggtattccggtcttctgttctgcactctacccaggacctgtttctgccctttttcagcaacgtgacatggttccacgccatccacgtgtctggcacaaacggcaccaagcggtttgataatcctgtgctccctttcaatgacggcgtgtacttcgcctctactgagaagagcaacatcatccggggctggatctttggcacaacactggactctaaaacccagagcctgctgatcgtgaacaacgccaccaacgtggtgattaaggtgtgcgagttccagttctgcaatgaccctttcctcggcgtgtactaccacaagaacaacaaaagttggatggaaagcgaattcagggtgtactcaagcgccaacaactgtaccttcgagtacgtgagccagcctttcctgatggacctagaaggtaagcagggcaatttcaagaacctcagagagttcgtgttcaagaatattgacggctacttcaaaatctacagcaagcacaccccaatcaacctggtgcgggacctgccccagggctttagcgcgctggagcctctggtggacctgcctatcggcatcaacatcacccggttccagacactgctggctctgcatagaagctacctgacacctggcgacagttcttctggctggacagccggcgccgccgcctactacgtgggctacctgcagcctagaacattcctgctgaaatacaacgagaacggcacgatcacagacgccgtggactgcgccctggatcccctgtctgagacaaagtgcaccctgaagtctttcaccgtggagaagggcatctaccagacctccaacttcagagtgcagcctaccgaatccatcgtgcgctttcccaacatcaccaacctgtgccccttcggcgaggtctttaatgccacgagattcgccagcgtgtatgcctggaacagaaagagaatcagcaactgcgtggccgactacagcgtgctgtacaacagcgcctctttcagcacatttaagtgctacggagtgtctcctaccaaactcaacgatctgtgcttcacgaacgtgtatgccgacagcttcgtgatccgaggagatgaggtgcggcagatcgctccaggacagacaggcaagatcgccgactacaactacaagctgcccgacgactttaccggctgcgtgatcgcttggaacagcaataacctggactcaaaggttggaggaaactacaactacctgtacagactgttcagaaagtccaacctgaagcccttcgagagagacatctctacagaaatctaccaggccggcagcaccccatgtaacggcgtggaaggcttcaactgctacttccctctgcagtcttatggcttccagcccacaaacggagtgggctatcagccttaccgcgtggttgtcctgagctttgagctgctgcatgcccctgctacggtgtgtggacctaagaagtccaccaacctggtgaagaacaagtgtgtgaacttcaacttcaacggcctgaccggcaccggggtgctgacagagtctaacaagaaattcctgccattccagcaattcggccgggacatcgccgacaccaccgacgccgtgcgggatcctcagaccctcgaaatcctggacatcaccccctgtagcttcggcggcgtgagcgtgatcacccctggcacaaacaccagcaatcaagtggctgtcctgtaccaggatgtcaattgcacagaagtgcctgtggccatccacgccgatcagctgacccccacctggcgggtgtactcgacaggaagcaacgtgtttcaaacaagagccggctgcctgatcggggccgagcacgtgaacaattcctacgagtgcgacatccccatcggcgccggcatctgtgcctcttaccagacacagaccaattcccctggtagtgcaagttccgtggccagccagagcatcatcgcctacaccatgagcctgggcgccgaaaacagcgttgcatattccaacaacagcatcgccatccctaccaacttcaccatcagcgtgaccacagaaatcctgcctgtgtccatgaccaagacaagcgttgattgcaccatgtacatctgcggcgatagcacagagtgcagcaatctgctgctgcagtacggtagcttctgcacccagctgaatagagccctgaccggcatcgctgtggaacaggacaaaaacacccaggaggtcttcgcccaggtgaagcaaatctacaagacccctccaatcaaggacttcggaggctttaactttagccagatcctgcctgatccctccaagcctagcaaacggagtcctatcgaggacctgctcttcaacaaggtgaccctggctgacgccggcttcattaagcagtacggcgattgcctcggcgacatcgctgcaagagacctgatctgcgcccagaagttcaacggcctgaccgtgctgcctcctctcctgacagacgagatgatcgcccagtacacctctgcccttctggctggcaccatcaccagcggatggacctttggagccggacctgccctgcagatccctttccctatgcagatggcctacagattcaacgggatcggagtgacccaaaacgtgctgtatgaaaaccagaaactgatcgccaatcagtttaacagcgccatcggcaaaatccaggatagcctgtccagcaccccaagcgccctcggcaagctgcaagatgtggtgaatcaaaatgcccaagccctgaacacactggtgaagcagctgagcagcaacttcggcgccatcagcagcgtgctgaacgacatcctgagcagactggacccacctgaagccgaggtgcagatcgacagactgatcacaggcagactgcagtccctgcagacctacgtgacccagcagttgattagagccgctgagattagagccagtgccaacctggctgccacaaagatgtcagaatgcgtgctgggccagagcaagagagtggacttctgcggcaaaggctaccacctgatgagctttcctcagtctgcaccccacggcgtggtgtttctccacgtgacatacgtgcccgcgcaagaaaagaactttacaaccgccccagcgatctgccacgacggcaaggcccacttccctcgggagggtgtgttcgtgagcaatggaacacactggttcgtcacccagcggaacttctacgagcctcagatcattaccaccgacaacaccttcgtgagcggcaactgtgacgtcgttatcggcatcgtgaacaataccgtgtacgaccccctgcagcctgagctggatagcttcaaagaggaactggacaagtacttcaagaaccacacaagccccgacgtggacctaggcgacatctctggaatcaacgccagcgtggtgaacatccaaaaggaaatcgacagactgaacgaggtggccaagaatctgaatgaaagcctgatcgatctgcaggagctgggcaagtacgagcagggtggcggtggctcgctgattacctatatcgtcctgactattatctccctggtgtttggcattctgtccctgattctggcctgttacctgatgtacaagcagaaggcccagcagaagaccctgctgtggctgggcaataatacactggatcagatgcgggctacaactaagatgtga

SEQ ID NO: 41 - protein, 1266 aa, Artificial
  Synthetic Construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGGGSLITYIVLTIISLVFGILSLILACYLMYKQKAQQKTLLWLGNNTLDQMRATTKM

SEQ ID NO: 42 - DNA, 15468 bp, Artificial
  Synthetic Construct
taatacgactcactatagggaccaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttccccctctagattagaaaaaatacgggtagaaccgccaccacgcgtacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagttaattaaatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcactggcaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacgtatatcgttttgactatcatatctcttgtttttggtatacttagcctgattctagcatgctacctaatgtacaagcaaaaggcgcaacaaaagaccttattatggcttgggaataatacactcgatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagggcgcgccttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtggccggcatggtcccagcctcctcgctggcgccggctgggcaacattccgaggggaccgtcccctcggtaatggcgaatgggacgtcgactgctaacaaagcccgaaaggaagctgagttggctgctgccaccgctgagcaataactagcataaccccttggggcctctaaacgggtcttgaggggttttttgctgaaaggaggaactata